(19) United States
(12) Patent Application Publication
(10) Pub. No.: US 2022/0315541 A1
(43) Pub. Date: Oct. 6, 2022

(54) COMPOUND AND ORGANIC LIGHT-EMITTING DEVICE COMPRISING SAME

(71) Applicant: LT MATERIALS CO., LTD., Yongin-City, Gyeonggi-do (KR)

(72) Inventors: Geon-Yu PARK, Yongin-si (KR); Dong-Jun KIM, Yongin-si (KR)

(73) Assignee: LT MATERIALS CO., LTD., Yongin-City, Gyeonggi-do (KR)

(21) Appl. No.: 17/616,304

(22) PCT Filed: Nov. 16, 2020
(86) PCT No.: PCT/KR2020/016071

§ 371 Date: Dec. 3, 2021

(30) Foreign Application Priority Data
Nov. 25, 2019 (KR) 10-2019-0152361

(51) Int. Cl. C07D 251/24; C07D 405/10; C07D 403/10; C07F 7/08; C07F 9/28; C07D 409/10; C07D 487/04; C07D 491/048; C07D 495/04; C07D 471/04; C07D 213/16; C07D 239/26; C07D 235/08; C07C 15/38; C07D 307/91; C07D 333/76; C07D 209/82; C07C 255/50; C07C 211/54; C07D 409/14; H01L 51/00

(52) U.S. Cl. CPC C07D 251/24; C07D 405/10; C07D 403/10; C07F 7/0812; C07F 9/28; C07D 409/10; C07D 487/04; C07D 491/048; C07D 495/04; C07D 471/04; C07D 213/16; C07D 239/26; C07D 235/08; C07C 15/38; C07D 307/91; C07D 333/76; C07D 209/82; C07C 255/50; C07C 211/54; C07D 409/14; H01L 51/0059; H01L 51/006; H01L 51/0064; H01L 51/0062; H01L 51/0071; H01L 51/0067; H01L 51/0052; H01L 51/0072; H01L 51/0073; H01L 51/0074; H01L 51/0054; H01L 51/0055; H01L 51/0056; C07B 2200/05

(57) ABSTRACT

The present specification relates to a compound represented by Chemical Formula 1, and an organic light emitting device including the same.

### TECHNICAL FIELD

The present specification relates to a compound, and an organic light emitting device including the same.

The present specification claims priority to and the benefits of Korean Patent Application No. 10-2019-0152361, filed with the Korean Intellectual Property Office on Nov. 25, 2019, the entire contents of which are incorporated herein by reference.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### DISCLOSURE

### Technical Problem

The present specification is directed to providing a compound, and an organic light emitting device including the same.

### Technical Solution

One embodiment of the present specification provides a compound represented by the following Chemical Formula 1.

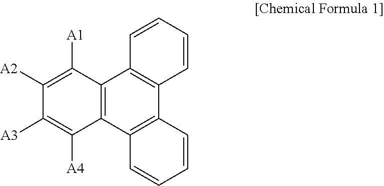

In Chemical Formula 1,

one of A1 and A2 is (L1)ₐ-Q1,

the other one of A1 and A2, and A3 and A4 are each independently hydrogen; deuterium; or (L2)_{b}-Q2, and at least one thereof is (L2)_{b}-Q2,

a and b are each independently an integer of 1 to 5,

when a and b are each 2 or greater, substituents in the parentheses are the same as or different from each other,

L1 and L2 are each independently a direct bond; a substituted or unsubstituted C6 to C60 arylene group; or a substituted or unsubstituted C2 to C60 heteroarylene group,

Q1 is a substituted or unsubstituted C6 to C20 aryl group; or a substituted or unsubstituted C2 to C20 heteroaryl group including N,

Q2 is a cyano group; a substituted or unsubstituted silyl group; a substituted or unsubstituted amine group; a substituted or unsubstituted C1 to C20 alkyl group; a substituted or unsubstituted C6 to C30 aryl group; a substituted or unsubstituted C2 to C30 heteroaryl group; or a substituted or unsubstituted phosphine oxide group,

when A2 and A3 are hydrogen, Q1 is a phenyl group and Q2 includes pyridine or triazine, L1 is a substituted or unsubstituted C6 to C60 arylene group; or a substituted or unsubstituted C2 to C60 heteroarylene group, and

when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a dicyclic or higher aryl group unsubstituted or substituted with an alkyl group or an aryl group is satisfied.

Another embodiment of the present application provides an organic light emitting device including a first electrode; a second electrode provided opposite to the first electrode; and an organic material layer provided between the first electrode and the second electrode, wherein the organic material layer includes one or more types of the compound represented by Chemical Formula 1.

### Advantageous Effects

A compound described in the present specification can be used as a material of an organic material layer of an organic light emitting device. The compound is capable of performing a role of a hole injection material, a hole transfer material, a light emitting material, an electron transfer material, an electron injection material or the like in the organic light emitting device. Particularly, the compound can be used as a light emitting layer material of the organic light emitting device.

In addition, when using two types of the compound of Chemical Formula 1 or using both the compound of Chemical Formula 1 and the compound of Chemical Formula 2 as a light emitting layer material of the organic light emitting device, a driving voltage of the device can be lowered, light efficiency can be enhanced, and lifetime properties of the device can be enhanced.

### DESCRIPTION OF DRAWINGS

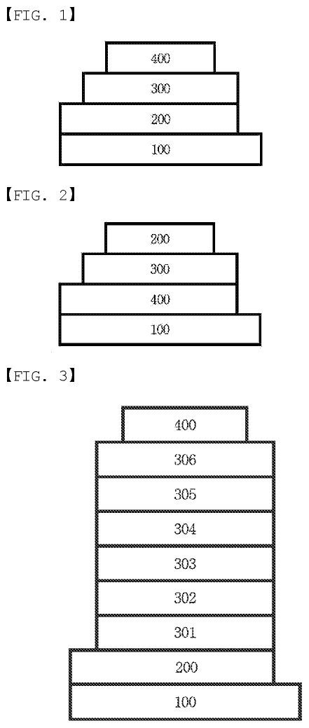

FIG. 1 to FIG. 3 are diagrams each illustrating a lamination structure of an organic light emitting device according to one embodiment of the present specification.
100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present specification will be described in more detail.

In the present specification, a certain part “including” certain constituents means capable of further including other constituents, and does not exclude other constituents unless particularly stated on the contrary.

The term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification,

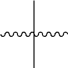

means a substituted position.

In the present specification, “substituted or unsubstituted” means being substituted with one or more substituents selected from the group consisting of deuterium; a halogen group; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; a silyl group; a phosphine oxide group; and an amine group, or being substituted with a substituent linking two or more substituents selected from among the substituents illustrated above, or being unsubstituted.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or ²H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

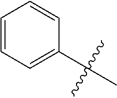 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

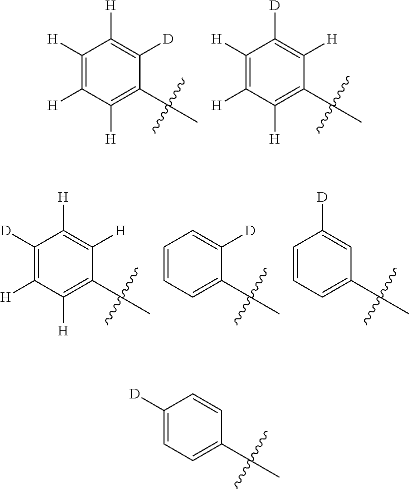

In addition, in one embodiment of the present application, “a phenyl group having a deuterium content of 0%” may mean a phenyl group that does not include a deuterium atom, that is, a phenyl group that has 5 hydrogen atoms.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the fluorenyl group may be substituted, and adjacent substituents may bond to each other to form a ring.

When the fluorenyl group is substituted,

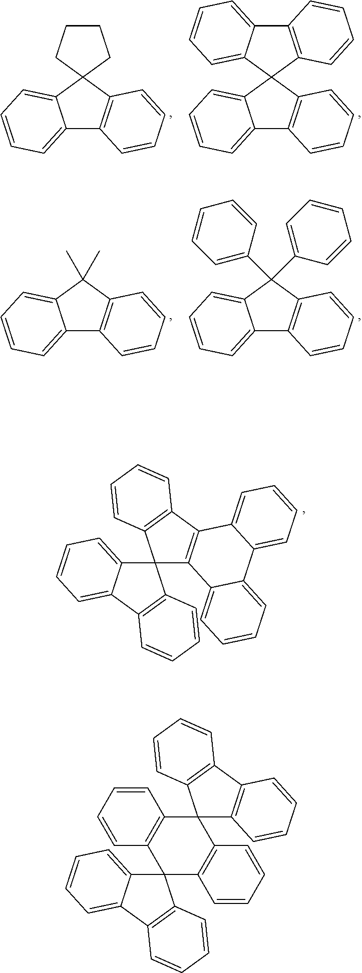

and the like may be included, however, the structure is not limited thereto.

In the present specification, the heteroaryl group includes O, S, SO₂, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a benzofuro[2,3-d]pyrimidyl group; a benzothieno[2,3-d]pyrimidyl group; a benzofuro[2,3-a]carbazolyl group, a benzothieno[2,3-a]carbazolyl group, a 1,3-dihydroindolo[2,3-a]carbazolyl group, a benzofuro[3,2-a]carbazolyl group, a benzothieno[3,2-a]carbazolyl group, a 1,3-dihydroindolo[3,2-a]carbazolyl group, a benzofuro[2,3-b]carbazolyl group, a benzothieno[2,3-b]carbazolyl group, a 1,3-dihydroindolo[2,3-b]carbazolyl group, a benzofuro[3,2-b]carbazolyl group, a benzothieno[3,2-b]carbazolyl group, a 1,3-dihydroindolo[3,2-b]carbazolyl group, a benzofuro[2,3-c]carbazolyl group, a benzothieno[2,3-c]carbazolyl group, a 1,3-dihydroindolo[2,3-c]carbazolyl group, a benzofuro[3,2-c]carbazolyl group, a benzothieno[3,2-c]carbazolyl group, a 1,3-dihydroindolo[3,2-c]carbazolyl group, a 1,3-dihydroindeno[2,1-b]carbazolyl group, a 5,11-dihydroindeno[1,2-b]carbazolyl group, a 5,12-dihydroindeno[1,2-c]carbazolyl group, a 5,8-dihydroindeno[2,1-c]carbazolyl group, a 7,12-dihydroindeno[1,2-a]carbazolyl group, a 11,12-dihydroindeno[2,1-a]carbazolyl group and the like, but are not limited thereto.

In the present specification, the silyl group is a substituent including Si, having the Si atom directly linked as a radical, and is represented by —Si(R101) (R102) (R103). R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heteroaryl group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the phosphine oxide group is represented by —P(═O) (R104) (R105), and R104 and R105 are the same as or different from each other and may be each independently a substituent formed with at least one of hydrogen; deuterium; a halogen group; an alkyl group; an alkenyl group; an alkoxy group; a cycloalkyl group; an aryl group; and a heteroaryl group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the amine group is represented by —N(R106) (R107), and R106 and R107 are the same as or different from each other and may be each independently a substituent formed with at least one of hydrogen; deuterium; a halogen group; an alkyl group; an alkenyl group; an alkoxy group; a cycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the examples of the aryl group described above may be applied to the arylene group except that the arylene group is a divalent group.

In the present specification, the examples of the heteroaryl group described above may be applied to the heteroarylene group except that the heteroarylene group is a divalent group.

One embodiment of the present specification provides a compound represented by Chemical Formula 1.

In the compound represented by Chemical Formula 1, one benzene ring of the triphenylene is substituted with two substituents including N, that is, a heteroaryl group and an aryl group, and, compared to a compound having a structure substituted with just one heteroaryl group, the HOMO orbital is delocalized to the aryl-based substituent effectively stabilizing holes, and, compared to a compound having a structure substituted with just one aryl group, higher electron mobility is obtained leading to an enhanced device lifetime. By substituting one benzene ring of the triphenylene with two aryl groups as another structure, the HOMO orbital is delocalized to the two substituents and the triphenylene compared to a compound having a structure substituted with just one aryl group, and holes may be effectively stabilized.

In one embodiment of the present specification, L1 and L2 are each independently a direct bond; a substituted or unsubstituted C6 to C60 arylene group; or a substituted or unsubstituted C2 to C60 heteroarylene group.

In one embodiment of the present specification, L1 and L2 are each independently a direct bond; a substituted or unsubstituted C6 to C40 arylene group; or a substituted or unsubstituted C2 to C40 heteroarylene group.

In one embodiment of the present specification, L1 and L2 are each independently a direct bond; a substituted or unsubstituted C6 to C20 arylene group; or a substituted or unsubstituted C2 to C20 heteroarylene group.

In one embodiment of the present specification, L1 and L2 are each independently a direct bond; a substituted or unsubstituted phenylene group; a substituted or unsubstituted biphenylene group; a substituted or unsubstituted terphenylene group; a substituted or unsubstituted divalent pyridine group; a substituted or unsubstituted divalent pyrimidine group; a substituted or unsubstituted divalent triazine group; or a substituted or unsubstituted divalent carbazolyl group.

In one embodiment of the present specification, L1 and L2 are each independently a direct bond; a phenylene group; a biphenylene group; a terphenylene group; a divalent pyridine group unsubstituted or substituted with an aryl group; a divalent pyrimidine group unsubstituted or substituted with an aryl group; a divalent triazine group unsubstituted or substituted with an aryl group; or a divalent carbazolyl group.

In one embodiment of the present specification, Q1 is a substituted or unsubstituted C6 to C20 aryl group; or a substituted or unsubstituted C2 to C20 heteroaryl group including N.

In one embodiment of the present specification, Q1 is a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted pyridine group; a substituted or unsubstituted pyrimidine group; a substituted or unsubstituted triazine group; a substituted or unsubstituted benzimidazole group; a substituted or unsubstituted quinazolyl group; a substituted or unsubstituted benzofuro[2,3-d]pyrimidyl group; a substituted or unsubstituted benzothieno[2,3-d]pyrimidyl group; or a substituted or unsubstituted phenanthroline group.

In one embodiment of the present specification, Q1 is a phenyl group; a biphenyl group; a terphenyl group; a naphthyl group; a triphenylene group; a pyridine group unsubstituted or substituted with an aryl group; a pyrimidine group unsubstituted or substituted with an aryl group; a triazine group unsubstituted or substituted with an aryl group; a benzimidazole group unsubstituted or substituted with an aryl group; a quinazolyl group unsubstituted or substituted with an aryl group; a benzofuro[2,3-d]pyrimidyl group unsubstituted or substituted with an aryl group; a benzothieno[2,3-d]pyrimidyl group unsubstituted or substituted with an aryl group; or a phenanthroline group.

In one embodiment of the present specification, Q2 is a cyano group; a substituted or unsubstituted silyl group; a substituted or unsubstituted amine group; a substituted or unsubstituted C1 to C20 alkyl group; a substituted or unsubstituted C6 to C30 aryl group; a substituted or unsubstituted C2 to C30 heteroaryl group; or a substituted or unsubstituted phosphine oxide group.

In one embodiment of the present specification, Q2 is a cyano group; a substituted or unsubstituted silyl group; a substituted or unsubstituted amine group; a substituted or unsubstituted C1 to C10 alkyl group; a substituted or unsubstituted C6 to C30 aryl group; a substituted or unsubstituted C2 to C30 heteroaryl group; or a substituted or unsubstituted phosphine oxide group.

In one embodiment of the present specification, Q2 is a cyano group; a substituted or unsubstituted silyl group; a substituted or unsubstituted amine group; a substituted or unsubstituted methyl group; a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted pyrenyl group; a substituted or unsubstituted fluorenyl group; a substituted or unsubstituted pyridine group; a substituted or unsubstituted pyrimidine group; a substituted or unsubstituted triazine group; a substituted or unsubstituted quinolinyl group; a substituted or unsubstituted quinazolinyl group; a substituted or unsubstituted phenanthrolinyl group; a substituted or unsubstituted carbazolyl group; a substituted or unsubstituted benzocarbazolyl group; a substituted or unsubstituted dibenzofuranyl group; a substituted or unsubstituted dibenzothiophenyl group; a substituted or unsubstituted benzimidazolyl group; a phosphine oxide group unsubstituted or substituted with an aryl group; or selected from among the following structural formulae.

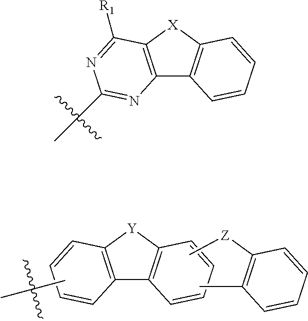

In the structural formulae,

X, Y and Z are each O; S; C(R2) (R3); or N(R4), and

R1 to R4 are each independently hydrogen; deuterium; a substituted or unsubstituted C1 to C10 alkyl group; a substituted or unsubstituted C6 to C20 aryl group; or a substituted or unsubstituted C2 to C20 heteroaryl group.

In one embodiment of the present specification, Q2 is a cyano group; a silyl group unsubstituted or substituted with an aryl group; an amine group unsubstituted or substituted with an aryl group; a methyl group unsubstituted or substituted with an aryl group; a phenyl group unsubstituted or substituted with a cyano group, an aryl group or a heteroaryl group; a biphenyl group; a terphenyl group; a naphthyl group; a phenanthrenyl group; a triphenylenyl group; a pyrenyl group; a fluorenyl group unsubstituted or substituted with an alkyl group or an aryl group; a spirobifluorenyl group; a pyridine group unsubstituted or substituted with an aryl group; a pyrimidine group unsubstituted or substituted with an aryl group; a triazine group unsubstituted or substituted with an aryl group or a heteroaryl group; a quinolinyl group unsubstituted or substituted with an aryl group; a quinazolinyl group unsubstituted or substituted with an aryl group; a substituted or unsubstituted phenanthrolinyl group; a carbazolyl group unsubstituted or substituted with an aryl group; a benzocarbazolyl group; a dibenzofuranyl group; a dibenzothiophenyl group; a benzimidazolyl group unsubstituted or substituted with an aryl group; a phosphine oxide group unsubstituted or substituted with an aryl group; or any one selected from among the following structural formulae.

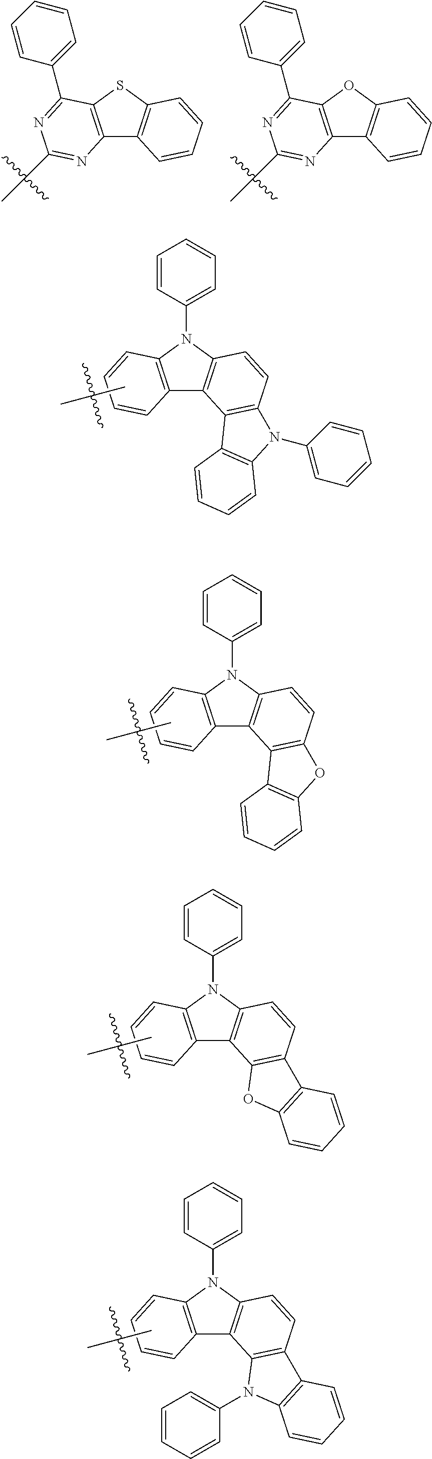

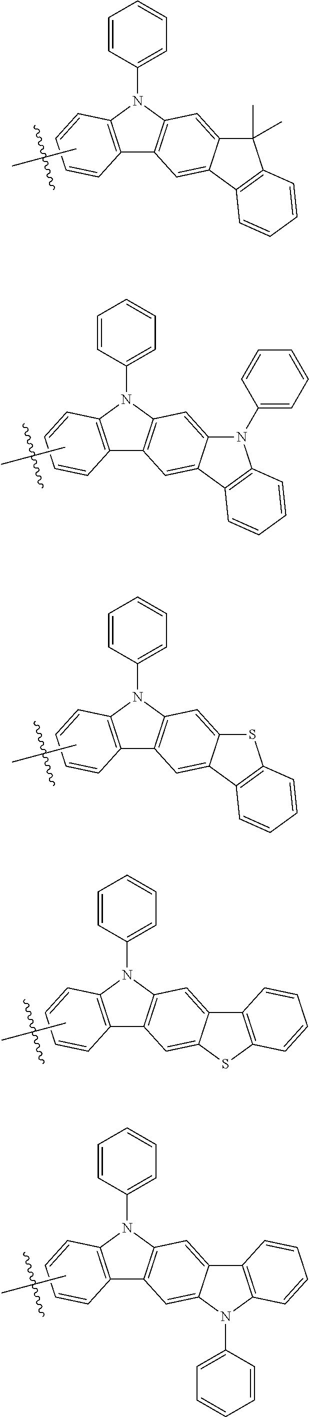

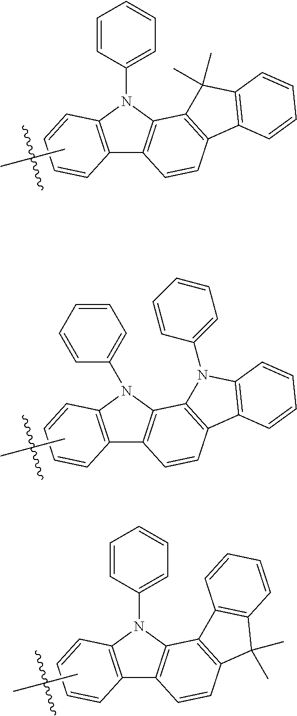

In one embodiment of the present specification, Chemical Formula 1 may be represented by the following Chemical Formula 1-1.

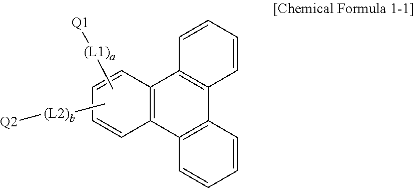

In Chemical Formula 1-1,

each substituent has the same definition as in Chemical Formula 1.

In one embodiment of the present specification, when A2 and A4 are hydrogen and Q1 is a triazine group unsubstituted or substituted with an aryl group, Q2 is a cyano group; a silyl group unsubstituted or substituted with an aryl group; an amine group unsubstituted or substituted with an aryl group; a methyl group unsubstituted or substituted with an aryl group; a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted pyrenyl group; a substituted or unsubstituted fluorenyl group; a substituted or unsubstituted pyridine group; a substituted or unsubstituted pyrimidine group; a substituted or unsubstituted triazine group; a substituted or unsubstituted quinolinyl group; a substituted or unsubstituted quinazolinyl group; a substituted or unsubstituted phenanthrolinyl group; a substituted or unsubstituted carbazolyl group; a substituted or unsubstituted benzocarbazolyl group; a substituted or unsubstituted dibenzofuranyl group; a substituted or unsubstituted dibenzothiophenyl group; a substituted or unsubstituted benzimidazolyl group; a substituted or unsubstituted phosphine oxide group; or any one selected from among the following structural formulae.

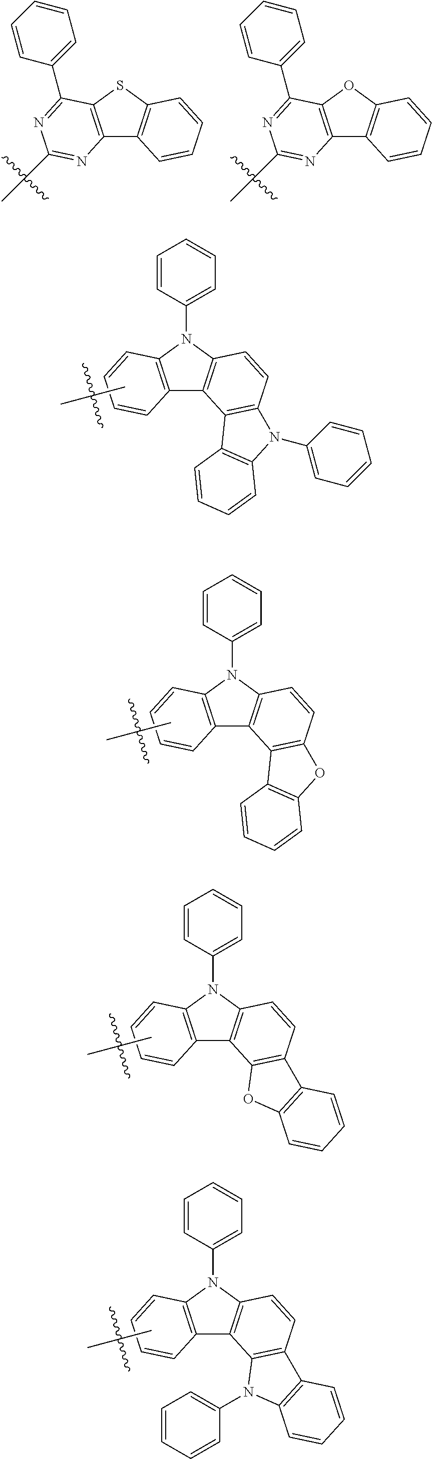

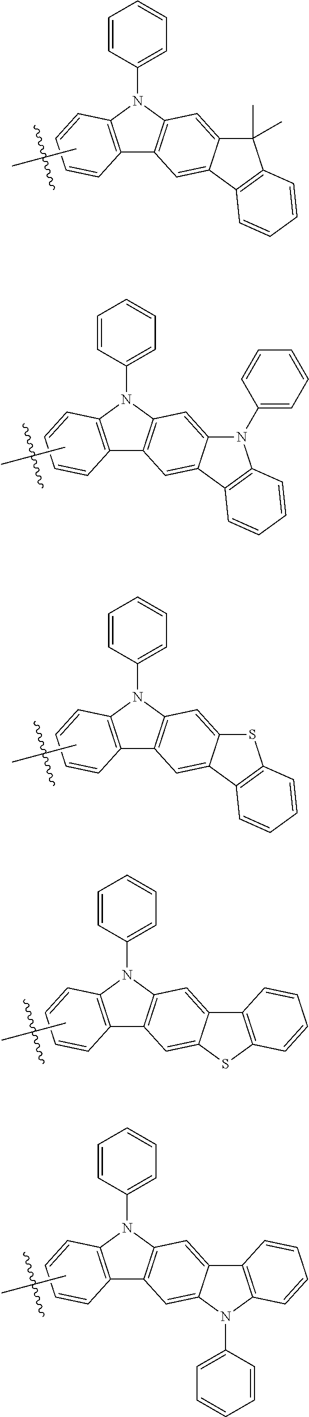

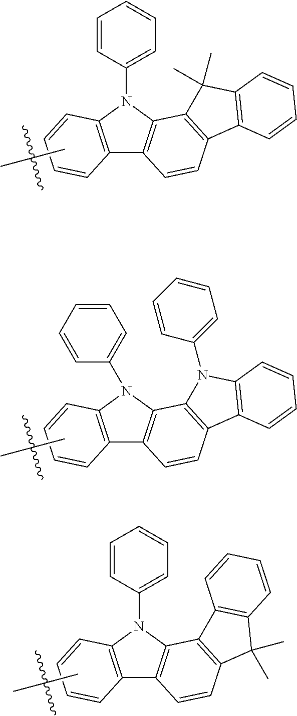

In one embodiment of the present specification, when A2 and A4 are hydrogen and Q1 is a pyridine group unsubstituted or substituted with an aryl group; a pyrimidine group unsubstituted or substituted with an aryl group; a quinazolinyl group unsubstituted or substituted with an aryl group; a phenanthrolinyl group unsubstituted or substituted with an aryl group;

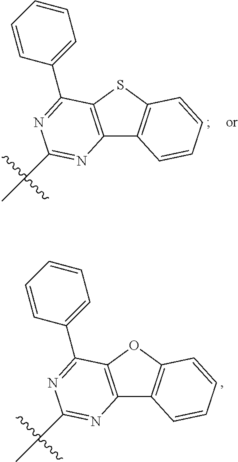 [SMILES: *c1nc(-c2ccccc2)c2sc3ccccc3c2n1 ; *c1nc(-c2ccccc2)c2oc3ccccc3c2n1]

Q2 is a substituted or unsubstituted phenyl group; a substituted or unsubstituted biphenyl group; a substituted or unsubstituted terphenyl group; a substituted or unsubstituted triphenylenyl group; a substituted or unsubstituted fluorenyl group; a substituted or unsubstituted dibenzofuranyl group; a substituted or unsubstituted dibenzothiophenyl group; or a substituted or unsubstituted carbazolyl group.

In one embodiment of the present specification, when A3 and A4 are hydrogen, one of Q1 and Q2 is a triazine group unsubstituted or substituted with an aryl group, and the other one is a cyano group; a substituted or unsubstituted phenyl group; a substituted or unsubstituted biphenyl group; a substituted or unsubstituted terphenyl group; a substituted or unsubstituted naphthyl group; a substituted or unsubstituted triphenylenyl group; or a substituted or unsubstituted fluorenyl group.

In one embodiment of the present specification, when A1 and A4 are hydrogen, Q1 is a triazine group unsubstituted or substituted with an aryl group, and Q2 is a substituted or unsubstituted silyl group; a substituted or unsubstituted phenyl group; a substituted or unsubstituted biphenyl group; a substituted or unsubstituted terphenyl group; a substituted or unsubstituted naphthyl group; or a substituted or unsubstituted dibenzofuranyl group.

In one embodiment of the present specification, when A2 and A3 are hydrogen, one of Q1 and Q2 is a triazine group unsubstituted or substituted with an aryl group, and the other one is a substituted or unsubstituted triphenylenyl group; or a substituted or unsubstituted carbazolyl group.

In one embodiment of the present specification, when A1 is (L1)ₐ-Q1 or (L2)_{b}-Q2, A4 may be hydrogen.

In one embodiment of the present specification, when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a dicyclic or higher aryl group unsubstituted or substituted with an alkyl group or an aryl group is satisfied.

In one embodiment of the present specification, when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a biphenyl group; a terphenyl group; a naphthyl group; a phenanthrenyl group; a triphenylenyl group; a pyrenyl group; or a fluorenyl group substituted with an alkyl group or an aryl group is satisfied.

In one embodiment of the present specification, when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a biphenyl group; a terphenyl group; a naphthyl group; a phenanthrenyl group; a triphenylenyl group; a pyrenyl group; dimethylfluorene; or diphenylfluorene is satisfied.

In one embodiment of the present specification, the compound represented by Chemical Formula 1 does not include an anthracene structure.

In one embodiment of the present specification, Chemical Formula 1 may be represented by the following Chemical Formula 1-N or 1-P.

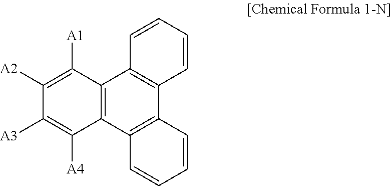

In Chemical Formula 1-N,

A1 to A4 have the same definitions as in Chemical Formula 1, but, include at least one heteroaryl group including a pyridine ring, a pyrimidine ring, a triazine ring or an imidazole ring,

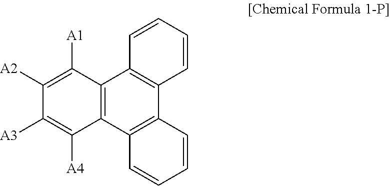

in Chemical Formula 1-P,

A1 to A4 have the same definitions as in Chemical Formula 1, but do not include a heteroaryl group including a pyridine ring, a pyrimidine ring, a triazine ring or an imidazole ring.

Specifically, among the compounds of Chemical Formula 1, the compound including a heteroaryl group including a pyridine ring, a pyrimidine ring, a triazine ring or an imidazole ring may be represented by Chemical Formula 1-N, and, among the compounds of Chemical Formula 1, the compound that does not include a heteroaryl group including a pyridine ring, a pyrimidine ring, a triazine ring or an imidazole ring may be represented by Chemical Formula 1-P.

In one embodiment of the present specification, Chemical Formula 1 may be represented by any one of the following compounds, but is not limited thereto.

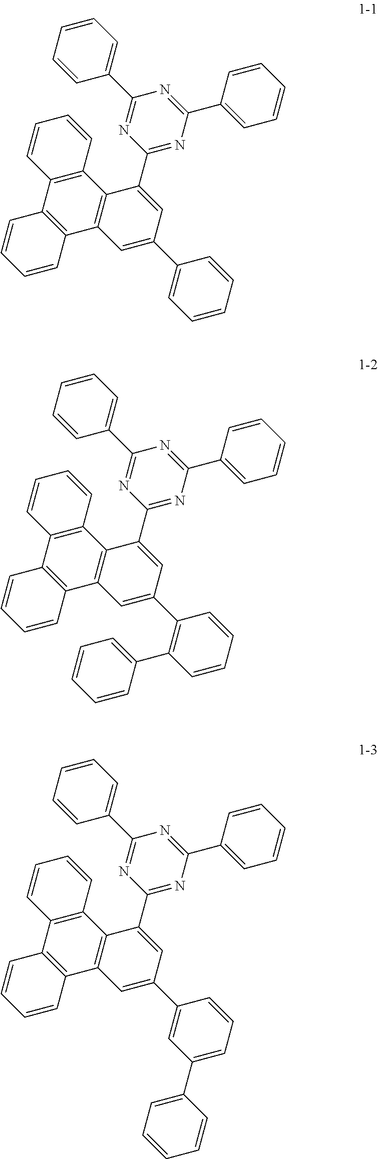 [SMILES: c1ccc(-c2cc(-c3nc(-c4ccccc4)nc(-c4ccccc4)n3)c3c4ccccc4c4ccccc4c3c2)cc1 ; c1ccc(-c2nc(-c3ccccc3)nc(-c3cc(-c4ccccc4-c4ccccc4)cc4c5ccccc5c5ccccc5c34)n2)cc1 ; c1ccc(-c2cccc(-c3cc(-c4nc(-c5ccccc5)nc(-c5ccccc5)n4)c4c5ccccc5c5ccccc5c4c3)c2)cc1]

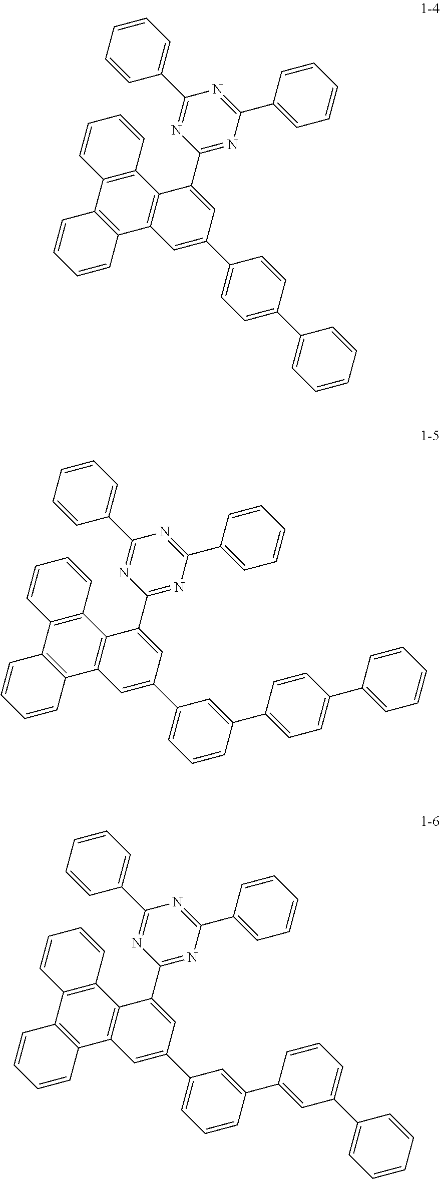 [SMILES: c1ccc(-c2ccc(-c3cc(-c4nc(-c5ccccc5)nc(-c5ccccc5)n4)c4c5ccccc5c5ccccc5c4c3)cc2)cc1 ; c1ccc(-c2ccc(-c3cccc(-c4cc(-c5nc(-c6ccccc6)nc(-c6ccccc6)n5)c5c6ccccc6c6ccccc6c5c4)c3)cc2)cc1 ; c1ccc(-c2cccc(-c3cccc(-c4cc(-c5nc(-c6ccccc6)nc(-c6ccccc6)n5)c5c6ccccc6c6ccccc6c5c4)c3)c2)cc1]

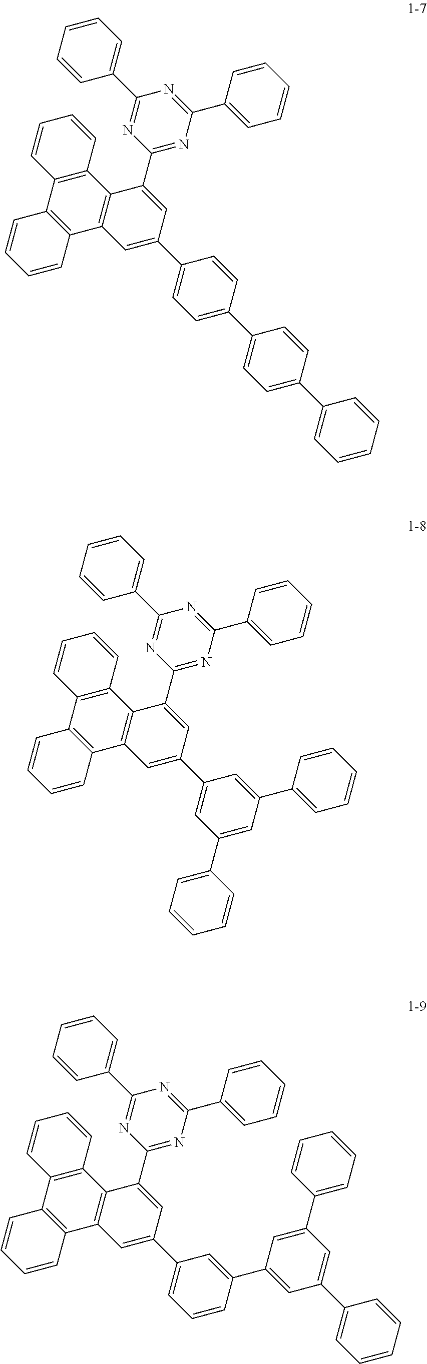 [SMILES: c1ccc(-c2ccc(-c3ccc(-c4cc(-c5nc(-c6ccccc6)nc(-c6ccccc6)n5)c5c6ccccc6c6ccccc6c5c4)cc3)cc2)cc1 ; c1ccc(-c2cc(-c3ccccc3)cc(-c3cc(-c4nc(-c5ccccc5)nc(-c5ccccc5)n4)c4c5ccccc5c5ccccc5c4c3)c2)cc1 ; c1ccc(-c2cc(-c3ccccc3)cc(-c3cccc(-c4cc(-c5nc(-c6ccccc6)nc(-c6ccccc6)n5)c5c6ccccc6c6ccccc6c5c4)c3)c2)cc1]

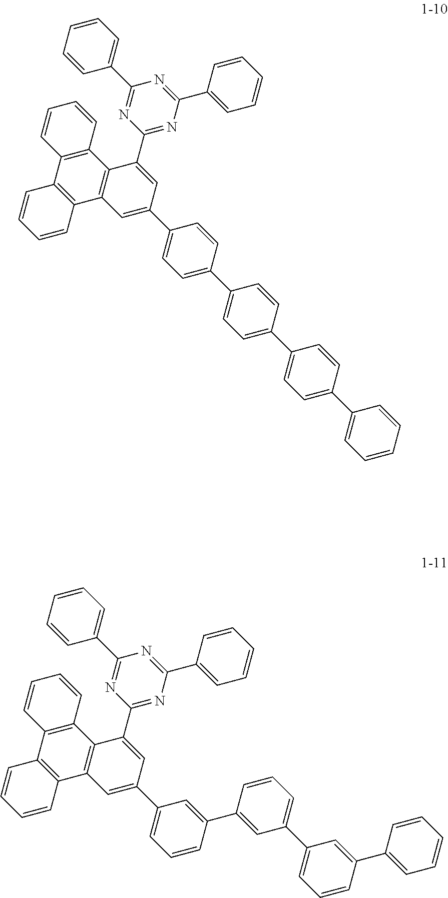 [SMILES: c1ccc(-c2ccc(-c3ccc(-c4ccc(-c5cc(-c6nc(-c7ccccc7)nc(-c7ccccc7)n6)c6c7ccccc7c7ccccc7c6c5)cc4)cc3)cc2)cc1 ; c1ccc(-c2cccc(-c3cccc(-c4cccc(-c5cc(-c6nc(-c7ccccc7)nc(-c7ccccc7)n6)c6c7ccccc7c7ccccc7c6c5)c4)c3)c2)cc1]

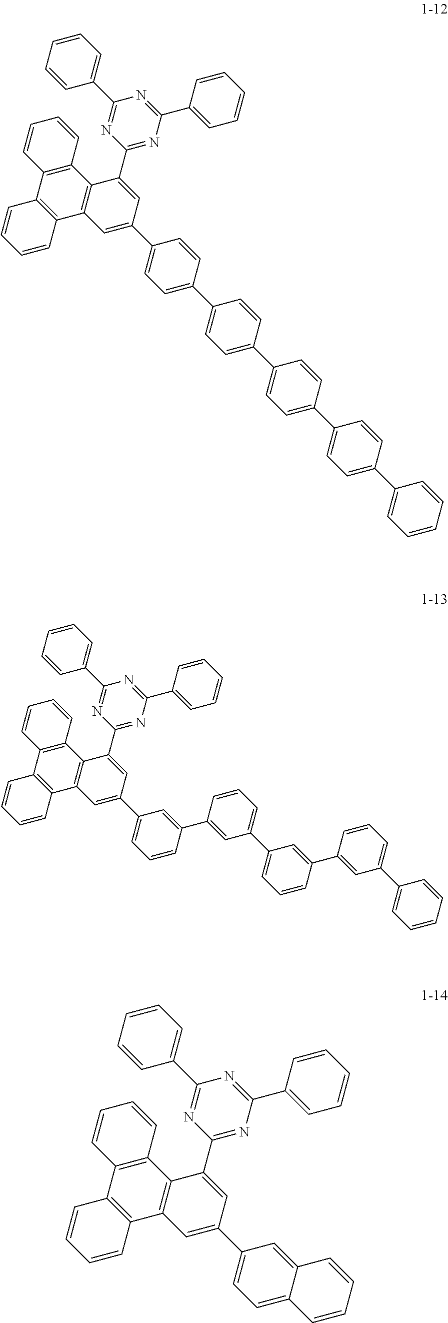 [SMILES: c1ccc(-c2ccc(-c3ccc(-c4ccc(-c5ccc(-c6cc(-c7nc(-c8ccccc8)nc(-c8ccccc8)n7)c7c8ccccc8c8ccccc8c7c6)cc5)cc4)cc3)cc2)cc1 ; c1ccc(-c2cccc(-c3cccc(-c4cccc(-c5cccc(-c6cc(-c7nc(-c8ccccc8)nc(-c8ccccc8)n7)c7c8ccccc8c8ccccc8c7c6)c5)c4)c3)c2)cc1 ; c1ccc(-c2nc(-c3ccccc3)nc(-c3cc(-c4ccc5ccccc5c4)cc4c5ccccc5c5ccccc5c34)n2)cc1]

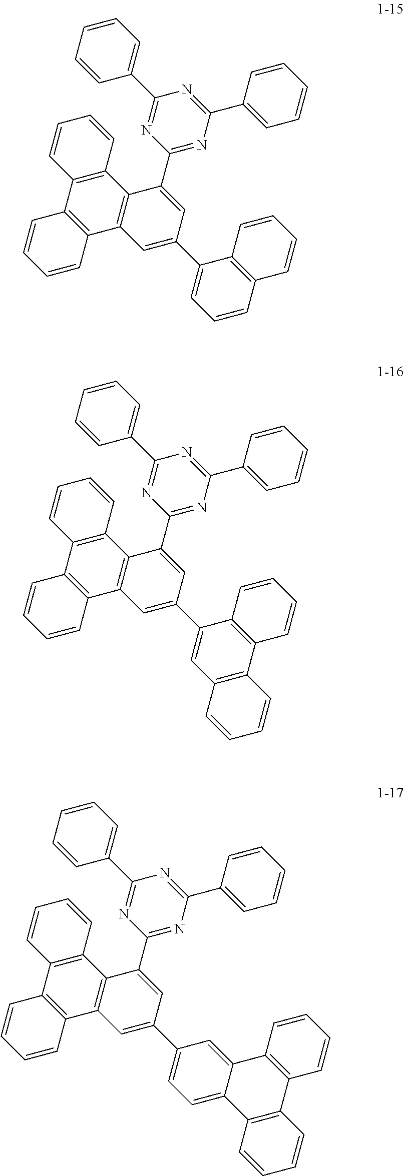 [SMILES: c1ccc(-c2nc(-c3ccccc3)nc(-c3cc(-c4cccc5ccccc45)cc4c5ccccc5c5ccccc5c34)n2)cc1 ; c1ccc(-c2nc(-c3ccccc3)nc(-c3cc(-c4cc5ccccc5c5ccccc45)cc4c5ccccc5c5ccccc5c34)n2)cc1 ; c1ccc(-c2nc(-c3ccccc3)nc(-c3cc(-c4ccc5c6ccccc6c6ccccc6c5c4)cc4c5ccccc5c5ccccc5c34)n2)cc1]

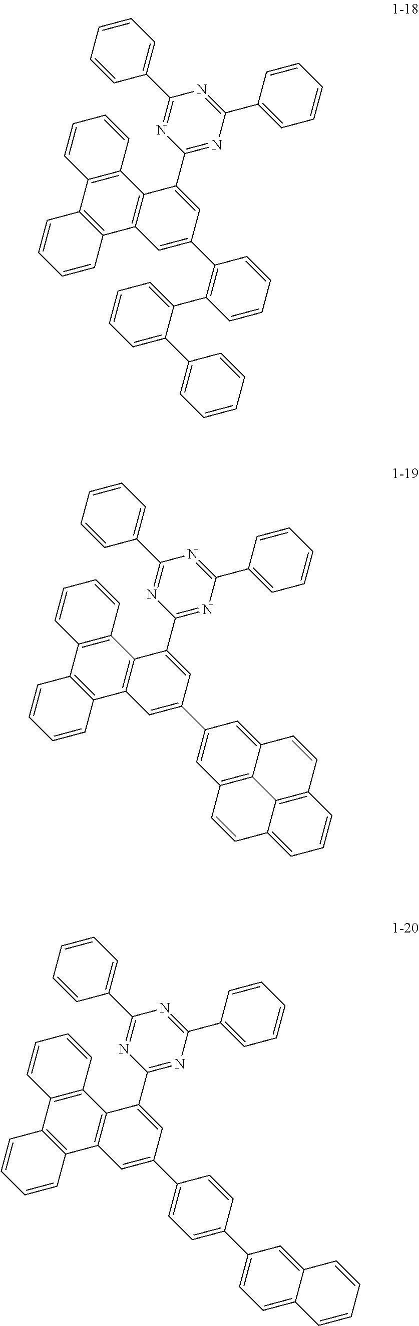 [SMILES: c1ccc(-c2nc(-c3ccccc3)nc(-c3cc(-c4ccccc4-c4ccccc4-c4ccccc4)cc4c5ccccc5c5ccccc5c34)n2)cc1 ; c1ccc(-c2nc(-c3ccccc3)nc(-c3cc(-c4cc5ccc6cccc7ccc(c4)c5c67)cc4c5ccccc5c5ccccc5c34)n2)cc1 ; c1ccc(-c2nc(-c3ccccc3)nc(-c3cc(-c4ccc(-c5ccc6ccccc6c5)cc4)cc4c5ccccc5c5ccccc5c34)n2)cc1]

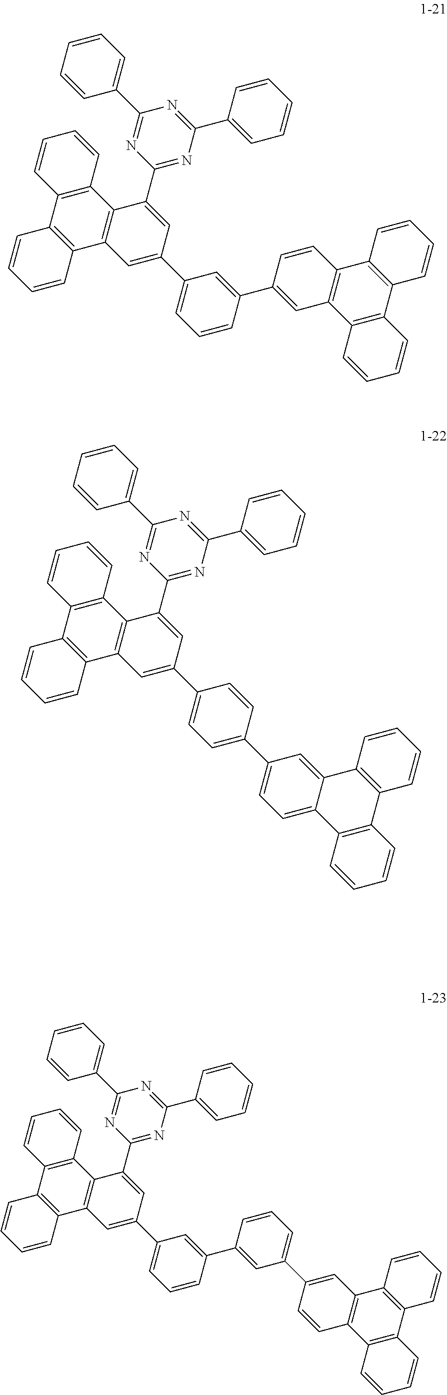 [SMILES: c1ccc(-c2nc(-c3ccccc3)nc(-c3cc(-c4cccc(-c5ccc6c7ccccc7c7ccccc7c6c5)c4)cc4c5ccccc5c5ccccc5c34)n2)cc1 ; c1ccc(-c2nc(-c3ccccc3)nc(-c3cc(-c4ccc(-c5ccc6c7ccccc7c7ccccc7c6c5)cc4)cc4c5ccccc5c5ccccc5c34)n2)cc1 ; c1ccc(-c2nc(-c3ccccc3)nc(-c3cc(-c4cccc(-c5cccc(-c6ccc7c8ccccc8c8ccccc8c7c6)c5)c4)cc4c5ccccc5c5ccccc5c34)n2)cc1]

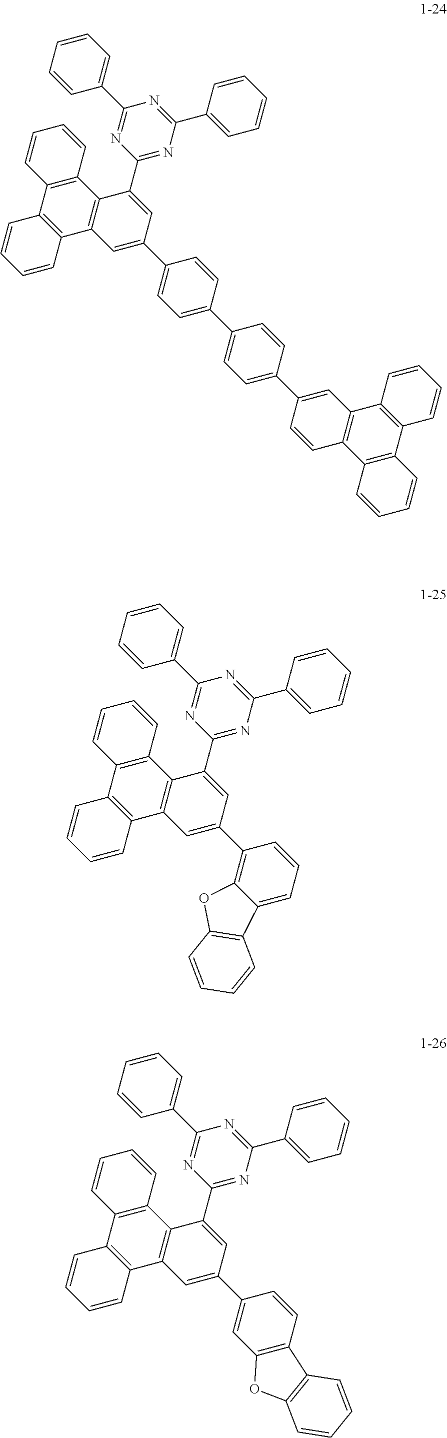 [SMILES: c1ccc(-c2nc(-c3ccccc3)nc(-c3cc(-c4ccc(-c5ccc(-c6ccc7c8ccccc8c8ccccc8c7c6)cc5)cc4)cc4c5ccccc5c5ccccc5c34)n2)cc1 ; c1ccc(-c2nc(-c3ccccc3)nc(-c3cc(-c4cccc5c4oc4ccccc45)cc4c5ccccc5c5ccccc5c34)n2)cc1 ; c1ccc(-c2nc(-c3ccccc3)nc(-c3cc(-c4ccc5c(c4)oc4ccccc45)cc4c5ccccc5c5ccccc5c34)n2)cc1]

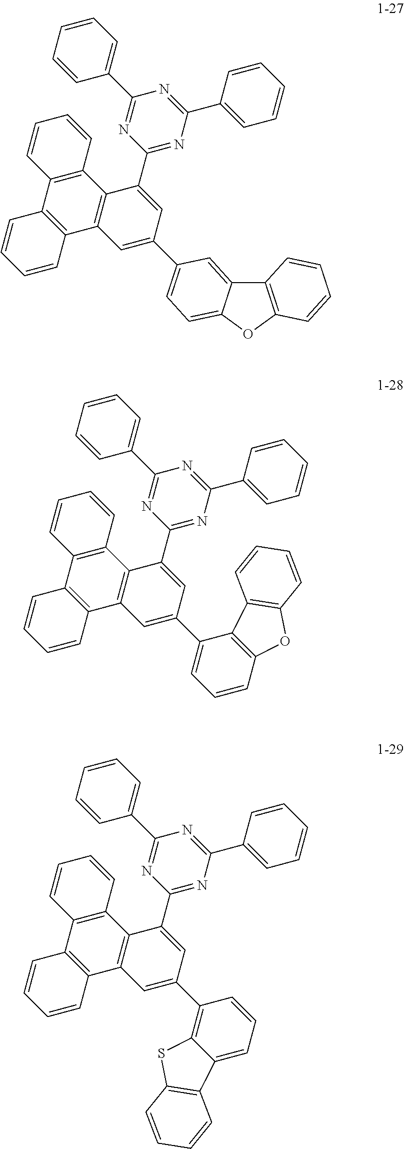 [SMILES: c1ccc(-c2nc(-c3ccccc3)nc(-c3cc(-c4ccc5oc6ccccc6c5c4)cc4c5ccccc5c5ccccc5c34)n2)cc1 ; c1ccc(-c2nc(-c3ccccc3)nc(-c3cc(-c4cccc5oc6ccccc6c45)cc4c5ccccc5c5ccccc5c34)n2)cc1 ; c1ccc(-c2nc(-c3ccccc3)nc(-c3cc(-c4cccc5c4sc4ccccc45)cc4c5ccccc5c5ccccc5c34)n2)cc1]

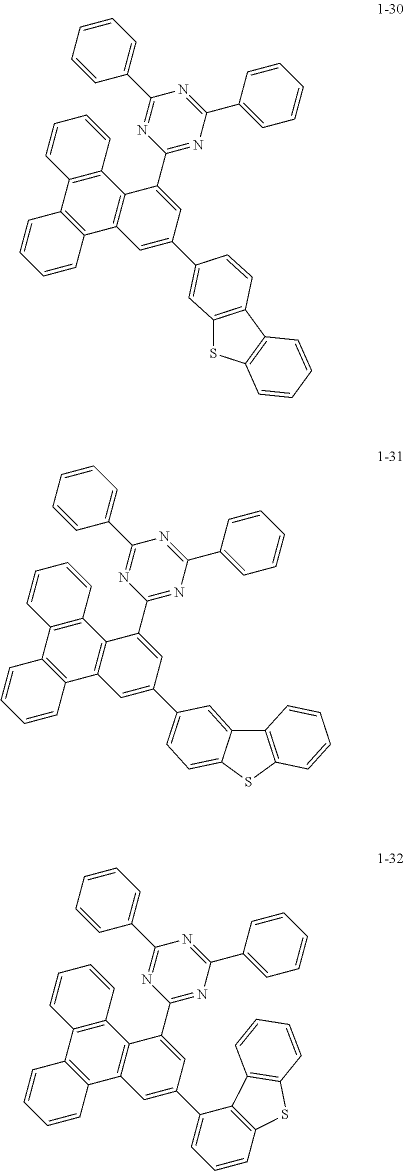 [SMILES: c1ccc(-c2nc(-c3ccccc3)nc(-c3cc(-c4ccc5c(c4)sc4ccccc45)cc4c5ccccc5c5ccccc5c34)n2)cc1 ; c1ccc(-c2nc(-c3ccccc3)nc(-c3cc(-c4ccc5sc6ccccc6c5c4)cc4c5ccccc5c5ccccc5c34)n2)cc1 ; c1ccc(-c2nc(-c3ccccc3)nc(-c3cc(-c4cccc5sc6ccccc6c45)cc4c5ccccc5c5ccccc5c34)n2)cc1]

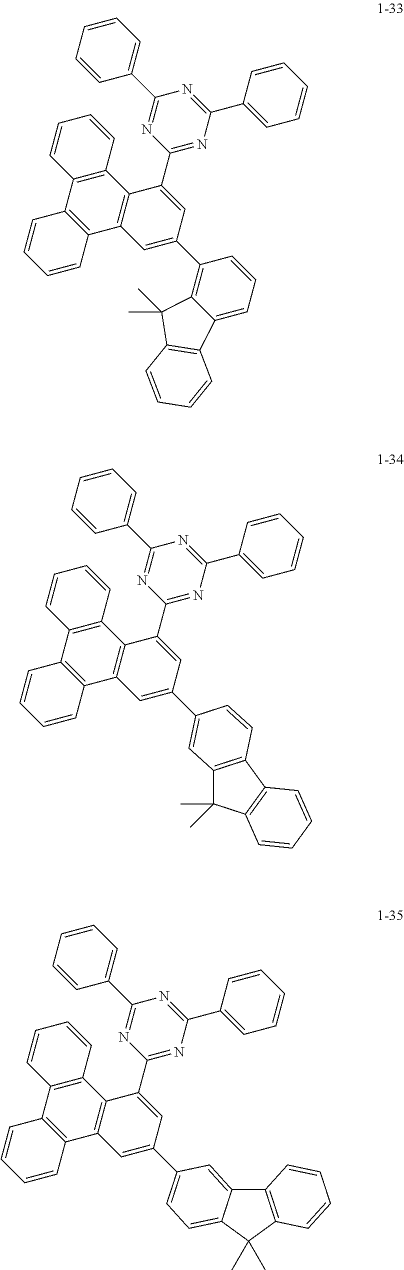 [SMILES: CC1(C)c2ccccc2-c2cccc(-c3cc(-c4nc(-c5ccccc5)nc(-c5ccccc5)n4)c4c5ccccc5c5ccccc5c4c3)c21 ; CC1(C)c2ccccc2-c2ccc(-c3cc(-c4nc(-c5ccccc5)nc(-c5ccccc5)n4)c4c5ccccc5c5ccccc5c4c3)cc21 ; CC1(C)c2ccccc2-c2cc(-c3cc(-c4nc(-c5ccccc5)nc(-c5ccccc5)n4)c4c5ccccc5c5ccccc5c4c3)ccc21]

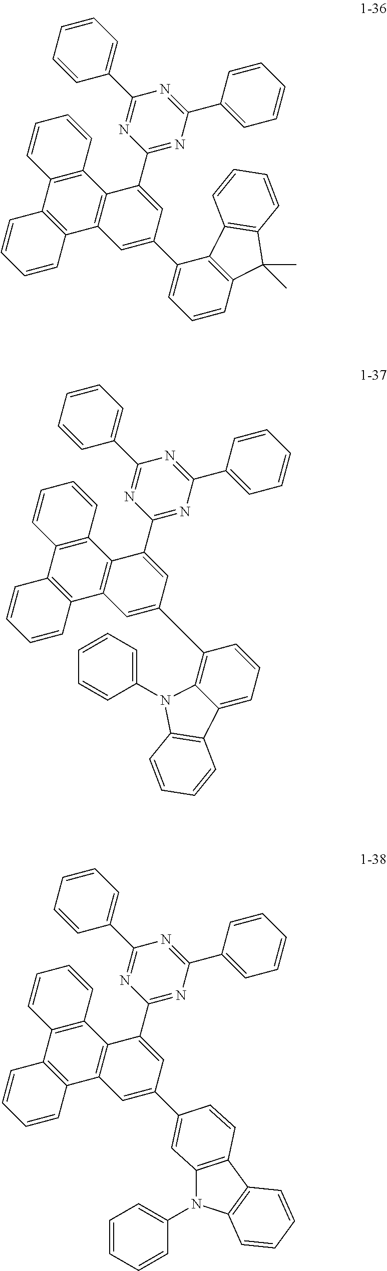 [SMILES: CC1(C)c2ccccc2-c2c(-c3cc(-c4nc(-c5ccccc5)nc(-c5ccccc5)n4)c4c5ccccc5c5ccccc5c4c3)cccc21 ; c1ccc(-c2nc(-c3ccccc3)nc(-c3cc(-c4cccc5c6ccccc6n(-c6ccccc6)c45)cc4c5ccccc5c5ccccc5c34)n2)cc1 ; c1ccc(-c2nc(-c3ccccc3)nc(-c3cc(-c4ccc5c6ccccc6n(-c6ccccc6)c5c4)cc4c5ccccc5c5ccccc5c34)n2)cc1]

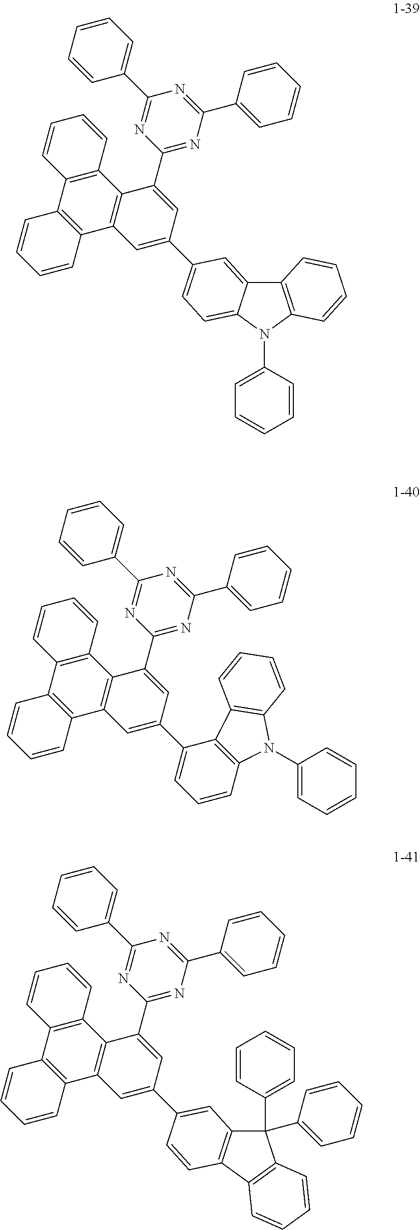 [SMILES: c1ccc(-c2nc(-c3ccccc3)nc(-c3cc(-c4ccc5c(c4)c4ccccc4n5-c4ccccc4)cc4c5ccccc5c5ccccc5c34)n2)cc1 ; c1ccc(-c2nc(-c3ccccc3)nc(-c3cc(-c4cccc5c4c4ccccc4n5-c4ccccc4)cc4c5ccccc5c5ccccc5c34)n2)cc1 ; c1ccc(-c2nc(-c3ccccc3)nc(-c3cc(-c4ccc5c(c4)C(c4ccccc4)(c4ccccc4)c4ccccc4-5)cc4c5ccccc5c5ccccc5c34)n2)cc1]

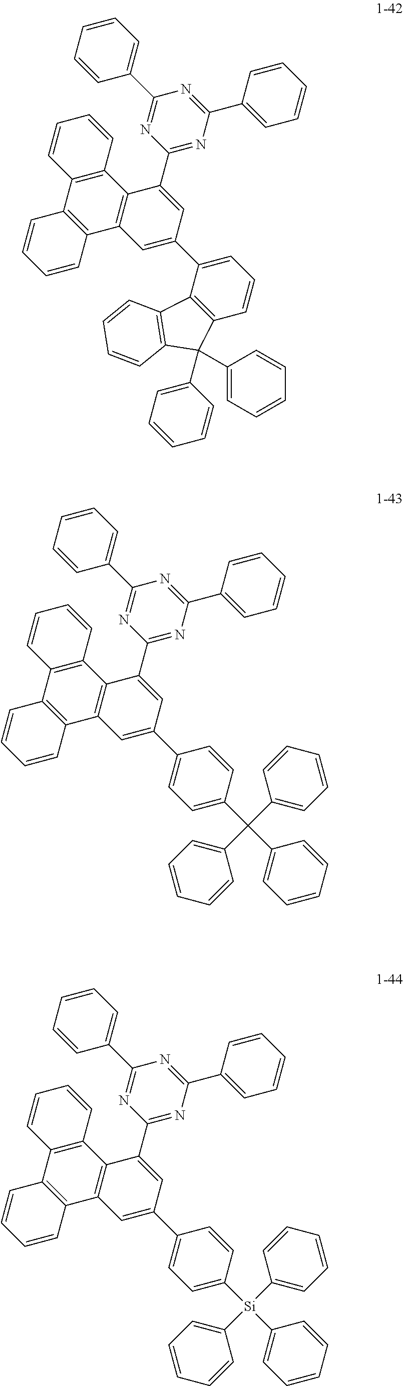 [SMILES: c1ccc(-c2nc(-c3ccccc3)nc(-c3cc(-c4cccc5c4-c4ccccc4C5(c4ccccc4)c4ccccc4)cc4c5ccccc5c5ccccc5c34)n2)cc1 ; c1ccc(-c2nc(-c3ccccc3)nc(-c3cc(-c4ccc(C(c5ccccc5)(c5ccccc5)c5ccccc5)cc4)cc4c5ccccc5c5ccccc5c34)n2)cc1 ; c1ccc(-c2nc(-c3ccccc3)nc(-c3cc(-c4ccc([Si](c5ccccc5)(c5ccccc5)c5ccccc5)cc4)cc4c5ccccc5c5ccccc5c34)n2)cc1]

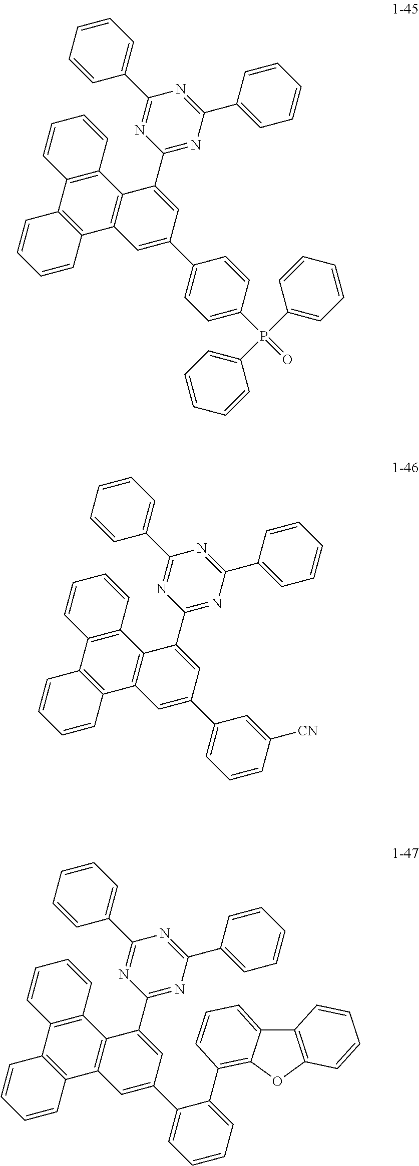 [SMILES: O=P(c1ccccc1)(c1ccccc1)c1ccc(-c2cc(-c3nc(-c4ccccc4)nc(-c4ccccc4)n3)c3c4ccccc4c4ccccc4c3c2)cc1 ; N#Cc1cccc(-c2cc(-c3nc(-c4ccccc4)nc(-c4ccccc4)n3)c3c4ccccc4c4ccccc4c3c2)c1 ; c1ccc(-c2nc(-c3ccccc3)nc(-c3cc(-c4ccccc4-c4cccc5c4oc4ccccc45)cc4c5ccccc5c5ccccc5c34)n2)cc1]

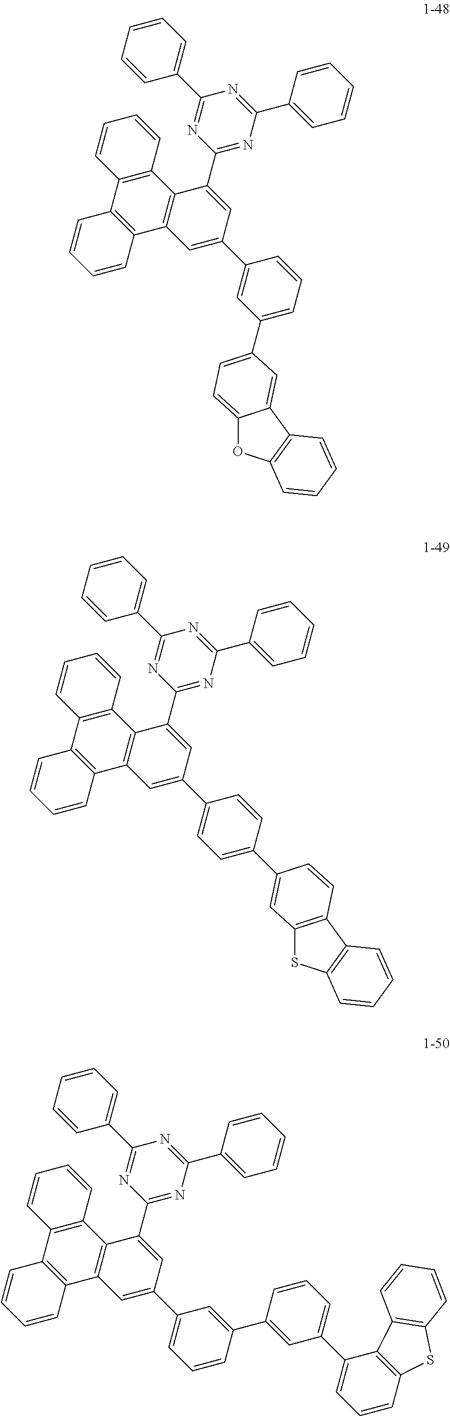 [SMILES: c1ccc(-c2nc(-c3ccccc3)nc(-c3cc(-c4cccc(-c5ccc6oc7ccccc7c6c5)c4)cc4c5ccccc5c5ccccc5c34)n2)cc1 ; c1ccc(-c2nc(-c3ccccc3)nc(-c3cc(-c4ccc(-c5ccc6c(c5)sc5ccccc56)cc4)cc4c5ccccc5c5ccccc5c34)n2)cc1 ; c1ccc(-c2nc(-c3ccccc3)nc(-c3cc(-c4cccc(-c5cccc(-c6cccc7sc8ccccc8c67)c5)c4)cc4c5ccccc5c5ccccc5c34)n2)cc1]

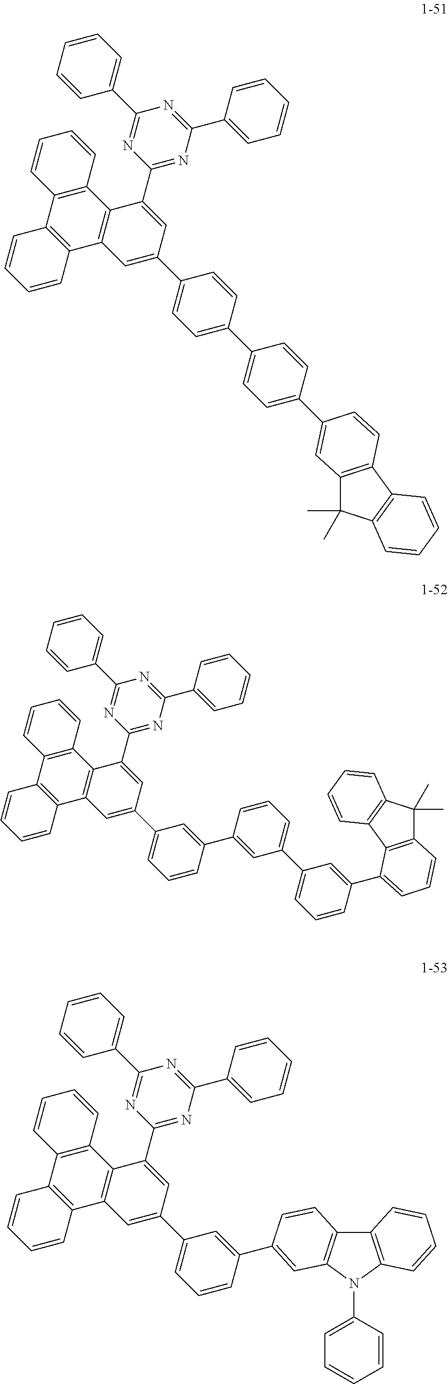 [SMILES: CC1(C)c2ccccc2-c2ccc(-c3ccc(-c4ccc(-c5cc(-c6nc(-c7ccccc7)nc(-c7ccccc7)n6)c6c7ccccc7c7ccccc7c6c5)cc4)cc3)cc21 ; CC1(C)c2ccccc2-c2c(-c3cccc(-c4cccc(-c5cccc(-c6cc(-c7nc(-c8ccccc8)nc(-c8ccccc8)n7)c7c8ccccc8c8ccccc8c7c6)c5)c4)c3)cccc21 ; c1ccc(-c2nc(-c3ccccc3)nc(-c3cc(-c4cccc(-c5ccc6c7ccccc7n(-c7ccccc7)c6c5)c4)cc4c5ccccc5c5ccccc5c34)n2)cc1]

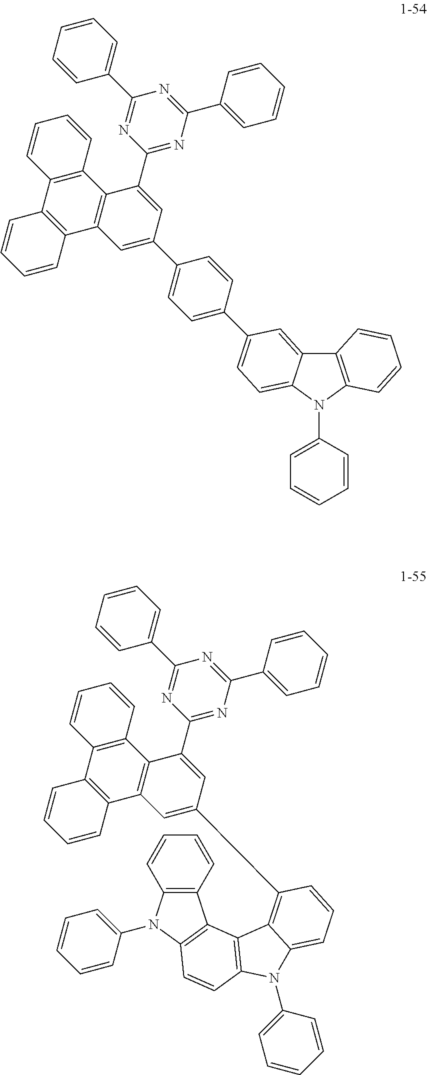 [SMILES: c1ccc(-c2nc(-c3ccccc3)nc(-c3cc(-c4ccc(-c5ccc6c(c5)c5ccccc5n6-c5ccccc5)cc4)cc4c5ccccc5c5ccccc5c34)n2)cc1 ; c1ccc(-c2nc(-c3ccccc3)nc(-c3cc(-c4cccc5c4c4c6c7ccccc7n(-c7ccccc7)c6ccc4n5-c4ccccc4)cc4c5ccccc5c5ccccc5c34)n2)cc1]

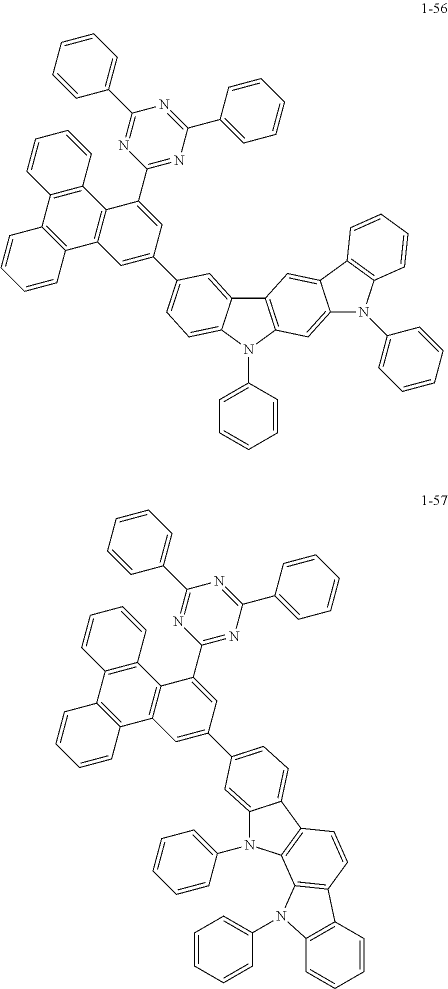 [SMILES: c1ccc(-c2nc(-c3ccccc3)nc(-c3cc(-c4ccc5c(c4)c4cc6c7ccccc7n(-c7ccccc7)c6cc4n5-c4ccccc4)cc4c5ccccc5c5ccccc5c34)n2)cc1 ; c1ccc(-c2nc(-c3ccccc3)nc(-c3cc(-c4ccc5c6ccc7c8ccccc8n(-c8ccccc8)c7c6n(-c6ccccc6)c5c4)cc4c5ccccc5c5ccccc5c34)n2)cc1]

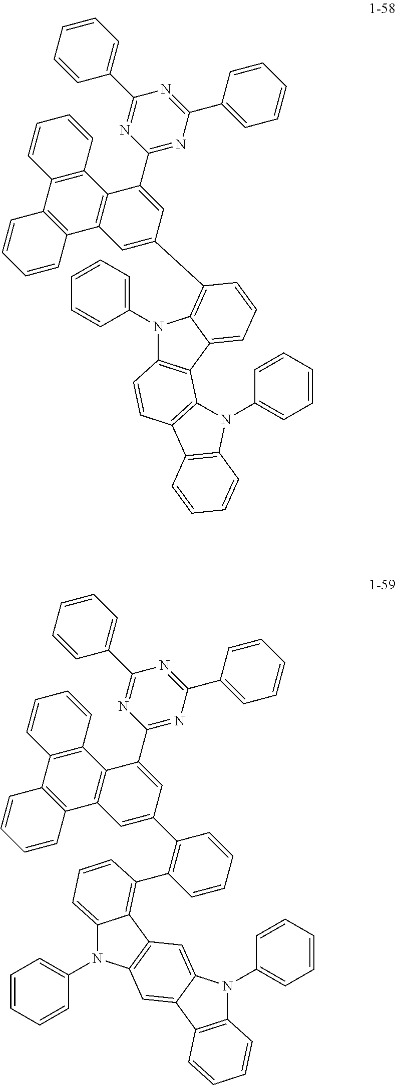 [SMILES: c1ccc(-c2nc(-c3ccccc3)nc(-c3cc(-c4cccc5c6c(ccc7c8ccccc8n(-c8ccccc8)c76)n(-c6ccccc6)c45)cc4c5ccccc5c5ccccc5c34)n2)cc1 ; c1ccc(-c2nc(-c3ccccc3)nc(-c3cc(-c4ccccc4-c4cccc5c4c4cc6c(cc4n5-c4ccccc4)c4ccccc4n6-c4ccccc4)cc4c5ccccc5c5ccccc5c34)n2)cc1]

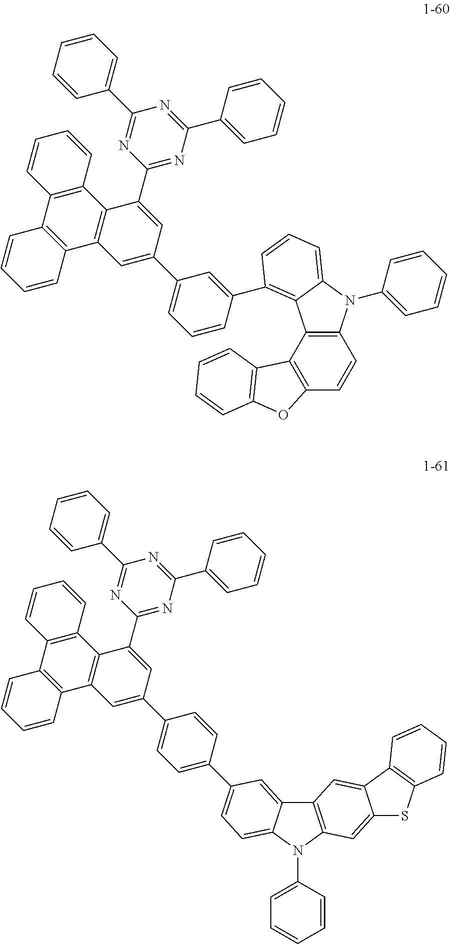 [SMILES: c1ccc(-c2nc(-c3ccccc3)nc(-c3cc(-c4cccc(-c5cccc6c5c5c7c(ccc5n6-c5ccccc5)oc5ccccc57)c4)cc4c5ccccc5c5ccccc5c34)n2)cc1 ; c1ccc(-c2nc(-c3ccccc3)nc(-c3cc(-c4ccc(-c5ccc6c(c5)c5cc7c(cc5n6-c5ccccc5)sc5ccccc57)cc4)cc4c5ccccc5c5ccccc5c34)n2)cc1]

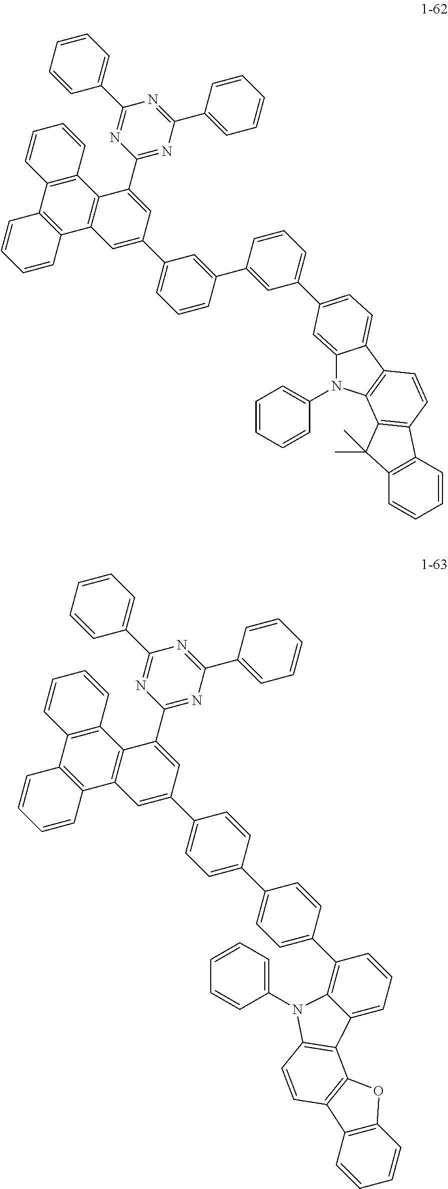 [SMILES: CC1(C)c2ccccc2-c2ccc3c4ccc(-c5cccc(-c6cccc(-c7cc(-c8nc(-c9ccccc9)nc(-c9ccccc9)n8)c8c9ccccc9c9ccccc9c8c7)c6)c5)cc4n(-c4ccccc4)c3c21 ; c1ccc(-c2nc(-c3ccccc3)nc(-c3cc(-c4ccc(-c5ccc(-c6cccc7c8c9oc%10ccccc%10c9ccc8n(-c8ccccc8)c67)cc5)cc4)cc4c5ccccc5c5ccccc5c34)n2)cc1]

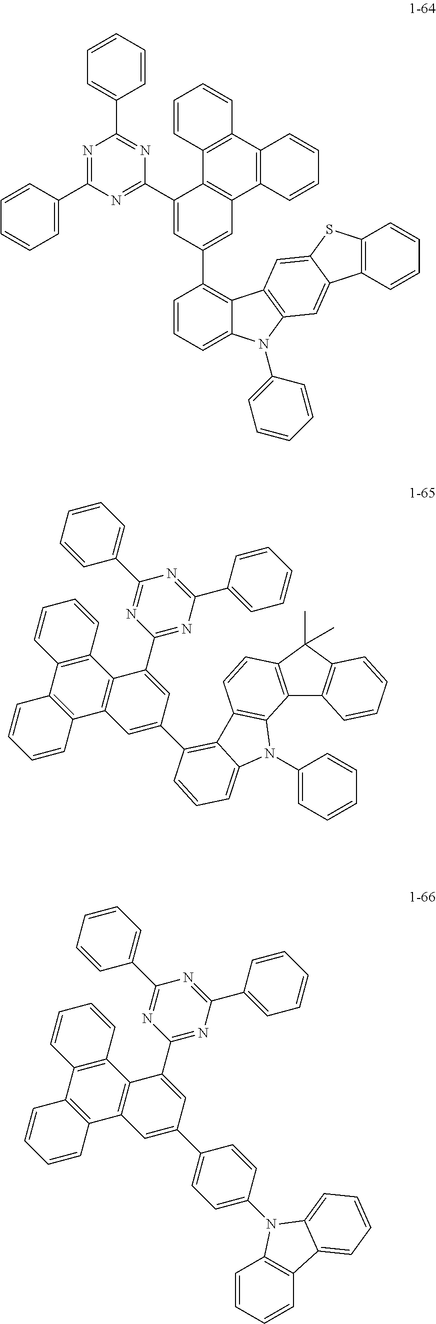 [SMILES: c1ccc(-c2nc(-c3ccccc3)nc(-c3cc(-c4cccc5c4c4cc6sc7ccccc7c6cc4n5-c4ccccc4)cc4c5ccccc5c5ccccc5c34)n2)cc1 ; CC1(C)c2ccccc2-c2c1ccc1c3c(-c4cc(-c5nc(-c6ccccc6)nc(-c6ccccc6)n5)c5c6ccccc6c6ccccc6c5c4)cccc3n(-c3ccccc3)c21 ; c1ccc(-c2nc(-c3ccccc3)nc(-c3cc(-c4ccc(-n5c6ccccc6c6ccccc65)cc4)cc4c5ccccc5c5ccccc5c34)n2)cc1]

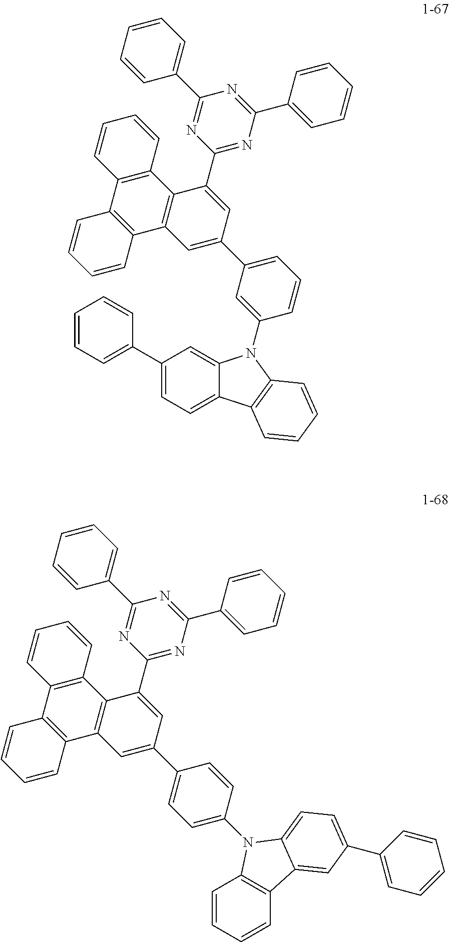 [SMILES: c1ccc(-c2ccc3c4ccccc4n(-c4cccc(-c5cc(-c6nc(-c7ccccc7)nc(-c7ccccc7)n6)c6c7ccccc7c7ccccc7c6c5)c4)c3c2)cc1 ; c1ccc(-c2ccc3c(c2)c2ccccc2n3-c2ccc(-c3cc(-c4nc(-c5ccccc5)nc(-c5ccccc5)n4)c4c5ccccc5c5ccccc5c4c3)cc2)cc1]

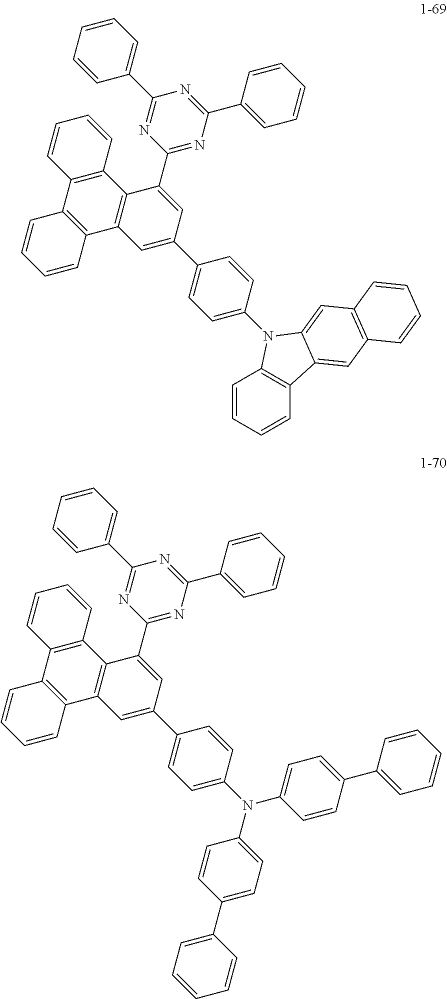 [SMILES: c1ccc(-c2nc(-c3ccccc3)nc(-c3cc(-c4ccc(-n5c6ccccc6c6cc7ccccc7cc65)cc4)cc4c5ccccc5c5ccccc5c34)n2)cc1 ; c1ccc(-c2ccc(N(c3ccc(-c4ccccc4)cc3)c3ccc(-c4cc(-c5nc(-c6ccccc6)nc(-c6ccccc6)n5)c5c6ccccc6c6ccccc6c5c4)cc3)cc2)cc1]

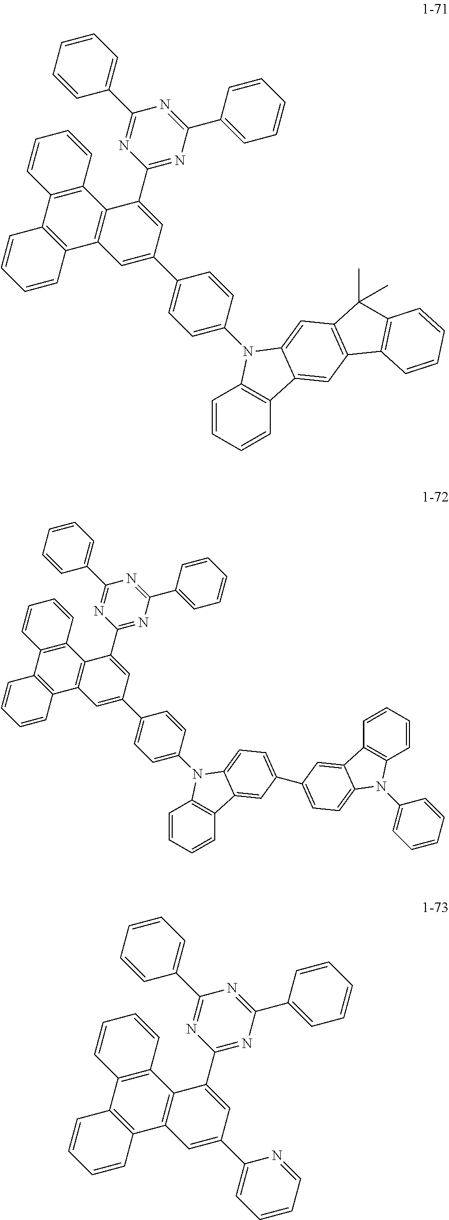 [SMILES: CC1(C)c2ccccc2-c2cc3c4ccccc4n(-c4ccc(-c5cc(-c6nc(-c7ccccc7)nc(-c7ccccc7)n6)c6c7ccccc7c7ccccc7c6c5)cc4)c3cc21 ; c1ccc(-c2nc(-c3ccccc3)nc(-c3cc(-c4ccc(-n5c6ccccc6c6cc(-c7ccc8c(c7)c7ccccc7n8-c7ccccc7)ccc65)cc4)cc4c5ccccc5c5ccccc5c34)n2)cc1 ; c1ccc(-c2nc(-c3ccccc3)nc(-c3cc(-c4ccccn4)cc4c5ccccc5c5ccccc5c34)n2)cc1]

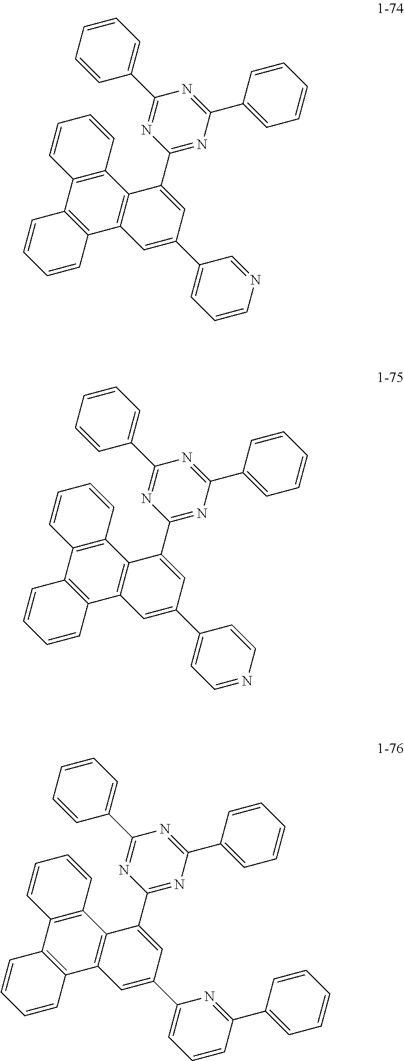 [SMILES: c1ccc(-c2nc(-c3ccccc3)nc(-c3cc(-c4cccnc4)cc4c5ccccc5c5ccccc5c34)n2)cc1 ; c1ccc(-c2nc(-c3ccccc3)nc(-c3cc(-c4ccncc4)cc4c5ccccc5c5ccccc5c34)n2)cc1 ; c1ccc(-c2cccc(-c3cc(-c4nc(-c5ccccc5)nc(-c5ccccc5)n4)c4c5ccccc5c5ccccc5c4c3)n2)cc1]

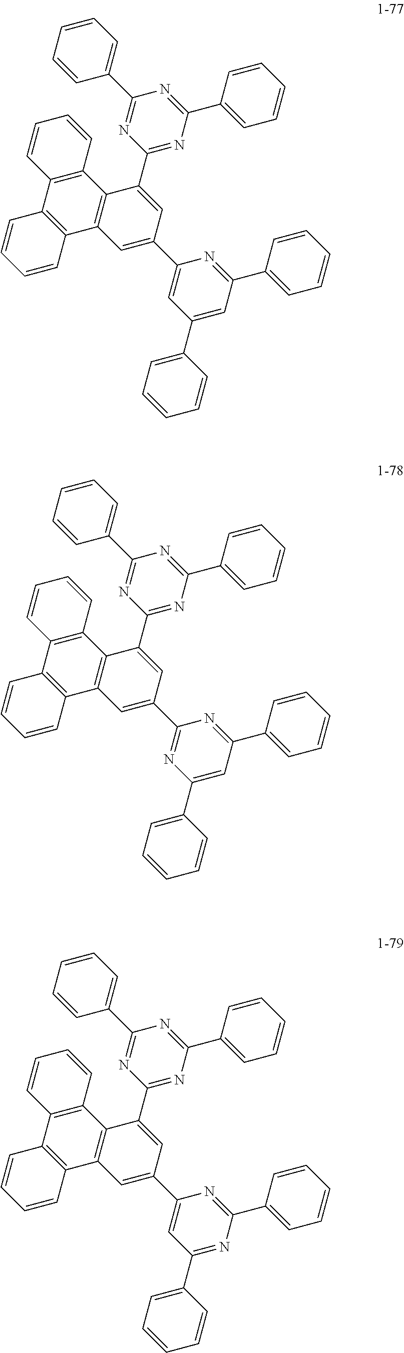 [SMILES: c1ccc(-c2cc(-c3ccccc3)nc(-c3cc(-c4nc(-c5ccccc5)nc(-c5ccccc5)n4)c4c5ccccc5c5ccccc5c4c3)c2)cc1 ; c1ccc(-c2cc(-c3ccccc3)nc(-c3cc(-c4nc(-c5ccccc5)nc(-c5ccccc5)n4)c4c5ccccc5c5ccccc5c4c3)n2)cc1 ; c1ccc(-c2cc(-c3cc(-c4nc(-c5ccccc5)nc(-c5ccccc5)n4)c4c5ccccc5c5ccccc5c4c3)nc(-c3ccccc3)n2)cc1]

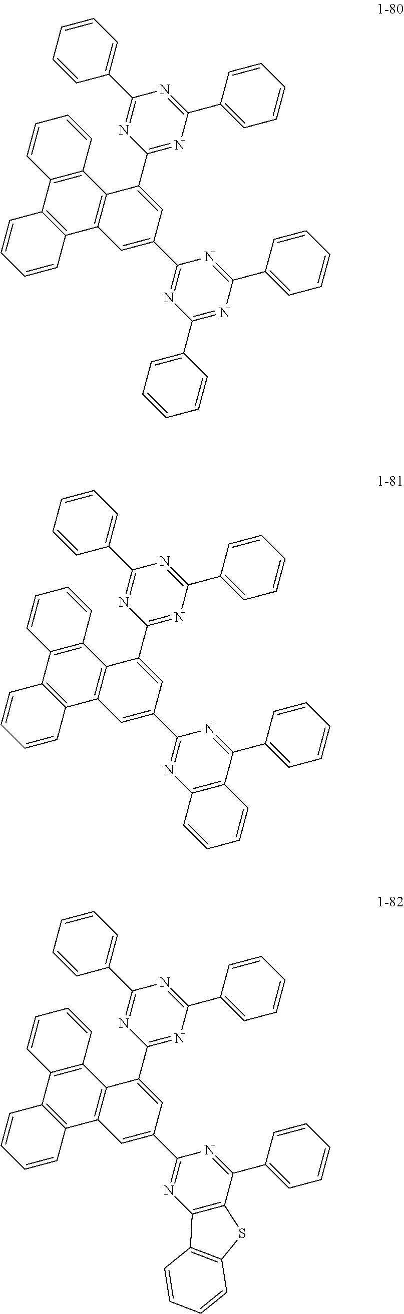 [SMILES: c1ccc(-c2nc(-c3ccccc3)nc(-c3cc(-c4nc(-c5ccccc5)nc(-c5ccccc5)n4)c4c5ccccc5c5ccccc5c4c3)n2)cc1 ; c1ccc(-c2nc(-c3ccccc3)nc(-c3cc(-c4nc(-c5ccccc5)c5ccccc5n4)cc4c5ccccc5c5ccccc5c34)n2)cc1 ; c1ccc(-c2nc(-c3ccccc3)nc(-c3cc(-c4nc(-c5ccccc5)c5sc6ccccc6c5n4)cc4c5ccccc5c5ccccc5c34)n2)cc1]

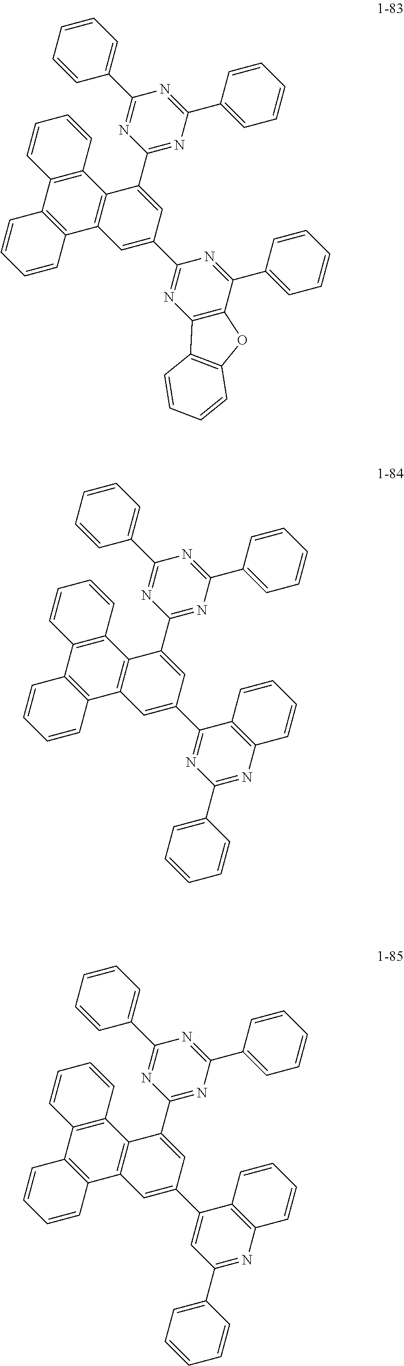 [SMILES: c1ccc(-c2nc(-c3ccccc3)nc(-c3cc(-c4nc(-c5ccccc5)c5oc6ccccc6c5n4)cc4c5ccccc5c5ccccc5c34)n2)cc1 ; c1ccc(-c2nc(-c3ccccc3)nc(-c3cc(-c4nc(-c5ccccc5)nc5ccccc45)cc4c5ccccc5c5ccccc5c34)n2)cc1 ; c1ccc(-c2cc(-c3cc(-c4nc(-c5ccccc5)nc(-c5ccccc5)n4)c4c5ccccc5c5ccccc5c4c3)c3ccccc3n2)cc1]

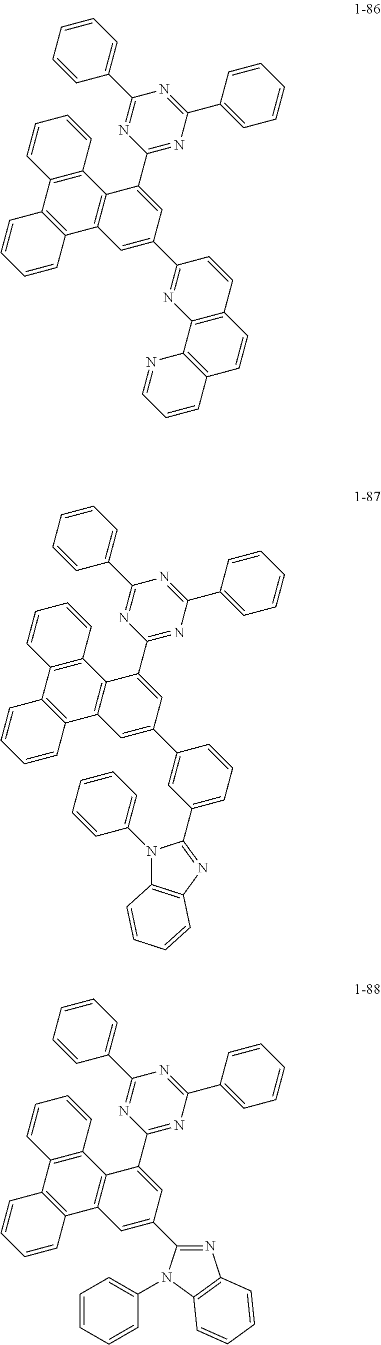 [SMILES: c1ccc(-c2nc(-c3ccccc3)nc(-c3cc(-c4ccc5ccc6cccnc6c5n4)cc4c5ccccc5c5ccccc5c34)n2)cc1 ; c1ccc(-c2nc(-c3ccccc3)nc(-c3cc(-c4cccc(-c5nc6ccccc6n5-c5ccccc5)c4)cc4c5ccccc5c5ccccc5c34)n2)cc1 ; c1ccc(-c2nc(-c3ccccc3)nc(-c3cc(-c4nc5ccccc5n4-c4ccccc4)cc4c5ccccc5c5ccccc5c34)n2)cc1]

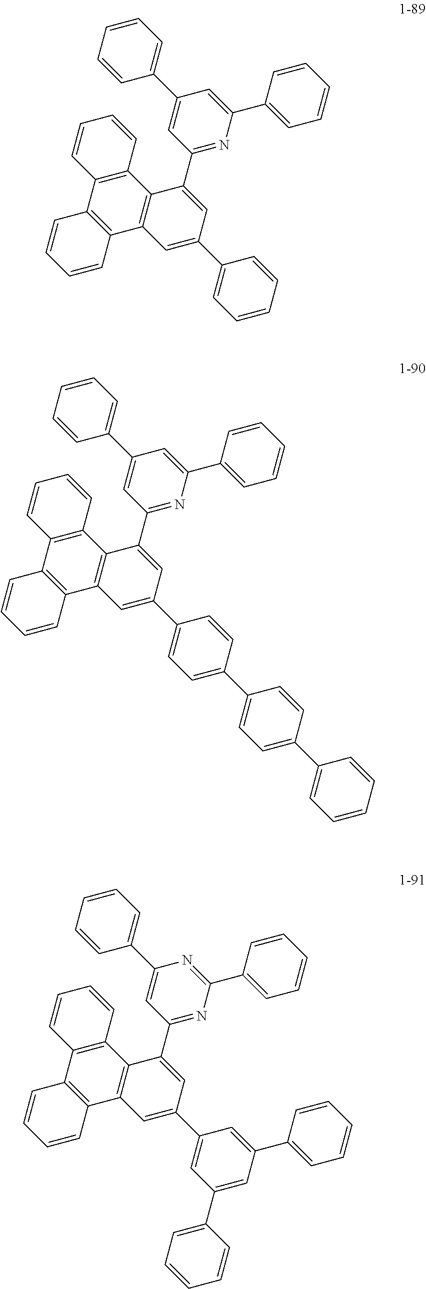 [SMILES: c1ccc(-c2cc(-c3ccccc3)nc(-c3cc(-c4ccccc4)cc4c5ccccc5c5ccccc5c34)c2)cc1 ; c1ccc(-c2ccc(-c3ccc(-c4cc(-c5cc(-c6ccccc6)cc(-c6ccccc6)n5)c5c6ccccc6c6ccccc6c5c4)cc3)cc2)cc1 ; c1ccc(-c2cc(-c3ccccc3)cc(-c3cc(-c4cc(-c5ccccc5)nc(-c5ccccc5)n4)c4c5ccccc5c5ccccc5c4c3)c2)cc1]

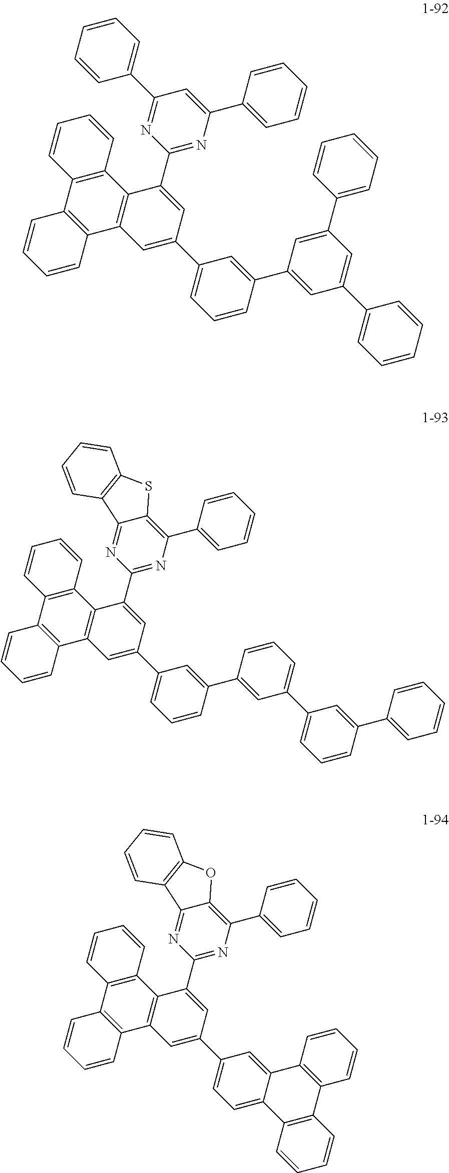 [SMILES: c1ccc(-c2cc(-c3ccccc3)cc(-c3cccc(-c4cc(-c5nc(-c6ccccc6)cc(-c6ccccc6)n5)c5c6ccccc6c6ccccc6c5c4)c3)c2)cc1 ; c1ccc(-c2cccc(-c3cccc(-c4cccc(-c5cc(-c6nc(-c7ccccc7)c7sc8ccccc8c7n6)c6c7ccccc7c7ccccc7c6c5)c4)c3)c2)cc1 ; c1ccc(-c2nc(-c3cc(-c4ccc5c6ccccc6c6ccccc6c5c4)cc4c5ccccc5c5ccccc5c34)nc3c2oc2ccccc23)cc1]

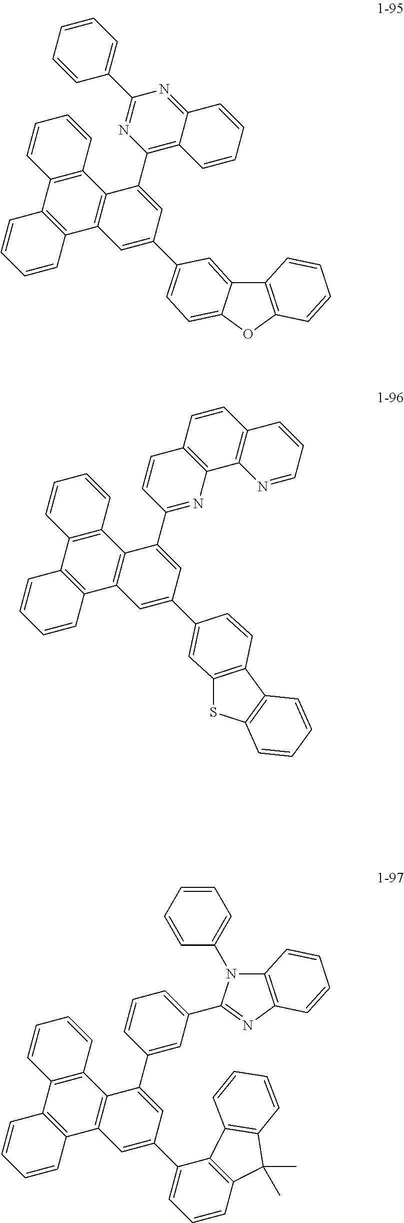 [SMILES: c1ccc(-c2nc(-c3cc(-c4ccc5oc6ccccc6c5c4)cc4c5ccccc5c5ccccc5c34)c3ccccc3n2)cc1 ; c1cnc2c(c1)ccc1ccc(-c3cc(-c4ccc5c(c4)sc4ccccc45)cc4c5ccccc5c5ccccc5c34)nc12 ; CC1(C)c2ccccc2-c2c(-c3cc(-c4cccc(-c5nc6ccccc6n5-c5ccccc5)c4)c4c5ccccc5c5ccccc5c4c3)cccc21]

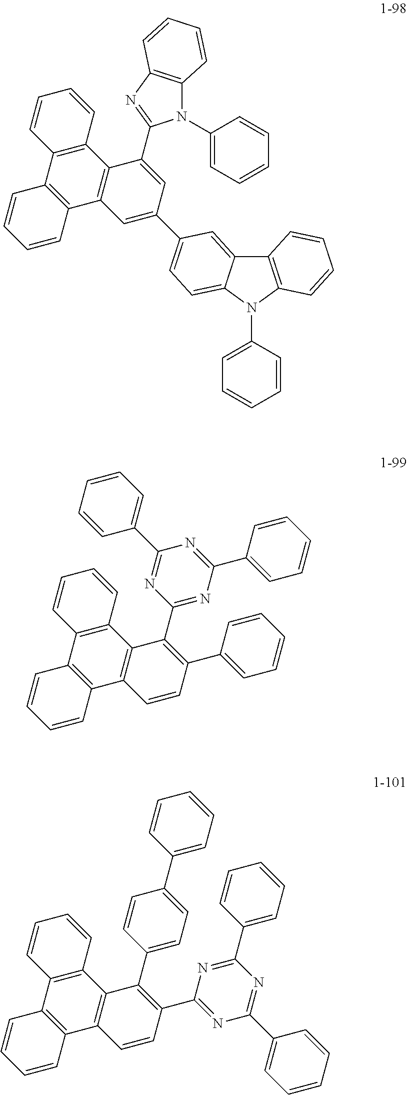 [SMILES: c1ccc(-n2c(-c3cc(-c4ccc5c(c4)c4ccccc4n5-c4ccccc4)cc4c5ccccc5c5ccccc5c34)nc3ccccc32)cc1 ; c1ccc(-c2nc(-c3ccccc3)nc(-c3c(-c4ccccc4)ccc4c5ccccc5c5ccccc5c34)n2)cc1 ; c1ccc(-c2ccc(-c3c(-c4nc(-c5ccccc5)nc(-c5ccccc5)n4)ccc4c5ccccc5c5ccccc5c34)cc2)cc1]

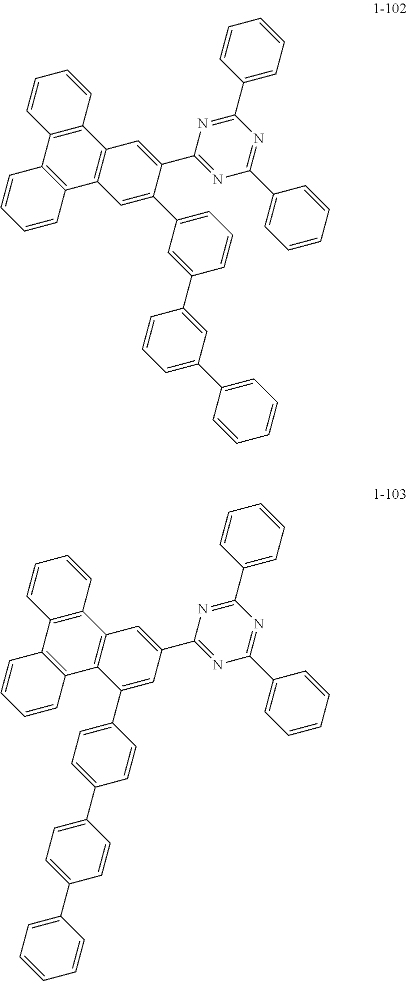 [SMILES: c1ccc(-c2cccc(-c3cccc(-c4cc5c6ccccc6c6ccccc6c5cc4-c4nc(-c5ccccc5)nc(-c5ccccc5)n4)c3)c2)cc1 ; c1ccc(-c2ccc(-c3ccc(-c4cc(-c5nc(-c6ccccc6)nc(-c6ccccc6)n5)cc5c6ccccc6c6ccccc6c45)cc3)cc2)cc1]

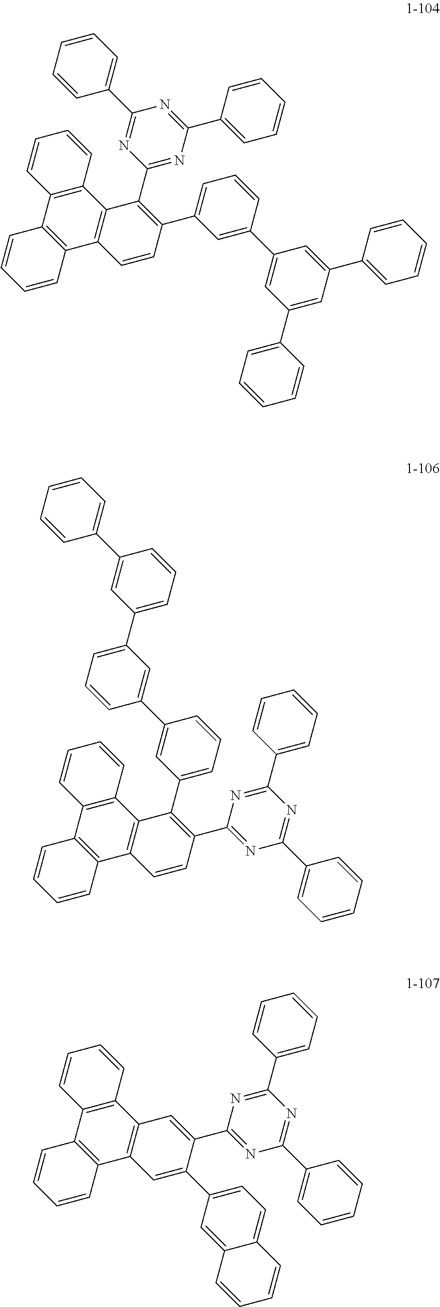 [SMILES: c1ccc(-c2cc(-c3ccccc3)cc(-c3cccc(-c4ccc5c6ccccc6c6ccccc6c5c4-c4nc(-c5ccccc5)nc(-c5ccccc5)n4)c3)c2)cc1 ; c1ccc(-c2cccc(-c3cccc(-c4cccc(-c5c(-c6nc(-c7ccccc7)nc(-c7ccccc7)n6)ccc6c7ccccc7c7ccccc7c56)c4)c3)c2)cc1 ; c1ccc(-c2nc(-c3ccccc3)nc(-c3cc4c5ccccc5c5ccccc5c4cc3-c3ccc4ccccc4c3)n2)cc1]

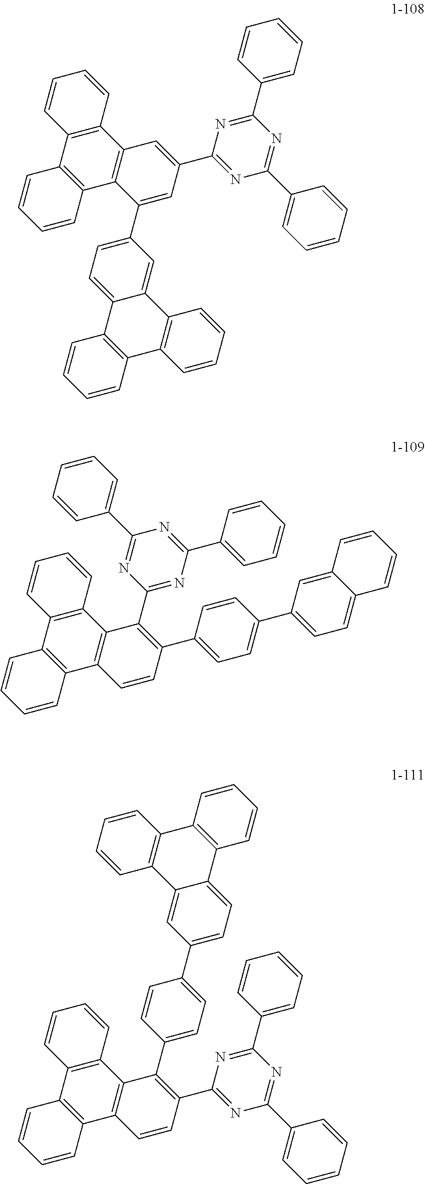 [SMILES: c1ccc(-c2nc(-c3ccccc3)nc(-c3cc(-c4ccc5c6ccccc6c6ccccc6c5c4)c4c5ccccc5c5ccccc5c4c3)n2)cc1 ; c1ccc(-c2nc(-c3ccccc3)nc(-c3c(-c4ccc(-c5ccc6ccccc6c5)cc4)ccc4c5ccccc5c5ccccc5c34)n2)cc1 ; c1ccc(-c2nc(-c3ccccc3)nc(-c3ccc4c5ccccc5c5ccccc5c4c3-c3ccc(-c4ccc5c6ccccc6c6ccccc6c5c4)cc3)n2)cc1]

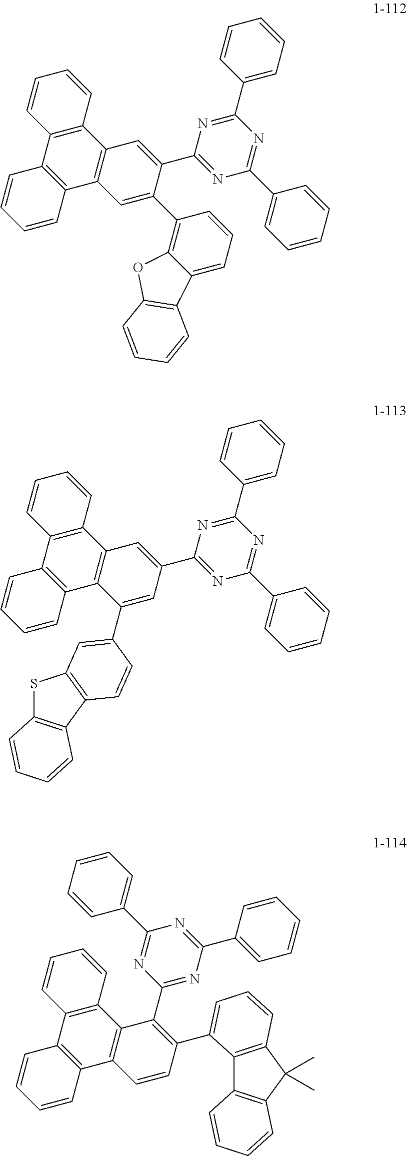 [SMILES: c1ccc(-c2nc(-c3ccccc3)nc(-c3cc4c5ccccc5c5ccccc5c4cc3-c3cccc4c3oc3ccccc34)n2)cc1 ; c1ccc(-c2nc(-c3ccccc3)nc(-c3cc(-c4ccc5c(c4)sc4ccccc45)c4c5ccccc5c5ccccc5c4c3)n2)cc1 ; CC1(C)c2ccccc2-c2c(-c3ccc4c5ccccc5c5ccccc5c4c3-c3nc(-c4ccccc4)nc(-c4ccccc4)n3)cccc21]

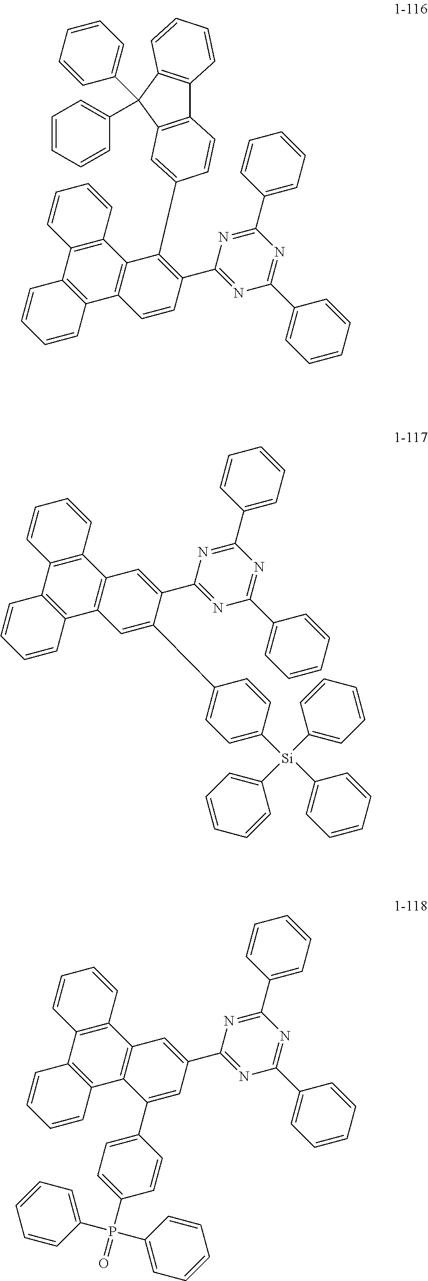 [SMILES: c1ccc(-c2nc(-c3ccccc3)nc(-c3ccc4c5ccccc5c5ccccc5c4c3-c3ccc4c(c3)C(c3ccccc3)(c3ccccc3)c3ccccc3-4)n2)cc1 ; c1ccc(-c2nc(-c3ccccc3)nc(-c3cc4c5ccccc5c5ccccc5c4cc3-c3ccc([Si](c4ccccc4)(c4ccccc4)c4ccccc4)cc3)n2)cc1 ; O=P(c1ccccc1)(c1ccccc1)c1ccc(-c2cc(-c3nc(-c4ccccc4)nc(-c4ccccc4)n3)cc3c4ccccc4c4ccccc4c23)cc1]

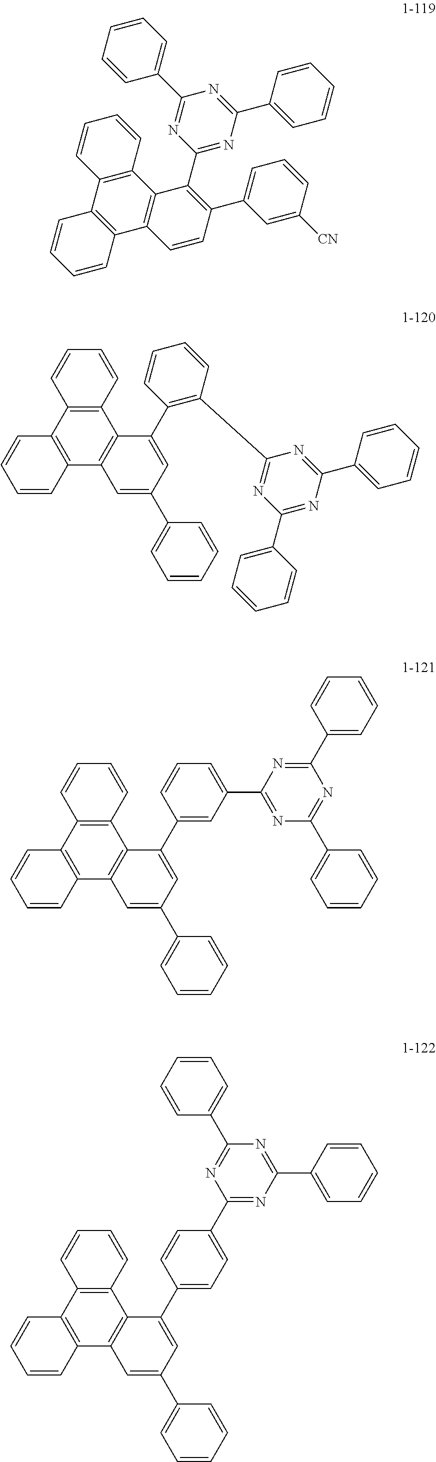 [SMILES: N#Cc1cccc(-c2ccc3c4ccccc4c4ccccc4c3c2-c2nc(-c3ccccc3)nc(-c3ccccc3)n2)c1 ; c1ccc(-c2cc(-c3ccccc3-c3nc(-c4ccccc4)nc(-c4ccccc4)n3)c3c4ccccc4c4ccccc4c3c2)cc1 ; c1ccc(-c2cc(-c3cccc(-c4nc(-c5ccccc5)nc(-c5ccccc5)n4)c3)c3c4ccccc4c4ccccc4c3c2)cc1 ; c1ccc(-c2cc(-c3ccc(-c4nc(-c5ccccc5)nc(-c5ccccc5)n4)cc3)c3c4ccccc4c4ccccc4c3c2)cc1]

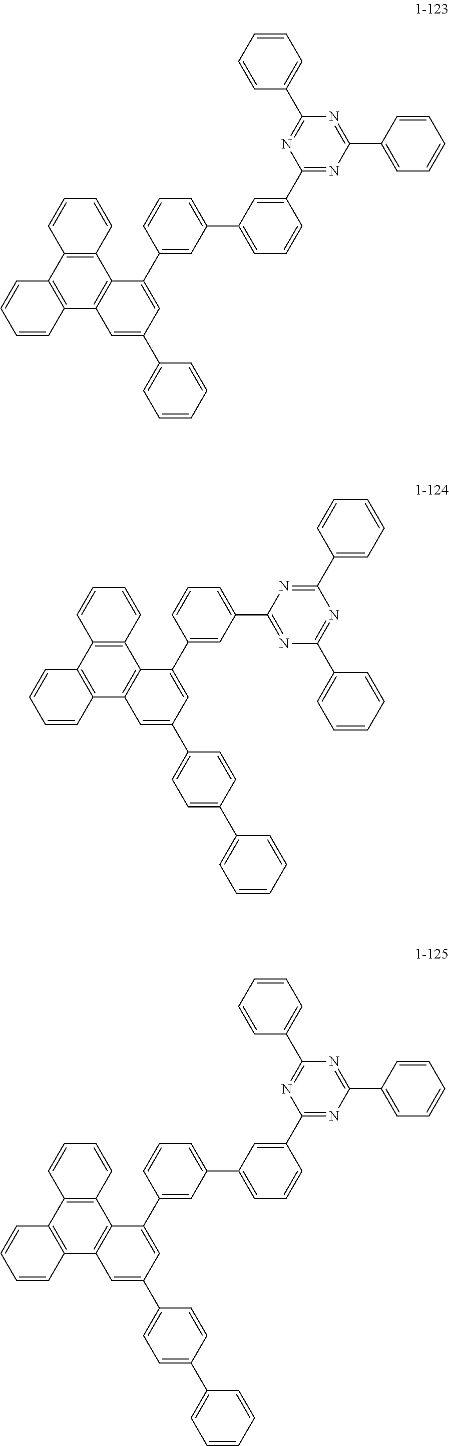 [SMILES: c1ccc(-c2cc(-c3cccc(-c4cccc(-c5nc(-c6ccccc6)nc(-c6ccccc6)n5)c4)c3)c3c4ccccc4c4ccccc4c3c2)cc1 ; c1ccc(-c2ccc(-c3cc(-c4cccc(-c5nc(-c6ccccc6)nc(-c6ccccc6)n5)c4)c4c5ccccc5c5ccccc5c4c3)cc2)cc1 ; c1ccc(-c2ccc(-c3cc(-c4cccc(-c5cccc(-c6nc(-c7ccccc7)nc(-c7ccccc7)n6)c5)c4)c4c5ccccc5c5ccccc5c4c3)cc2)cc1]

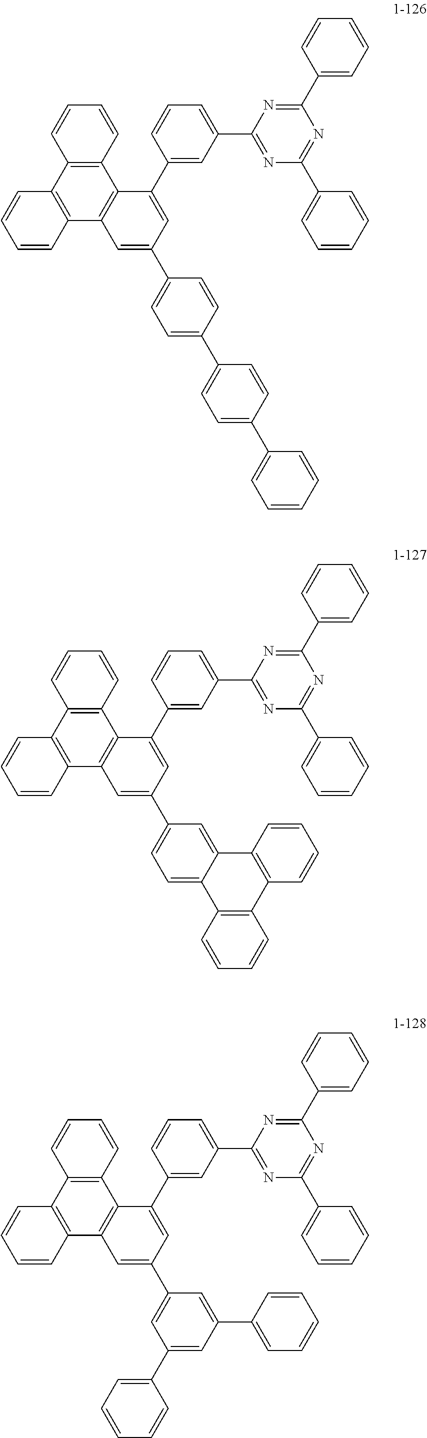 [SMILES: c1ccc(-c2ccc(-c3ccc(-c4cc(-c5cccc(-c6nc(-c7ccccc7)nc(-c7ccccc7)n6)c5)c5c6ccccc6c6ccccc6c5c4)cc3)cc2)cc1 ; c1ccc(-c2nc(-c3ccccc3)nc(-c3cccc(-c4cc(-c5ccc6c7ccccc7c7ccccc7c6c5)cc5c6ccccc6c6ccccc6c45)c3)n2)cc1 ; c1ccc(-c2cc(-c3ccccc3)cc(-c3cc(-c4cccc(-c5nc(-c6ccccc6)nc(-c6ccccc6)n5)c4)c4c5ccccc5c5ccccc5c4c3)c2)cc1]

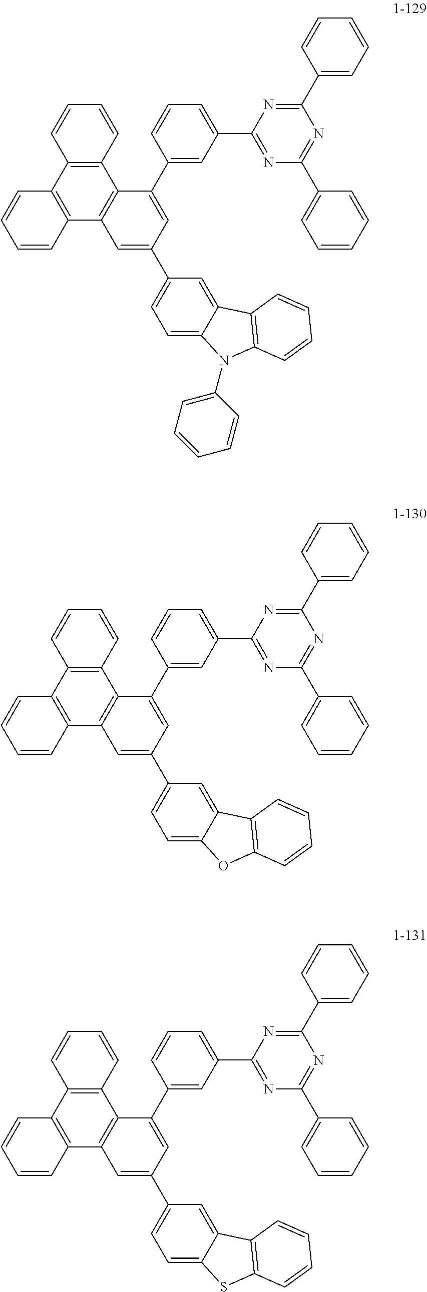 [SMILES: c1ccc(-c2nc(-c3ccccc3)nc(-c3cccc(-c4cc(-c5ccc6c(c5)c5ccccc5n6-c5ccccc5)cc5c6ccccc6c6ccccc6c45)c3)n2)cc1 ; c1ccc(-c2nc(-c3ccccc3)nc(-c3cccc(-c4cc(-c5ccc6oc7ccccc7c6c5)cc5c6ccccc6c6ccccc6c45)c3)n2)cc1 ; c1ccc(-c2nc(-c3ccccc3)nc(-c3cccc(-c4cc(-c5ccc6sc7ccccc7c6c5)cc5c6ccccc6c6ccccc6c45)c3)n2)cc1]

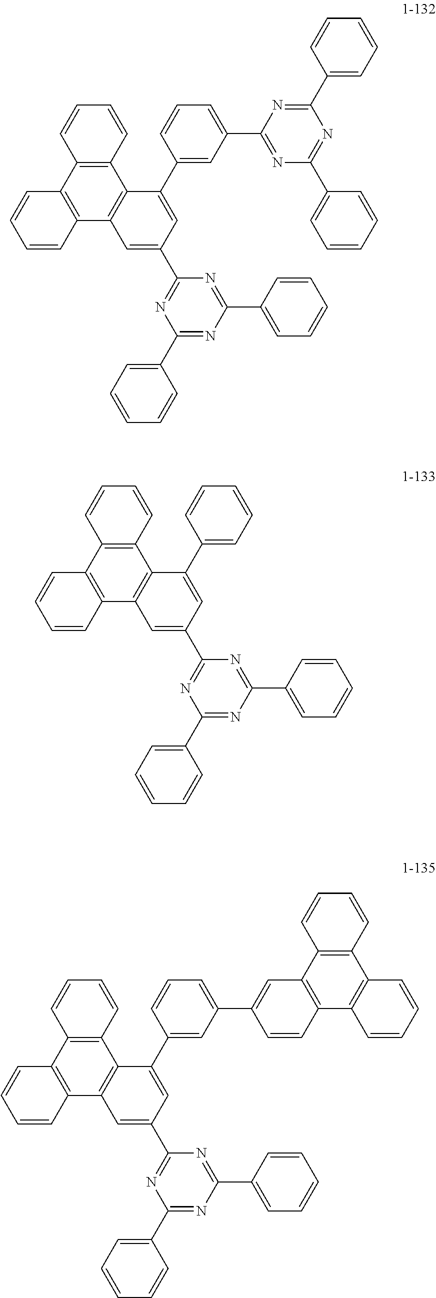 [SMILES: c1ccc(-c2nc(-c3ccccc3)nc(-c3cccc(-c4cc(-c5nc(-c6ccccc6)nc(-c6ccccc6)n5)cc5c6ccccc6c6ccccc6c45)c3)n2)cc1 ; c1ccc(-c2nc(-c3ccccc3)nc(-c3cc(-c4ccccc4)c4c5ccccc5c5ccccc5c4c3)n2)cc1 ; c1ccc(-c2nc(-c3ccccc3)nc(-c3cc(-c4cccc(-c5ccc6c7ccccc7c7ccccc7c6c5)c4)c4c5ccccc5c5ccccc5c4c3)n2)cc1]

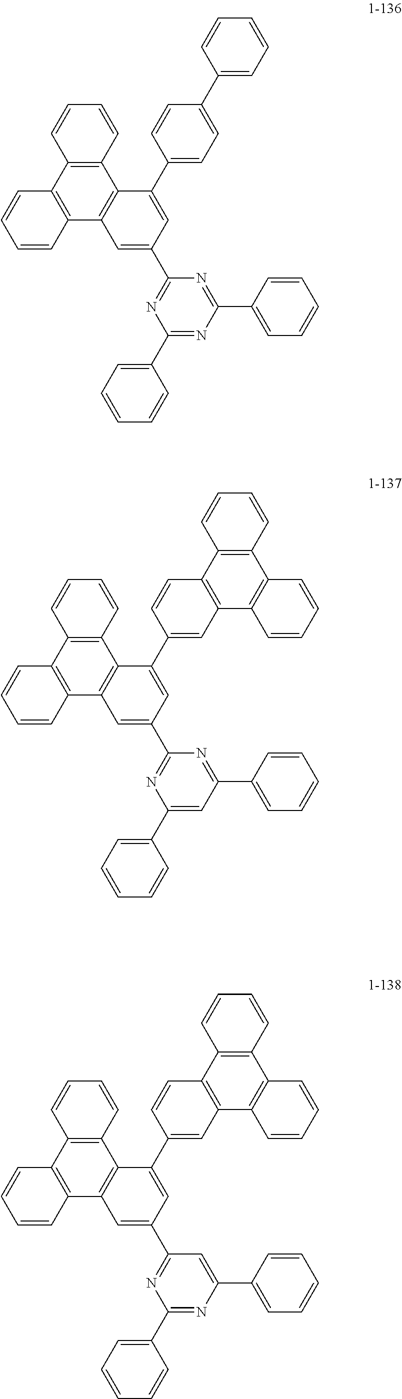 [SMILES: c1ccc(-c2ccc(-c3cc(-c4nc(-c5ccccc5)nc(-c5ccccc5)n4)cc4c5ccccc5c5ccccc5c34)cc2)cc1 ; c1ccc(-c2cc(-c3ccccc3)nc(-c3cc(-c4ccc5c6ccccc6c6ccccc6c5c4)c4c5ccccc5c5ccccc5c4c3)n2)cc1 ; c1ccc(-c2cc(-c3cc(-c4ccc5c6ccccc6c6ccccc6c5c4)c4c5ccccc5c5ccccc5c4c3)nc(-c3ccccc3)n2)cc1]

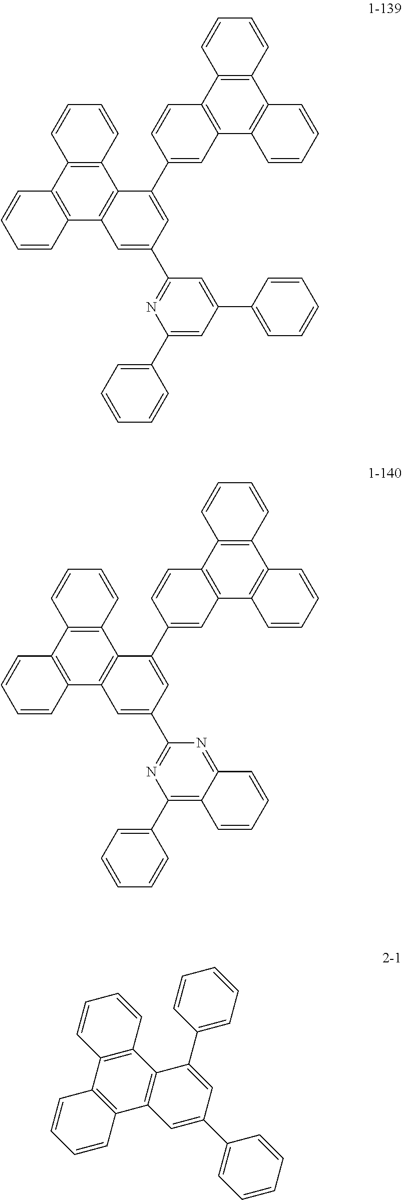

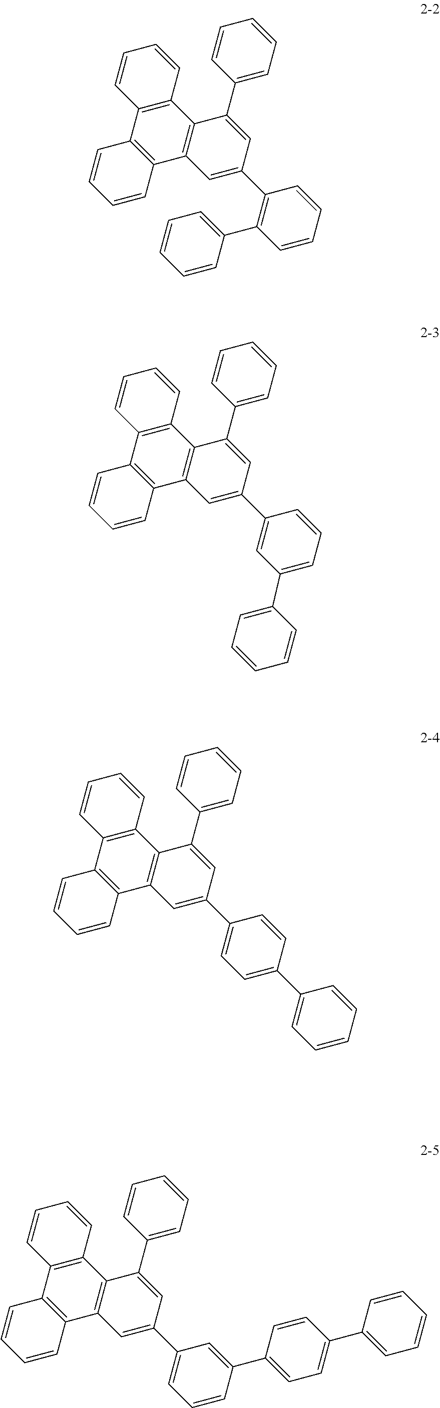

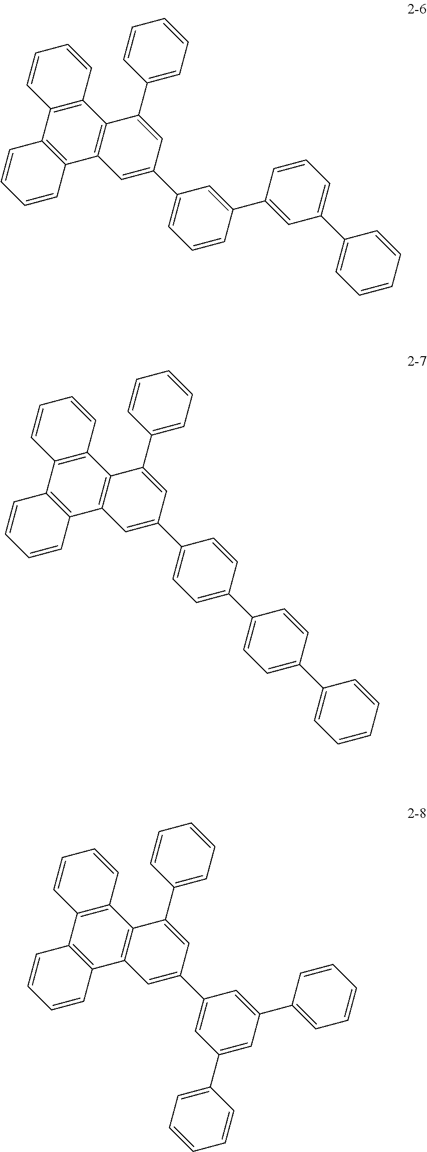

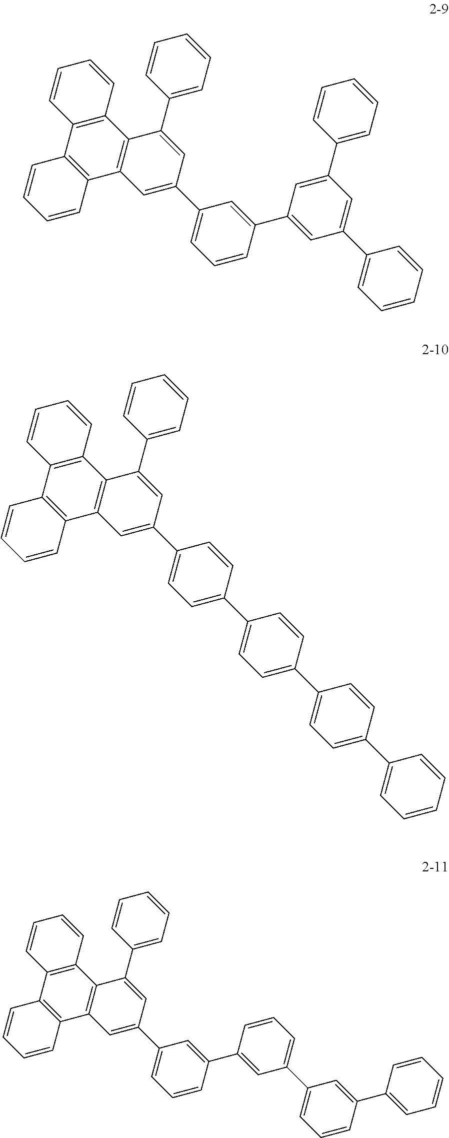

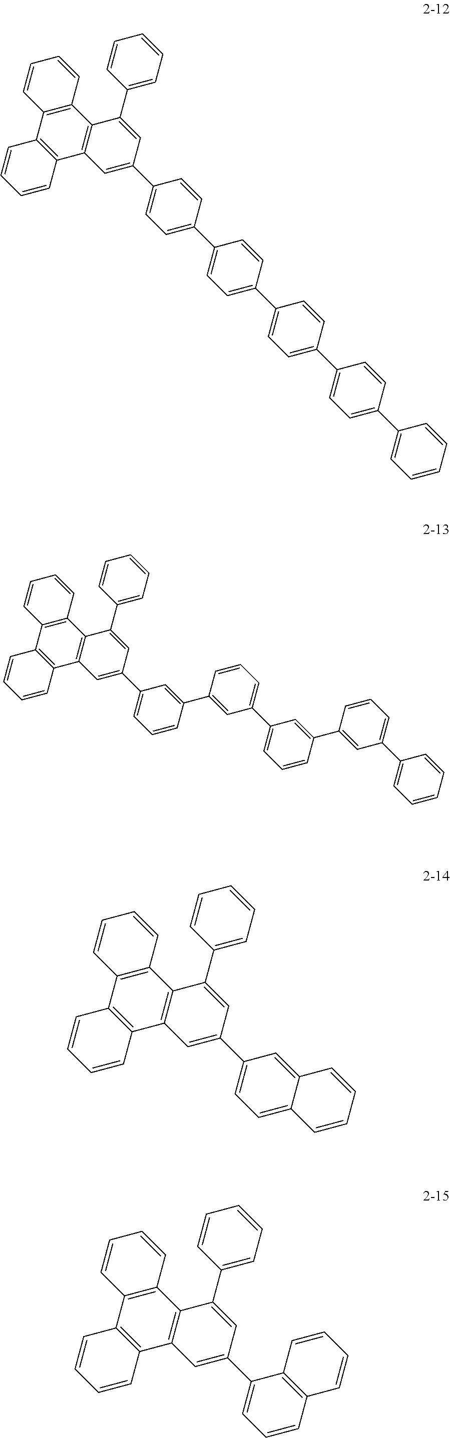

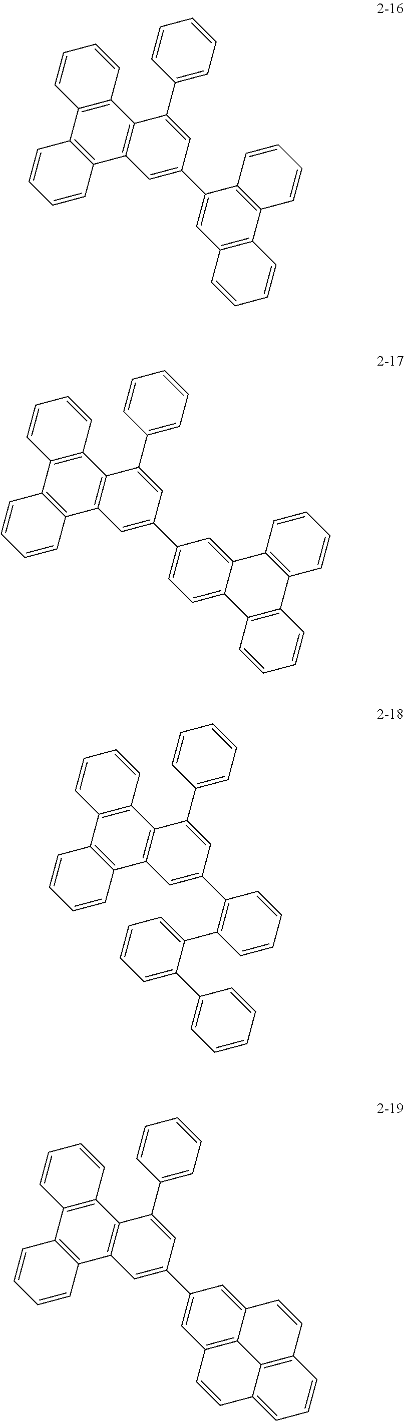

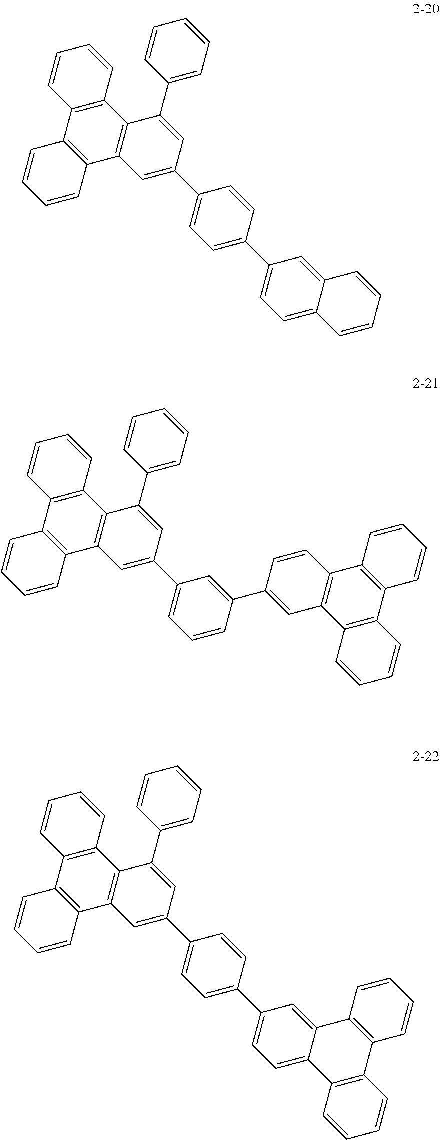

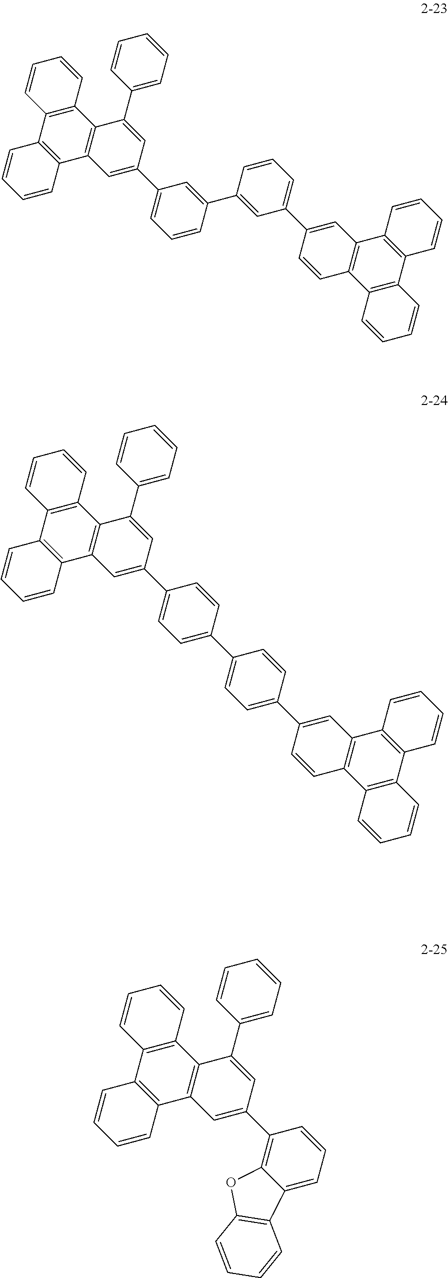

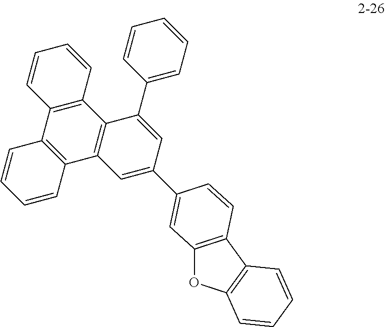 [SMILES: c1ccc(-c2cc(-c3ccc4c(c3)oc3ccccc34)cc3c4ccccc4c4ccccc4c23)cc1]

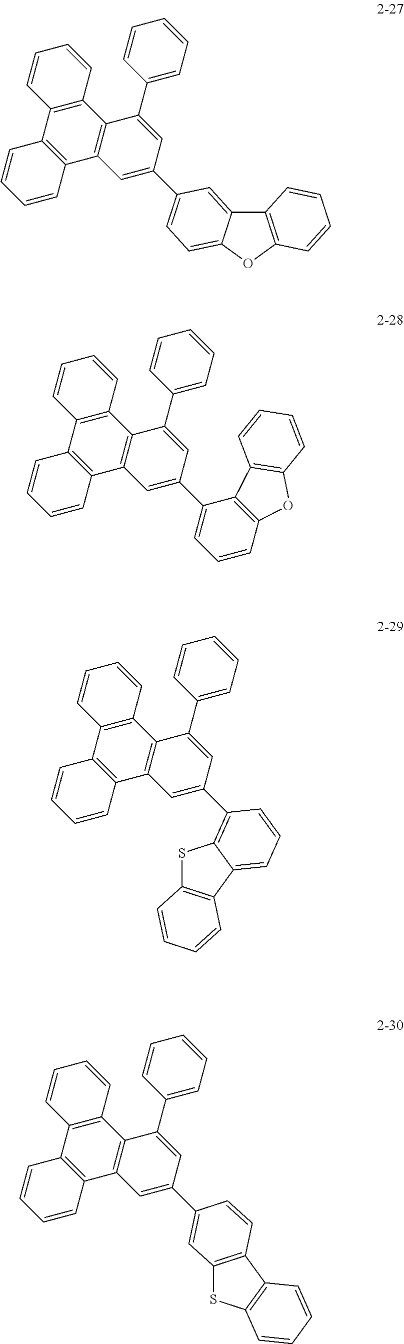 [SMILES: c1ccc(-c2cc(-c3ccc4oc5ccccc5c4c3)cc3c4ccccc4c4ccccc4c23)cc1 ; c1ccc(-c2cc(-c3cccc4oc5ccccc5c34)cc3c4ccccc4c4ccccc4c23)cc1 ; c1ccc(-c2cc(-c3cccc4c3sc3ccccc34)cc3c4ccccc4c4ccccc4c23)cc1 ; c1ccc(-c2cc(-c3ccc4c(c3)sc3ccccc34)cc3c4ccccc4c4ccccc4c23)cc1]

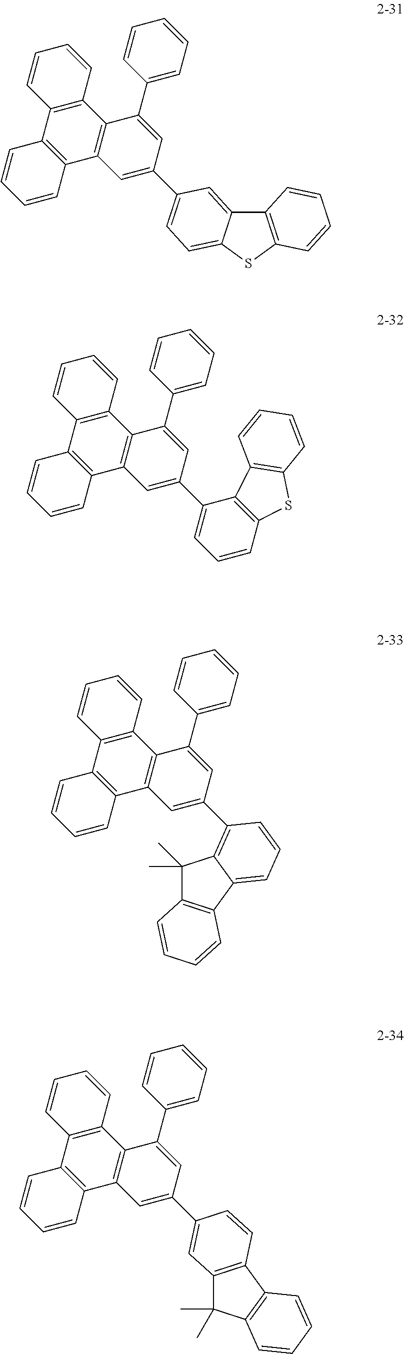

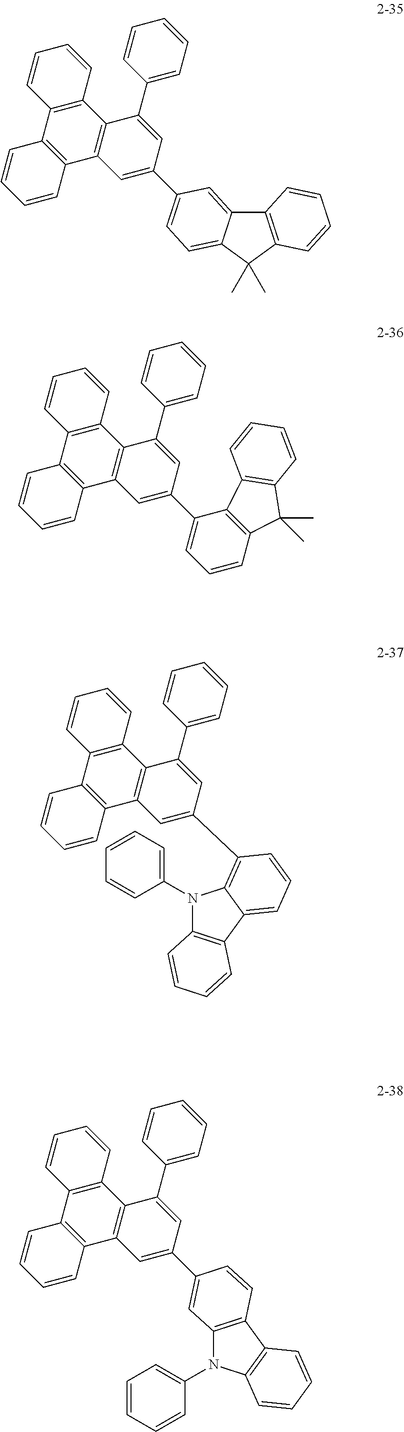

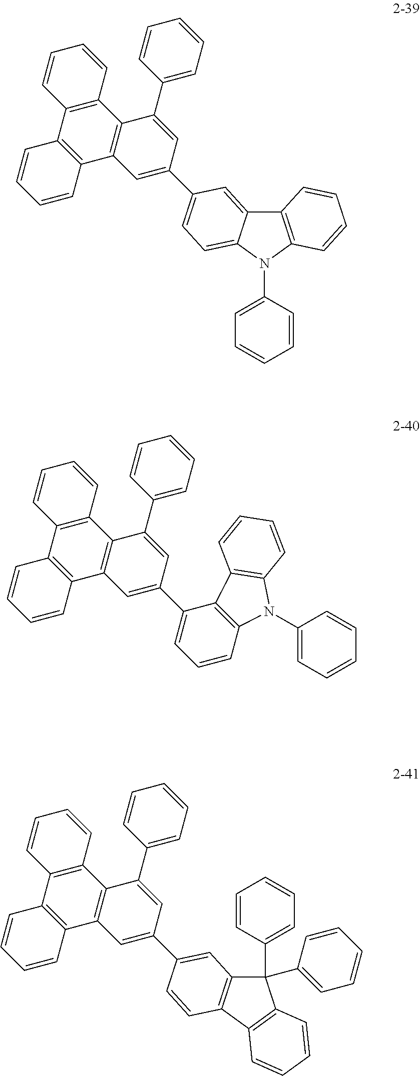

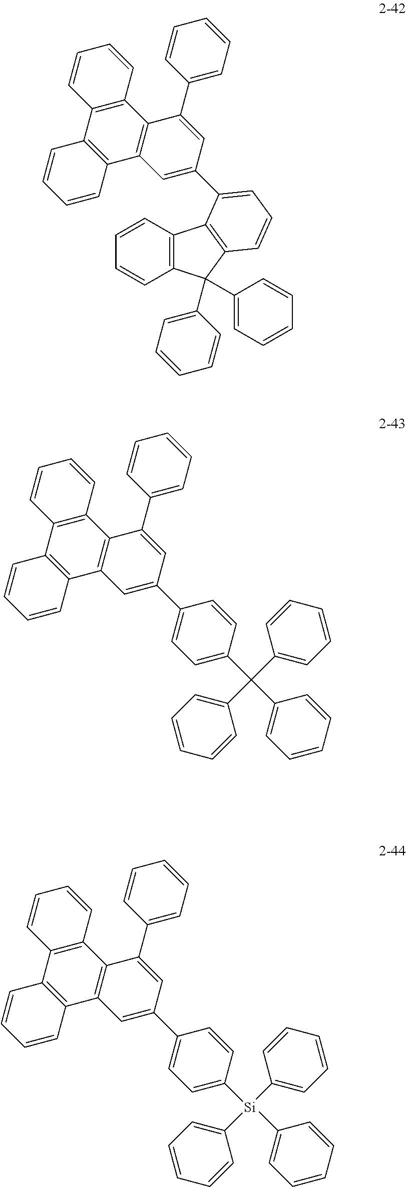

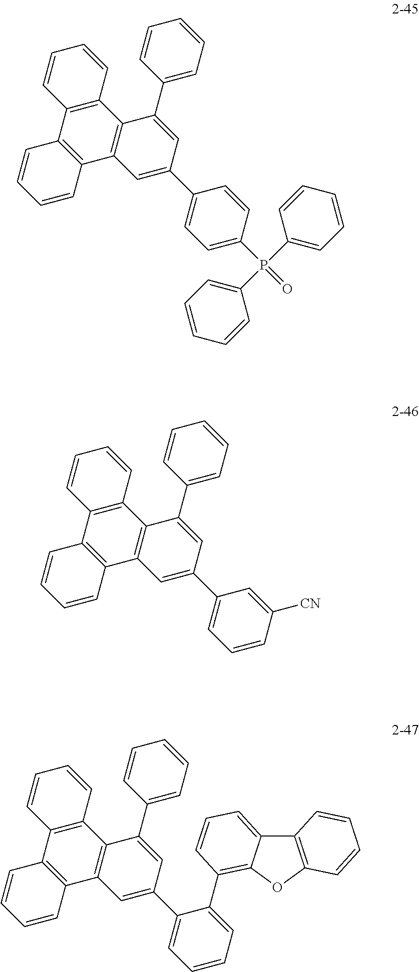 [SMILES: O=P(c1ccccc1)(c1ccccc1)c1ccc(-c2cc(-c3ccccc3)c3c4ccccc4c4ccccc4c3c2)cc1 ; N#Cc1cccc(-c2cc(-c3ccccc3)c3c4ccccc4c4ccccc4c3c2)c1 ; c1ccc(-c2cc(-c3ccccc3-c3cccc4c3oc3ccccc34)cc3c4ccccc4c4ccccc4c23)cc1]

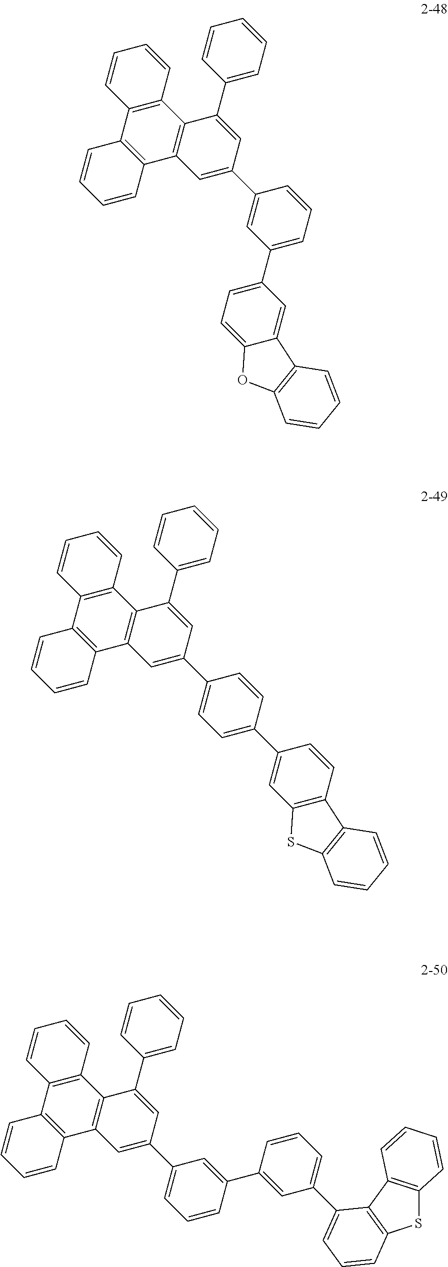 [SMILES: c1ccc(-c2cc(-c3cccc(-c4ccc5oc6ccccc6c5c4)c3)cc3c4ccccc4c4ccccc4c23)cc1 ; c1ccc(-c2cc(-c3ccc(-c4ccc5c(c4)sc4ccccc45)cc3)cc3c4ccccc4c4ccccc4c23)cc1 ; c1ccc(-c2cc(-c3cccc(-c4cccc(-c5cccc6sc7ccccc7c56)c4)c3)cc3c4ccccc4c4ccccc4c23)cc1]

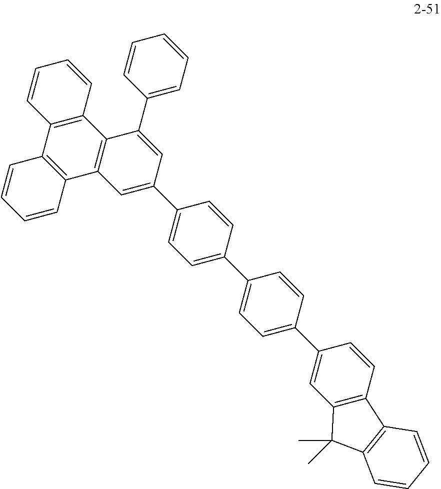 [SMILES: CC1(C)c2ccccc2-c2ccc(-c3ccc(-c4ccc(-c5cc(-c6ccccc6)c6c7ccccc7c7ccccc7c6c5)cc4)cc3)cc21]

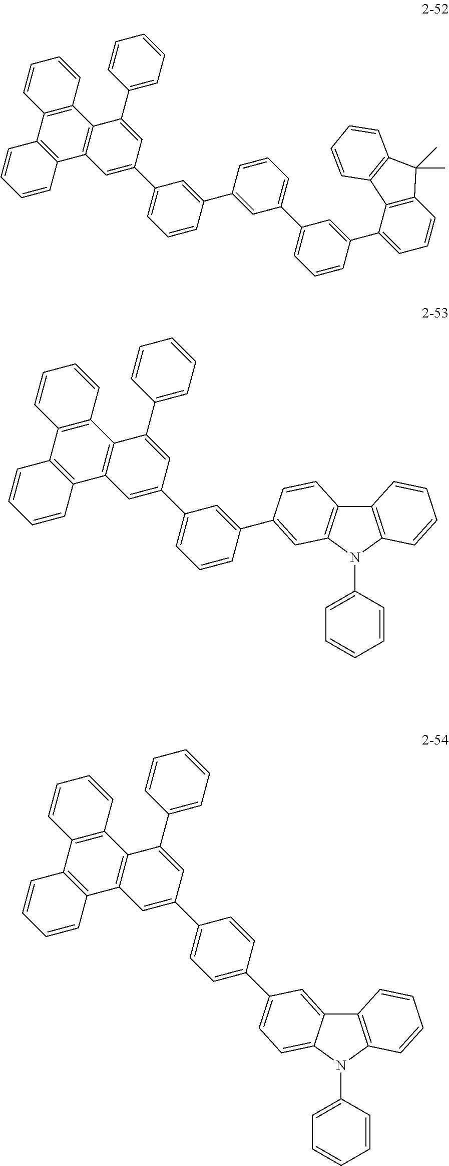

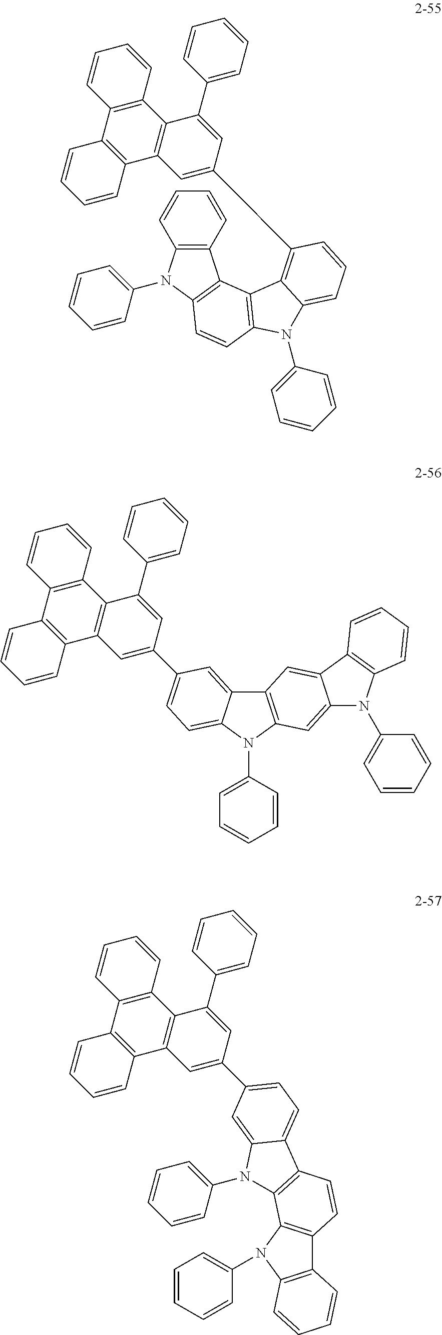 [SMILES: c1ccc(-c2cc(-c3cccc4c3c3c5c6ccccc6n(-c6ccccc6)c5ccc3n4-c3ccccc3)cc3c4ccccc4c4ccccc4c23)cc1 ; c1ccc(-c2cc(-c3ccc4c(c3)c3cc5c6ccccc6n(-c6ccccc6)c5cc3n4-c3ccccc3)cc3c4ccccc4c4ccccc4c23)cc1 ; c1ccc(-c2cc(-c3ccc4c5ccc6c7ccccc7n(-c7ccccc7)c6c5n(-c5ccccc5)c4c3)cc3c4ccccc4c4ccccc4c23)cc1]

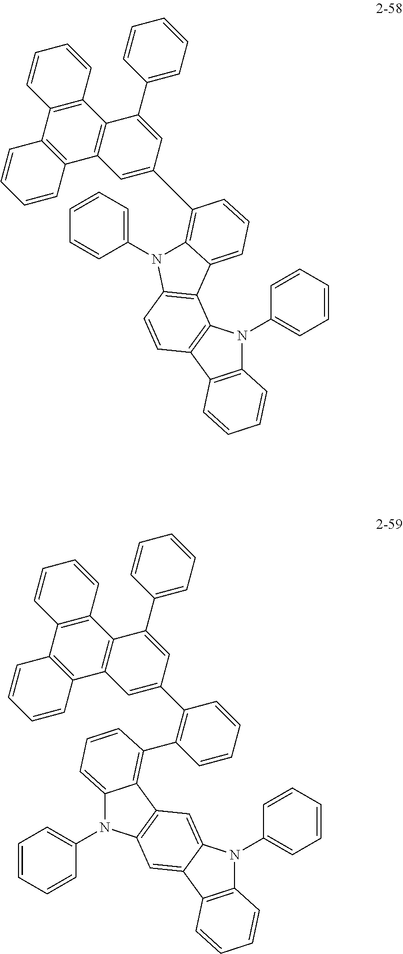 [SMILES: c1ccc(-c2cc(-c3cccc4c5c(ccc6c7ccccc7n(-c7ccccc7)c65)n(-c5ccccc5)c34)cc3c4ccccc4c4ccccc4c23)cc1 ; c1ccc(-c2cc(-c3ccccc3-c3cccc4c3c3cc5c(cc3n4-c3ccccc3)c3ccccc3n5-c3ccccc3)cc3c4ccccc4c4ccccc4c23)cc1]

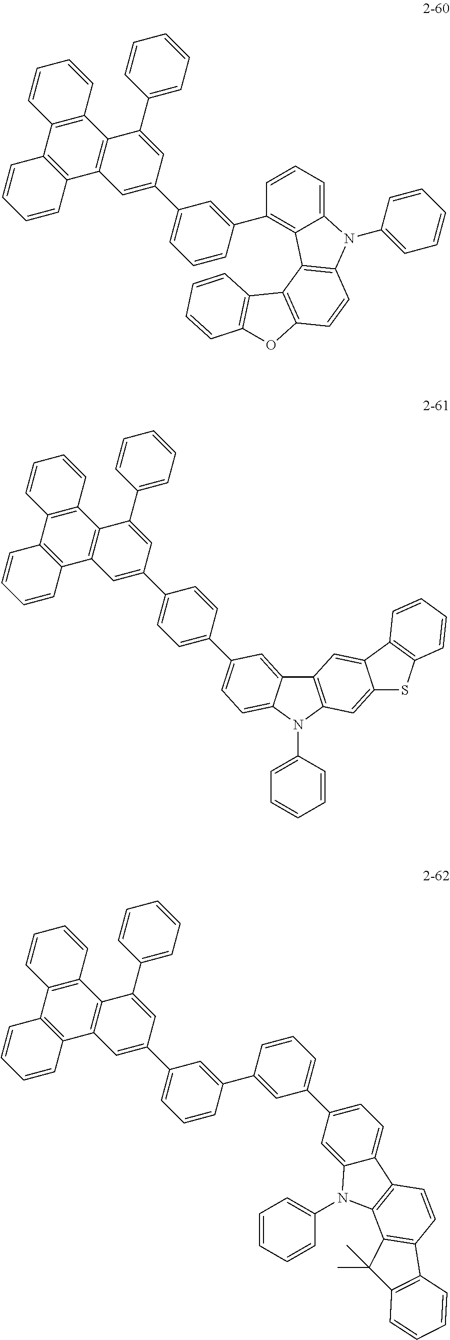 [SMILES: c1ccc(-c2cc(-c3cccc(-c4cccc5c4c4c6c(ccc4n5-c4ccccc4)oc4ccccc46)c3)cc3c4ccccc4c4ccccc4c23)cc1 ; c1ccc(-c2cc(-c3ccc(-c4ccc5c(c4)c4cc6c(cc4n5-c4ccccc4)sc4ccccc46)cc3)cc3c4ccccc4c4ccccc4c23)cc1 ; CC1(C)c2ccccc2-c2ccc3c4ccc(-c5cccc(-c6cccc(-c7cc(-c8ccccc8)c8c9ccccc9c9ccccc9c8c7)c6)c5)cc4n(-c4ccccc4)c3c21]

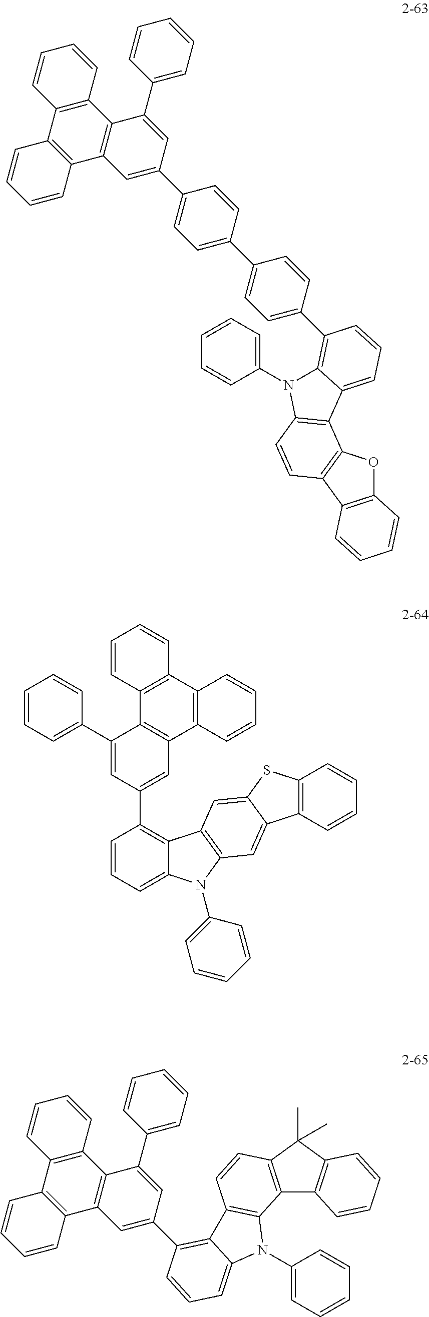 [SMILES: c1ccc(-c2cc(-c3ccc(-c4ccc(-c5cccc6c7c8oc9ccccc9c8ccc7n(-c7ccccc7)c56)cc4)cc3)cc3c4ccccc4c4ccccc4c23)cc1 ; c1ccc(-c2cc(-c3cccc4c3c3cc5sc6ccccc6c5cc3n4-c3ccccc3)cc3c4ccccc4c4ccccc4c23)cc1 ; CC1(C)c2ccccc2-c2c1ccc1c3c(-c4cc(-c5ccccc5)c5c6ccccc6c6ccccc6c5c4)cccc3n(-c3ccccc3)c21]

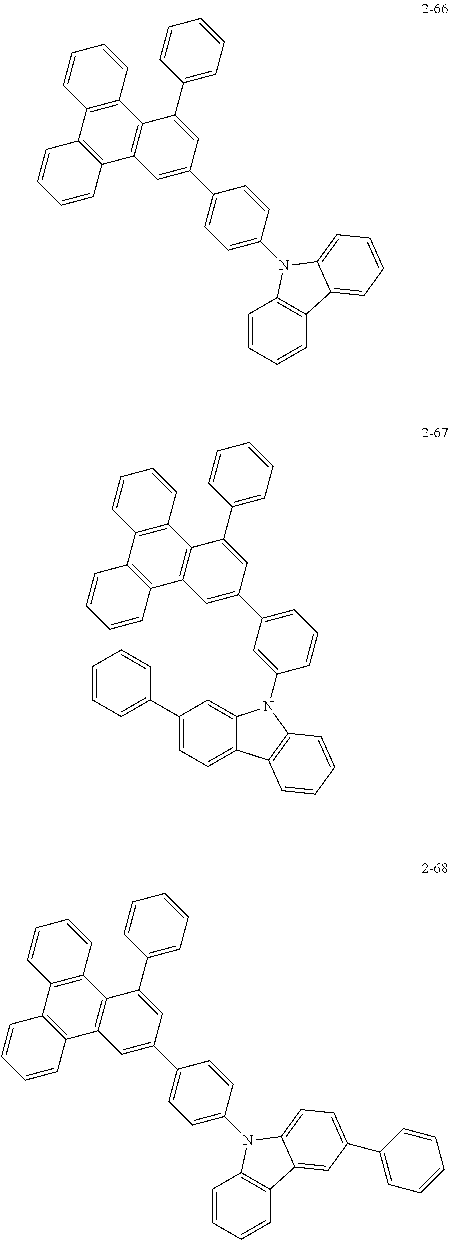 [SMILES: c1ccc(-c2cc(-c3ccc(-n4c5ccccc5c5ccccc54)cc3)cc3c4ccccc4c4ccccc4c23)cc1 ; c1ccc(-c2ccc3c4ccccc4n(-c4cccc(-c5cc(-c6ccccc6)c6c7ccccc7c7ccccc7c6c5)c4)c3c2)cc1 ; c1ccc(-c2ccc3c(c2)c2ccccc2n3-c2ccc(-c3cc(-c4ccccc4)c4c5ccccc5c5ccccc5c4c3)cc2)cc1]

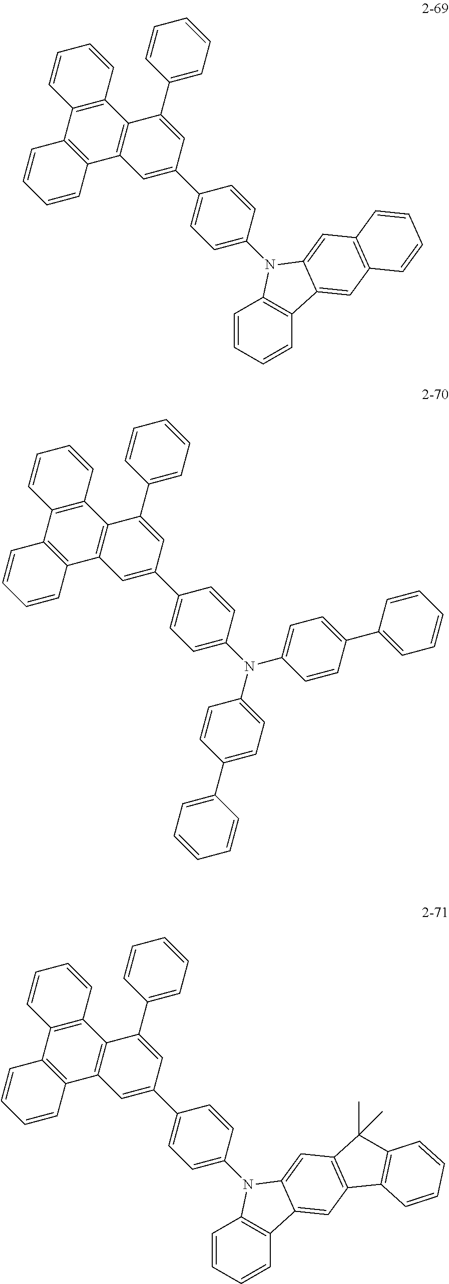 [SMILES: c1ccc(-c2cc(-c3ccc(-n4c5ccccc5c5cc6ccccc6cc54)cc3)cc3c4ccccc4c4ccccc4c23)cc1 ; c1ccc(-c2ccc(N(c3ccc(-c4ccccc4)cc3)c3ccc(-c4cc(-c5ccccc5)c5c6ccccc6c6ccccc6c5c4)cc3)cc2)cc1 ; CC1(C)c2ccccc2-c2cc3c4ccccc4n(-c4ccc(-c5cc(-c6ccccc6)c6c7ccccc7c7ccccc7c6c5)cc4)c3cc21]

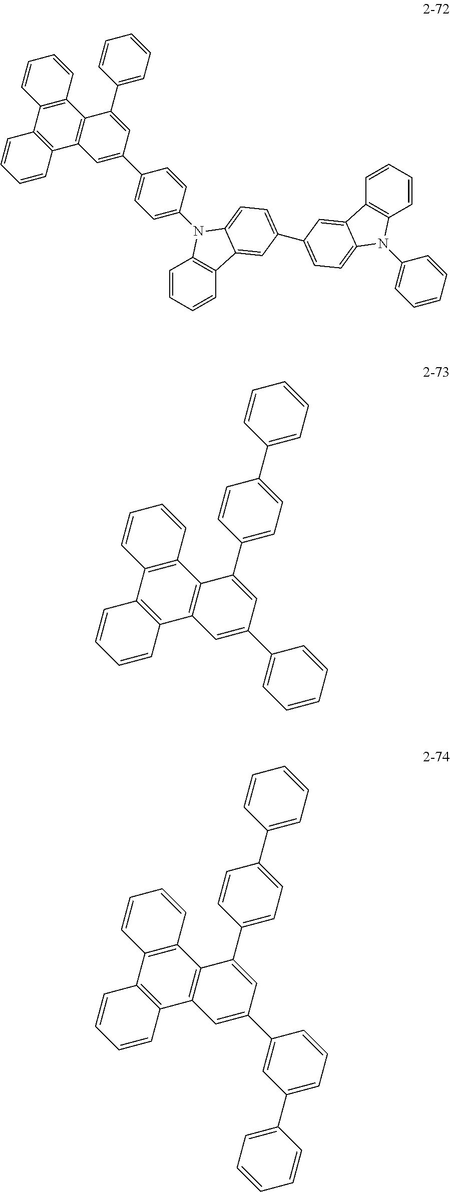

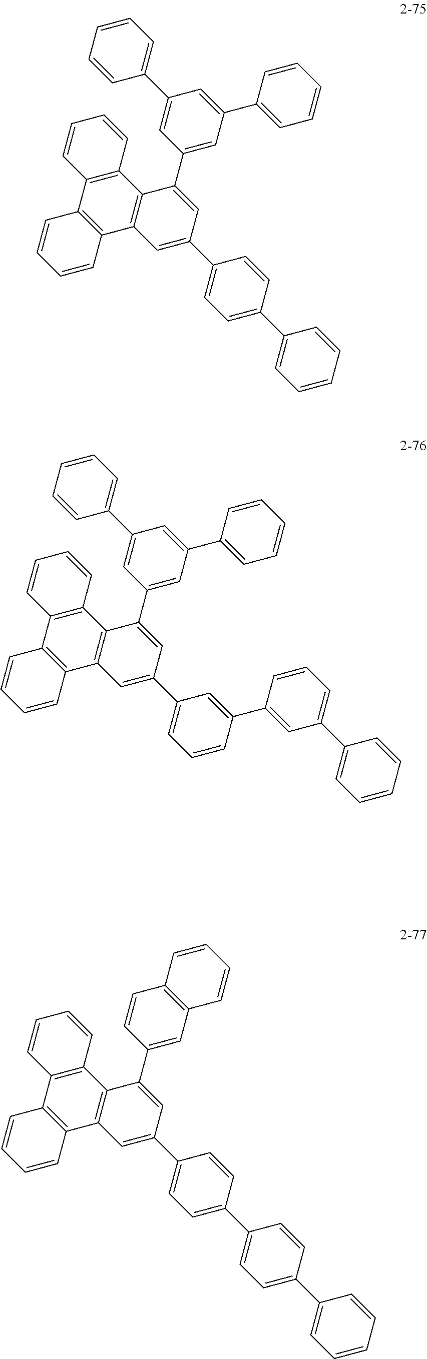

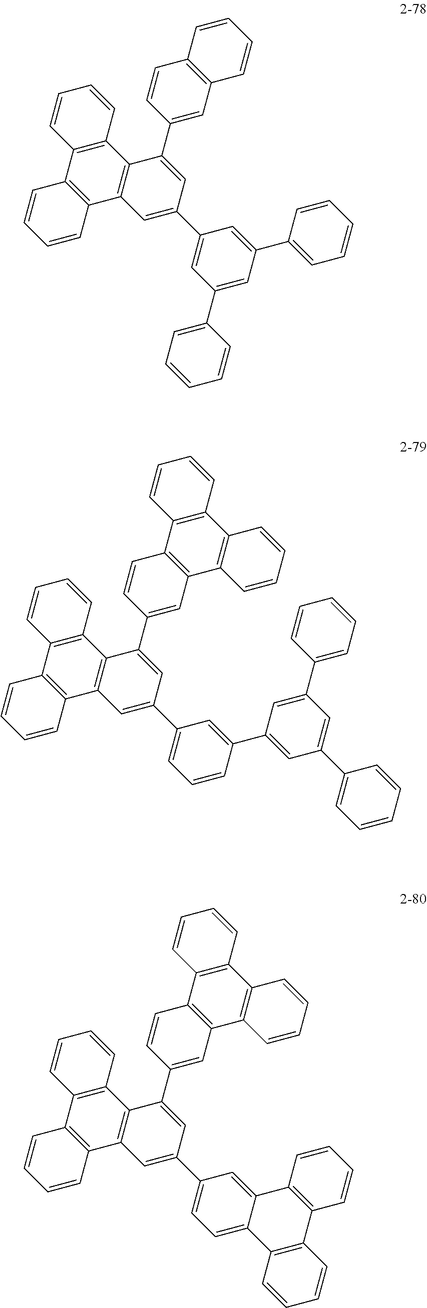

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

One embodiment of the present specification provides an organic light emitting device including a first electrode; a second electrode; and one or more organic material layers provided between the first electrode and the second electrode, wherein one or more layers of the organic material layers include one or more types of the compound represented by Chemical Formula 1.

In one embodiment of the present specification, one or more layers of the organic material layers include one type of the compound represented by Chemical Formula 1.

In another embodiment, one or more layers of the organic material layers include two types of the compound represented by Chemical Formula 1

In one embodiment of the present specification, the organic material layer further includes a compound represented by the following Chemical Formula 2.

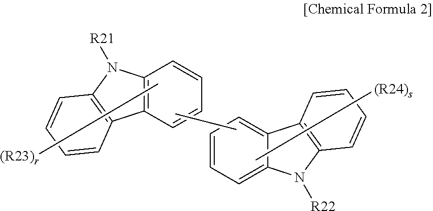

In Chemical Formula 2,

R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,

R23 and R24 are each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,

r and s are each an integer of 0 to 7, and

when r and s are each 2 or greater, substituents in the parentheses are the same as or different from each other.

In one embodiment of the present specification, Chemical Formula 2 may be represented by any one of the following Chemical Formulae 2-1 to 2-4.

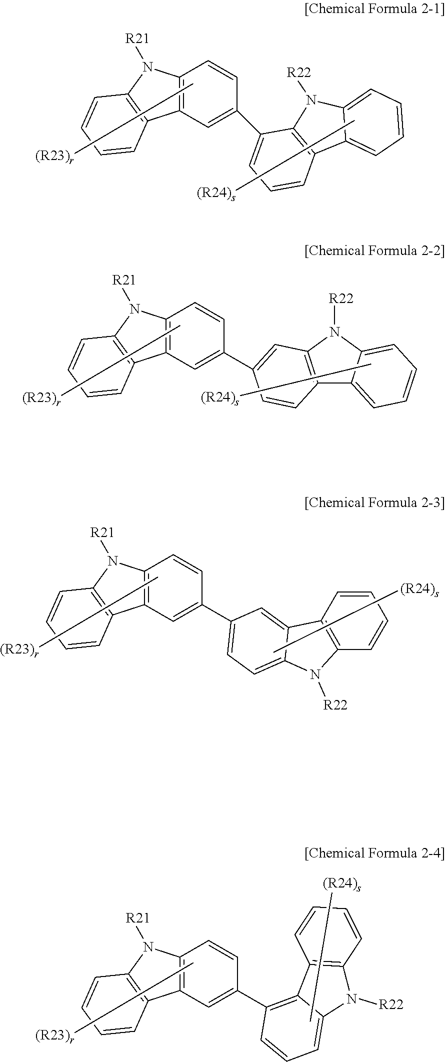

In Chemical Formulae 2-1 to 2-4,

each substituent has the same definition as in Chemical Formula 2.

In one embodiment of the present specification, R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group.

In one embodiment of the present specification, R21 and R22 are each independently a substituted or unsubstituted C1 to C40 alkyl group; a substituted or unsubstituted C3 to C40 cycloalkyl group; a substituted or unsubstituted C6 to C40 aryl group; or a substituted or unsubstituted C2 to C40 heteroaryl group.

In one embodiment of the present specification, R21 and R22 are each independently a substituted or unsubstituted phenyl group; a substituted or unsubstituted biphenyl group; a substituted or unsubstituted terphenyl group; a substituted or unsubstituted naphthyl group; a substituted or unsubstituted triphenylenyl group; a substituted or unsubstituted fluorenyl group; 9,9′-spirobi[fluorene]; or a substituted or unsubstituted dibenzothiophene group.

In one embodiment of the present specification, R21 and R22 are each independently a phenyl group substituted with a cyano group or a triphenylsilyl group; a biphenyl group; a terphenyl group; a naphthyl group; a triphenylenyl group; a fluorenyl group unsubstituted or substituted with a methyl group or a phenyl group; 9,9′-spirobi[fluorene]; or a dibenzothiophene group unsubstituted or substituted with a phenyl group, a biphenyl group, a naphthyl group, 9,9-dimethyl-9H-fluorene, a dibenzofuran group or a dibenzothiophene group.

In one embodiment of the present specification, R22 is a substituted or unsubstituted aryl group having 6 to 30 carbon atoms.

In one embodiment of the present specification, R22 is a substituted or unsubstituted phenyl group; a substituted or unsubstituted biphenyl group; a substituted or unsubstituted terphenyl group; a substituted or unsubstituted naphthyl group; a substituted or unsubstituted triphenylenyl group; or a substituted or unsubstituted fluorenyl group.

In one embodiment of the present specification, R22 is a phenyl group substituted with a cyano group or a triphenylsilyl group; a biphenyl group; a terphenyl group; a naphthyl group; a triphenylenyl group; or a fluorenyl group unsubstituted or substituted with a methyl group or a phenyl group.

In one embodiment of the present specification, R23 and R24 are each independently hydrogen; or deuterium.

In one embodiment of the present specification, Chemical Formula 2 may be represented by any one of the following compounds, but is not limited thereto.

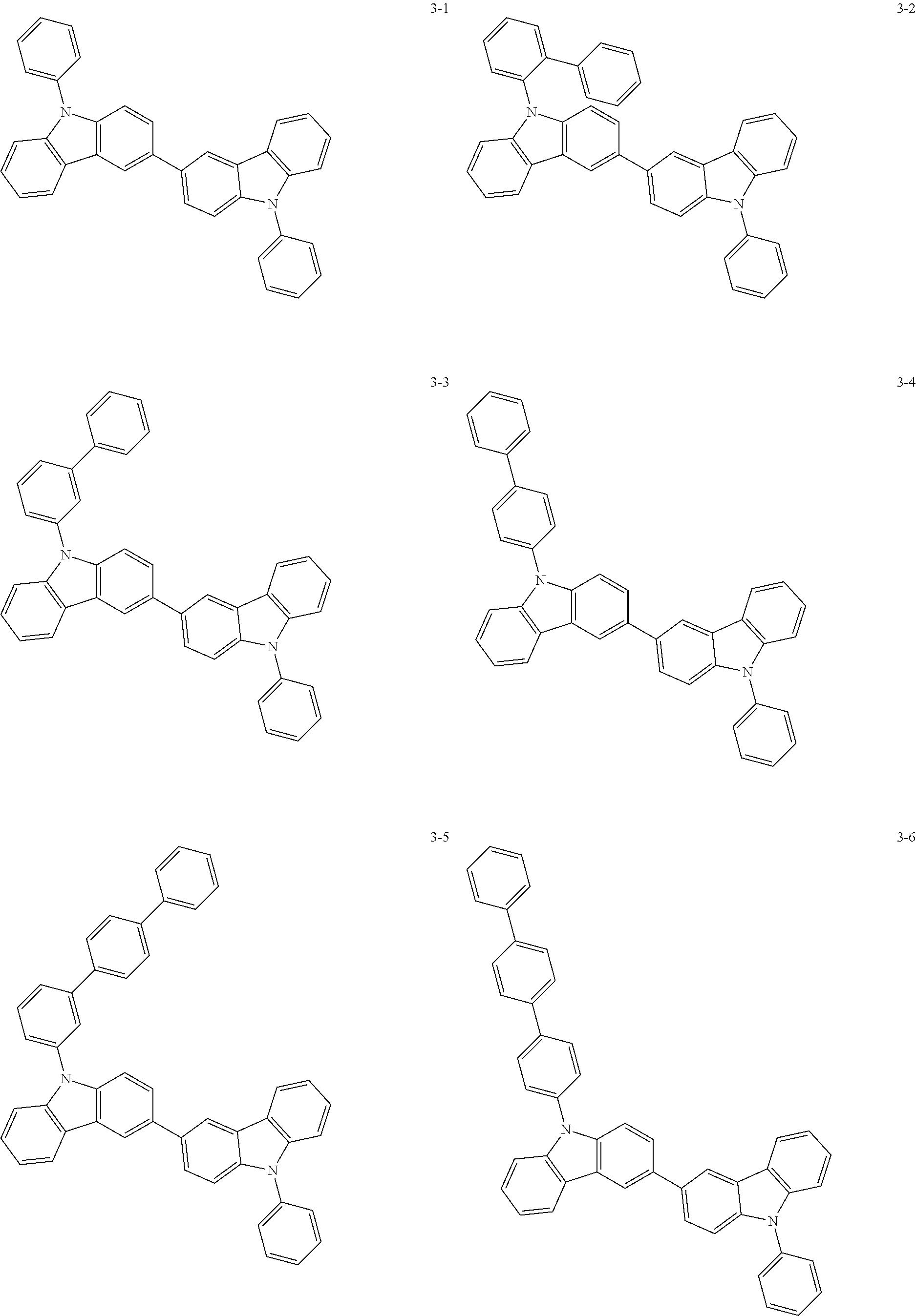 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

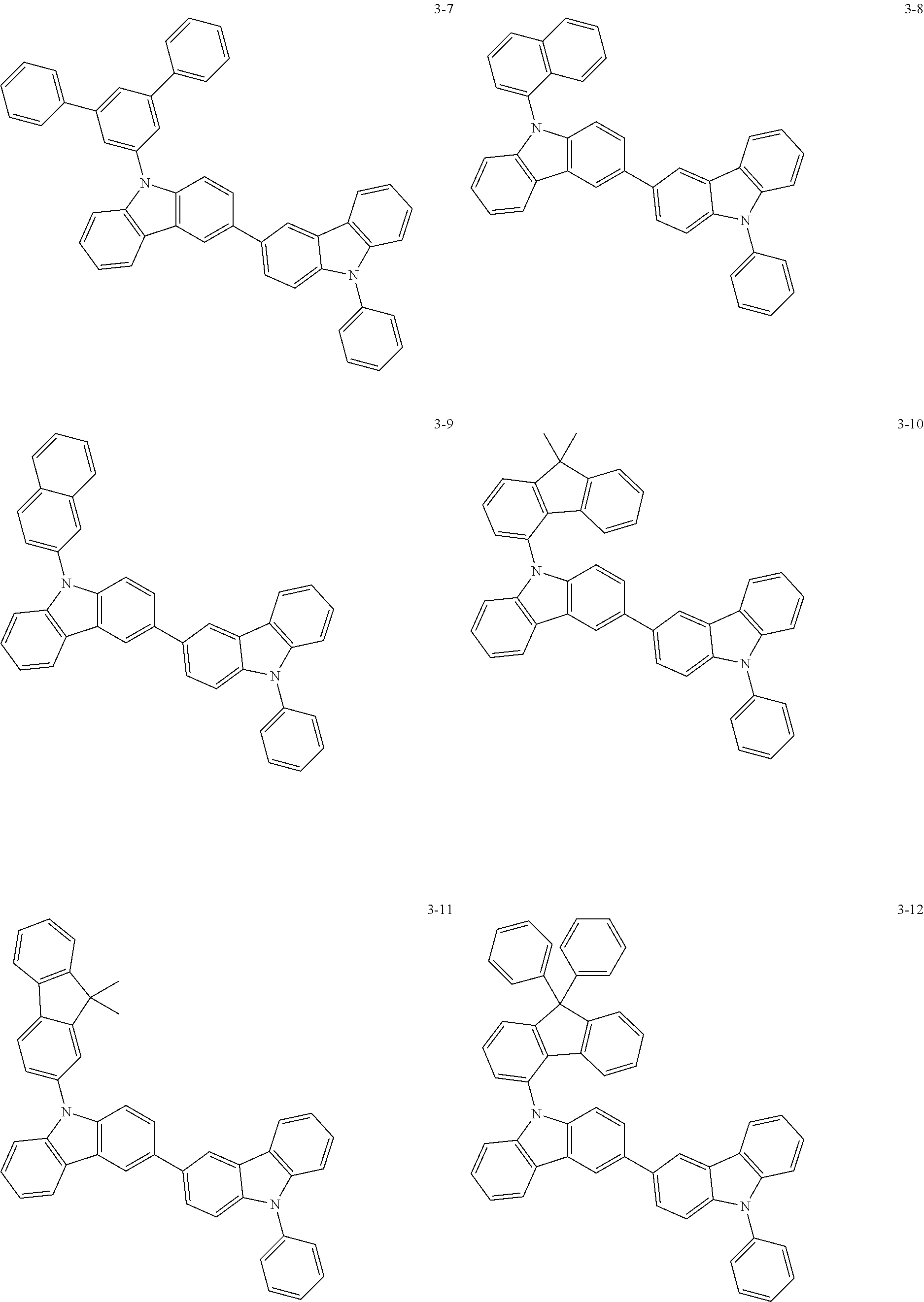 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

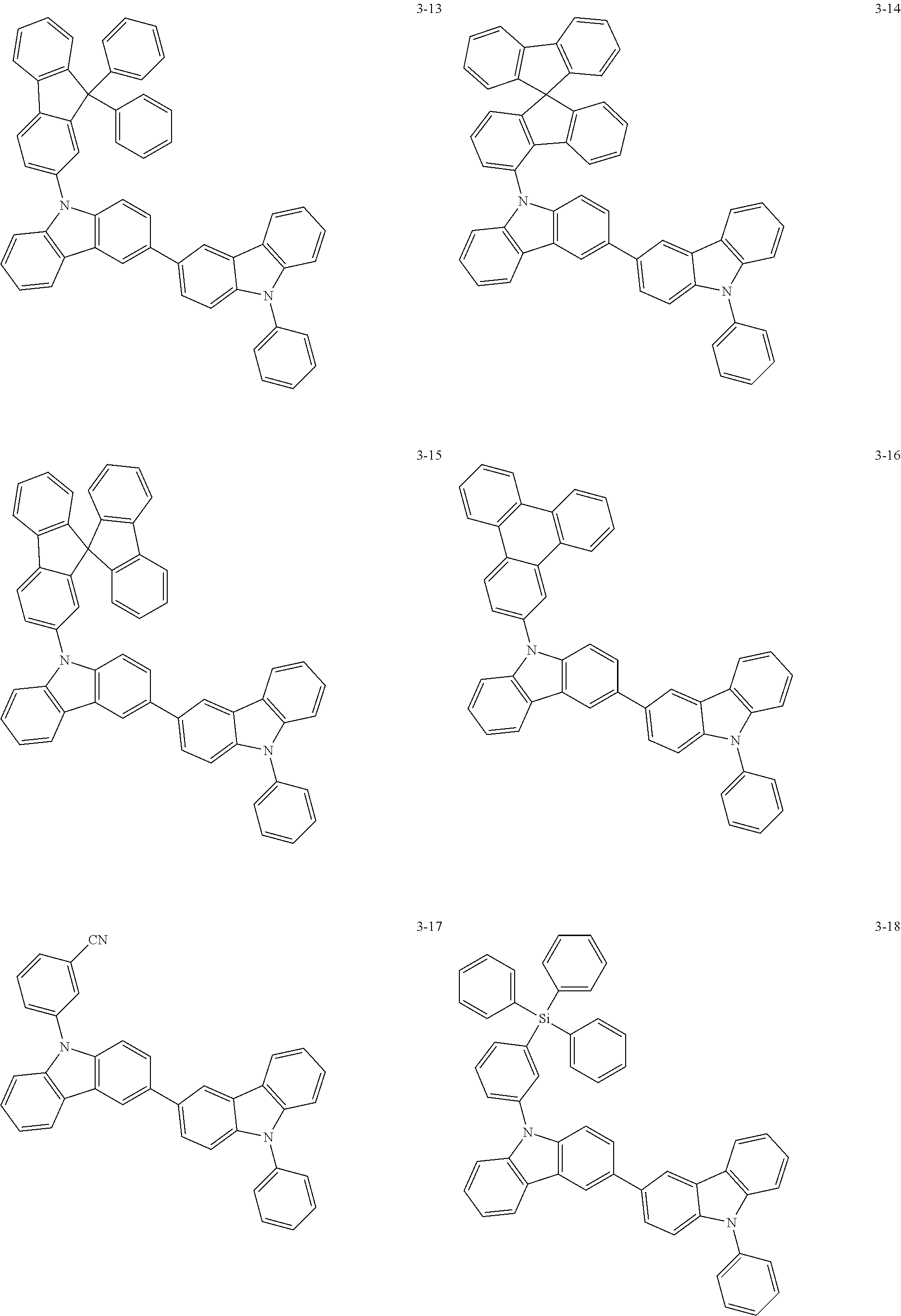

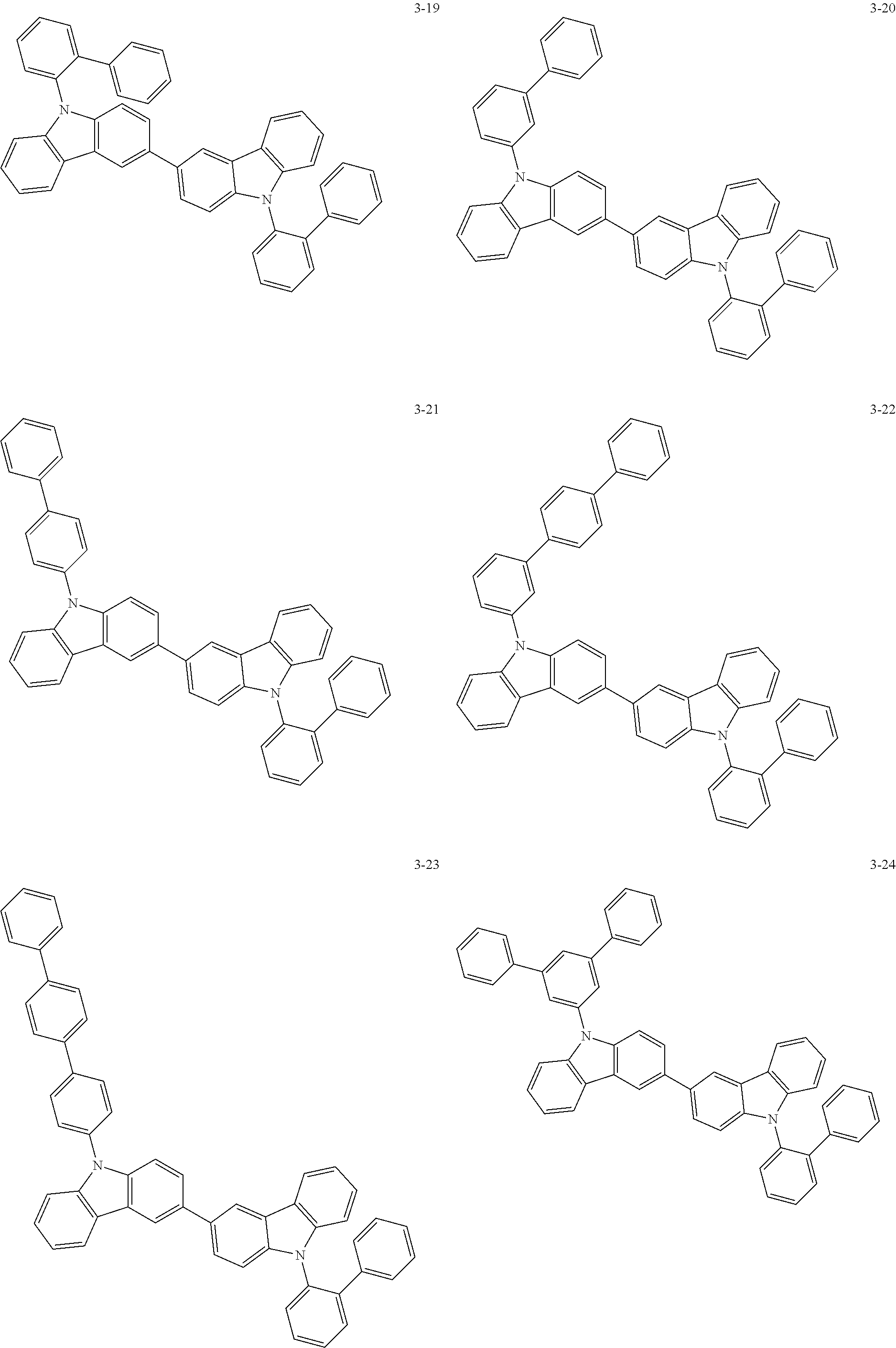

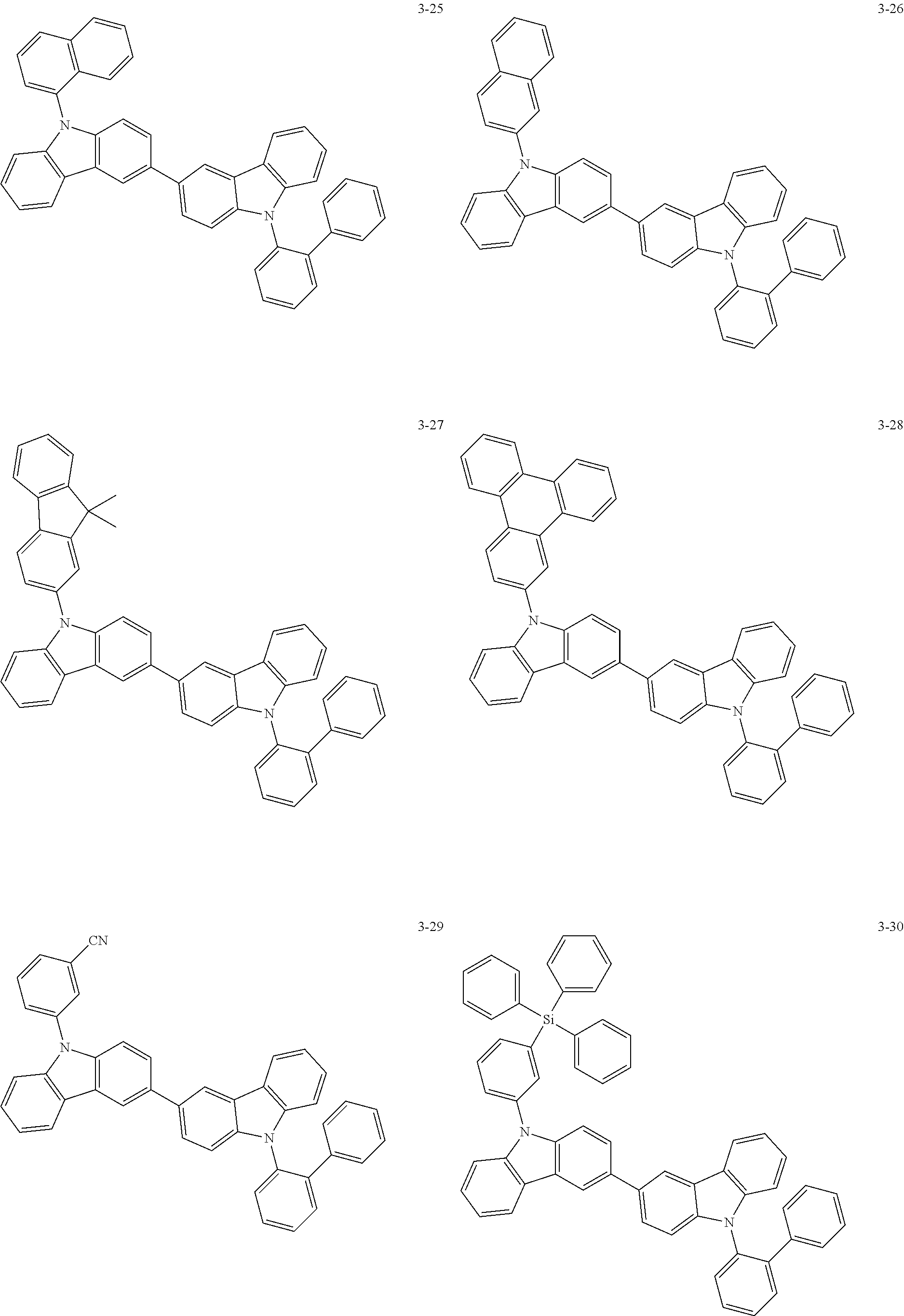

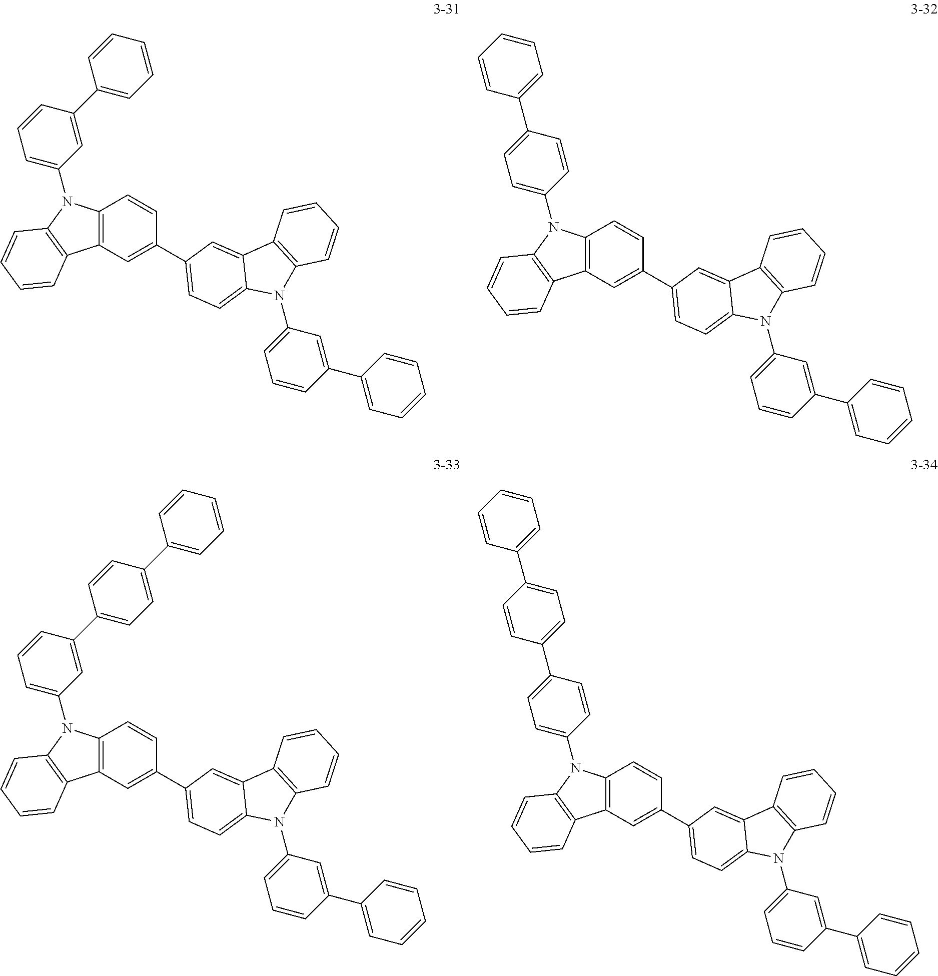 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

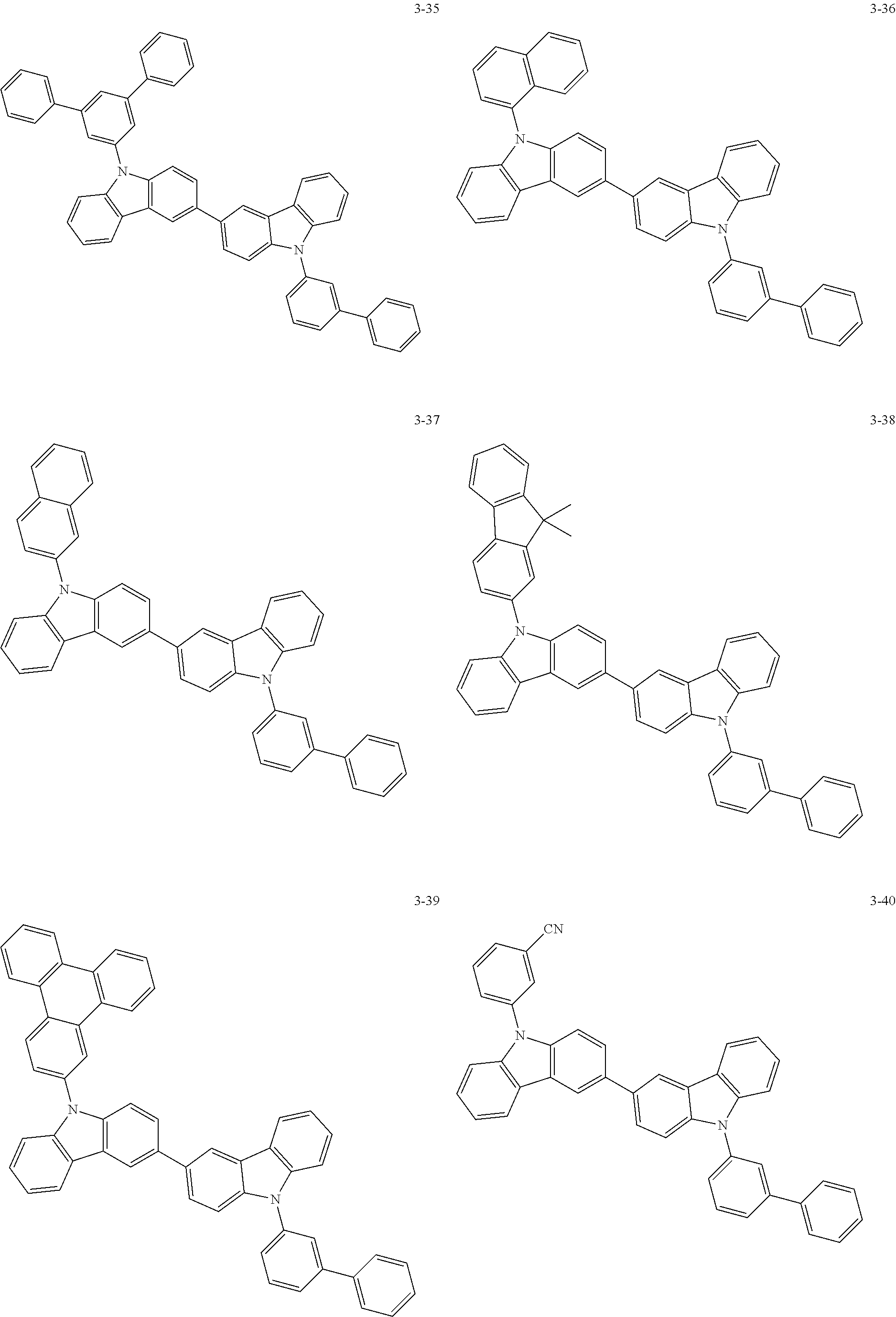

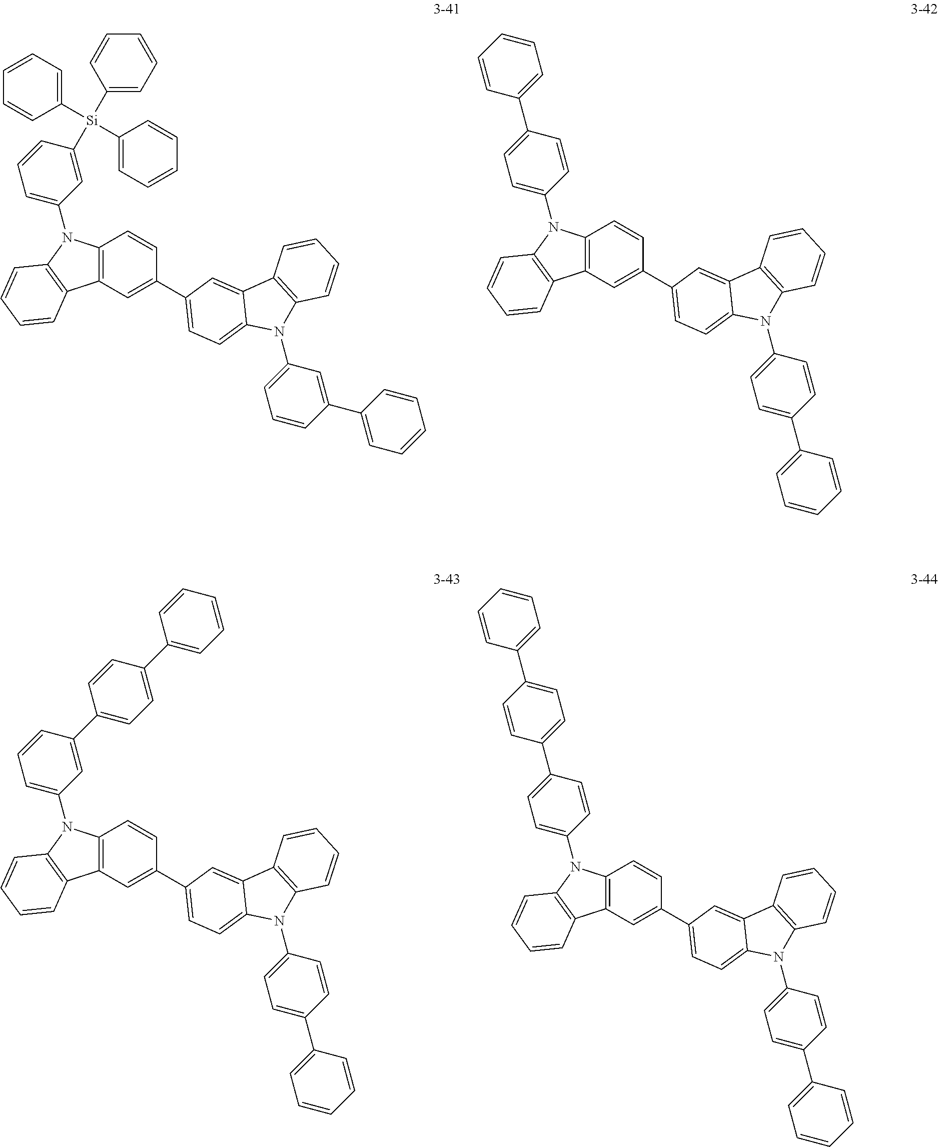 [SMILES: c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

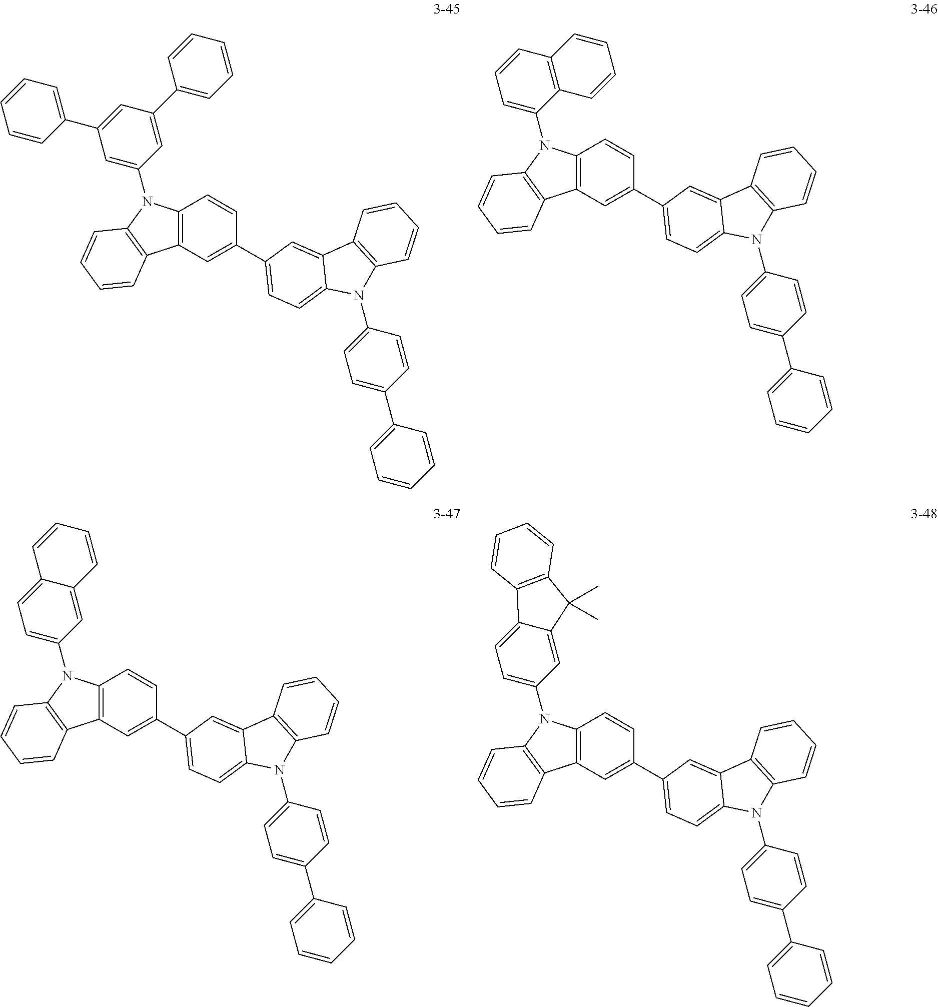 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

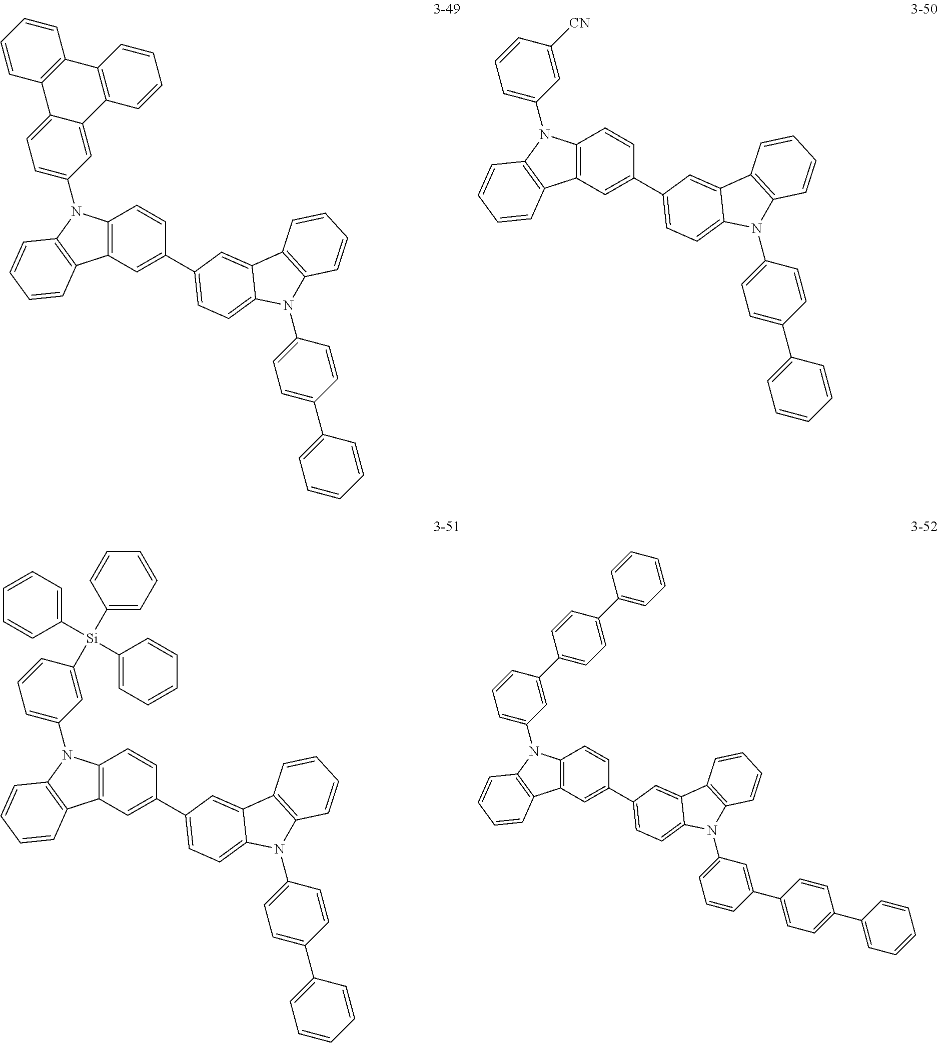 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

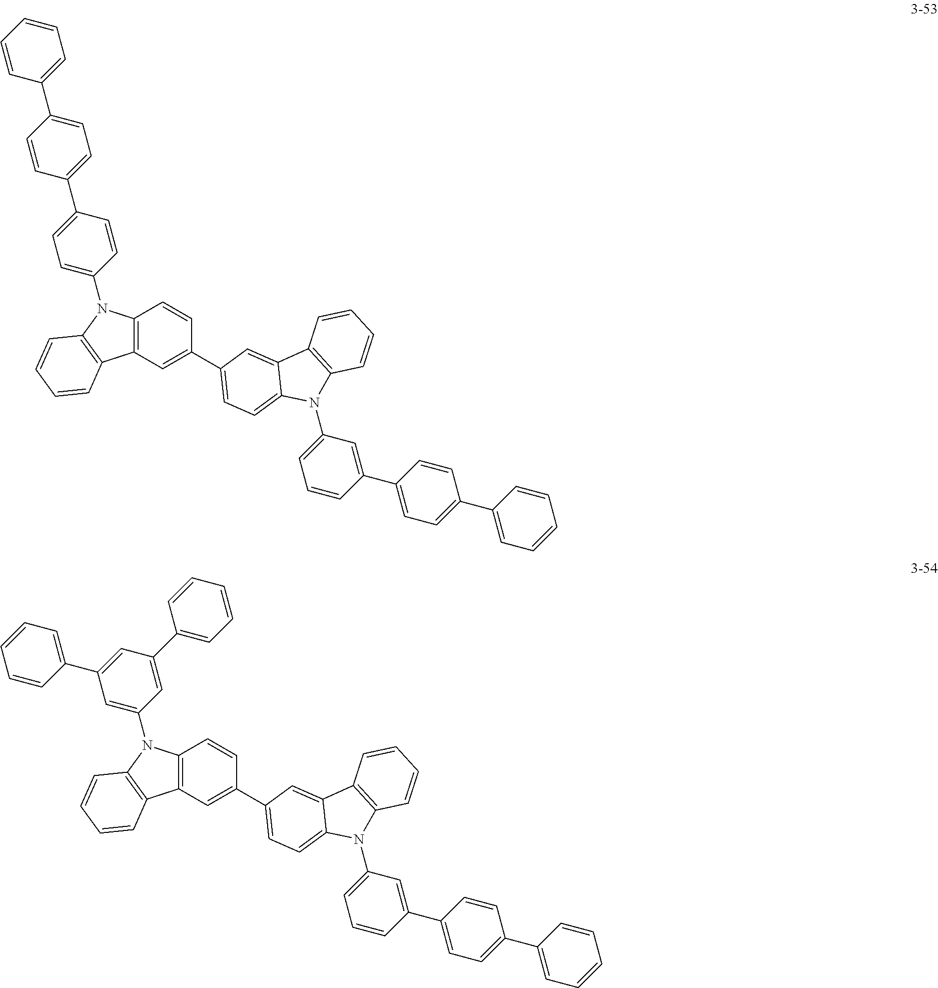 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

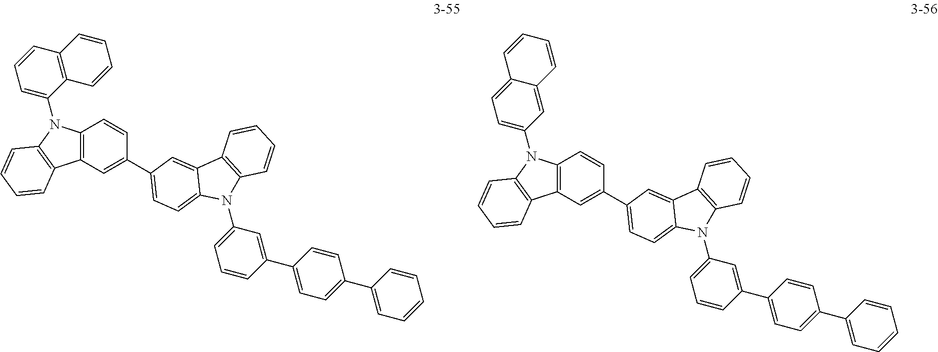 [SMILES: c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

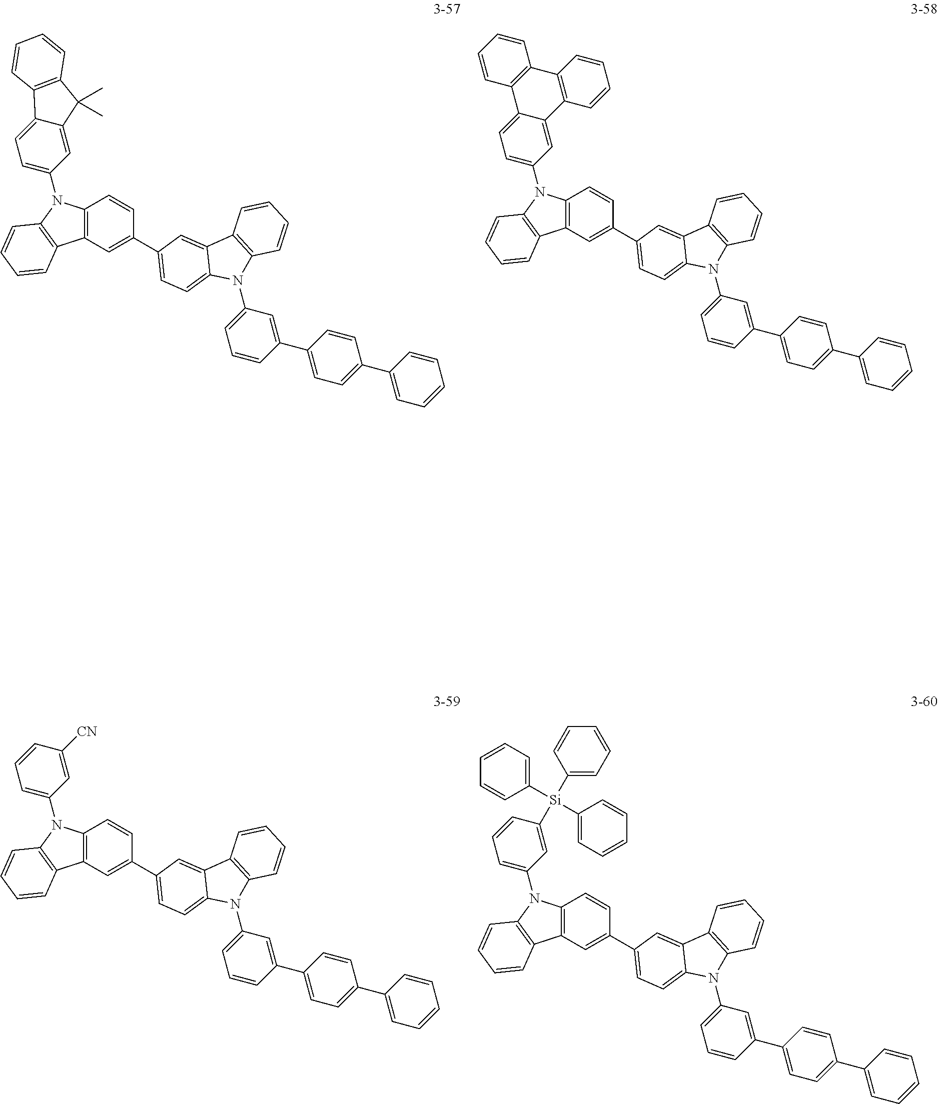 [SMILES: CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21 ; c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

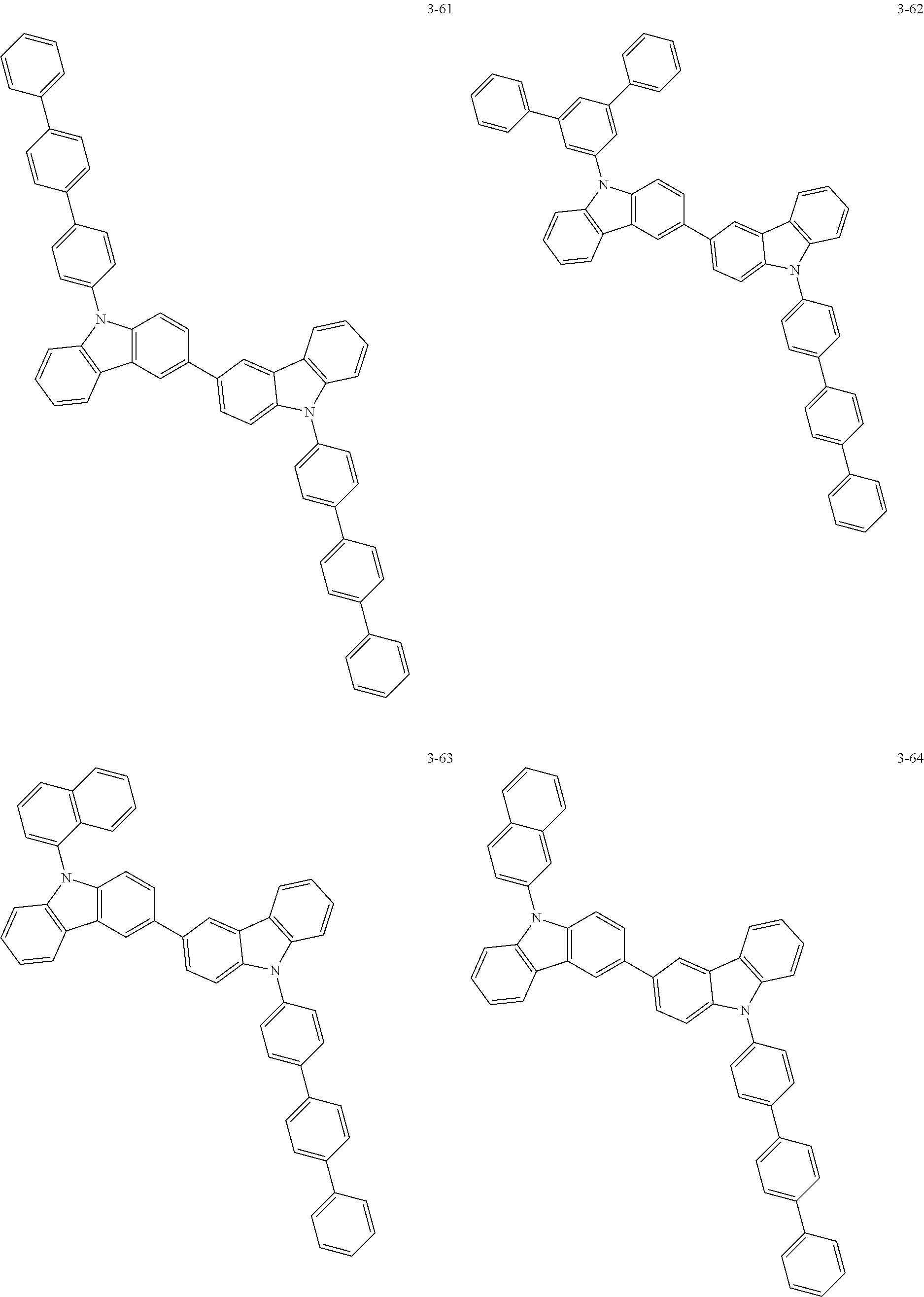 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

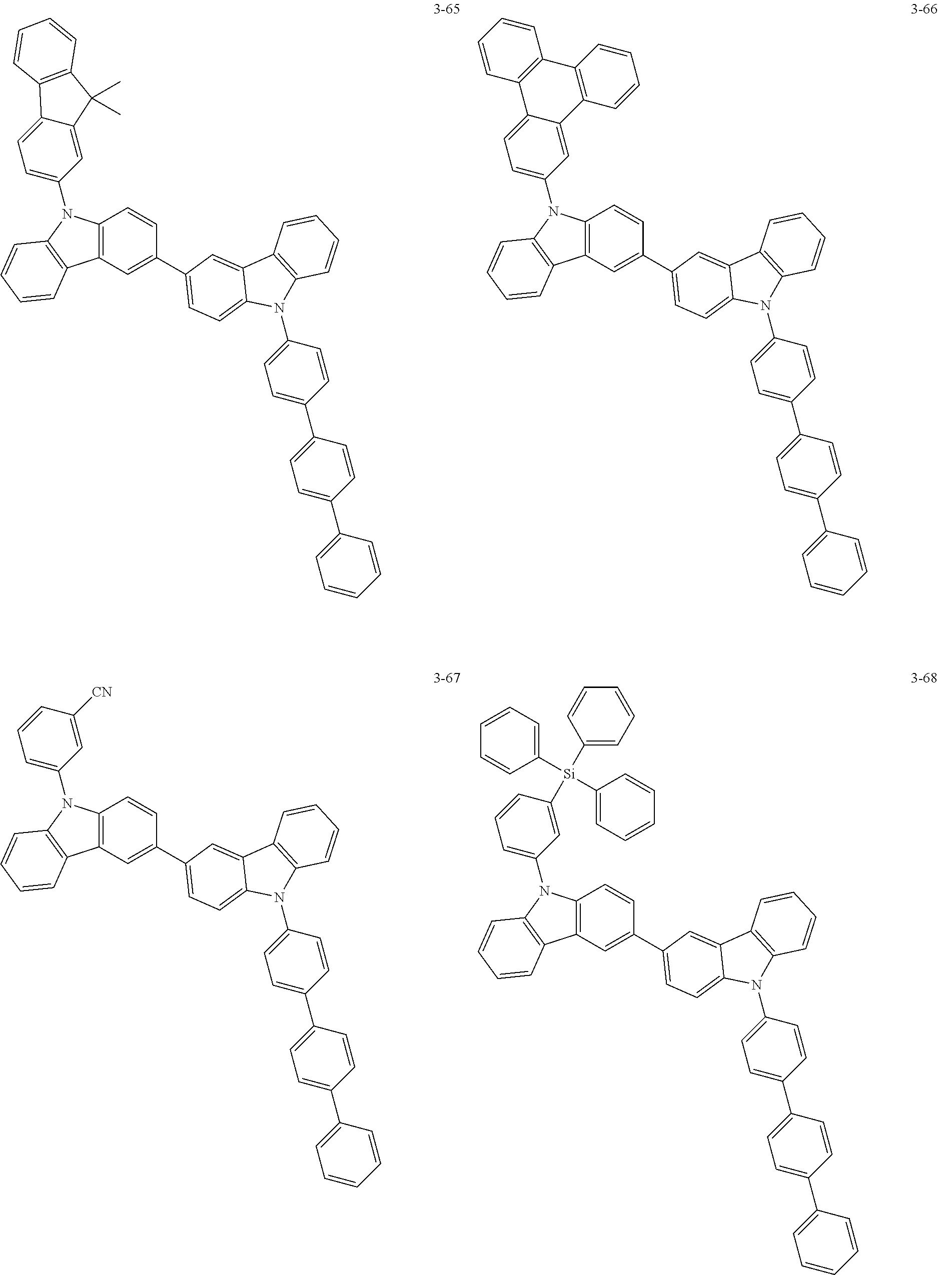 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

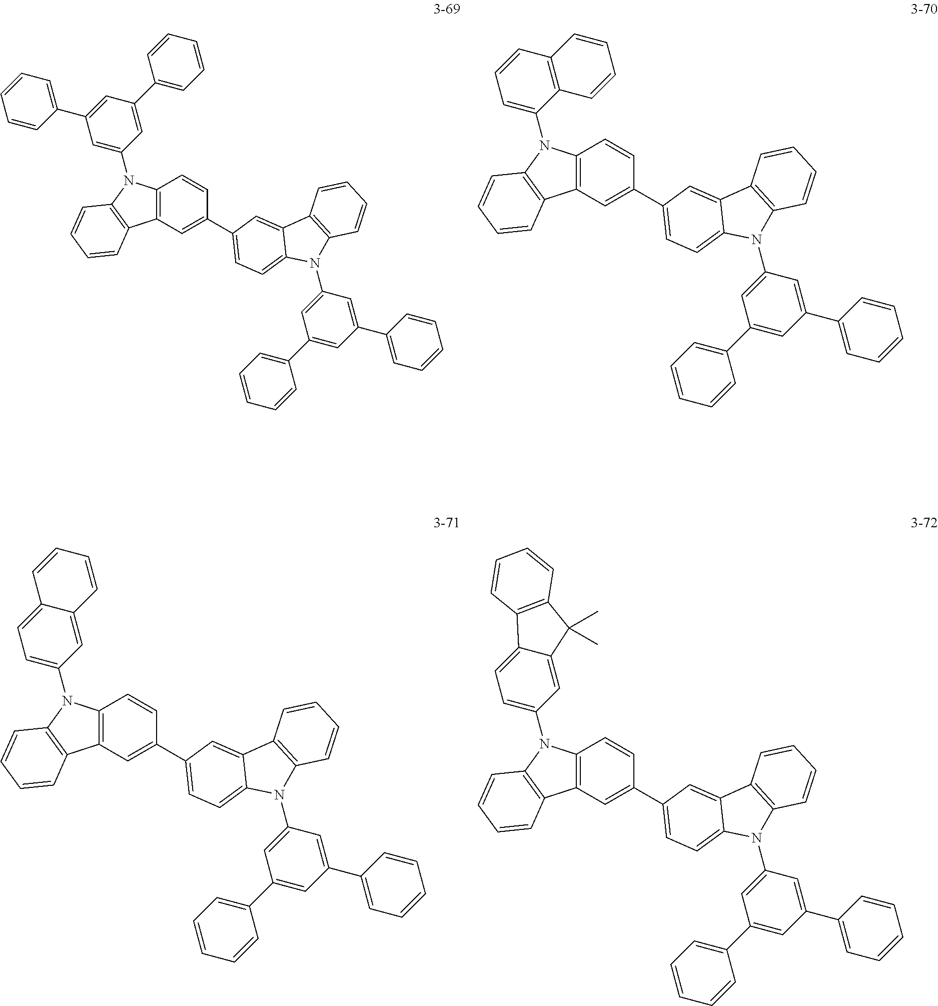 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

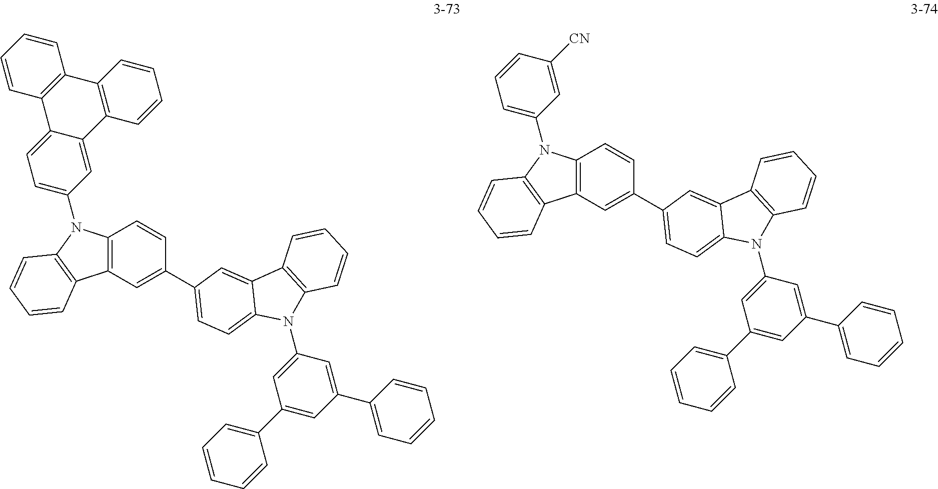 [SMILES: c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cc(-c5ccccc5)cc(-c5ccccc5)c4)ccc32)c1]

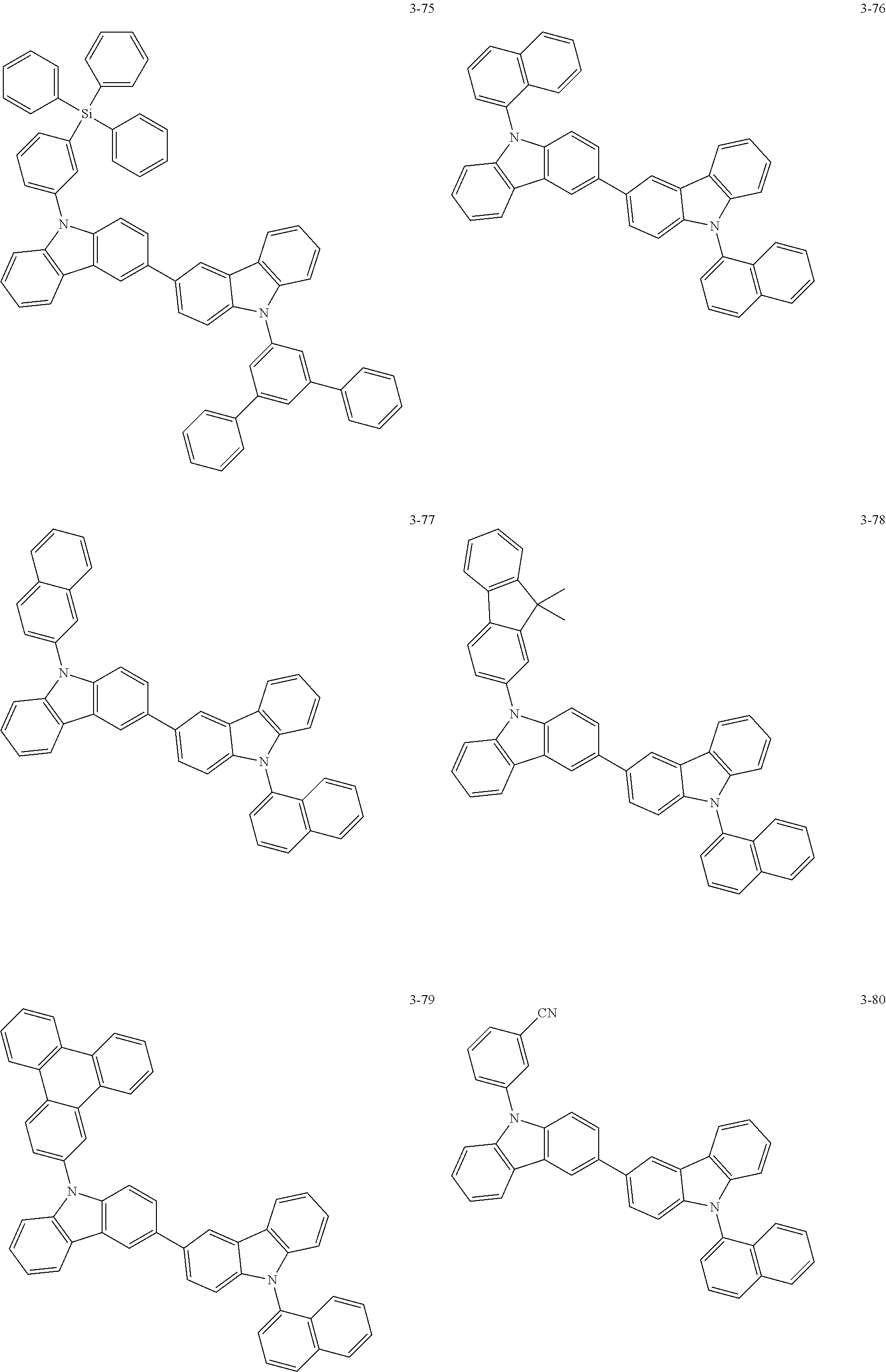

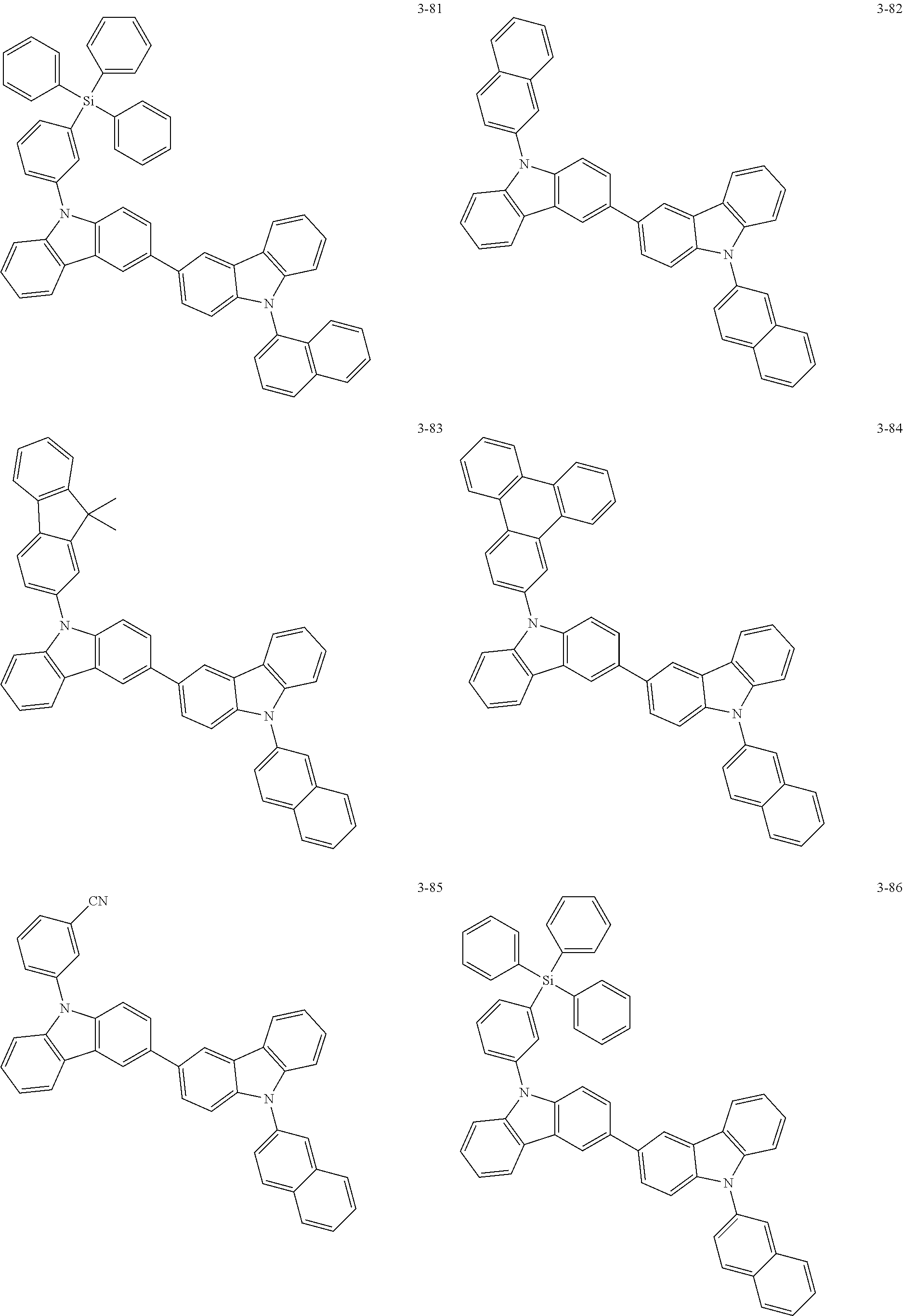

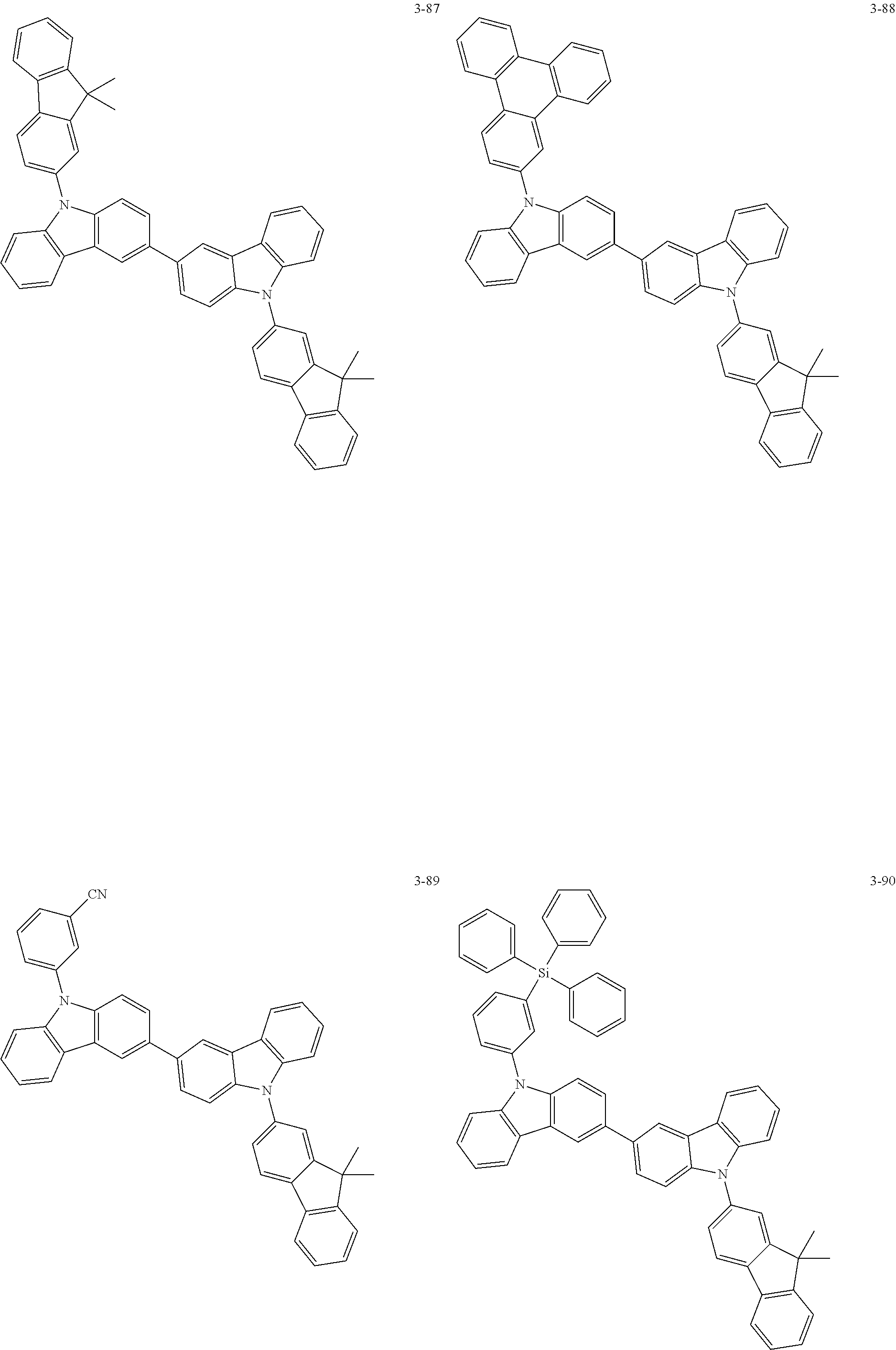 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

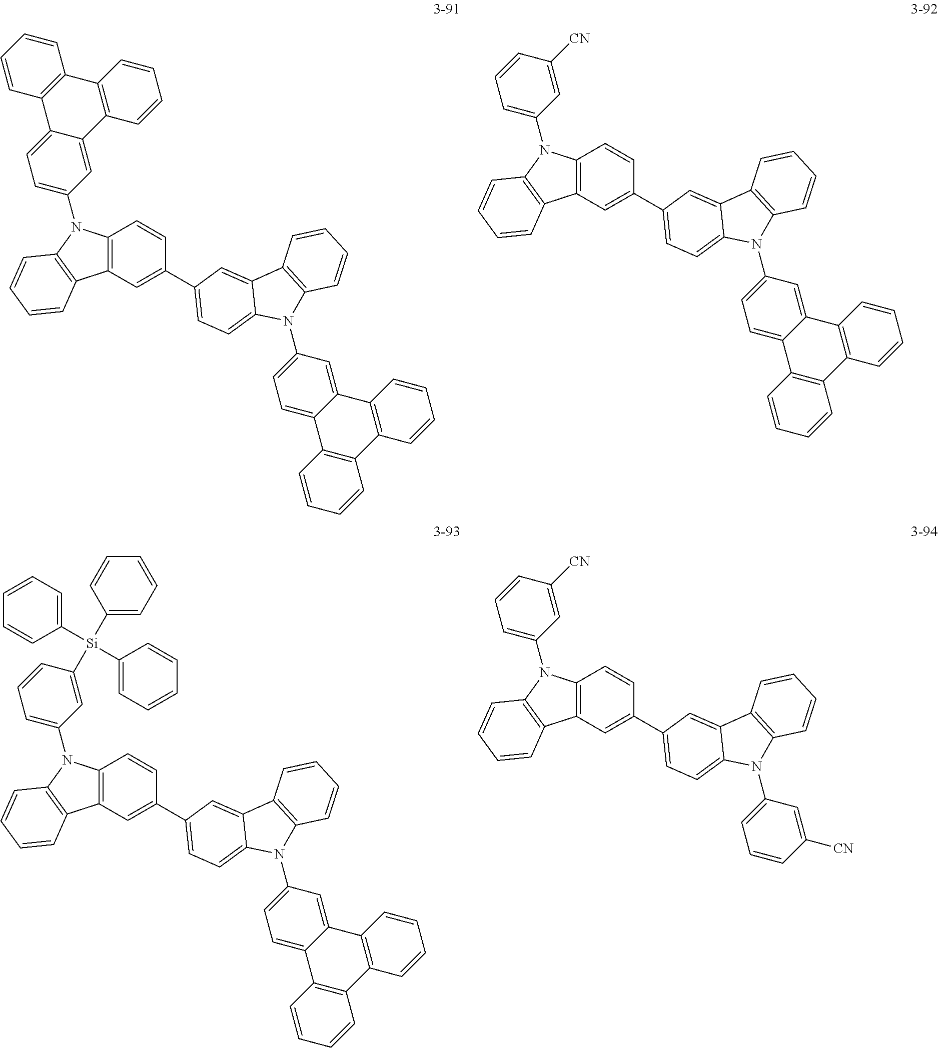 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1 ; c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1]

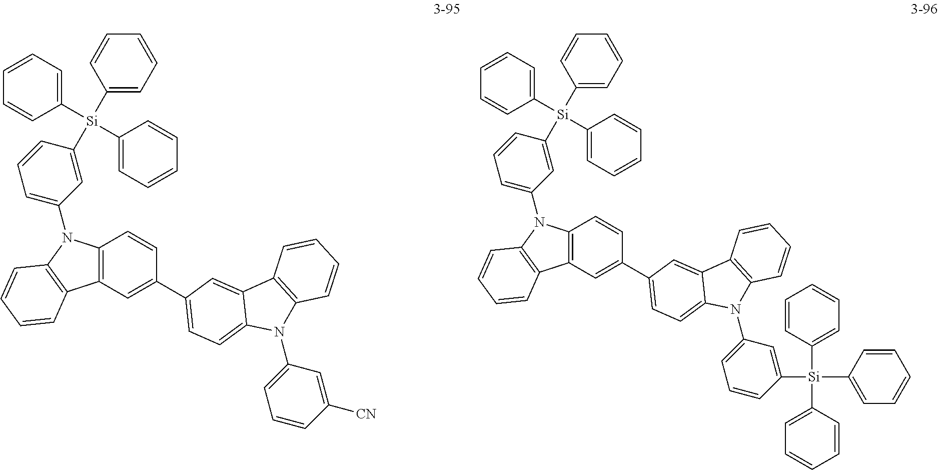 [SMILES: N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1 ; c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

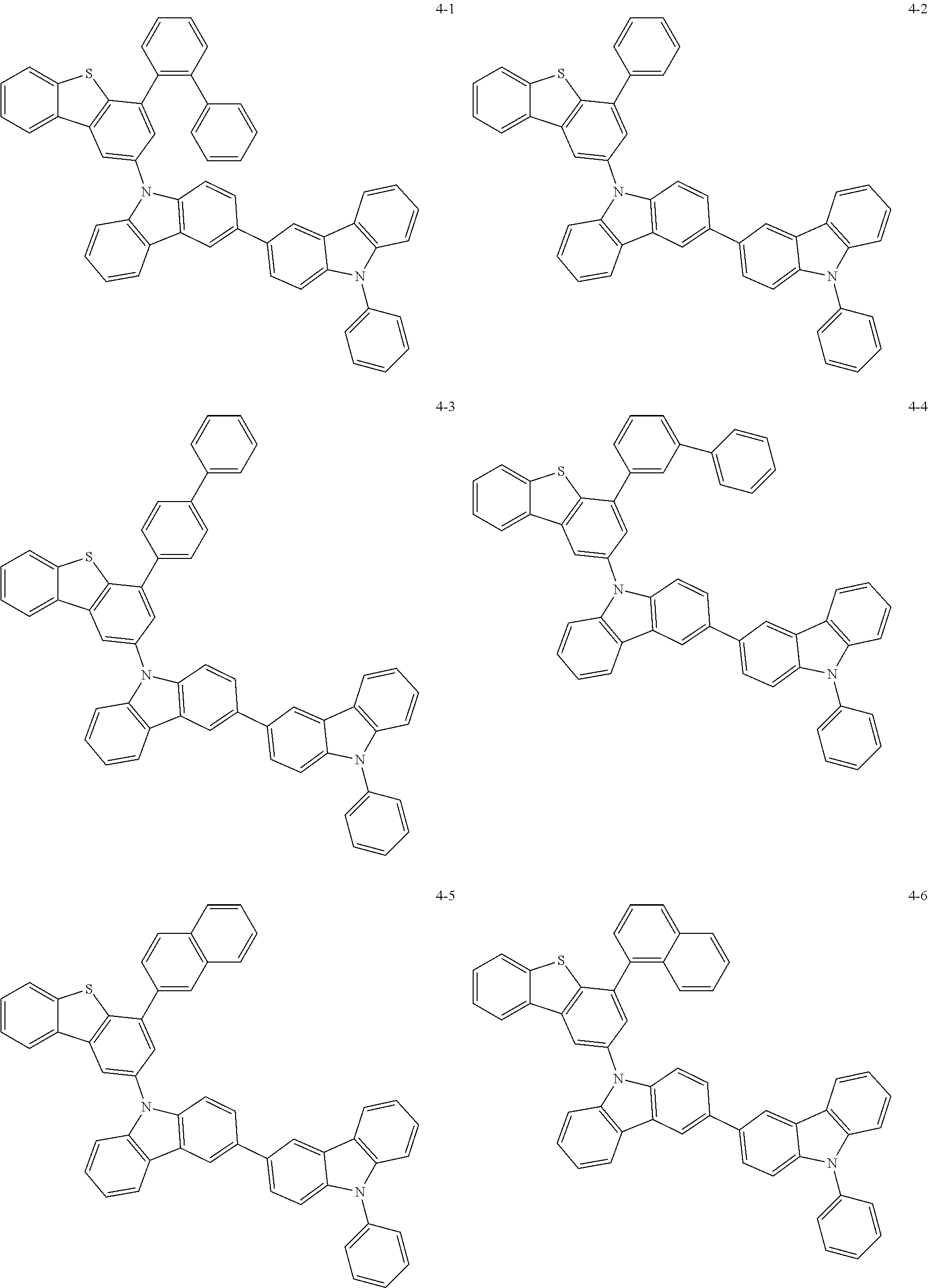

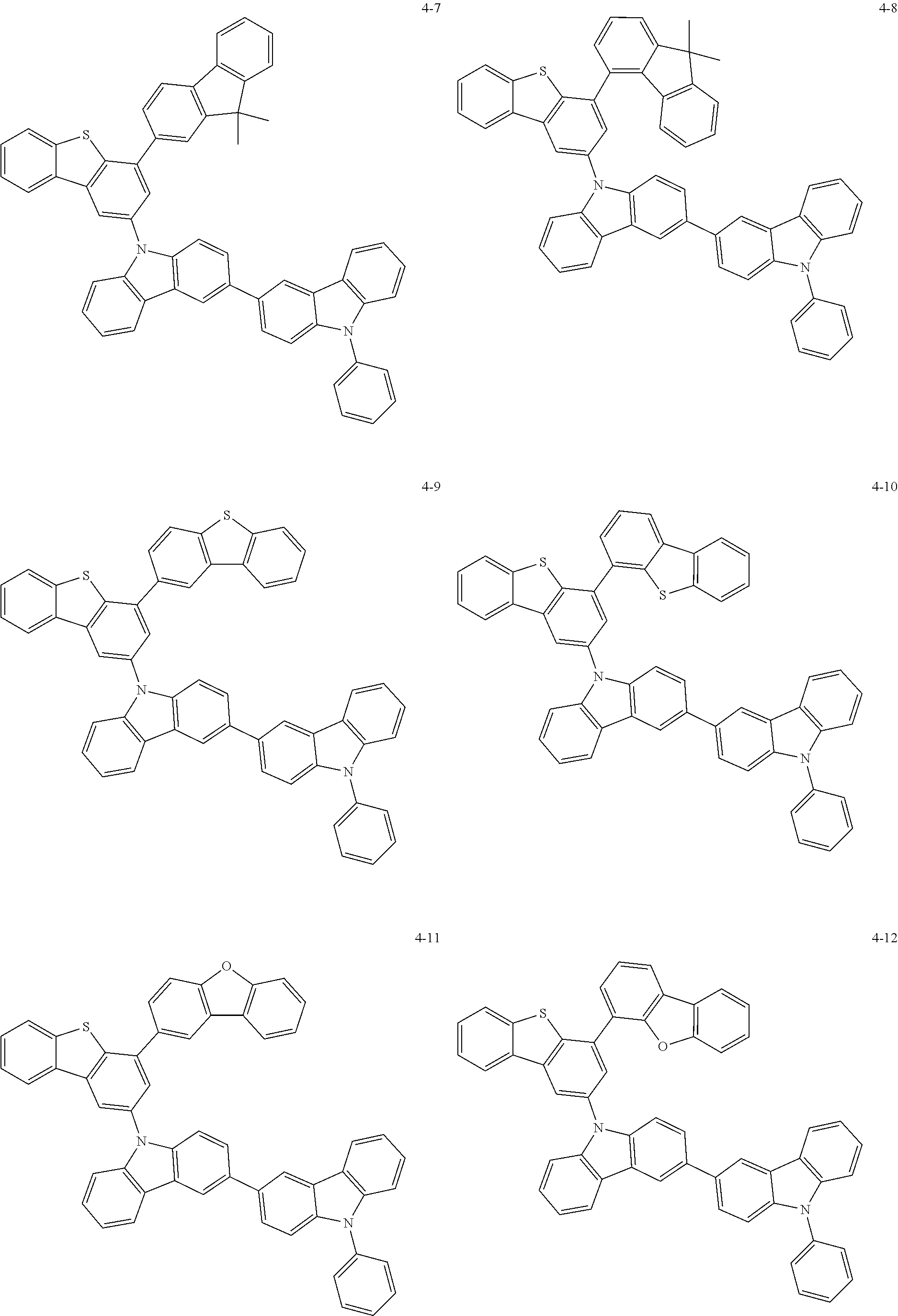

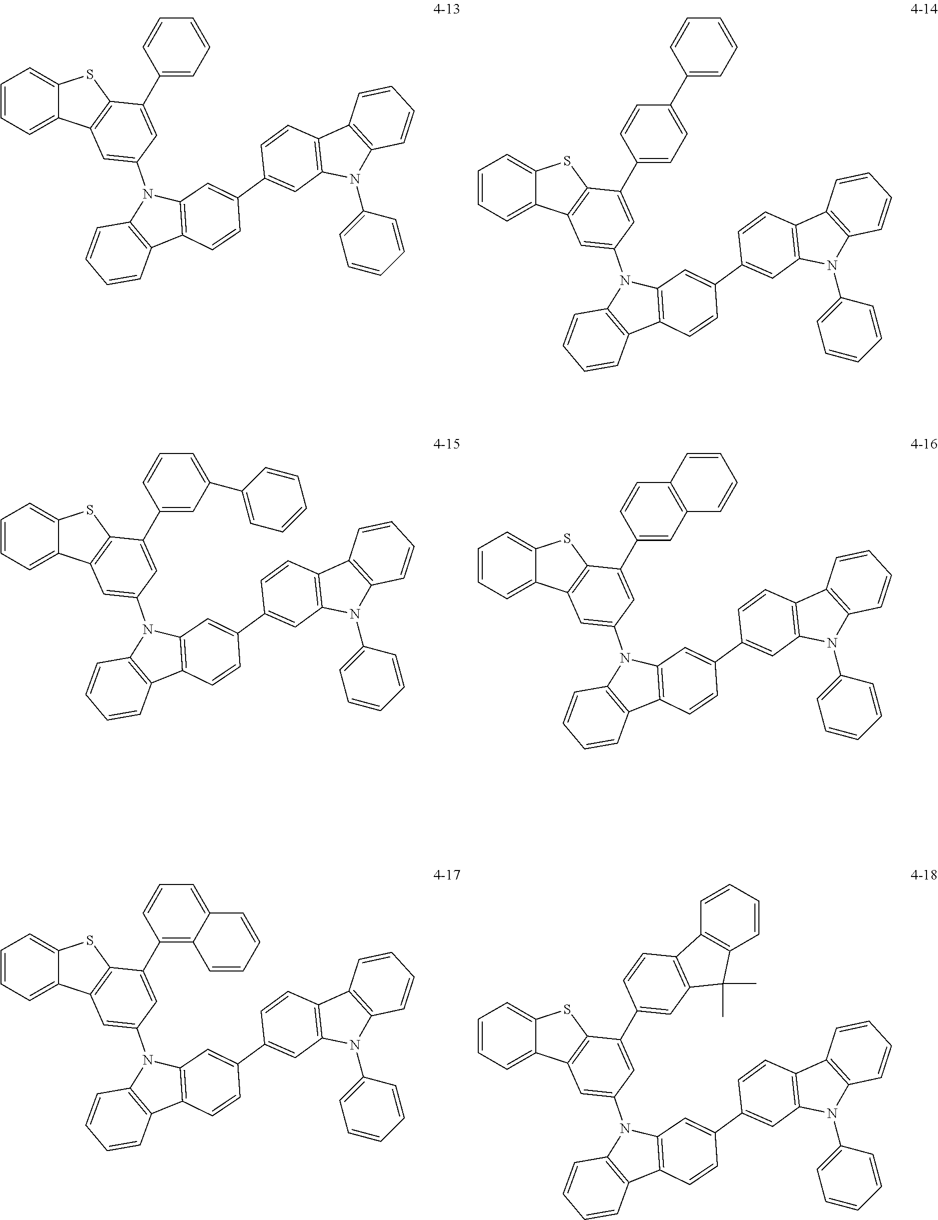

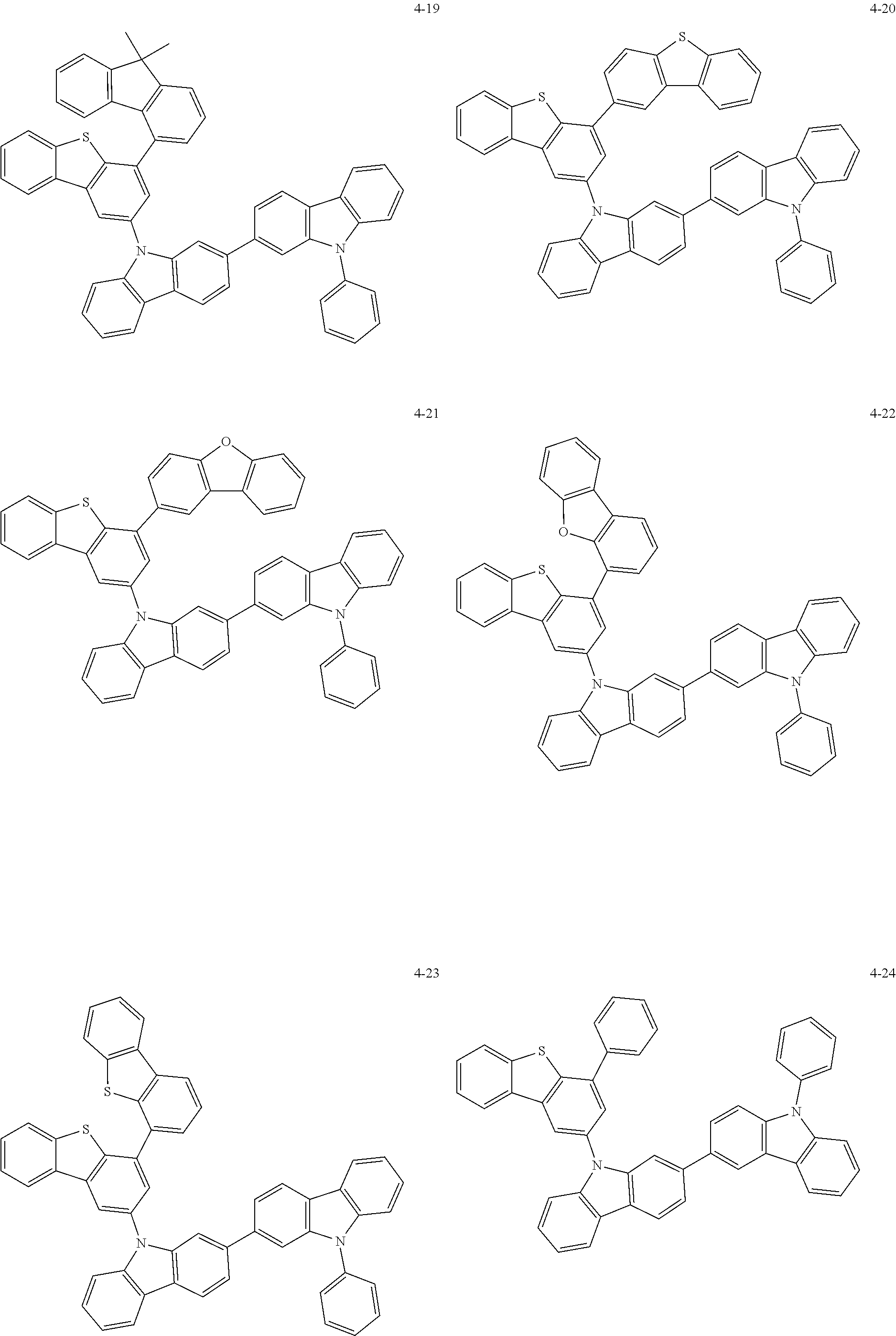

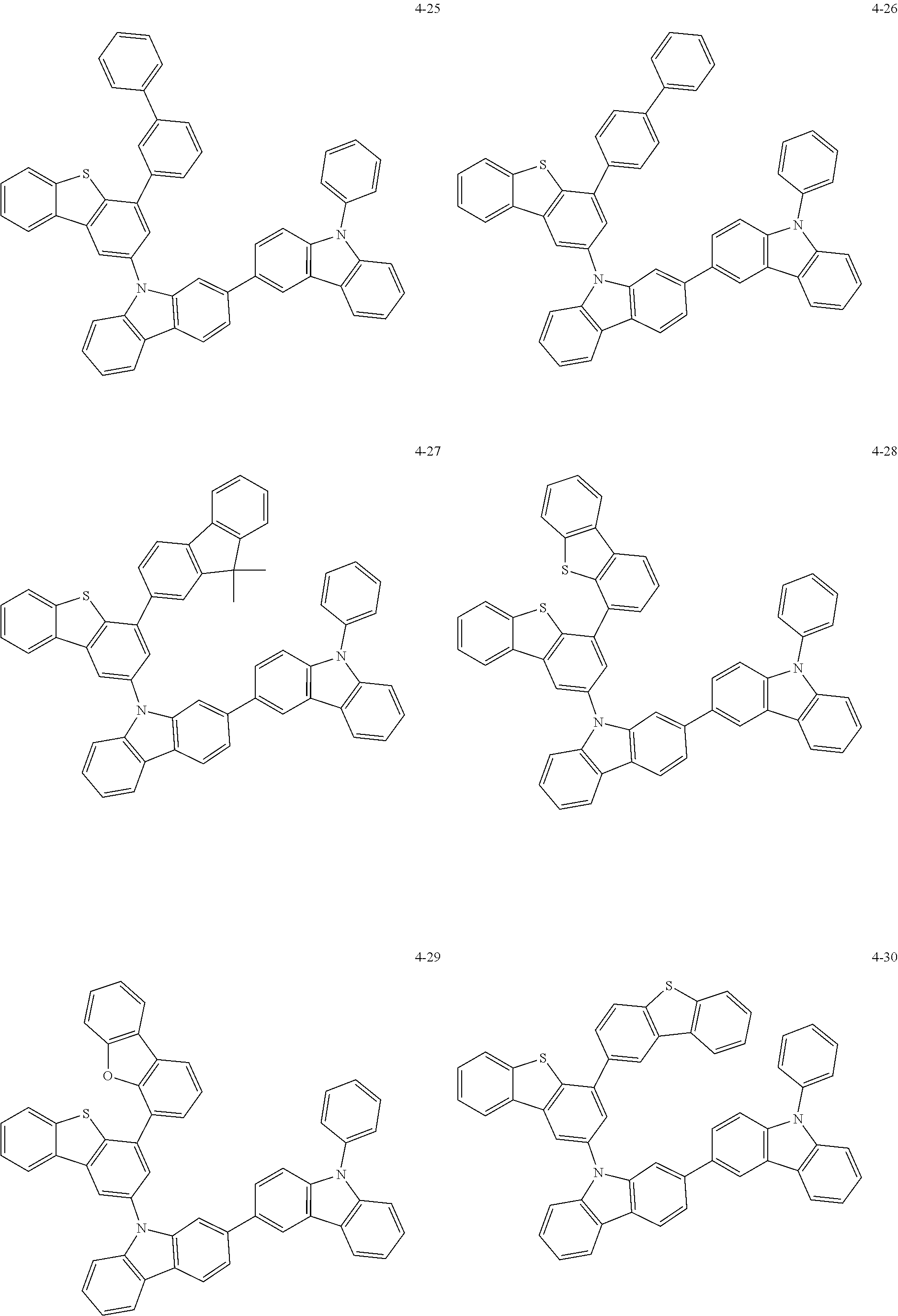

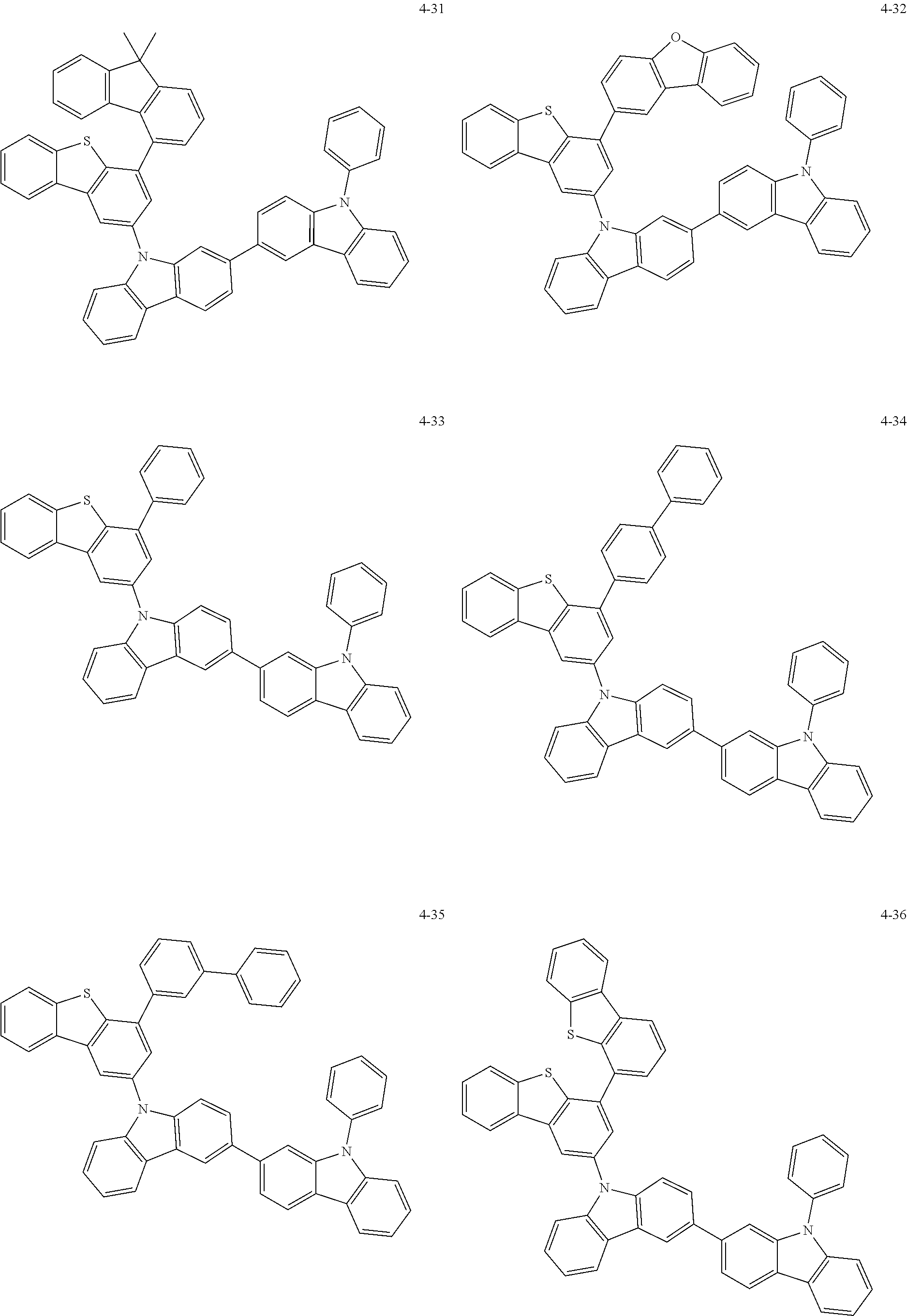

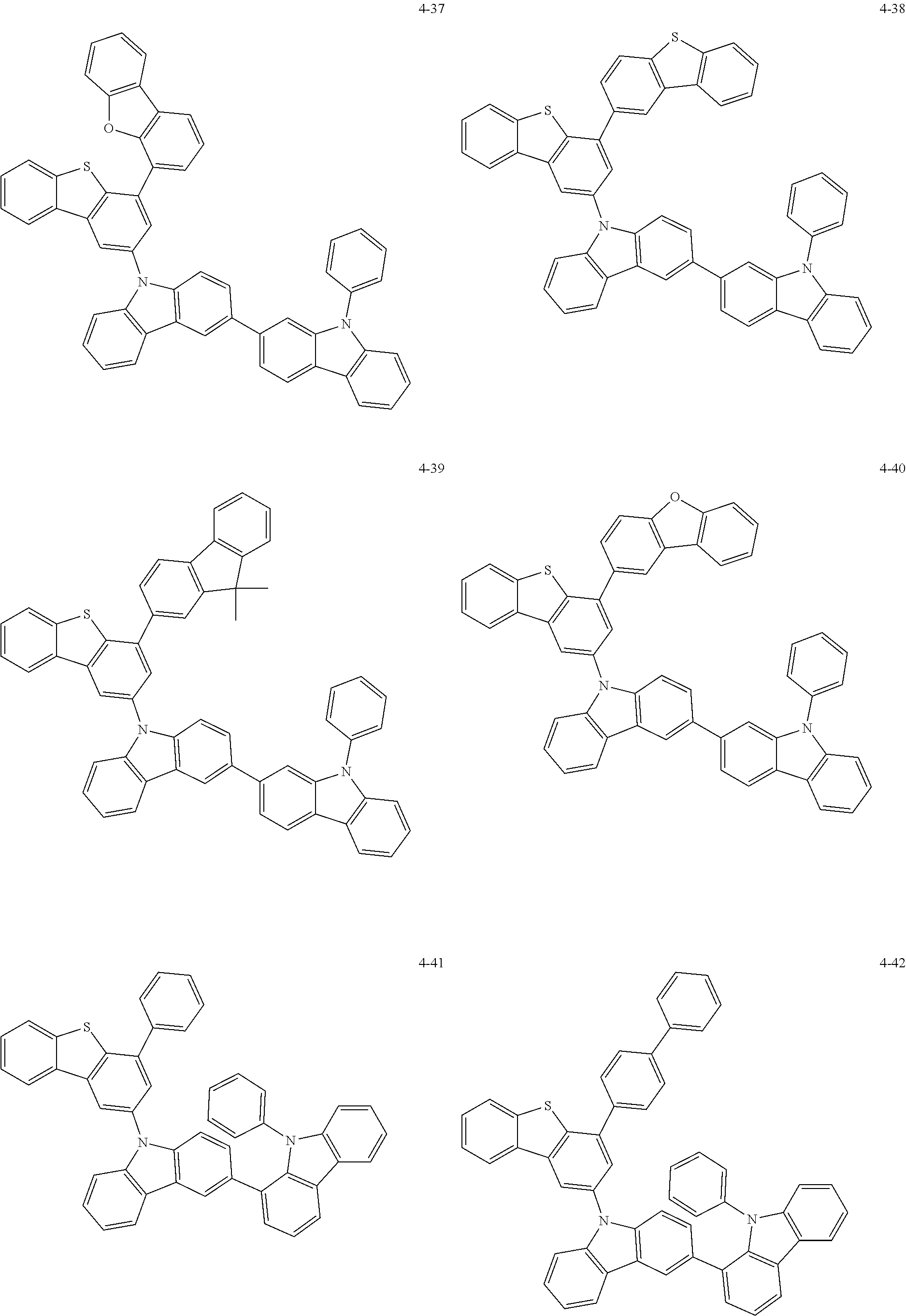

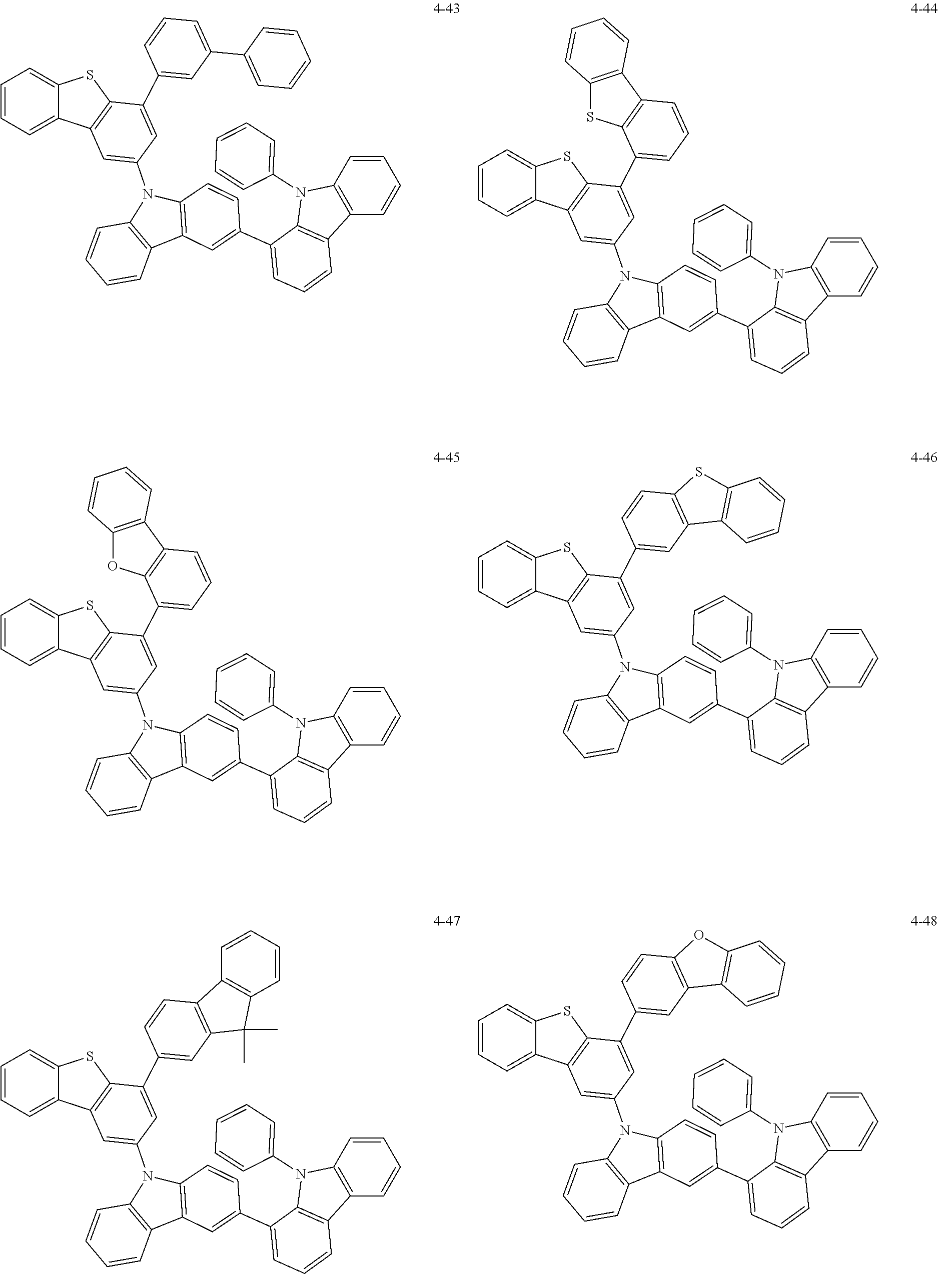

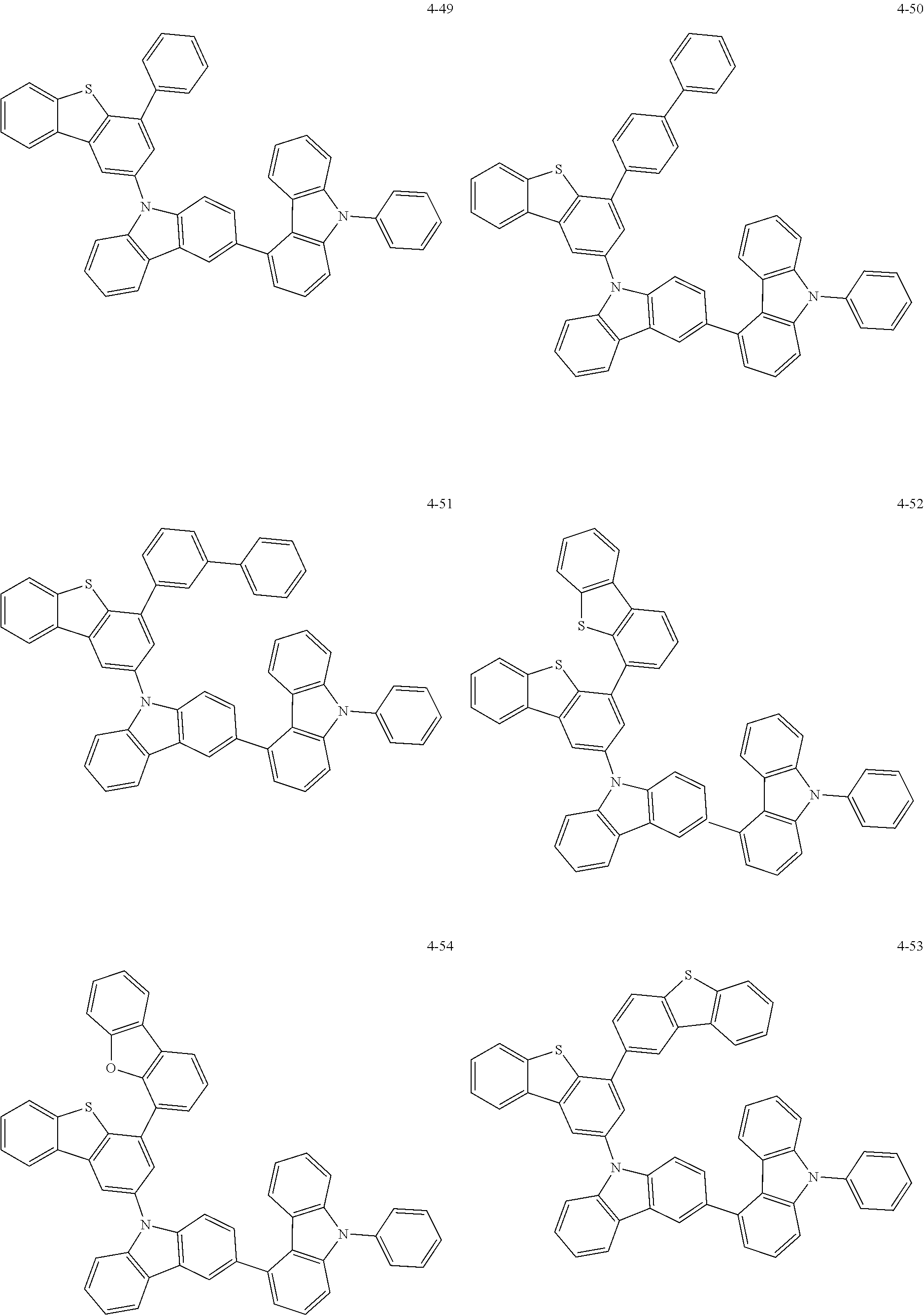

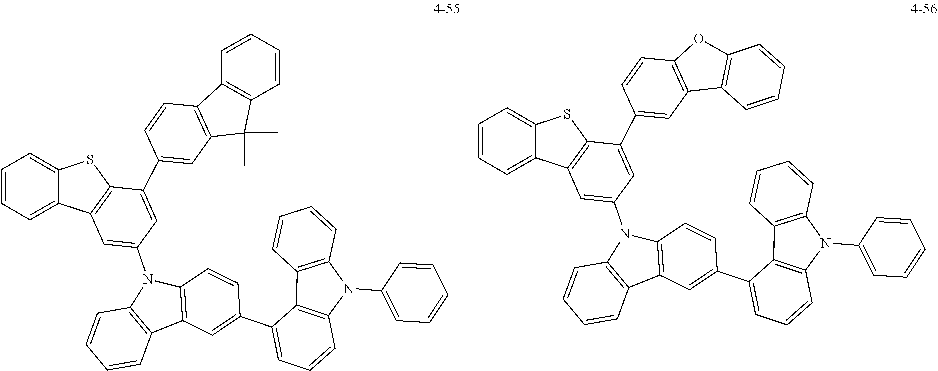 [SMILES: CC1(C)c2ccccc2-c2ccc(-c3cc(-n4c5ccccc5c5cc(-c6cccc7c6c6ccccc6n7-c6ccccc6)ccc54)cc4c3sc3ccccc34)cc21 ; c1ccc(-n2c3ccccc3c3c(-c4ccc5c(c4)c4ccccc4n5-c4cc(-c5ccc6oc7ccccc7c6c5)c5sc6ccccc6c5c4)cccc32)cc1]

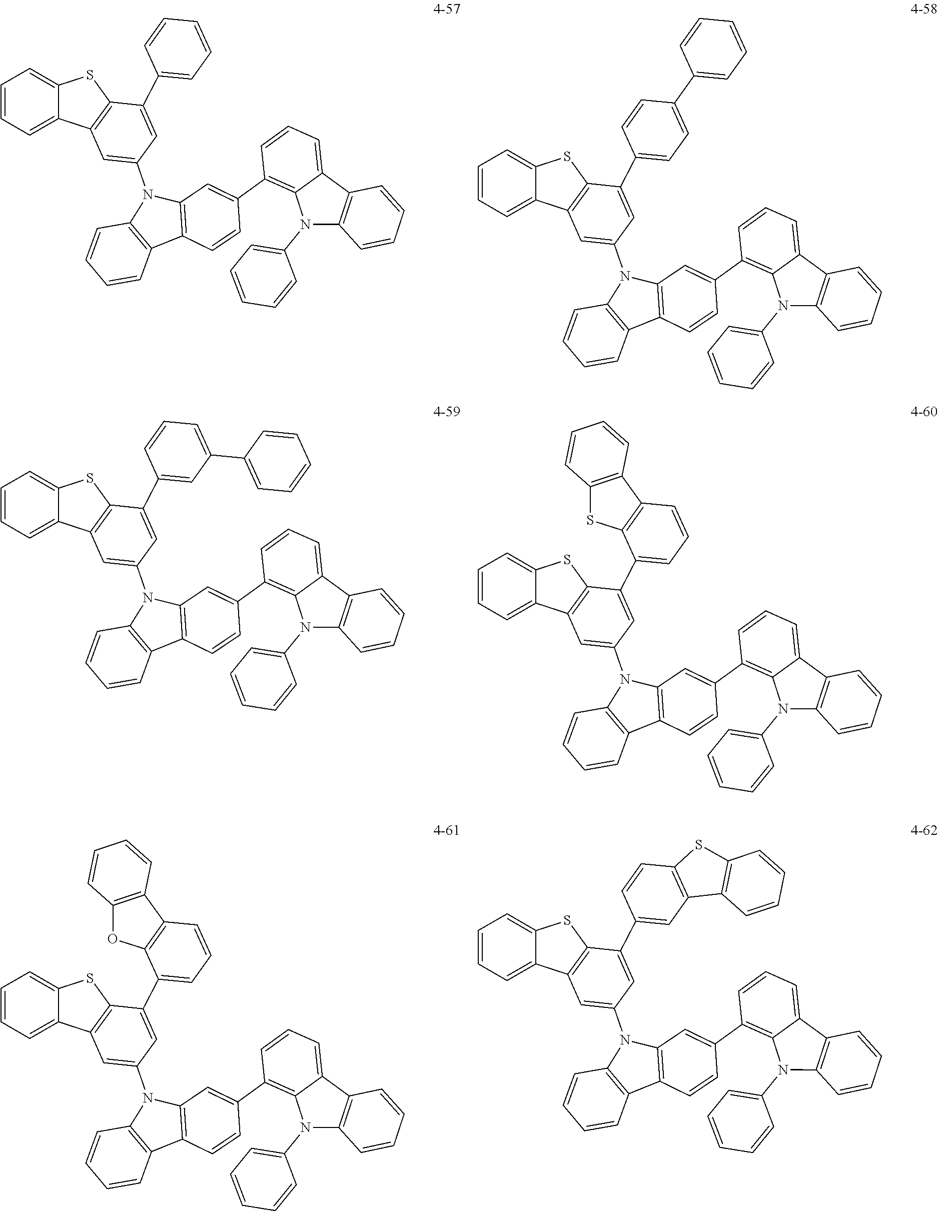

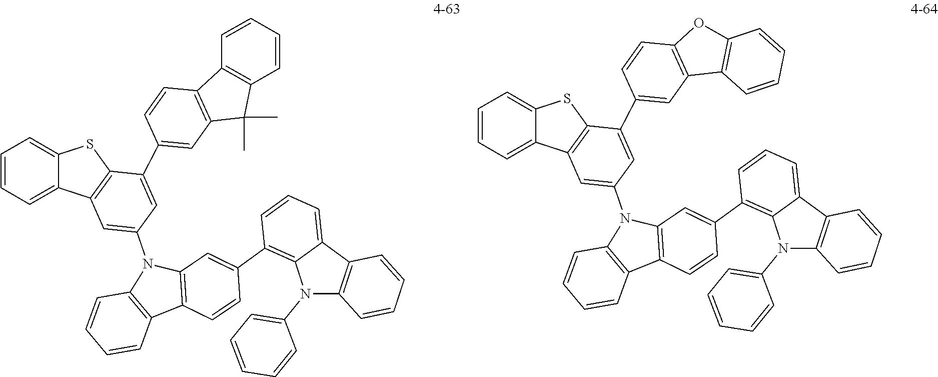 [SMILES: CC1(C)c2ccccc2-c2ccc(-c3cc(-n4c5ccccc5c5ccc(-c6cccc7c8ccccc8n(-c8ccccc8)c67)cc54)cc4c3sc3ccccc34)cc21 ; c1ccc(-n2c3ccccc3c3cccc(-c4ccc5c6ccccc6n(-c6cc(-c7ccc8oc9ccccc9c8c7)c7sc8ccccc8c7c6)c5c4)c32)cc1]

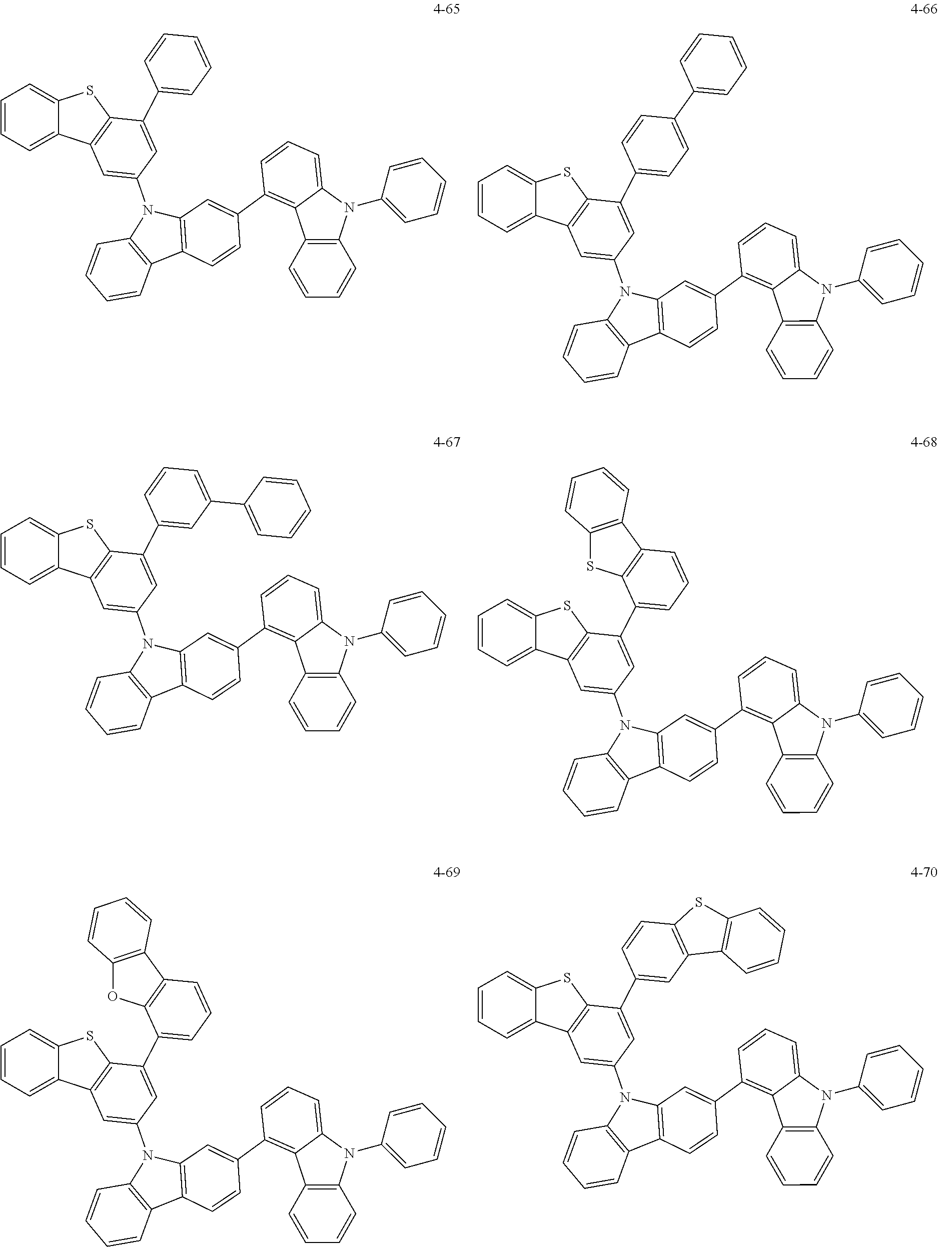

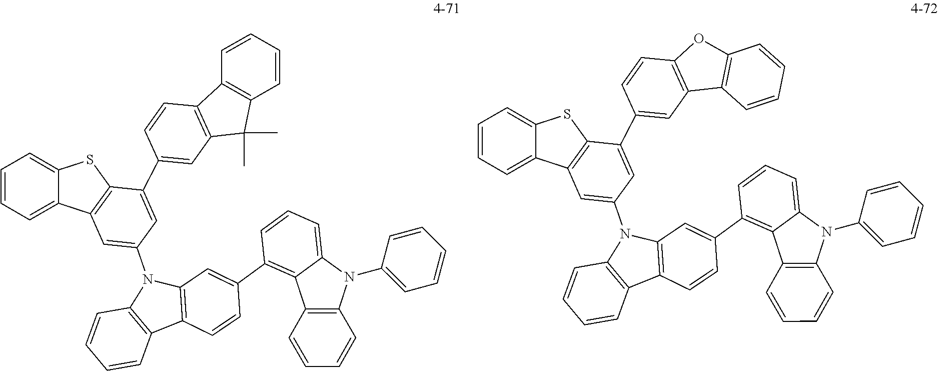 [SMILES: CC1(C)c2ccccc2-c2ccc(-c3cc(-n4c5ccccc5c5ccc(-c6cccc7c6c6ccccc6n7-c6ccccc6)cc54)cc4c3sc3ccccc34)cc21 ; c1ccc(-n2c3ccccc3c3c(-c4ccc5c6ccccc6n(-c6cc(-c7ccc8oc9ccccc9c8c7)c7sc8ccccc8c7c6)c5c4)cccc32)cc1]

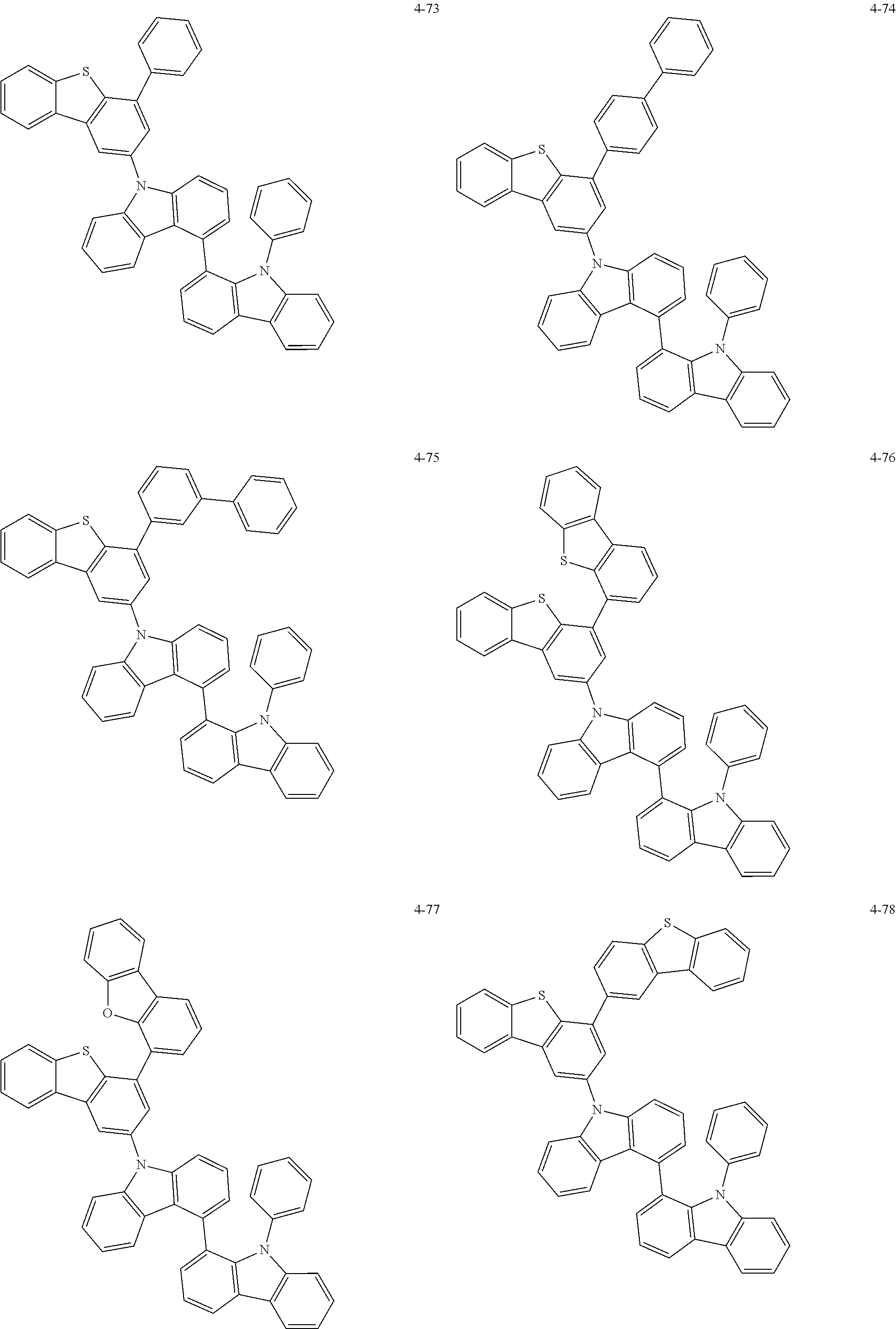

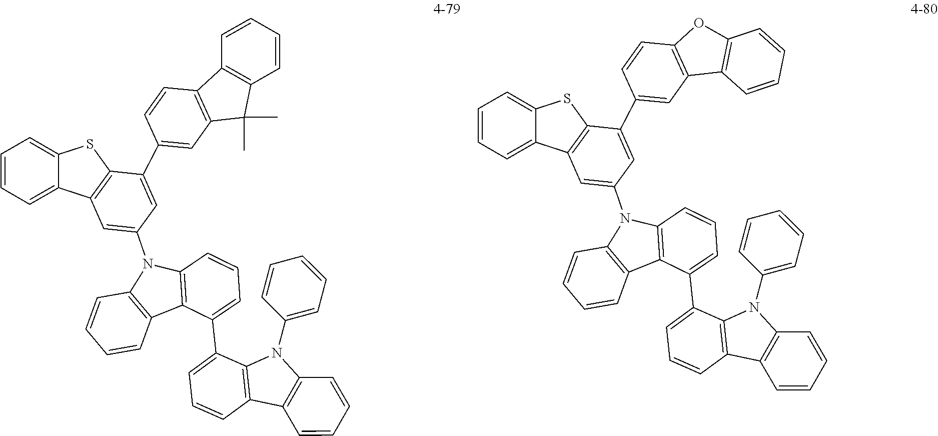 [SMILES: CC1(C)c2ccccc2-c2ccc(-c3cc(-n4c5ccccc5c5c(-c6cccc7c8ccccc8n(-c8ccccc8)c67)cccc54)cc4c3sc3ccccc34)cc21 ; c1ccc(-n2c3ccccc3c3cccc(-c4cccc5c4c4ccccc4n5-c4cc(-c5ccc6oc7ccccc7c6c5)c5sc6ccccc6c5c4)c32)cc1]

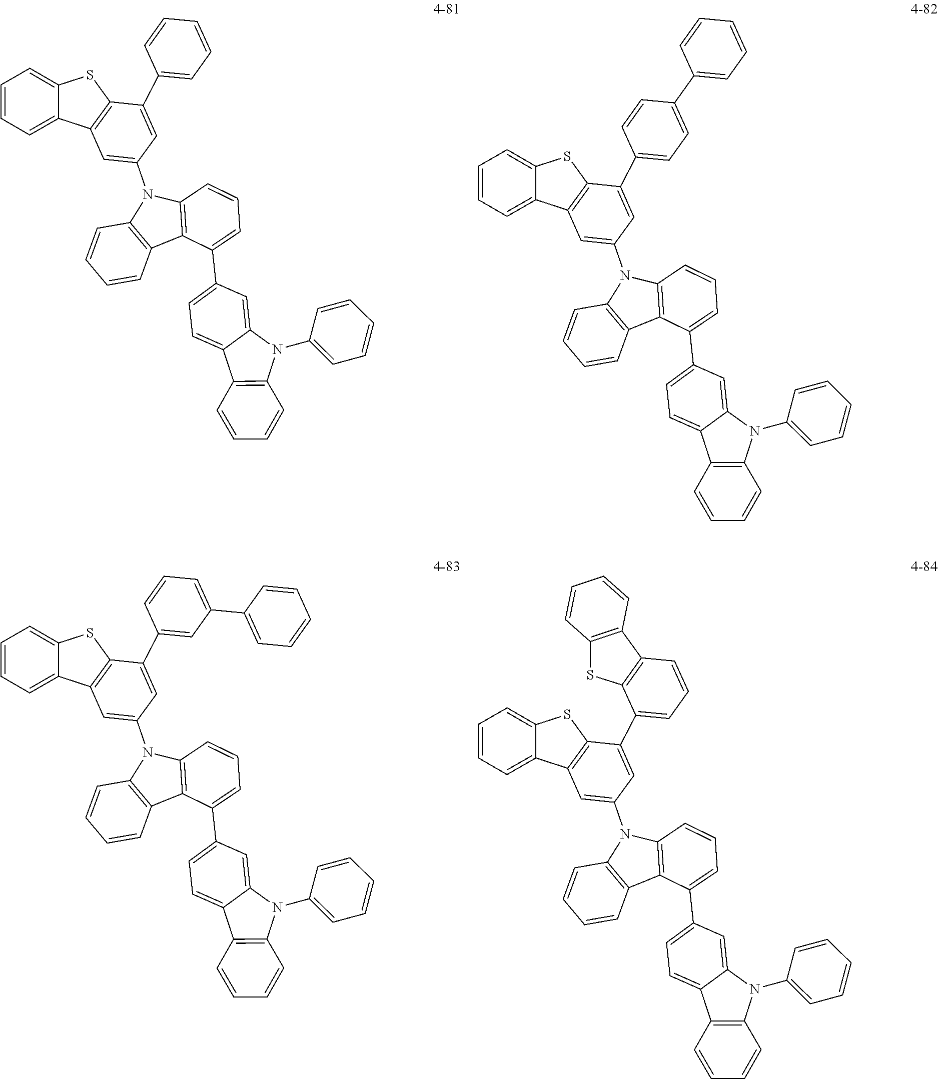 [SMILES: c1ccc(-c2cc(-n3c4ccccc4c4c(-c5ccc6c7ccccc7n(-c7ccccc7)c6c5)cccc43)cc3c2sc2ccccc23)cc1 ; c1ccc(-c2ccc(-c3cc(-n4c5ccccc5c5c(-c6ccc7c8ccccc8n(-c8ccccc8)c7c6)cccc54)cc4c3sc3ccccc34)cc2)cc1 ; c1ccc(-c2cccc(-c3cc(-n4c5ccccc5c5c(-c6ccc7c8ccccc8n(-c8ccccc8)c7c6)cccc54)cc4c3sc3ccccc34)c2)cc1 ; c1ccc(-n2c3ccccc3c3ccc(-c4cccc5c4c4ccccc4n5-c4cc(-c5cccc6c5sc5ccccc56)c5sc6ccccc6c5c4)cc32)cc1]

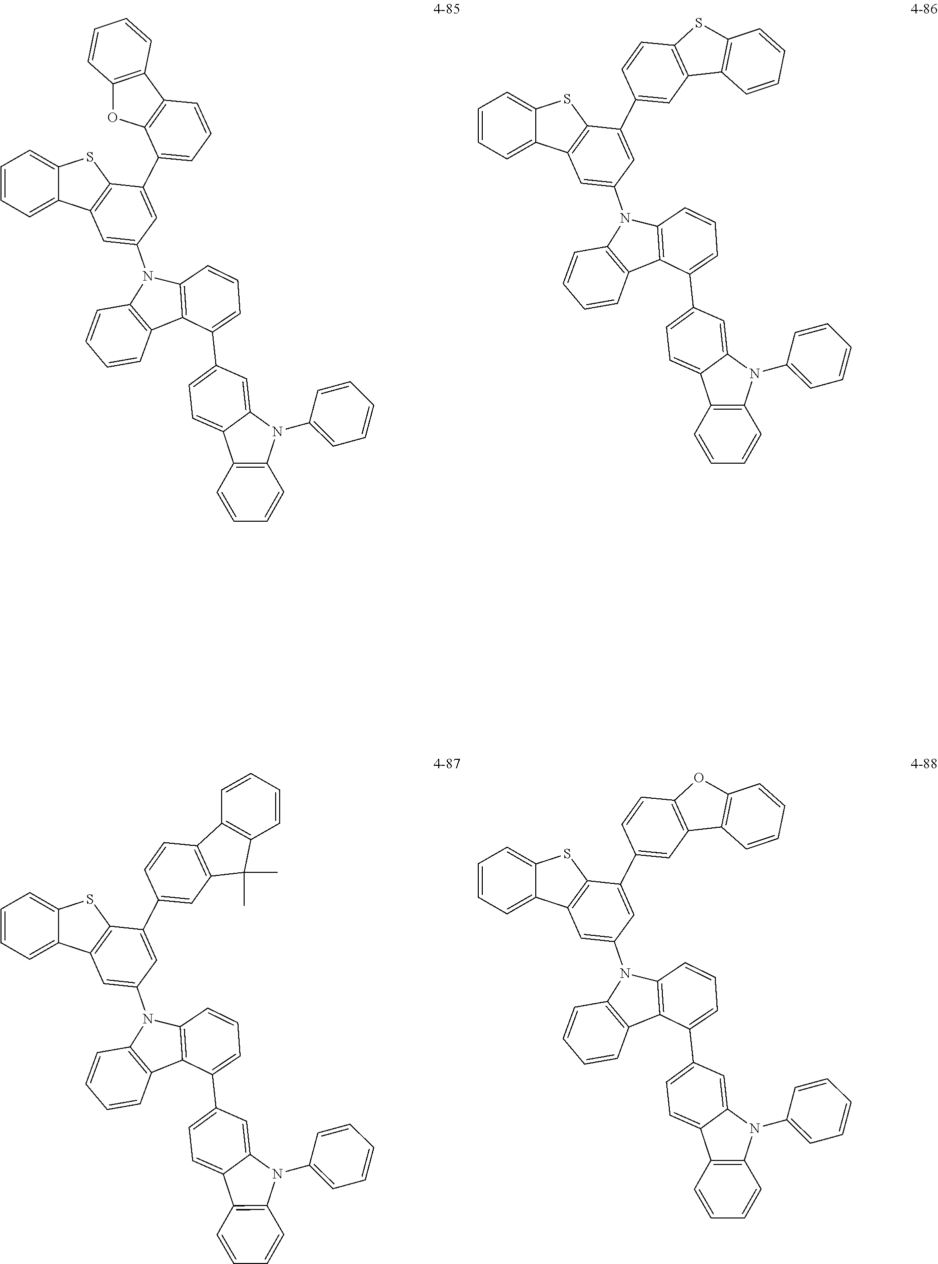 [SMILES: c1ccc(-n2c3ccccc3c3ccc(-c4cccc5c4c4ccccc4n5-c4cc(-c5cccc6c5oc5ccccc56)c5sc6ccccc6c5c4)cc32)cc1 ; c1ccc(-n2c3ccccc3c3ccc(-c4cccc5c4c4ccccc4n5-c4cc(-c5ccc6sc7ccccc7c6c5)c5sc6ccccc6c5c4)cc32)cc1 ; CC1(C)c2ccccc2-c2ccc(-c3cc(-n4c5ccccc5c5c(-c6ccc7c8ccccc8n(-c8ccccc8)c7c6)cccc54)cc4c3sc3ccccc34)cc21 ; c1ccc(-n2c3ccccc3c3ccc(-c4cccc5c4c4ccccc4n5-c4cc(-c5ccc6oc7ccccc7c6c5)c5sc6ccccc6c5c4)cc32)cc1]

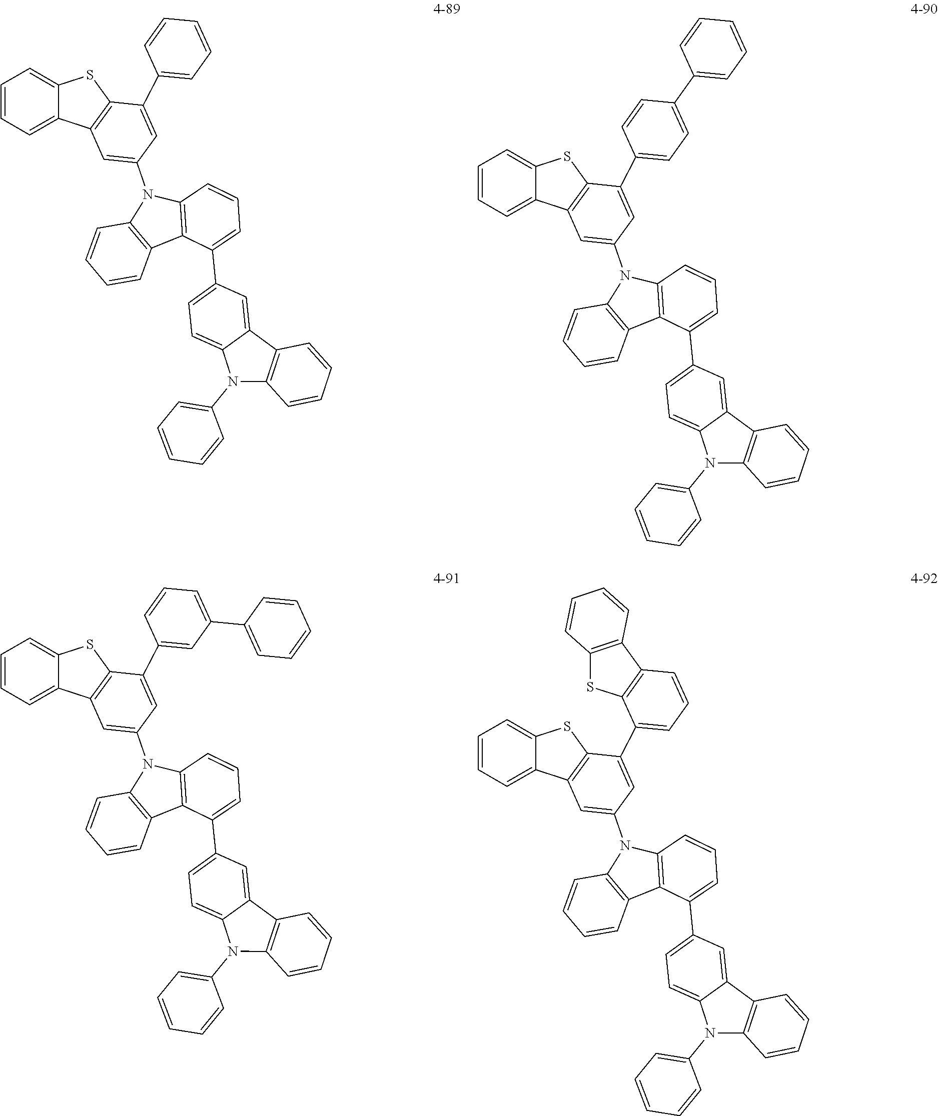 [SMILES: c1ccc(-c2cc(-n3c4ccccc4c4c(-c5ccc6c(c5)c5ccccc5n6-c5ccccc5)cccc43)cc3c2sc2ccccc23)cc1 ; c1ccc(-c2ccc(-c3cc(-n4c5ccccc5c5c(-c6ccc7c(c6)c6ccccc6n7-c6ccccc6)cccc54)cc4c3sc3ccccc34)cc2)cc1 ; c1ccc(-c2cccc(-c3cc(-n4c5ccccc5c5c(-c6ccc7c(c6)c6ccccc6n7-c6ccccc6)cccc54)cc4c3sc3ccccc34)c2)cc1 ; c1ccc(-n2c3ccccc3c3cc(-c4cccc5c4c4ccccc4n5-c4cc(-c5cccc6c5sc5ccccc56)c5sc6ccccc6c5c4)ccc32)cc1]

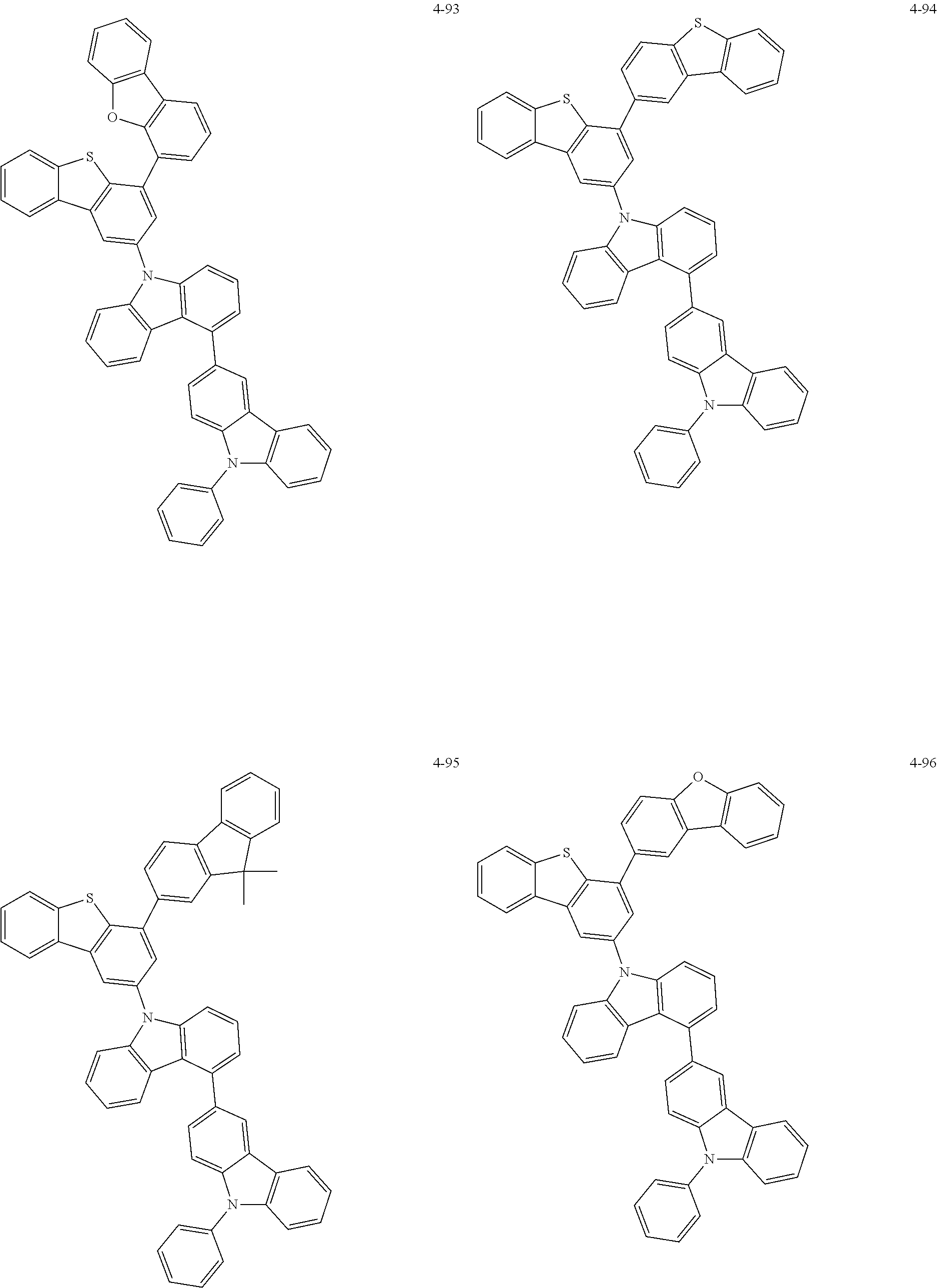 [SMILES: c1ccc(-n2c3ccccc3c3cc(-c4cccc5c4c4ccccc4n5-c4cc(-c5cccc6c5oc5ccccc56)c5sc6ccccc6c5c4)ccc32)cc1 ; c1ccc(-n2c3ccccc3c3cc(-c4cccc5c4c4ccccc4n5-c4cc(-c5ccc6sc7ccccc7c6c5)c5sc6ccccc6c5c4)ccc32)cc1 ; CC1(C)c2ccccc2-c2ccc(-c3cc(-n4c5ccccc5c5c(-c6ccc7c(c6)c6ccccc6n7-c6ccccc6)cccc54)cc4c3sc3ccccc34)cc21 ; c1ccc(-n2c3ccccc3c3cc(-c4cccc5c4c4ccccc4n5-c4cc(-c5ccc6oc7ccccc7c6c5)c5sc6ccccc6c5c4)ccc32)cc1]

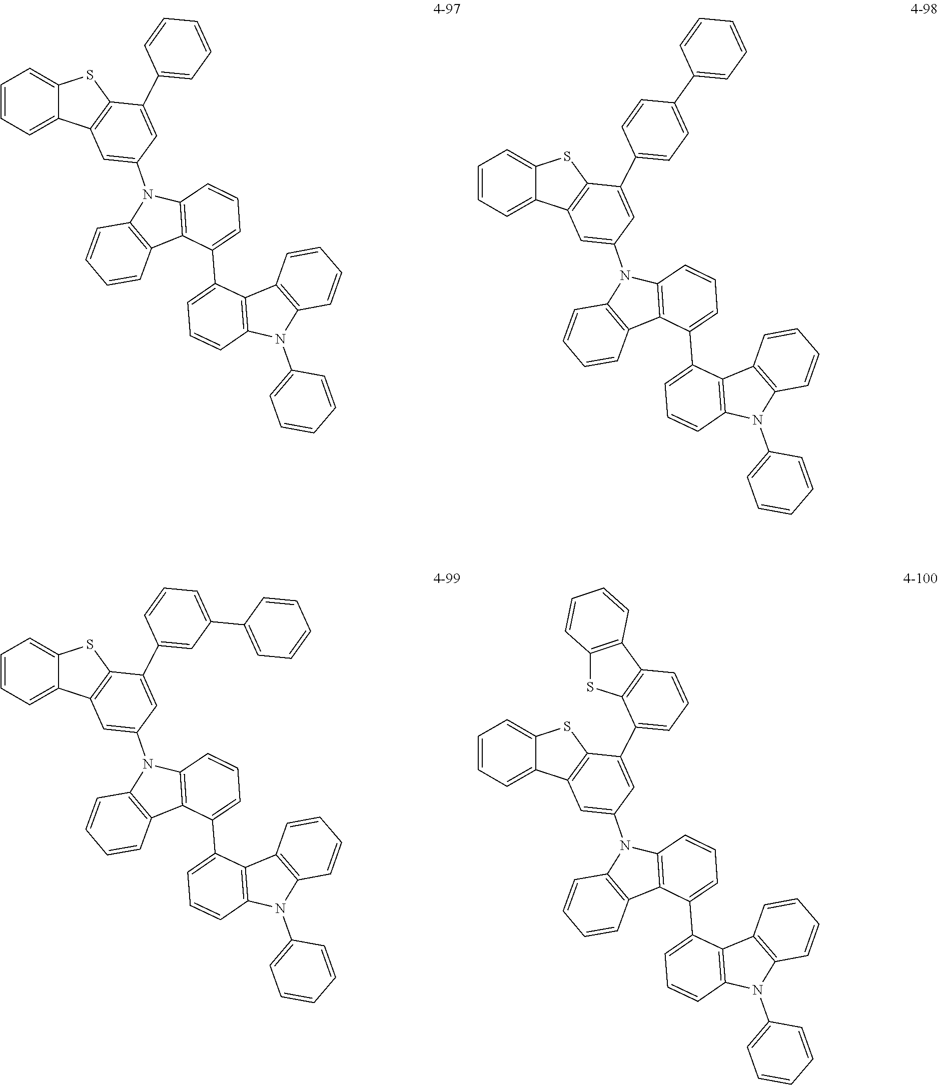 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-c3cc(-n4c5ccccc5c5c(-c6cccc7c6c6ccccc6n7-c6ccccc6)cccc54)cc4c3sc3ccccc34)cc2)cc1 ; c1ccc(-c2cccc(-c3cc(-n4c5ccccc5c5c(-c6cccc7c6c6ccccc6n7-c6ccccc6)cccc54)cc4c3sc3ccccc34)c2)cc1 ; c1ccc(-n2c3ccccc3c3c(-c4cccc5c4c4ccccc4n5-c4cc(-c5cccc6c5sc5ccccc56)c5sc6ccccc6c5c4)cccc32)cc1]

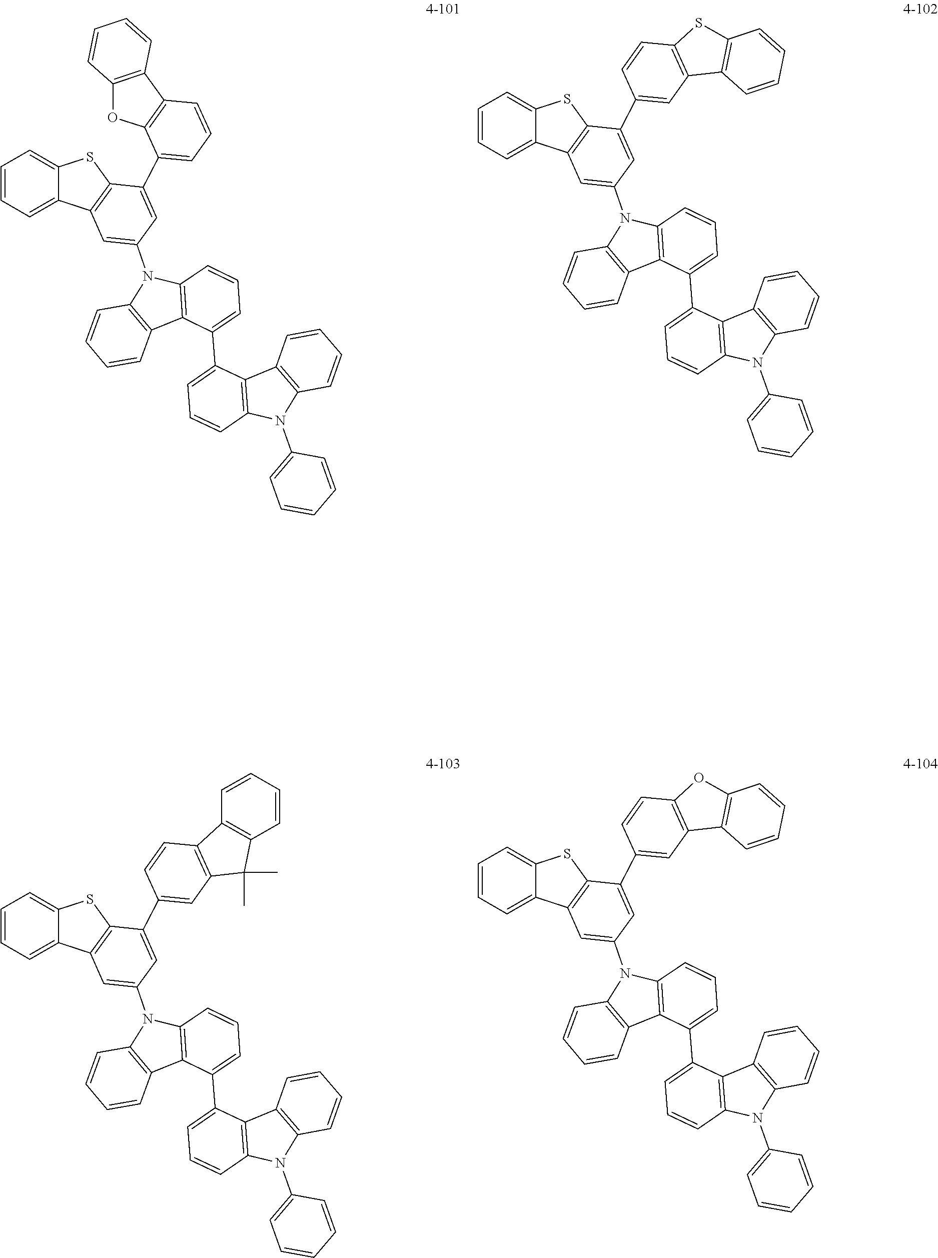 [SMILES: c1ccc(-n2c3ccccc3c3c(-c4cccc5c4c4ccccc4n5-c4cc(-c5cccc6c5oc5ccccc56)c5sc6ccccc6c5c4)cccc32)cc1 ; c1ccc(-n2c3ccccc3c3c(-c4cccc5c4c4ccccc4n5-c4cc(-c5ccc6sc7ccccc7c6c5)c5sc6ccccc6c5c4)cccc32)cc1 ; CC1(C)c2ccccc2-c2ccc(-c3cc(-n4c5ccccc5c5c(-c6cccc7c6c6ccccc6n7-c6ccccc6)cccc54)cc4c3sc3ccccc34)cc21 ; c1ccc(-n2c3ccccc3c3c(-c4cccc5c4c4ccccc4n5-c4cc(-c5ccc6oc7ccccc7c6c5)c5sc6ccccc6c5c4)cccc32)cc1]

In one embodiment of the present specification, the first electrode may be an anode, and the second electrode may be a cathode.

In another embodiment of the present specification, the first electrode may be a cathode, and the second electrode may be an anode.

In one embodiment of the present specification, the organic light emitting device may be a blue organic light emitting device, and the compound represented by Chemical Formula 1 may be used as a material of the blue organic light emitting device. For example, the compound represented by Chemical Formula 1 may be included in a light emitting layer of the blue organic light emitting device.

In one embodiment of the present specification, the organic light emitting device may be a green organic light emitting device, and the compound represented by Chemical Formula 1 may be used as a material of the green organic light emitting device. For example, the compound represented by Chemical Formula 1 may be included in a light emitting layer of the green organic light emitting device.

In one embodiment of the present specification, the organic light emitting device may be a red organic light emitting device, and the compound represented by Chemical Formula 1 may be used as a material of the red organic light emitting device. For example, the compound represented by Chemical Formula 1 may be included in a light emitting layer of the red organic light emitting device.

The organic light emitting device of the present specification may be manufactured using common organic light emitting device manufacturing methods and materials except that one or more of the organic material layers are formed using the compound described above.

The compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present specification may be formed in a single layer structure, but may be formed in a multilayer structure in which two or more organic material layers are laminated. For example, the organic light emitting device of the present disclosure may have a structure including a hole injection layer, a hole transfer layer, a light emitting layer, an electron transfer layer, an electron injection layer and the like as the organic material layer. However, the structure of the organic light emitting device is not limited thereto, and may include a smaller number of organic material layers.

In the organic light emitting device of the present specification, the organic material layer includes a light emitting layer, and the light emitting layer may include one or more types of the compound represented by Chemical Formula 1.

In the organic light emitting device of the present specification, the organic material layer includes a light emitting layer, and the light emitting layer may include one type of the compound represented by Chemical Formula 1.

In the organic light emitting device of the present specification, the organic material layer includes a light emitting layer, and the light emitting layer may include two types of the compound represented by Chemical Formula 1.

In the organic light emitting device of the present specification, the organic material layer includes a light emitting layer, and the light emitting layer may include the compound represented by Chemical Formula 1 and the compound represented by Chemical Formula 2.

In the organic light emitting device of the present specification, the organic material layer includes a light emitting layer, the light emitting layer includes a host material, and the host material may include the compound represented by Chemical Formula 1.

The organic light emitting device of the present disclosure may further include one, two or more layers selected from the group consisting of a light emitting layer, a hole injection layer, a hole transfer layer. an electron injection layer, an electron transfer layer, an electron blocking layer and a hole blocking layer.

FIG. 1 to FIG. 3 illustrate a lamination order of electrodes and organic material layers of an organic light emitting device according to one embodiment of the present specification. However, the scope of the present application is not limited to these diagrams, and structures of organic light emitting devices known in the art may also be used in the present application.

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates cases of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer including the compound represented by Chemical Formula 1 may further include other materials as necessary.

In the organic light emitting device according to one embodiment of the present specification, materials other than the compounds represented by Chemical Formulae 1 and 2 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

The organic light emitting device according to one embodiment of the present specification may be a top-emission type, a bottom-emission type or a dual-emission type depending on the materials used.

The compound according to one embodiment of the present specification may also be used in an organic electronic device including an organic solar cell, an organic photo conductor, an organic transistor and the like under a similar principle used in the organic light emitting device.

One embodiment of the present specification provides a composition for forming an organic material layer, the composition including a) a compound represented by the following Chemical Formula N, and b) a compound represented by the following Chemical Formula P or Chemical Formula 2.

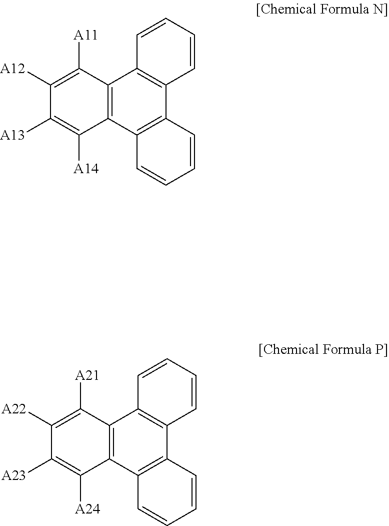

In Chemical Formula N and Chemical Formula P,

at least one of A11 to A14 includes at least one heteroaryl group including a pyridine ring, a pyrimidine ring, a triazine ring or an imidazole ring,

A21 to A24 do not include a heteroaryl group including a pyridine ring, a pyrimidine ring, a triazine ring or an imidazole ring,

one of A11 and A12 is (L1)ₐ-Q1, the other one of A11 and A12, and A13 and A14 are each independently hydrogen or (L2)_{b}-Q2, and at least one thereof is (L2)_{b}-Q2,

one of A21 and A22 is (L1)ₐ-Q1, the other one of A21 and A22, and A23 and A24 are each independently hydrogen or (L2)_{b}-Q2, and at least one thereof is (L2)_{b}-Q2,

a and b are each independently an integer of 1 to 5,

when a and b are each 2 or greater, substituents in the parentheses are the same as or different from each other,

L1 and L2 are each independently a direct bond; a substituted or unsubstituted C6 to C60 arylene group; or a substituted or unsubstituted C2 to C60 heteroarylene group,

Q1 is a substituted or unsubstituted C6 to C20 aryl group; or a substituted or unsubstituted C2 to C20 heteroaryl group including N,

Q2 is a cyano group; a substituted or unsubstituted silyl group; a substituted or unsubstituted amine group; a substituted or unsubstituted C1 to C20 alkyl group; a substituted or unsubstituted C6 to C30 aryl group; a substituted or unsubstituted C2 to C30 heteroaryl group; or a substituted or unsubstituted phosphine oxide group,

when A12 and A13 are hydrogen, Q1 is a phenyl group and Q2 includes pyridine or triazine, L1 is a substituted or unsubstituted C6 to C60 arylene group; or a substituted or unsubstituted C2 to C60 heteroarylene group, and

when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a dicyclic or higher aryl group unsubstituted or substituted with an alkyl group or an aryl group is satisfied,

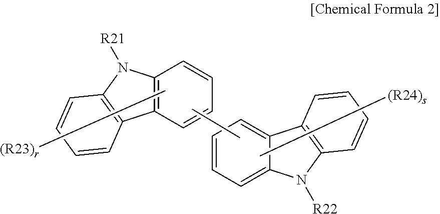

in Chemical Formula 2,

R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,

R23 and R24 are each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,

r and s are each an integer of 0 to 7, and

when r and s are each 2 or greater, substituents in the parentheses are the same as or different from each other.

The composition for forming an organic material layer according to one embodiment of the present specification may include a) the compound represented by Chemical Formula N and b) the compound represented by Chemical Formula P or Chemical Formula 2 in a weight ratio of 1:10 to 10:1, 1:8 to 8:1, 1:5 to 5:1 or 1:2 to 2:1, and the weight ratio may be preferably 1:2.

In the present specification, Chemical Formula N is the same as Chemical Formula 1-N, and Chemical Formula P is the same as Chemical Formula 1-P.

Hereinafter, the present specification will be described in more detail with reference to examples, however, these are for illustrative purposes only, and the scope of the present application is not limited thereto.

### PREPARATION EXAMPLE

### <Preparation Example 1> Preparation of Compound 1-1

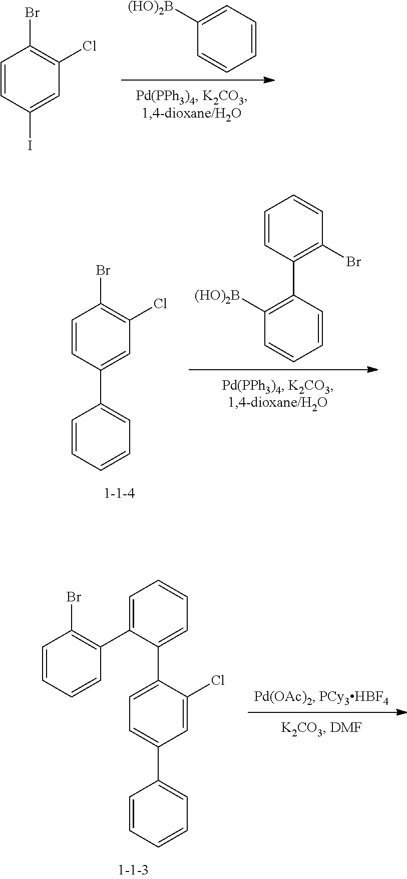 [SMILES: Clc1cc(I)ccc1Br ; OB(O)c1ccccc1 ; Clc1cc(-c2ccccc2)ccc1Br ; OB(O)c1ccccc1-c1ccccc1Br ; Clc1cc(-c2ccccc2)ccc1-c1ccccc1-c1ccccc1Br]

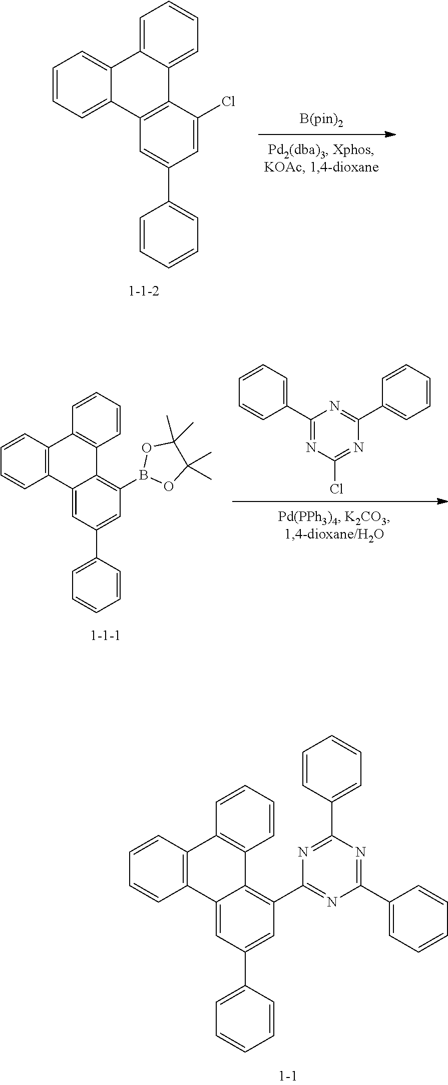 [SMILES: Clc1cc(-c2ccccc2)cc2c3ccccc3c3ccccc3c12 ; CC1(C)OB(c2cc(-c3ccccc3)cc3c4ccccc4c4ccccc4c23)OC1(C)C ; Clc1nc(-c2ccccc2)nc(-c2ccccc2)n1 ; c1ccc(-c2cc(-c3nc(-c4ccccc4)nc(-c4ccccc4)n3)c3c4ccccc4c4ccccc4c3c2)cc1]

1) Preparation of Compound 1-1-4

After dissolving 1-bromo-2-chloro-4-iodobenzene (18.2 g, 57.4 mM), phenylboronic acid (7.7 g, 63.1 mM), Pd(PPh₃)₄ (tetrakis(triphenylphosphine)palladium(0)) (3.3 g, 2.9 mM) and K₂CO₃ (15.9 g, 114.8 mM) in 1,4-dioxane/H₂O (200 mL/40 mL), the result was refluxed for 24 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex (hexane)=1:10) to obtain Compound 1-1-4 (12.3 g, 80%).

2) Preparation of Compound 1-1-3

After dissolving Compound 1-1-4 (10 g, 37.4 mM), (2′-bromo-[1,1′-biphenyl]-2-yl)boronic acid (10.4 g, 37.4 mM), Pd(PPh₃)₄ (2.2 g, 1.9 mM) and K₂CO₃ (10.3 g, 74.8 mM) in 1,4-dioxane/H₂O (200 mL/40 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:10) to obtain Compound 1-1-3 (13.3 g, 85%).

3) Preparation of Compound 1-1-2

After dissolving Compound 1-1-3 (11.6 g, 27.7 mM), Pd(OAc)₂ (palladium(II) acetate) (622 mg, 2.8 mM), PCy₃.HBF₄ (tricyclohexylphosphine tetrafluoroborate (2.0 g, 5.5 mM) and K₂CO₃ (7.7 g, 55.4 mM) in N,N-dimethylformamide (DMF) (100 mL), the result was refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:10) to obtain Compound 1-1-2 (6.6 g, 70%).

4) Preparation of Compound 1-1-1

After dissolving Compound 1-1-2 (6.5 g, 19.2 mM), bis(pinacolato)diboron (7.3 g, 28.8 mM), Pd₂(dba)₃ (tris(dibenzylideneacetone)dipalladium(0)) (879 mg, 1.0 mM), Xphos (2-dicyclohexylphosphino-2′,4′,6′-triisopropylbiphenyl) (915 mg, 1.9 mM) and KOAc (potassium acetate) (5.6 g, 57.3 mM) in 1,4-dioxane (100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain Compound 1-1-1 (7 g, 85%).

5) Preparation of Compound 1-1

After dissolving Compound 1-1-1 (6.9 g, 16.1 mM), 2-chloro-4,6-diphenyl-1,3,5-triazine (4.7 g, 17.7 mM), Pd(PPh₃)₄ (0.9 g, 0.8 mM) and K₂CO₃ (4.5 g, 32.3 mM) in 1,4-dioxane/H₂O (200 mL/40 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 1-1 (7.1 g, 82%).

Target Compound A was synthesized in the same manner as in Preparation Example 1 except that Intermediate A of the following Table 1 was used instead of phenylboronic acid, and Intermediate B of the following Table 1 was used instead of 2-chloro-4,6-diphenyl-1,3,5-triazine.

TABLE 1

| Com- | | | | |
|---|---|---|---|---|
| pound | | | | |
| No. | Intermediate A | Intermediate B | Target Compound A | Yield |
| 1-4 | 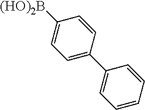 [SMILES: OB(O)c1ccc(-c2ccccc2)cc1] | 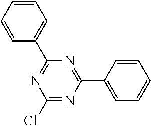 [SMILES: Clc1nc(-c2ccccc2)nc(-c2ccccc2)n1] | 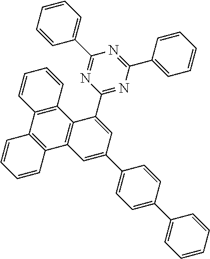 [SMILES: c1ccc(-c2ccc(-c3cc(-c4nc(-c5ccccc5)nc(-c5ccccc5)n4)c4c5ccccc5c5ccccc5c4c3)cc2)cc1] | 42% |
| 1-7 | 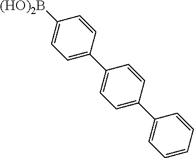 [SMILES: OB(O)c1ccc(-c2ccc(-c3ccccc3)cc2)cc1] | | 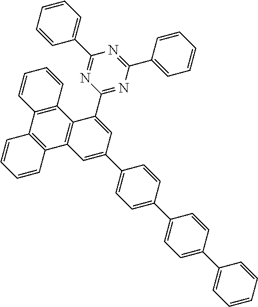 [SMILES: c1ccc(-c2ccc(-c3ccc(-c4cc(-c5nc(-c6ccccc6)nc(-c6ccccc6)n5)c5c6ccccc6c6ccccc6c5c4)cc3)cc2)cc1] | 46% |
| 1-8 | 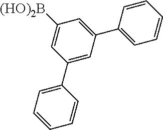 [SMILES: OB(O)c1cc(-c2ccccc2)cc(-c2ccccc2)c1] | | 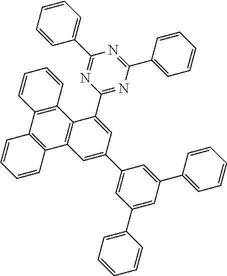 [SMILES: c1ccc(-c2cc(-c3ccccc3)cc(-c3cc(-c4nc(-c5ccccc5)nc(-c5ccccc5)n4)c4c5ccccc5c5ccccc5c4c3)c2)cc1] | 43% |
| 1-9 | 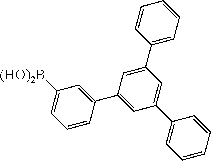 [SMILES: OB(O)c1cccc(-c2cc(-c3ccccc3)cc(-c3ccccc3)c2)c1] | | 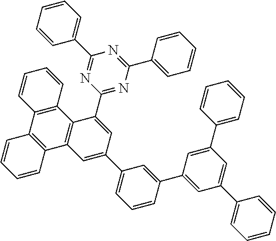 [SMILES: c1ccc(-c2cc(-c3ccccc3)cc(-c3cccc(-c4cc(-c5nc(-c6ccccc6)nc(-c6ccccc6)n5)c5c6ccccc6c6ccccc6c5c4)c3)c2)cc1] | 45% |
| 1-17 | 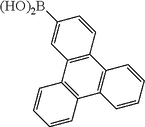 [SMILES: OB(O)c1ccc2c3ccccc3c3ccccc3c2c1] | | 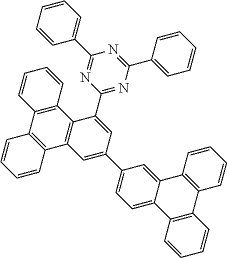 [SMILES: c1ccc(-c2nc(-c3ccccc3)nc(-c3cc(-c4ccc5c6ccccc6c6ccccc6c5c4)cc4c5ccccc5c5ccccc5c34)n2)cc1] | 44% |
| 1-21 | 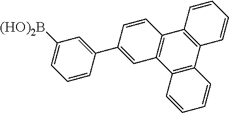 [SMILES: OB(O)c1cccc(-c2ccc3c4ccccc4c4ccccc4c3c2)c1] | | 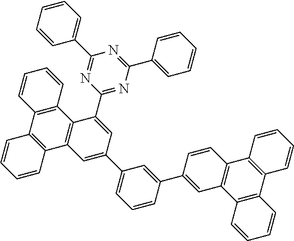 [SMILES: c1ccc(-c2nc(-c3ccccc3)nc(-c3cc(-c4cccc(-c5ccc6c7ccccc7c7ccccc7c6c5)c4)cc4c5ccccc5c5ccccc5c34)n2)cc1] | 42% |
| 1-27 | 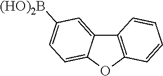 [SMILES: OB(O)c1ccc2oc3ccccc3c2c1] | | 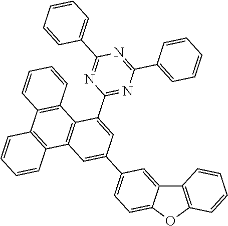 [SMILES: c1ccc(-c2nc(-c3ccccc3)nc(-c3cc(-c4ccc5oc6ccccc6c5c4)cc4c5ccccc5c5ccccc5c34)n2)cc1] | 46% |
| 1-28 | 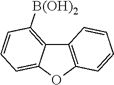 [SMILES: OB(O)c1cccc2oc3ccccc3c12] | | 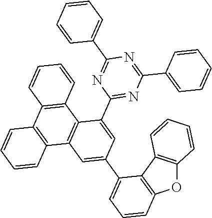 [SMILES: c1ccc(-c2nc(-c3ccccc3)nc(-c3cc(-c4cccc5oc6ccccc6c45)cc4c5ccccc5c5ccccc5c34)n2)cc1] | 43% |
| 1-31 | 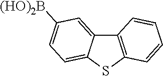 [SMILES: OB(O)c1ccc2sc3ccccc3c2c1] | | 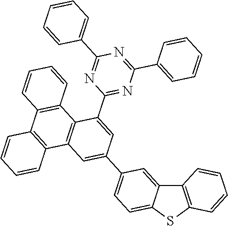 [SMILES: c1ccc(-c2nc(-c3ccccc3)nc(-c3cc(-c4ccc5sc6ccccc6c5c4)cc4c5ccccc5c5ccccc5c34)n2)cc1] | 45% |
| 1-34 | 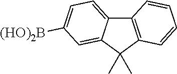 [SMILES: CC1(C)c2ccccc2-c2ccc(B(O)O)cc21] | | 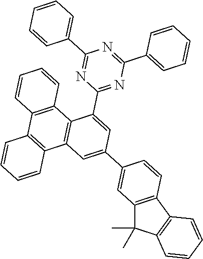 [SMILES: CC1(C)c2ccccc2-c2ccc(-c3cc(-c4nc(-c5ccccc5)nc(-c5ccccc5)n4)c4c5ccccc5c5ccccc5c4c3)cc21] | 44% |
| 1-39 | 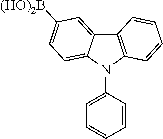 [SMILES: OB(O)c1ccc2c(c1)c1ccccc1n2-c1ccccc1] | | 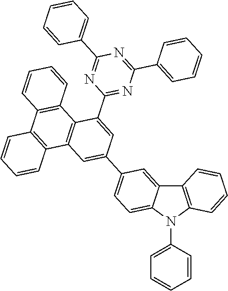 [SMILES: c1ccc(-c2nc(-c3ccccc3)nc(-c3cc(-c4ccc5c(c4)c4ccccc4n5-c4ccccc4)cc4c5ccccc5c5ccccc5c34)n2)cc1] | 44% |
| 1-44 | 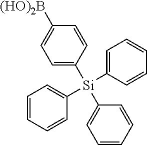 [SMILES: OB(O)c1ccc([Si](c2ccccc2)(c2ccccc2)c2ccccc2)cc1] | | 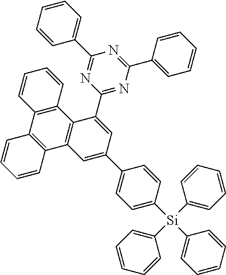 [SMILES: c1ccc(-c2nc(-c3ccccc3)nc(-c3cc(-c4ccc([Si](c5ccccc5)(c5ccccc5)c5ccccc5)cc4)cc4c5ccccc5c5ccccc5c34)n2)cc1] | 42% |
| 1-46 | 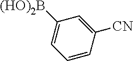 [SMILES: N#Cc1cccc(B(O)O)c1] | | 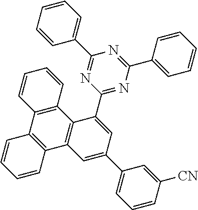 [SMILES: N#Cc1cccc(-c2cc(-c3nc(-c4ccccc4)nc(-c4ccccc4)n3)c3c4ccccc4c4ccccc4c3c2)c1] | 46% |
| 1-49 | 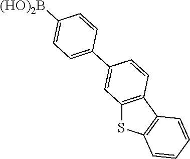 [SMILES: OB(O)c1ccc(-c2ccc3c(c2)sc2ccccc23)cc1] | | 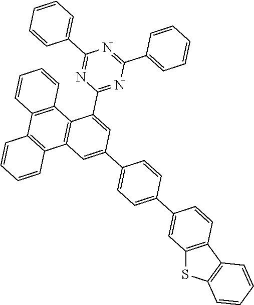 [SMILES: c1ccc(-c2nc(-c3ccccc3)nc(-c3cc(-c4ccc(-c5ccc6c(c5)sc5ccccc56)cc4)cc4c5ccccc5c5ccccc5c34)n2)cc1] | 43% |
| 1-56 | 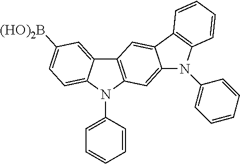 [SMILES: OB(O)c1ccc2c(c1)c1cc3c4ccccc4n(-c4ccccc4)c3cc1n2-c1ccccc1] | | 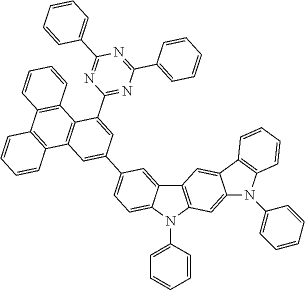 [SMILES: c1ccc(-c2nc(-c3ccccc3)nc(-c3cc(-c4ccc5c(c4)c4cc6c7ccccc7n(-c7ccccc7)c6cc4n5-c4ccccc4)cc4c5ccccc5c5ccccc5c34)n2)cc1] | 45% |
| 1-90 | 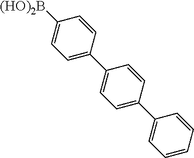 [SMILES: OB(O)c1ccc(-c2ccc(-c3ccccc3)cc2)cc1] | 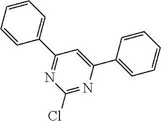 [SMILES: Clc1nc(-c2ccccc2)cc(-c2ccccc2)n1] | 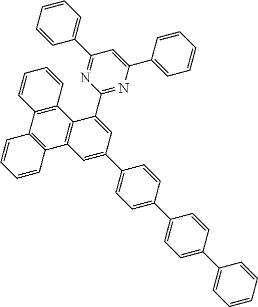 [SMILES: c1ccc(-c2ccc(-c3ccc(-c4cc(-c5nc(-c6ccccc6)cc(-c6ccccc6)n5)c5c6ccccc6c6ccccc6c5c4)cc3)cc2)cc1] | 41% |
| 1-92 | 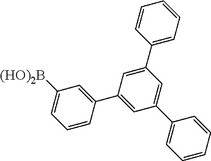 [SMILES: OB(O)c1cccc(-c2cc(-c3ccccc3)cc(-c3ccccc3)c2)c1] | 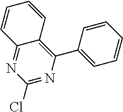 [SMILES: Clc1nc(-c2ccccc2)c2ccccc2n1] | 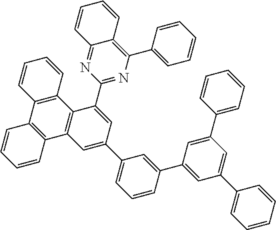 [SMILES: c1ccc(-c2cc(-c3ccccc3)cc(-c3cccc(-c4cc(-c5nc(-c6ccccc6)c6ccccc6n5)c5c6ccccc6c6ccccc6c5c4)c3)c2)cc1] | 42% |
| 1-121 | 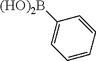 | 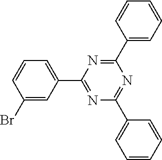 [SMILES: Brc1cccc(-c2nc(-c3ccccc3)nc(-c3ccccc3)n2)c1] | 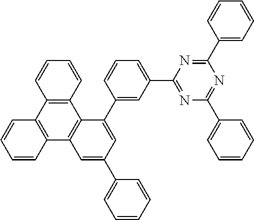 [SMILES: c1ccc(-c2cc(-c3cccc(-c4nc(-c5ccccc5)nc(-c5ccccc5)n4)c3)c3c4ccccc4c4ccccc4c3c2)cc1] | 43% |
| 1-122 | | 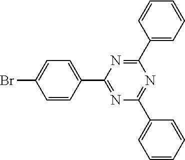 [SMILES: Brc1ccc(-c2nc(-c3ccccc3)nc(-c3ccccc3)n2)cc1] | 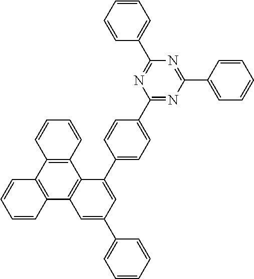 [SMILES: c1ccc(-c2cc(-c3ccc(-c4nc(-c5ccccc5)nc(-c5ccccc5)n4)cc3)c3c4ccccc4c4ccccc4c3c2)cc1] | 45% |
| 1-127 | 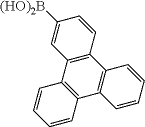 [SMILES: OB(O)c1ccc2c3ccccc3c3ccccc3c2c1] | 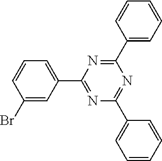 [SMILES: Brc1cccc(-c2nc(-c3ccccc3)nc(-c3ccccc3)n2)c1] | 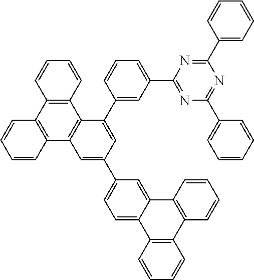 [SMILES: c1ccc(-c2nc(-c3ccccc3)nc(-c3cccc(-c4cc(-c5ccc6c7ccccc7c7ccccc7c6c5)cc5c6ccccc6c6ccccc6c45)c3)n2)cc1] | 44% |
| 2-7 | 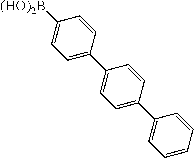 [SMILES: OB(O)c1ccc(-c2ccc(-c3ccccc3)cc2)cc1] | 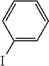 | 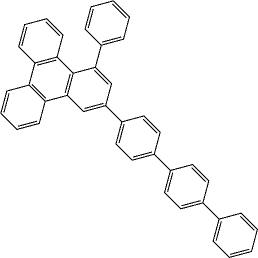 [SMILES: c1ccc(-c2ccc(-c3ccc(-c4cc(-c5ccccc5)c5c6ccccc6c6ccccc6c5c4)cc3)cc2)cc1] | 42% |
| 2-17 | 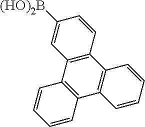 [SMILES: OB(O)c1ccc2c3ccccc3c3ccccc3c2c1] | | 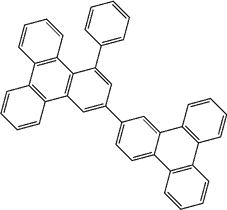 [SMILES: c1ccc(-c2cc(-c3ccc4c5ccccc5c5ccccc5c4c3)cc3c4ccccc4c4ccccc4c23)cc1] | 46% |
| 2-22 | 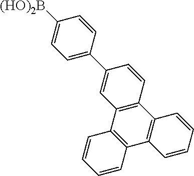 [SMILES: OB(O)c1ccc(-c2ccc3c4ccccc4c4ccccc4c3c2)cc1] | | 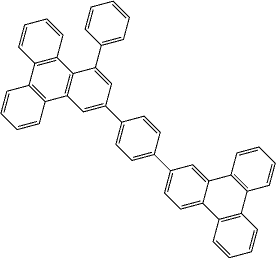 [SMILES: c1ccc(-c2cc(-c3ccc(-c4ccc5c6ccccc6c6ccccc6c5c4)cc3)cc3c4ccccc4c4ccccc4c23)cc1] | 43% |
| 2-27 | 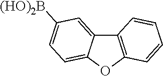 [SMILES: OB(O)c1ccc2oc3ccccc3c2c1] | | 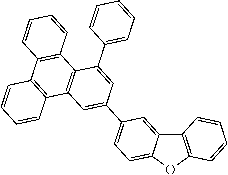 [SMILES: c1ccc(-c2cc(-c3ccc4oc5ccccc5c4c3)cc3c4ccccc4c4ccccc4c23)cc1] | 45% |
| 2-31 | 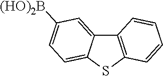 [SMILES: OB(O)c1ccc2sc3ccccc3c2c1] | | 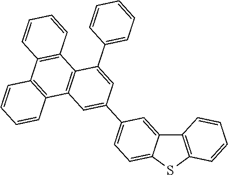 [SMILES: c1ccc(-c2cc(-c3ccc4sc5ccccc5c4c3)cc3c4ccccc4c4ccccc4c23)cc1] | 44% |
| 2-39 | 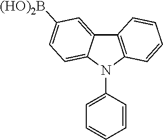 [SMILES: OB(O)c1ccc2c(c1)c1ccccc1n2-c1ccccc1] | | 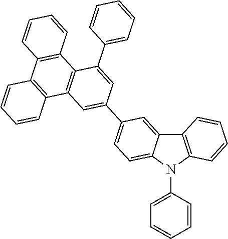 [SMILES: c1ccc(-c2cc(-c3ccc4c(c3)c3ccccc3n4-c3ccccc3)cc3c4ccccc4c4ccccc4c23)cc1] | 42% |
| 2-42 | 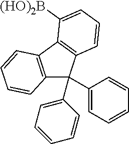 [SMILES: OB(O)c1cccc2c1-c1ccccc1C2(c1ccccc1)c1ccccc1] | | 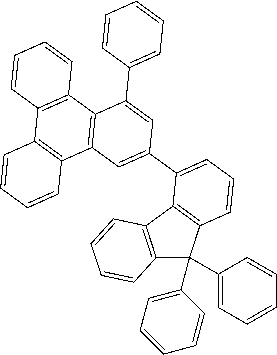 [SMILES: c1ccc(-c2cc(-c3cccc4c3-c3ccccc3C4(c3ccccc3)c3ccccc3)cc3c4ccccc4c4ccccc4c23)cc1] | 46% |
| 2-44 | 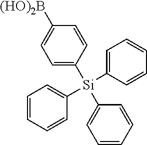 [SMILES: OB(O)c1ccc([Si](c2ccccc2)(c2ccccc2)c2ccccc2)cc1] | | 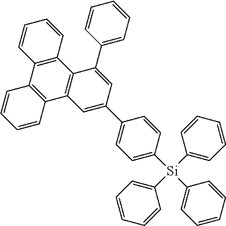 [SMILES: c1ccc(-c2cc(-c3ccc([Si](c4ccccc4)(c4ccccc4)c4ccccc4)cc3)cc3c4ccccc4c4ccccc4c23)cc1] | 43% |
| 2-56 | 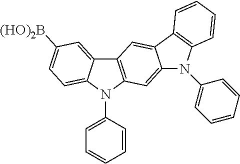 [SMILES: OB(O)c1ccc2c(c1)c1cc3c4ccccc4n(-c4ccccc4)c3cc1n2-c1ccccc1] | | 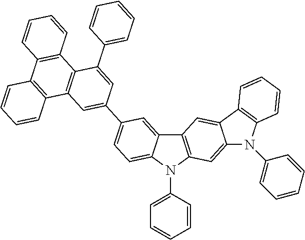 [SMILES: c1ccc(-c2cc(-c3ccc4c(c3)c3cc5c6ccccc6n(-c6ccccc6)c5cc3n4-c3ccccc3)cc3c4ccccc4c4ccccc4c23)cc1] | 45% |
| 2-70 | 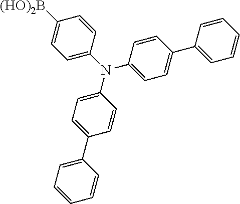 [SMILES: OB(O)c1ccc(N(c2ccc(-c3ccccc3)cc2)c2ccc(-c3ccccc3)cc2)cc1] | | 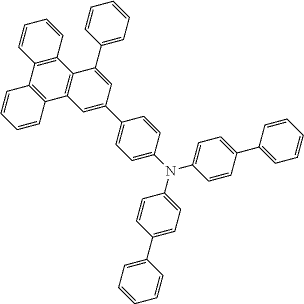 [SMILES: c1ccc(-c2ccc(N(c3ccc(-c4ccccc4)cc3)c3ccc(-c4cc(-c5ccccc5)c5c6ccccc6c6ccccc6c5c4)cc3)cc2)cc1] | 43% |
| 2-72 | 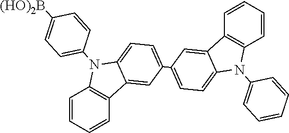 [SMILES: OB(O)c1ccc(-n2c3ccccc3c3cc(-c4ccc5c(c4)c4ccccc4n5-c4ccccc4)ccc32)cc1] | | 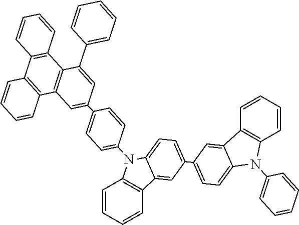 [SMILES: c1ccc(-c2cc(-c3ccc(-n4c5ccccc5c5cc(-c6ccc7c(c6)c6ccccc6n7-c6ccccc6)ccc54)cc3)cc3c4ccccc4c4ccccc4c23)cc1] | 45% |
| 2-80 | 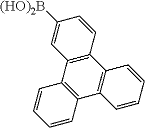 [SMILES: OB(O)c1ccc2c3ccccc3c3ccccc3c2c1] | 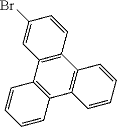 [SMILES: Brc1ccc2c3ccccc3c3ccccc3c2c1] | 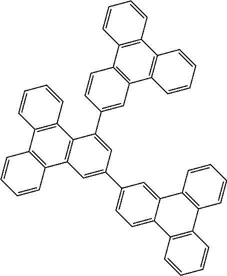 [SMILES: c1ccc2c(c1)c1ccccc1c1cc(-c3cc(-c4ccc5c6ccccc6c6ccccc6c5c4)c4c5ccccc5c5ccccc5c4c3)ccc21] | 44% |

Target Compound A was synthesized in the same manner as in Preparation Example 1 except that Intermediate C of the following Table 2 was used instead of 1-bromo-2-chloro-4-iodobenzene, Intermediate D was used instead of phenylboronic acid, and Intermediate E was used instead of 2-chloro-4,6-diphenyl-1,3,5-triazine.

TABLE 2

| Com- | | | | | |
|---|---|---|---|---|---|
| pound | Intermediate | Intermediate | Intermediate | Target | |
| No. | C | D | E | Compound A | Yield |
| 1-99 | 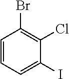 | 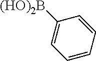 | 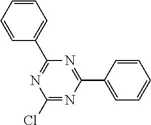 [SMILES: Clc1nc(-c2ccccc2)nc(-c2ccccc2)n1] | 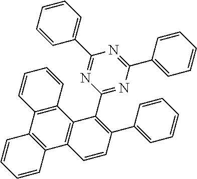 [SMILES: c1ccc(-c2nc(-c3ccccc3)nc(-c3c(-c4ccccc4)ccc4c5ccccc5c5ccccc5c34)n2)cc1] | 43% |
| 1-101 | 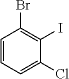 | 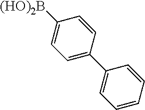 [SMILES: OB(O)c1ccc(-c2ccccc2)cc1] | | 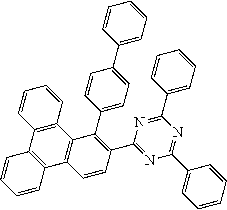 [SMILES: c1ccc(-c2ccc(-c3c(-c4nc(-c5ccccc5)nc(-c5ccccc5)n4)ccc4c5ccccc5c5ccccc5c34)cc2)cc1] | 42% |
| 1-108 | 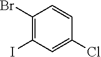 | 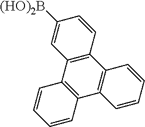 [SMILES: OB(O)c1ccc2c3ccccc3c3ccccc3c2c1] | | 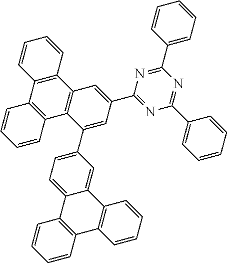 [SMILES: c1ccc(-c2nc(-c3ccccc3)nc(-c3cc(-c4ccc5c6ccccc6c6ccccc6c5c4)c4c5ccccc5c5ccccc5c4c3)n2)cc1] | 41% |

### <Preparation Example 2> Synthesis of Compound 3-3

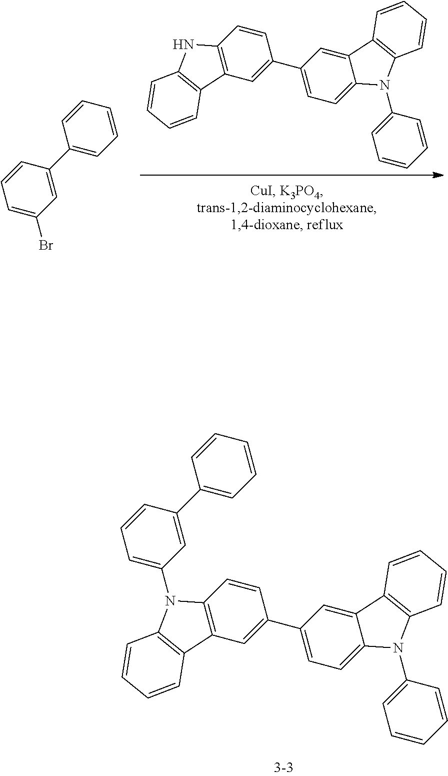

1) Preparation of Compound 3-3

After dissolving 3-bromo-1,1′-biphenyl (3.7 g, 15.8 mM), 9-phenyl-9H,9′H-3,3′-bicarbazole (6.5 g, 15.8 mM), CuI (3.0 g, 15.8 mM), trans-1,2-diaminocyclohexane (1.9 mL, 15.8 mM) and K₃PO₄ (3.3 g, 31.6 mM) in 1,4-dioxane (100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 3-3 (7.5 g, 85%).

Target Compound B was synthesized in the same manner as in Preparation Example 2 except that Intermediate F of the following Table 3 was used instead of 3-bromo-1,1′-biphenyl, and Intermediate G of the following Table 3 was used instead of 9-phenyl-9H,9′H-3,3′-bicarbazole.

TABLE 3

| Com- | | | | Over- |
|---|---|---|---|---|
| pound | Intermediate | | Target Compound | all |
| No. | F | Intermediate G | B | Yield |
| 3-4 | 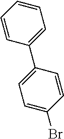 [SMILES: Brc1ccc(-c2ccccc2)cc1] | 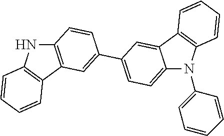 [SMILES: c1ccc(-n2c3ccccc3c3cc(-c4ccc5[nH]c6ccccc6c5c4)ccc32)cc1] | 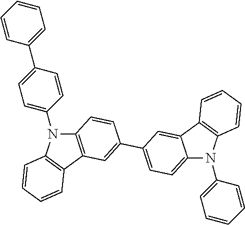 [SMILES: c1ccc(-c2ccc(-n3c4ccccc4c4cc(-c5ccc6c(c5)c5ccccc5n6-c5ccccc5)ccc43)cc2)cc1] | 83% |
| 3-7 | 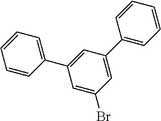 [SMILES: Brc1cc(-c2ccccc2)cc(-c2ccccc2)c1] | | 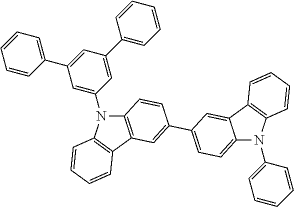 [SMILES: c1ccc(-c2cc(-c3ccccc3)cc(-n3c4ccccc4c4cc(-c5ccc6c(c5)c5ccccc5n6-c5ccccc5)ccc43)c2)cc1] | 84% |
| 3-31 | 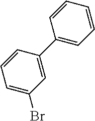 [SMILES: Brc1cccc(-c2ccccc2)c1] | 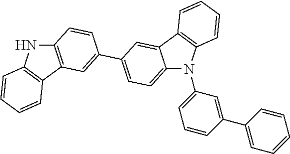 [SMILES: c1ccc(-c2cccc(-n3c4ccccc4c4cc(-c5ccc6[nH]c7ccccc7c6c5)ccc43)c2)cc1] | 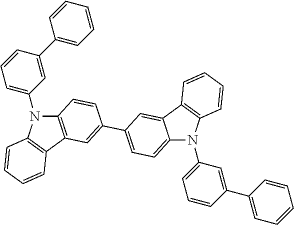 [SMILES: c1ccc(-c2cccc(-n3c4ccccc4c4cc(-c5ccc6c(c5)c5ccccc5n6-c5cccc(-c6ccccc6)c5)ccc43)c2)cc1] | 81% |
| 3-32 | 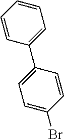 [SMILES: Brc1ccc(-c2ccccc2)cc1] | | 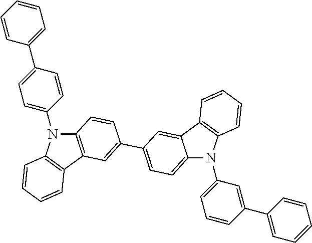 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 80% |
| 3-42 | 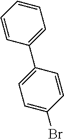 [SMILES: Brc1ccc(-c2ccccc2)cc1] | 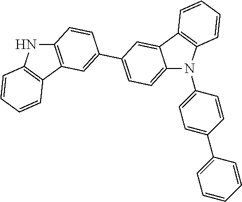 [SMILES: c1ccc(-c2ccc(-n3c4ccccc4c4cc(-c5ccc6[nH]c7ccccc7c6c5)ccc43)cc2)cc1] | 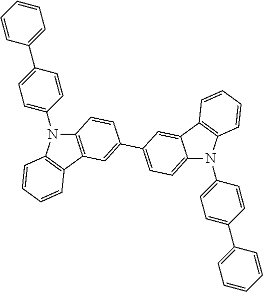 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 82% |

### <Preparation Example 3> Synthesis of Compound 4-2

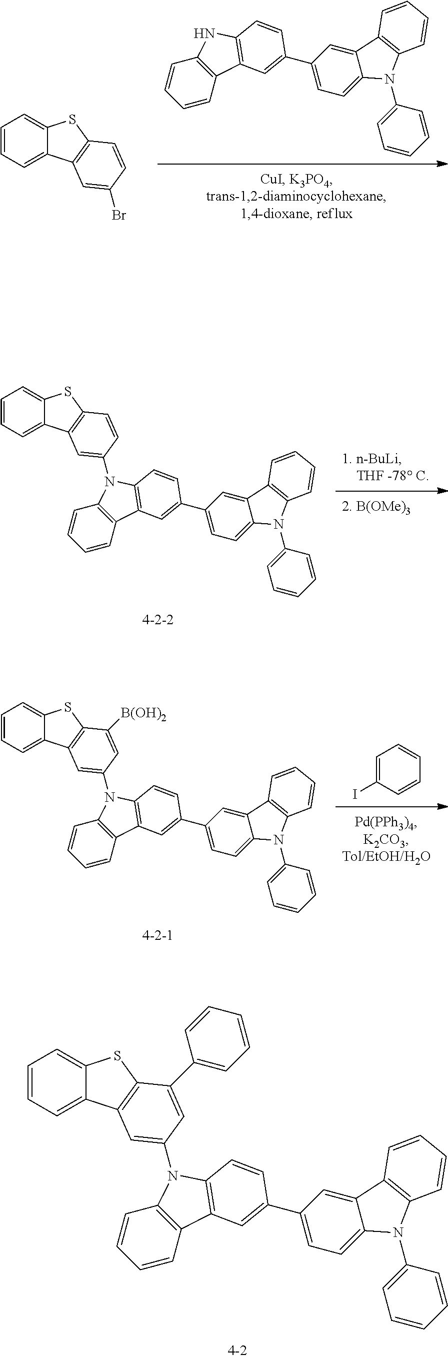 [SMILES: Brc1ccc2sc3ccccc3c2c1 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1 ; OB(O)c1cc(-n2c3ccccc3c3cc(-c4ccc5c(c4)c4ccccc4n5-c4ccccc4)ccc32)cc2c1sc1ccccc12 ; Ic1ccccc1 ; c1ccc(-c2cc(-n3c4ccccc4c4cc(-c5ccc6c(c5)c5ccccc5n6-c5ccccc5)ccc43)cc3c2sc2ccccc23)cc1]

1) Preparation of Compound 4-2-2

After dissolving 2-bromodibenzo[b,d]thiophene (4.2 g, 15.8 mM), 9-phenyl-9H,9′H-3,3′-bicarbazole (6.5 g, 15.8 mM), CuI (3.0 g, 15.8 mM), trans-1,2-diaminocyclohexane (1.9 mL, 15.8 mM) and K₃PO₄ (3.3 g, 31.6 mM) in 1,4-dioxane (100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 4-2-2 (7.9 g, 85%).

2) Preparation of Compound 4-2-1

To a mixture solution obtained by introducing Compound 4-2-2 (8.4 g, 14.3 mmol) and tetrahydrofuran (THF) (100 mL), 2.5 M n-BuLi (7.4 mL, 18.6 mmol) was added dropwise at −78° C., and the result was stirred for 1 hour at room temperature. To the reaction mixture, trimethyl borate (B(OMe)₃) (4.8 mL, 42.9 mmol) was added dropwise, and the result was stirred for 2 hours at room temperature. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:MeOH=100:3), and recrystallized with DCM to obtain target Compound 4-2-1 (3.9 g, 70%).

3) Preparation of Compound 4-2

After dissolving Compound 4-2-1 (6.7 g, 10.5 mM), iodobenzene (2.1 g, 10.5 mM), Pd(PPh₃)₄ (606 mg, 0.52 mM) and K₂CO₃ (2.9 g, 21.0 mM) in toluene/EtOH/H₂O (100 mL/20 mL/20 mL), the result was refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 4-2 (4.9 g, 70%).

### <Preparation Example 4> Synthesis of Compound 4-3

Target Compound 4-3 (83%) was obtained in the same manner as in Preparation of Compound 4-2 of Preparation Example 3 except that 4-iodo-1,1′-biphenyl was used instead of iodobenzene.

### <Preparation Example 5> Synthesis of Compound 4-12

Target Compound 4-12 (80%) was obtained in the same manner as in Preparation of Compound 4-2 of Preparation Example 3 except that 4-iododibenzo[b,d]furan was used instead of iodobenzene.

Compounds other than the compounds described in Preparation Examples 1 to 5 and Tables 1 to 3 were also prepared in the same manner as in the preparation examples described above.

Synthesis results of the compounds prepared above are shown in the following Tables 4 and 5.

TABLE 4

| Compound | |
|---|---|
| No. | ¹H NMR (CDCl₃, 200 Mz) |
| 1-1 | δ = 9.08 (1H, d), 8.98 (1H, d), 8.56 (2H, s), 8.36 (4H, m), 8.27 (1H, d), 7.75-7.64 |
| | (6H, m), 7.52-7.41 (10H, m) |
| 1-4 | δ = 9.08 (1H, d), 8.98 (1H, d), 8.56 (2H, s), 8.36 (4H, m), 8.27 (1H, d), 7.75-7.64 |
| | (6H, m), 7.52-7.41 (10H, m), 7.25 (4H, s) |
| 1-7 | δ = 9.08 (1H, d), 8.98 (1H, d), 8.56 (2H, s), 8.36 (4H, m), 8.27 (1H, d), 7.75-7.64 |
| | (6H, m), 7.52-7.41 (10H, m), 7.25 (8H, s) |
| 1-8 | δ = 9.08 (1H, d), 8.98 (1H, d), 8.56 (2H, s), 8.36 (4H, m), 8.27 (1H, d), 8.04 (2H, s), |
| | 7.75-7.64 (8H, m), 7.52-7.41 (12H, m) |
| 1-9 | δ = 9.08 (1H, d), 8.98 (1H, d), 8.56 (2H, s), 8.36 (4H, m), 8.27 (1H, d), 8.04 (3H, s), |
| | 7.94 (1H, s), 7.75-7.61 (11H, m), 7.52-7.41 (13H, m) |
| 1-17 | δ = 9.27 (1H, s), 9.08 (1H, d), 8.98 (1H, d), 8.79 (1H, d), 8.56 (2H, s), 8.37-8.27 |
| | (9H, m), 7.70-7.64 (8H, m), 7.50 (8H, m) |
| 1-21 | δ = 9.60 (1H, d), 9.27 (1H, s), 9.08 (1H, d), 8.98 (1H, d), 8.79 (1H, d), 8.56 (2H, s), |
| | 8.37-8.27 (8H, m), 7.94 (1H, s), 7.70-7.50 (20H, m) |
| 1-27 | δ = 9.08 (1H, d), 8.98 (1H, d), 8.56 (2H, s), 8.36 (4H, m), 8.27 (1H, d), 7.98 (1H, d), |
| | 7.88-7.79 (3H, m), 7.70-7.64 (4H, m), 7.54-7.50 (8H, m), 7.39-7.31 (2H, m) |
| 1-28 | δ = 9.08 (1H, d), 8.98 (1H, d), 8.56 (2H, s), 8.36 (4H, m), 8.27 (1H, d), 7.98 (1H, d), |
| | 7.82 (1H, d), 7.70-7.50 (14H, m), 7.39-7.31 (2H, m) |
| 1-31 | δ = 9.08 (1H, d), 8.98 (1H, d), 8.56 (2H, s), 8.45 (1H, d), 8.36 (4H, m), 8.27 (1H, d), |
| | 8.12 (2H, m), 7.99-7.93 (2H, m), 7.70-7.49 (13H, m) |
| 1-34 | δ = 9.08 (1H, d), 8.98 (1H, d), 8.56 (2H, s), 8.36 (4H, m), 8.27 (1H, d), 8.09 (1H, d), |
| | 7.90-7.89 (2H, m), 7.78-7.50 (13H, m), 7.38-7.28 (2H, m), 1.69 (6H, s) |
| 1-39 | δ = 9.08 (1H, d), 8.98 (1H, d), 8.56 (2H, s), 8.36-8.27 (6H, m), 8.19-8.13 (2H, m), |
| | 7.89 (1H, s), 7.70-7.50 (18H, m), 7.20 (1H, t) |
| 1-44 | δ = 9.08 (1H, d), 8.98 (1H, d), 8.56 (2H, s), 8.36 (4H, m), 8.27 (1H, d), 7.87 (2H, d), |
| | 7.70-7.64 (6H, m), 7.50-7.38 (22H, m) |
| 1-46 | δ = 9.08 (1H, d), 8.98 (1H, d), 8.56 (2H, s), 8.36 (4H, m), 8.27 (1H, d), 8.02 (1H, d), |
| | 7.85-7.64 (7H, m), 7.50 (4H, m) |
| 1-49 | δ = 9.08 (1H, d), 8.98 (1H, d), 8.56 (2H, s), 8.45 (1H, d), 8.36 (4H, m), 8.27-8.17 |
| | (4H, m), 7.93 (1H, d), 7.70-7.50 (13H, m), 7.25 (4H, s) |
| 1-56 | δ = 9.08 (1H, d), 8.98 (1H, d), 8.56 (2H, s), 8.36-8.27 (6H, m), 8.19-8.13 (1H, m), |
| | 7.89 (1H, s), 7.70-7.50 (25H, m), 7.40 (1H, s), 7.20 (1H, t) |
| 1-90 | δ = 9.08 (1H, d), 8.98 (1H, d), 8.56 (2H, s), 8.27-8.23 (2H, m), 7.94 (4H, m), 7.75- |
| | 7.41 (16H, m), 7.25 (8H, s) |
| 1-92 | δ = 9.08 (1H, d), 8.98 (1H, d), 8.56 (2H, s), 8.27 (1H, d), 8.13 (1H, d), 8.04 (3H, s), |
| | 7.94 (1H, s), 7.84-7.41 (26H, m) |
| 1-99 | δ = 9.08 (1H, d), 8.98 (1H, d), 8.40-8.27 (7H, m), 7.79-7.64 (6H, d), 7.52-7.41 |
| | (10H, d) |
| 1-101 | δ = 9.08 (1H, d), 8.98 (1H, d), 8.40-8.27 (7H, m), 7.75-7.64 (6H, m), 7.52-7.41 |
| | (10H, m), 7.25 (4H, s) |
| 1-108 | δ = 9.27 (1H, s), 9.08 (1H, d), 8.98 (1H, d), 8.79 (1H, d), 8.56 (2H, s), 8.36-8.27 |
| | (9H, m), 7.70-7.64 (8H, m) 7.52-7.50 (8H, m) |
| 1-121 | δ = 9.08 (1H, d), 8.98 (1H, d), 8.56 (2H, s), 8.38-8.36 (5H, m), 8.27 (1H, d), 7.94 |
| | (1H, s), 7.75-7.64 (7H, m), 7.50-7.41 (10H, m) |
| 1-122 | δ = 9.08 (1H, d), 8.98 (1H, d), 8.56 (2H, s), 8.36 (4H, m), 8.27 (1H, d), 7.96 (2H, d), |
| | 7.75-7.64 (6H, m), 7.50-7.41 (10H, m), 7.25 (2H, d) |
| 1-127 | δ = 9.27 (1H, s), 9.08 (1H, d), 8.98 (1H, d), 8.79 (1H, d), 8.56 (2H, s), 8.38-8.27 |
| | (10H, m), 7.94 (1H, s), 7.73-7.64 (9H, m), 7.52-7.50 (8H, m) |
| 2-7 | δ = 9.08 (1H, d), 8.98 (1H, d), 8.56 (2H, s), 8.27 (1H, d), 7.79-7.64 (8H, m), 7.52- |
| | 7.41 (7H, m), 7.25 (8H, s) |
| 2-17 | δ = 9.27 (1H, s), 9.08 (1H, d), 8.98 (1H, d), 8.56 (2H, s), 8.32-8.27 (5H, m), 7.79- |
| | 7.64 (9H, m), 7.52-7.41 (5H, m) |
| 2-22 | δ = 9.27 (1H, s), 9.08 (1H, d), 8.98 (1H, d), 8.79 (1H, d), 8.56 (2H, s), 8.37-8.27 |
| | (5H, m), 7.79-7.64 (10H, m), 7.52-7.41 (5H, m), 7.25 (4H, s) |
| 2-27 | δ = 9.08 (1H, d), 8.98 (1H, d), 8.56 (2H, s), 8.27 (1H, d), 7.98 (1H, d), 7.79-7.31 |
| | (16H, m) |
| 2-31 | δ = 9.08 (1H, d), 8.98 (1H, d), 8.56 (2H, s), 8.45 (1H, d), 8.27 (1H, d), 8.12 (2H, m), |
| | 7.99-7.93 (2H, m), 7.79-7.41 (12H, m) |
| 2-39 | δ = 9.08 (1H, d), 8.98 (1H, d), 8.56 (2H, s), 8.30-8.13 (4H, m), 7.89 (1H, s), 7.70- |
| | 7.41 (17H, m), 7.20 (1H, t) |
| 2-42 | δ = 9.08 (1H, d), 8.98 (1H, d), 8.56 (2H, s), 8.27 (1H, d), 7.90 (1H, d), 7.79-7.10 |
| | (26H, m) |
| 2-44 | δ = 9.08 (1H, d), 8.98 (1H, d), 8.56 (2H, s), 8.27 (1H, d), 7.87 (2H, d), 7.79 (2H, d), |
| | 7.70-7.64 (6H, m), 7.52-7.38 (19H, m) |
| 2-56 | δ = 9.08 (1H, d), 8.98 (1H, d), 8.56 (2H, s), 8.30-8.27 (2H, m), 8.19-8.13 (2H, m), |
| | 7.89 (1H, s), 7.79-7.41 (24H, m), 7.20 (1H, t) |
| 2-70 | δ = 9.08 (1H, d), 8.98 (1H, d), 8.56 (2H, s), 8.27 (1H, d), 7.79-7.37 (32H, m) |
| 2-72 | δ = 9.08 (1H, d), 8.98 (1H, d), 8.56 (2H, s), 8.30-8.27 (3H, m), 8.19-8.13 (4H, m), |
| | 7.92-7.89 (6H, m) 7.79-7.41 (19H, m), 7.20 (2H, t) |
| 2-80 | δ = 9.60 (1H, d), 9.27 (2H, s), 9.08 (1H, d), 8.98 (1H, d), 8.79 (1H, d), 8.37-8.27 |
| | (8H, s), 7.70-7.64 (12H, m), 7.52 (4H, m) |
| 3-3 | δ = 8.55 (1H, d), 8.30 (1H, d), 8.21-8.13 (3H, m), 7.99-7.89 (4H, m), 7.77-7.35 |
| | (17H, m), 7.20-7.16 (2H, m) |
| 3-4 | δ = 8.55 (1H, d), 8.30 (1H, d), 8.19-8.13 (2H, m), 7.99-7.89 (8H, m), 7.77-7.75 |
| | (3H, m), 7.62-7.35 (11H, m), 7.20-7.16 (2H, m) |
| 3-7 | δ = 8.55 (1H, d), 8.31-8.30 (3H, d), 8.19-8.13 (2H, m), 7.99-7.89 (5H, m), 7.77- |
| | 7.75 (5H, m), 7.62-7.35 (14H, m), 7.20-7.16 (2H, m) |
| 3-31 | δ = 8.55 (1H, d), 8.30 (1H, d), 8.21-8.13 (4H, m), 7.99-7.89 (4H, m), 7.77-7.35 |
| | (20H, m), 7.20-7.16 (2H, m) |
| 3-32 | δ = 8.55 (1H, d), 8.30 (1H, d), 8.21-8.13 (3H, m), 7.99-7.89 (8H, m), 7.77-7.35 |
| | (17H, m), 7.20-7.16 (2H, m) |
| 4-2 | δ = 8.55 (1H, d), 8.45 (1H, d), 8.30 (1H, d), 8.19 (1H, d), 8.13 (1H, d), 8.00~7.89 |
| | (6H, m), 7.77 (2H, m), 7.62~7.35 (15H, m), 7.20~7.16 (2H, m) |
| 4-3 | δ = 8.55 (1H, d), 8.45 (1H, d), 8.30 (1H, d), 8.19 (1H, d), 8.13 (1H, d), 8.00~7.89 |
| | (6H, m), 7.77-7.75 (4H, m), 7.62~7.35 (13H, m), 7.25~7.16 (6H, m) |
| 4-12 | δ = 8.55 (1H, d), 8.45 (1H, d), 8.30 (1H, d), 8.19~7.89 (11H, m), 7.77 (2H, m), |
| | 7.62~7.31 (14H, m), 7.20~7.16 (2H, m) |

TABLE 5

| Compound | FD-MS | Compound | FD-MS |
|---|---|---|---|
| 1-1 | m/z = 535.20 (C₃₉H₂₅N₃ = 535.65) | 1-4 | m/z = 611.24 (C₄₅H₂₉N₃ = 611.75) |
| 1-7 | m/z = 687.27 (C₅₁H₃₃N₃ = 687.85) | 1-8 | m/z = 687.27 (C₅₁H₃₃N₃ = 687.85) |
| 1-9 | m/z = 763.30 (C₅₇H₃₇N₃ = 763.94 ) | 1-17 | m/z = 685.25 (C₅₁H₃₁N₃ = 685.83) |
| 1-21 | m/z = 761.28 (C₅₇H₃₅N₃ = 761.93) | 1-27 | m/z = 625.22 (C₄₅H₂₇N₃O = 625.73) |
| 1-28 | m/z = 625.22 | 1-31 | m/z = 641.19 (C₄₅H₂₇N₃S = 641.79) |
| | (C₄₅H₂₇N₃O = 625.73) | | |
| 1-34 | m/z = 651.27 (C₄₈H₃₃N₃ = 651.81) | 1-39 | m/z = 700.26 (C₅₁H₃₂N₄ = 700.84) |
| 1-44 | m/z = 793.29 | 1-46 | m/z = 560.20 (C₄₀H₂₄N₄ = 560.66) |
| | (C₅₇H₃₉N₃Si = 794.04) | | |
| 1-49 | m/z = 717.22 | 1-56 | m/z = 865.32 (C₆₃H₃₉N₅ = 866.04) |
| | (C₅₁H₃₁N₃S = 717.89) | | |
| 1-90 | m/z = 686.27 (C₅₂H₃₄N₂ = 686.86) | 1-92 | m/z = 736.29 (C₅₆H₃₆N₂ = 736.92) |
| 1-99 | m/z = 535.20 (C₃₉H₂₅N₃ = 535.65) | 1-101 | m/z = 611.24 (C₄₅H₂₉N₃ = 611.75) |
| 1-108 | m/z = 685.25 (C₅₁H₃₁N₃ = 685.83) | 1-121 | m/z = 611.24 (C₄₅H₂₉N₃ = 611.75) |
| 1-122 | m/z = 611.24 (C₄₅H₂₉N₃ = 611.75) | 1-127 | m/z = 761.28 (C₅₇H₃₅N₃ = 761.93) |
| 2-7 | m/z = 532.22 (C₄₂H₂₈ = 532.69) | 2-17 | m/z = 530.20 (C₄₂H₂₆ = 530.67) |
| 2-22 | m/z = 606.23 (C₄₈H₃₀ = 606.77) | 2-27 | m/z = 470.17 (C₃₆H₂₂O = 470.57) |
| 2-31 | m/z = 486.14 (C₃₆H₂₂S = 486.63) | 2-39 | m/z = 545.21 (C₄₂H₂₇N = 545.68) |
| 2-42 | m/z = 620.25 (C₄₉H₃₂ = 620.25) | 2-44 | m/z = 638.24 (C₄₈H₃₄Si = 638.89) |
| 2-56 | m/z = 710.27 (C₅₄H₃₄N₂ = 710.88) | 2-70 | m/z = 699.29 (C₅₄H₃₇N = 699.90) |
| 2-72 | m/z = 786.30 (C₆₀H₃₈N₂ = 786.98) | 2-80 | m/z = 680.25 (C₅₄H₃₂ = 680.85) |
| 3-3 | m/z = 560.23 (C₄₂H₂₈N₂ = 560.70) | 3-4 | m/z = 560.23 (C₄₂H₂₈N₂ = 560.70) |
| 3-7 | m/z = 636.26 (C₄₈H₃₂N₂ = 636.80) | 3-31 | m/z = 636.26 (C₄₈H₃₂N₂ = 636.80) |
| 3-32 | m/z = 636.26 (C₄₈H₃₂N₂ = 636.80) | 4-2 | m/z = 666.84 (C₄₈H₃₀N₂ = 666.21) |
| 4-3 | m/z = 742.24 | 4-12 | m/z = 756.22 |
| | (C₅₄H₃₄N₂S = 742.94) | | (C₅₄H₃₂N₂OS = 756.92) |

### Experimental Example 1

1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in an ultraviolet (UV) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino] triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, a light emitting layer compound (compound of Chemical Formula 1, compound of Chemical Formula 2 or Ref. 1 to Ref. 5) described in the following Table 6 was deposited to 400 Å as a host, and, as a green phosphorescent dopant, Ir(ppy)₃ was doped and deposited by 7% with respect to the deposited thickness of the light emitting layer. After that, BCP (bathocuproine) was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

2) Evaluation on Organic Light Emitting Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 6.

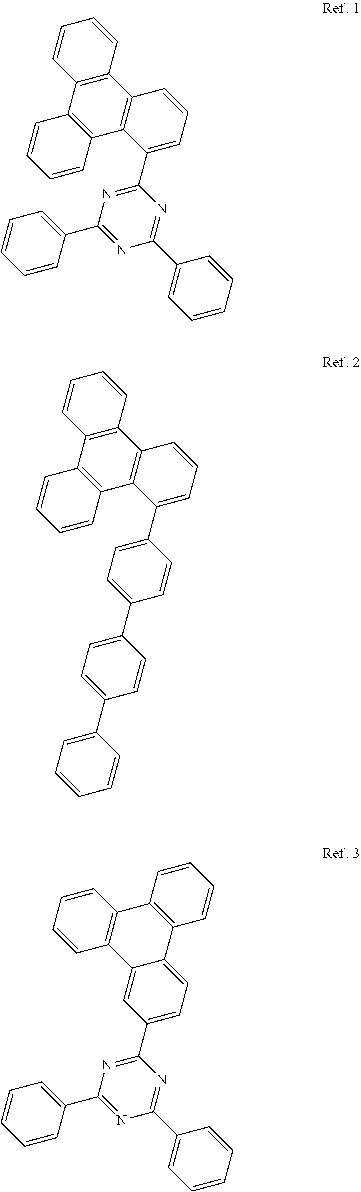

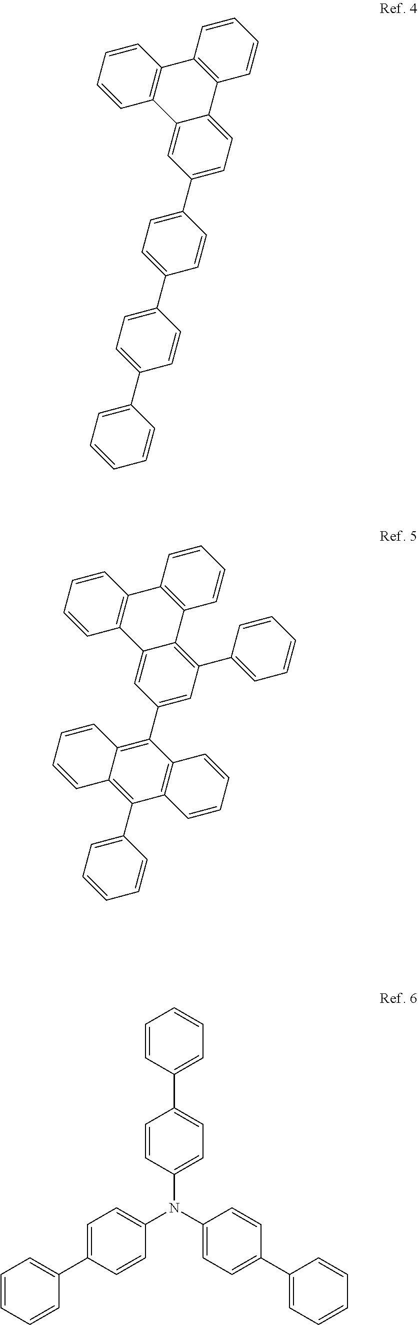

TABLE 6

| | Light | | | | |
|---|---|---|---|---|---|
| | Emitting | Driving | | Color | |
| | Layer | Voltage | Efficiency | Coordinate | Lifetime |
| | Compound | (V) | (cd/A) | (x, y) | (T₉₀) |
| Example 1 | 1-1 | 4.66 | 71.1 | Green | 352 |
| Example 2 | 1-4 | 4.31 | 79.2 | | 365 |
| Example 3 | 1-7 | 4.45 | 72.8 | | 370 |
| Example 4 | 1-8 | 4.67 | 71.2 | | 356 |
| Example 5 | 1-9 | 4.66 | 71.2 | | 360 |
| Example 6 | 1-17 | 4.35 | 79.2 | | 382 |
| Example 7 | 1-21 | 4.33 | 74.2 | | 405 |
| Example 8 | 1-27 | 4.69 | 69.2 | | 311 |
| Example 9 | 1-28 | 4.41 | 68.4 | | 321 |
| Example 10 | 1-31 | 4.11 | 72.2 | | 299 |
| Example 11 | 1-34 | 4.45 | 72.8 | | 338 |
| Example 12 | 1-39 | 4.66 | 71.1 | | 347 |
| Example 13 | 1-44 | 4.32 | 71.5 | | 289 |
| Example 14 | 1-46 | 4.67 | 71.2 | | 277 |
| Example 15 | 1-49 | 4.38 | 76.4 | | 293 |
| Example 16 | 1-56 | 4.66 | 71.1 | | 271 |
| Example 17 | 1-90 | 4.33 | 75.2 | | 246 |
| Example 18 | 1-92 | 4.13 | 79.2 | | 242 |
| Example 19 | 1-99 | 4.66 | 71.2 | | 251 |
| Example 20 | 1-101 | 4.41 | 75.8 | | 249 |
| Example 21 | 1-108 | 4.67 | 71.2 | | 390 |
| Example 22 | 1-121 | 4.31 | 79.2 | | 261 |
| Example 23 | 1-122 | 4.36 | 78.9 | | 256 |
| Example 24 | 1-127 | 4.69 | 66.2 | | 271 |
| Example 25 | 2-7 | 4.32 | 78.3 | | 226 |
| Example 26 | 2-17 | 4.41 | 75.8 | | 229 |
| Example 27 | 2-22 | 4.42 | 75.7 | | 237 |
| Example 28 | 2-27 | 4.45 | 72.8 | | 208 |
| Example 29 | 2-31 | 4.48 | 70.2 | | 205 |
| Example 30 | 2-39 | 4.69 | 77.2 | | 221 |
| Example 31 | 2-42 | 4.66 | 71.2 | | 218 |
| Example 32 | 2-44 | 4.83 | 65.9 | | 212 |
| Example 33 | 2-56 | 4.41 | 75.8 | | 225 |
| Example 34 | 2-70 | 4.67 | 71.2 | | 201 |
| Example 35 | 2-72 | 4.31 | 79.2 | | 226 |
| Example 36 | 2-80 | 4.36 | 78.9 | | 239 |
| Comparative | 3-3 | 5.21 | 57.0 | | 85 |
| Example 1 | | | | | |
| Comparative | 3-4 | 4.75 | 51.2 | | 79 |
| Example 2 | | | | | |
| Comparative | 3-7 | 4.48 | 55.2 | | 86 |
| Example 3 | | | | | |
| Comparative | 3-31 | 4.75 | 51.2 | | 71 |
| Example 4 | | | | | |
| Comparative | 3-32 | 4.48 | 50.2 | | 89 |
| Example 5 | | | | | |
| Comparative | 4-2 | 4.83 | 61.9 | | 121 |
| Example 6 | | | | | |
| Comparative | Ref. 1 | 5.14 | 48.9 | | 40 |
| Example 7 | | | | | |
| Comparative | Ref. 2 | 5.26 | 47.6 | | 31 |
| Example 8 | | | | | |
| Comparative | Ref. 3 | 5.64 | 43.9 | | 20 |
| Example 9 | | | | | |
| Comparative | Ref. 4 | 5.54 | 45.9 | | 26 |
| Example 10 | | | | | |
| Comparative | Ref. 5 | 4.71 | 66.8 | | 35 |
| Example 11 | | | | | |

As seen from the results of Table 6, it was identified that the organic electroluminescent device using the light emitting layer material of the organic electroluminescent device of the present disclosure had a lower driving voltage, enhanced light emission efficiency and significantly improved lifetime compared to Comparative Examples 1 to 11.

The HOMO orbital of Compound 1-7 according to the present disclosure was delocalized to the triphenylene and the aryl-based substituent. However, it was identified that, when there was no aryl-based substituent in the triphenylene group as in the compounds of Ref. 1 and Ref. 3, the HOMO was localized to the triphenylene failing to effectively stabilize holes, and thereby reducing the lifetime.

It was identified that, when there was no triazine in the triphenylene group as in the compounds of Ref. 2 and Ref. 4, electron mobility decreases and a balance between holes and electrons break in the light emitting layer, and thereby reducing the lifetime.

The HOMO orbital of Compound 2-7 was delocalized to the triphenylene and two substituents that are the phenyl group and the terphenyl group, which was able to effectively stabilize holes. However, it was identified that, when there was one substituent in the triphenylene group as in the compound of Ref. 4, the HOMO orbital was relatively localized failing to effectively stabilize holes, and thereby reducing the lifetime.

The compound of Ref. 5 has the same position of substitution as the compound of the present disclosure, however, has an anthracene substituent bonding thereto. In this compound, the HOMO and the LUMO orbitals were all localized to the anthracene. It was identified that this decreased stability of holes and electrons compared to when the HOMO and the LUMO orbitals were conjugated, and thereby reduced the lifetime.

### Experimental Example 2

1) Manufacture of Organic Light Emitting Device

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in an ultraviolet (UV) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino] triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, one type of compound of Chemical Formula 1-N and one type of compound of Chemical Formula 1-P (Examples 40 to 46), one type of compound of Chemical Formula 1-N and one type of compound of Chemical Formula 2 (Examples 47 to 67) or one type of compound of Chemical Formula 1-N and Ref. 6 (Comparative Examples 12 to 14) were premixed and then deposited to 400 Å in one source of supply as a host as described in the following Table 7, and Ir(ppy)₃ was doped and deposited by an amount of 7% with respect to the deposited thickness of the light emitting layer as a green phosphorescent dopant. After that, BCP (bathocuproine) was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the OLED manufacture.

2) Evaluation on Organic Light Emitting Device

For each of the organic electroluminescent devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 7.

TABLE 7

| | Light | | | | Color | |
|---|---|---|---|---|---|---|
| | Emitting | Ratio | Driving | | Coordi- | Life- |
| | Layer | (Weight | Voltage | Efficiency | nate | time |
| | Compound | Ratio) | (V) | (cd/A) | (x, y) | (T₉₀) |
| Example 40 | 1-4:2-80 | 1:8 | 4.73 | 54.2 | Green | 438 |
| Example 41 | | 1:5 | 4.71 | 57.2 | | 444 |
| Example 42 | | 1:2 | 4.38 | 76.4 | | 528 |
| Example 43 | | 1:1 | 4.45 | 72.8 | | 511 |
| Example 44 | | 2:1 | 4.66 | 71.1 | | 489 |
| Example 45 | | 5:1 | 4.32 | 68.3 | | 433 |
| Example 46 | | 8:1 | 4.21 | 67.0 | | 525 |
| Example 47 | 1-7:3-4 | 1:2 | 4.38 | 76.4 | | 539 |
| Example 48 | | 1:1 | 4.45 | 72.8 | | 522 |
| Example 49 | | 2:1 | 4.66 | 71.1 | | 500 |
| Example 50 | 1-17:3-7 | 1:2 | 4.33 | 74.2 | | 562 |
| Example 51 | | 1:1 | 4.42 | 72.2 | | 553 |
| Example 52 | | 2:1 | 4.66 | 71.2 | | 528 |
| Example 53 | 1-21:3-31 | 1:2 | 4.31 | 79.2 | | 586 |
| Example 54 | | 1:1 | 4.42 | 75.7 | | 558 |
| Example 55 | | 2:1 | 4.66 | 71.1 | | 536 |
| Example 56 | 1-108:3-32 | 1:2 | 4.33 | 75.2 | | 569 |
| Example 57 | | 1:1 | 4.48 | 70.2 | | 543 |
| Example 58 | | 2:1 | 4.69 | 69.2 | | 518 |
| Example 59 | 1-21:4-12 | 1:2 | 4.33 | 75.2 | | 679 |
| Example 60 | | 1:1 | 4.48 | 70.2 | | 546 |
| Example 61 | | 2:1 | 4.69 | 69.2 | | 525 |
| Example 62 | 1-21:4-3 | 1:2 | 4.33 | 75.2 | | 620 |
| Example 63 | | 1:1 | 4.48 | 70.2 | | 582 |
| Example 64 | | 2:1 | 4.69 | 69.2 | | 543 |
| Example 65 | 1-21:4-2 | 1:2 | 4.35 | 79.2 | | 599 |
| Example 66 | | 1:1 | 4.41 | 75.8 | | 591 |
| Example 67 | | 2:1 | 4.67 | 71.2 | | 540 |
| Comparative | 1-21:Ref. 6 | 1:2 | 5.34 | 68.2 | | 399 |
| Example 12 | | | | | | |
| Comparative | | 1:1 | 5.40 | 64.7 | | 391 |
| Example 13 | | | | | | |
| Comparative | | 2:1 | 5.66 | 60.1 | | 340 |
| Example 14 | | | | | | |

As seen from the results of Table 7, effects of more superior efficiency and lifetime were obtained when including both the compound of Chemical Formula 1-N and the compound of Chemical Formula 1-P, or both the compound of Chemical Formula 1-N and the compound of Chemical Formula 2. Such results may lead to a forecast that an exciplex phenomenon occurs when including the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may decrease, which resultantly helps with enhancement in the lifetime. In the invention of the present application, it was identified that excellent device properties were obtained when, as the light emitting layer host, the compound of Chemical Formula 1-P or the compound of Chemical Formula 2 performing a donor role and the compound of Chemical Formula 1-N performing an acceptor role were used.

1. A compound represented by the following Chemical Formula 1: 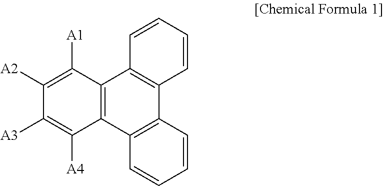
wherein, in Chemical Formula 1,
one of A1 and A2 is (L1)ₐ-Q1;
the other one of A1 and A2, and A3 and A4 are each independently hydrogen; deuterium; or (L2)_{b}-Q2, and at least one thereof is (L2)_{b}-Q2;
a and b are each independently an integer of 1 to 5;
when a and b are each 2 or greater, substituents in the parentheses are the same as or different from each other;
L1 and L2 are each independently a direct bond; a substituted or unsubstituted C6 to C60 arylene group; or a substituted or unsubstituted C2 to C60 heteroarylene group;
Q1 is a substituted or unsubstituted C6 to C20 aryl group; or a substituted or unsubstituted C2 to C20 heteroaryl group including N;
Q2 is a cyano group; a substituted or unsubstituted silyl group; a substituted or unsubstituted amine group; a substituted or unsubstituted C1 to C20 alkyl group; a substituted or unsubstituted C6 to C30 aryl group; a substituted or unsubstituted C2 to C30 heteroaryl group; or a substituted or unsubstituted phosphine oxide group;
when A2 and A3 are hydrogen, Q1 is a phenyl group and Q2 includes pyridine or triazine, L1 is a substituted or unsubstituted C6 to C60 arylene group; or a substituted or unsubstituted C2 to C60 heteroarylene group; and
when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a dicyclic or higher aryl group unsubstituted or substituted with an alkyl group or an aryl group is satisfied.

2. The compound of claim 1, wherein Chemical Formula 1 is represented by the following Chemical Formula 1-1: 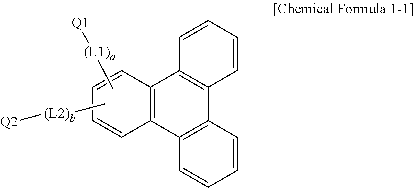
in Chemical Formula 1-1,
each substituent has the same definition as in Chemical Formula 1.

3. The compound of claim 1, wherein L1 and L2 are each independently a direct bond; a substituted or unsubstituted phenylene group; a substituted or unsubstituted biphenylene group; a substituted or unsubstituted terphenylene group; a substituted or unsubstituted divalent pyridine group; a substituted or unsubstituted divalent pyrimidine group; a substituted or unsubstituted divalent triazine group; or a substituted or unsubstituted divalent carbazolyl group.

4. The compound of claim 1, wherein Q2 is a cyano group; a silyl group unsubstituted or substituted with an aryl group; an amine group unsubstituted or substituted with an aryl group; a C1 to C20 alkyl group substituted with an aryl group; a C6 to C30 aryl group unsubstituted or substituted with a cyano group, an alkyl group, an aryl group or a heteroaryl group; a C2 to C30 heteroaryl group unsubstituted or substituted with an alkyl group, an aryl group or a heteroaryl group; or a phosphine oxide group unsubstituted or substituted with an aryl group.

5. The compound of claim 1, wherein Chemical Formula 1 is represented by any one of the following compounds:

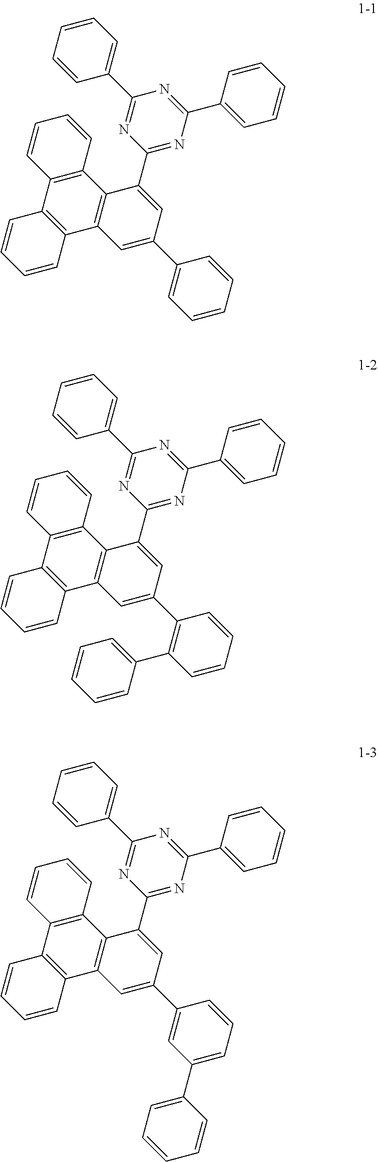 [SMILES: c1ccc(-c2cc(-c3nc(-c4ccccc4)nc(-c4ccccc4)n3)c3c4ccccc4c4ccccc4c3c2)cc1 ; c1ccc(-c2nc(-c3ccccc3)nc(-c3cc(-c4ccccc4-c4ccccc4)cc4c5ccccc5c5ccccc5c34)n2)cc1 ; c1ccc(-c2cccc(-c3cc(-c4nc(-c5ccccc5)nc(-c5ccccc5)n4)c4c5ccccc5c5ccccc5c4c3)c2)cc1]

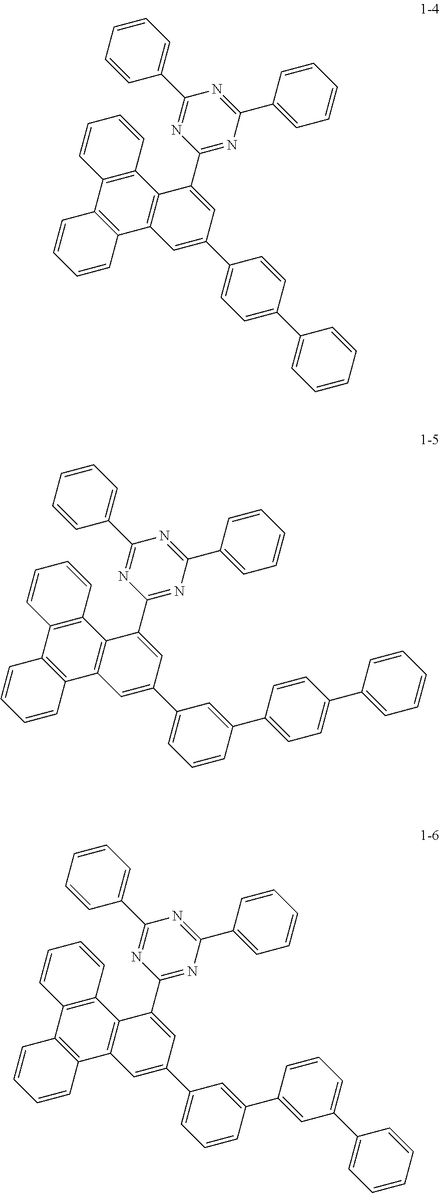 [SMILES: c1ccc(-c2ccc(-c3cc(-c4nc(-c5ccccc5)nc(-c5ccccc5)n4)c4c5ccccc5c5ccccc5c4c3)cc2)cc1 ; c1ccc(-c2ccc(-c3cccc(-c4cc(-c5nc(-c6ccccc6)nc(-c6ccccc6)n5)c5c6ccccc6c6ccccc6c5c4)c3)cc2)cc1 ; c1ccc(-c2cccc(-c3cccc(-c4cc(-c5nc(-c6ccccc6)nc(-c6ccccc6)n5)c5c6ccccc6c6ccccc6c5c4)c3)c2)cc1]

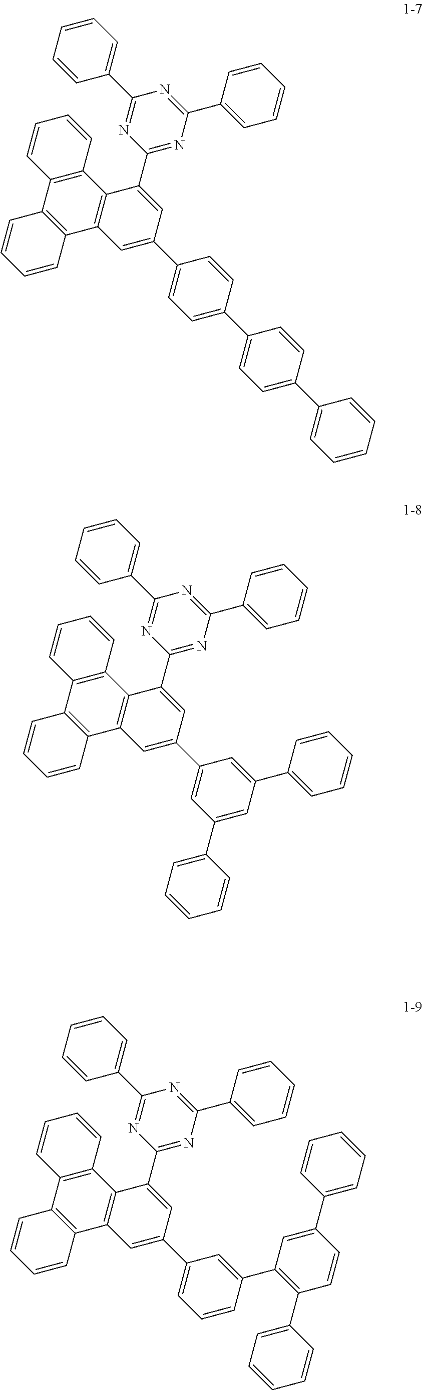 [SMILES: c1ccc(-c2ccc(-c3ccc(-c4cc(-c5nc(-c6ccccc6)nc(-c6ccccc6)n5)c5c6ccccc6c6ccccc6c5c4)cc3)cc2)cc1 ; c1ccc(-c2cc(-c3ccccc3)cc(-c3cc(-c4nc(-c5ccccc5)nc(-c5ccccc5)n4)c4c5ccccc5c5ccccc5c4c3)c2)cc1 ; c1ccc(-c2ccc(-c3ccccc3)c(-c3cccc(-c4cc(-c5nc(-c6ccccc6)nc(-c6ccccc6)n5)c5c6ccccc6c6ccccc6c5c4)c3)c2)cc1]

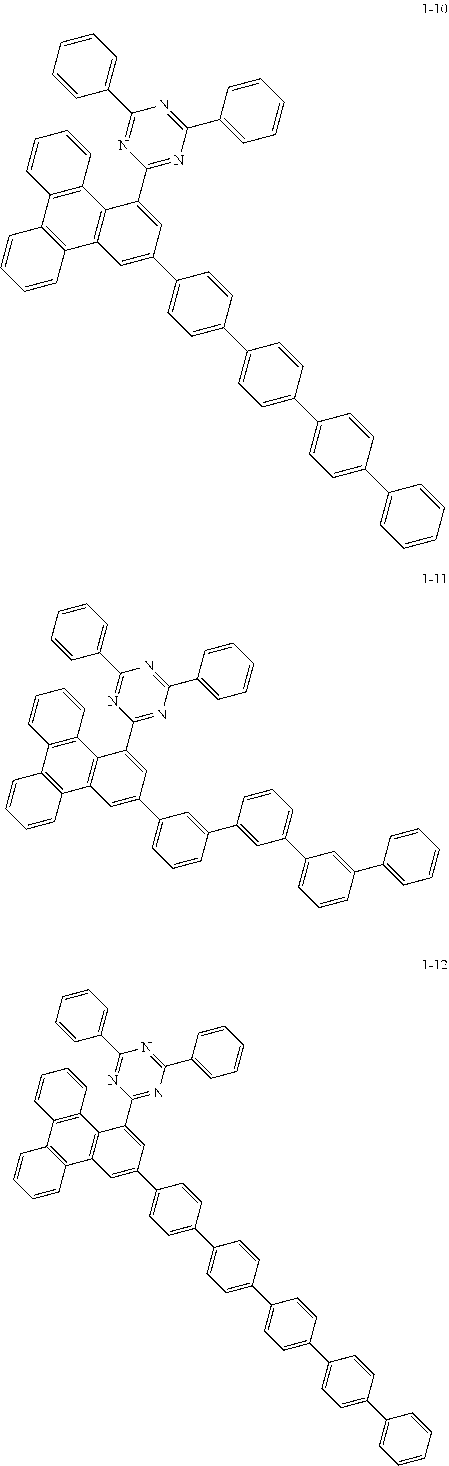 [SMILES: c1ccc(-c2ccc(-c3ccc(-c4ccc(-c5cc(-c6nc(-c7ccccc7)nc(-c7ccccc7)n6)c6c7ccccc7c7ccccc7c6c5)cc4)cc3)cc2)cc1 ; c1ccc(-c2cccc(-c3cccc(-c4cccc(-c5cc(-c6nc(-c7ccccc7)nc(-c7ccccc7)n6)c6c7ccccc7c7ccccc7c6c5)c4)c3)c2)cc1 ; c1ccc(-c2ccc(-c3ccc(-c4ccc(-c5ccc(-c6cc(-c7nc(-c8ccccc8)nc(-c8ccccc8)n7)c7c8ccccc8c8ccccc8c7c6)cc5)cc4)cc3)cc2)cc1]

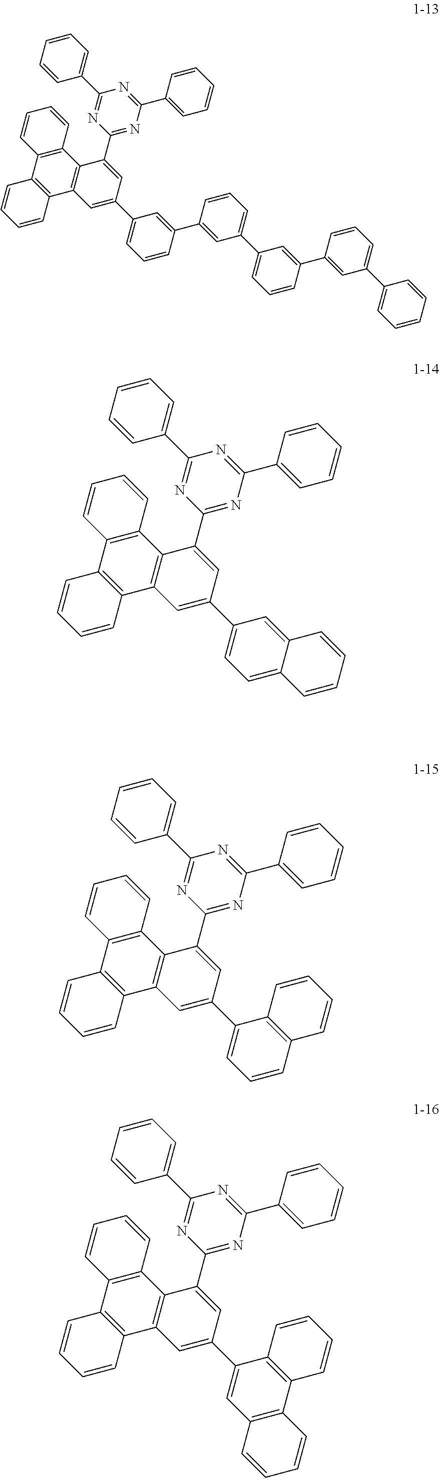 [SMILES: c1ccc(-c2cccc(-c3cccc(-c4cccc(-c5cccc(-c6cc(-c7nc(-c8ccccc8)nc(-c8ccccc8)n7)c7c8ccccc8c8ccccc8c7c6)c5)c4)c3)c2)cc1 ; c1ccc(-c2nc(-c3ccccc3)nc(-c3cc(-c4ccc5ccccc5c4)cc4c5ccccc5c5ccccc5c34)n2)cc1 ; c1ccc(-c2nc(-c3ccccc3)nc(-c3cc(-c4cccc5ccccc45)cc4c5ccccc5c5ccccc5c34)n2)cc1 ; c1ccc(-c2nc(-c3ccccc3)nc(-c3cc(-c4cc5ccccc5c5ccccc45)cc4c5ccccc5c5ccccc5c34)n2)cc1]

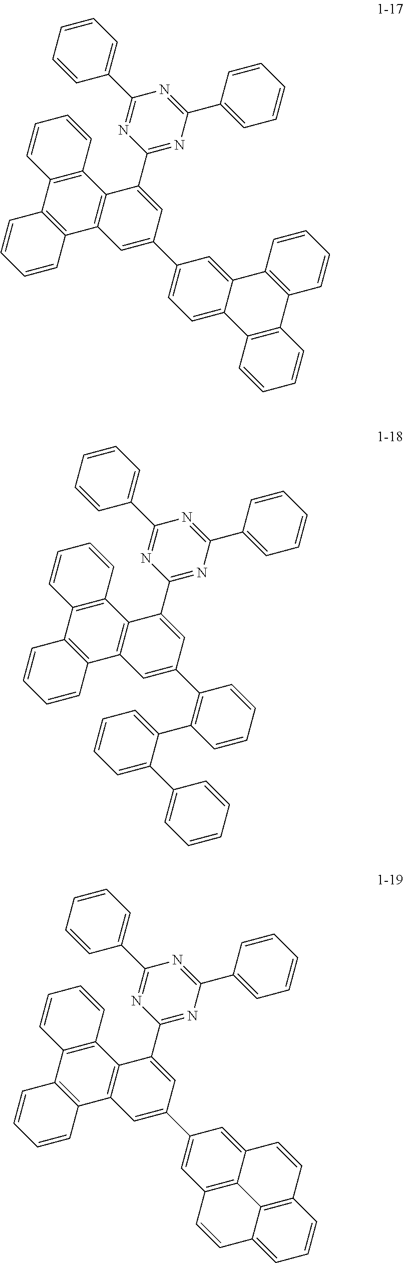 [SMILES: c1ccc(-c2nc(-c3ccccc3)nc(-c3cc(-c4ccc5c6ccccc6c6ccccc6c5c4)cc4c5ccccc5c5ccccc5c34)n2)cc1 ; c1ccc(-c2nc(-c3ccccc3)nc(-c3cc(-c4ccccc4-c4ccccc4-c4ccccc4)cc4c5ccccc5c5ccccc5c34)n2)cc1 ; c1ccc(-c2nc(-c3ccccc3)nc(-c3cc(-c4cc5ccc6cccc7ccc(c4)c5c67)cc4c5ccccc5c5ccccc5c34)n2)cc1]

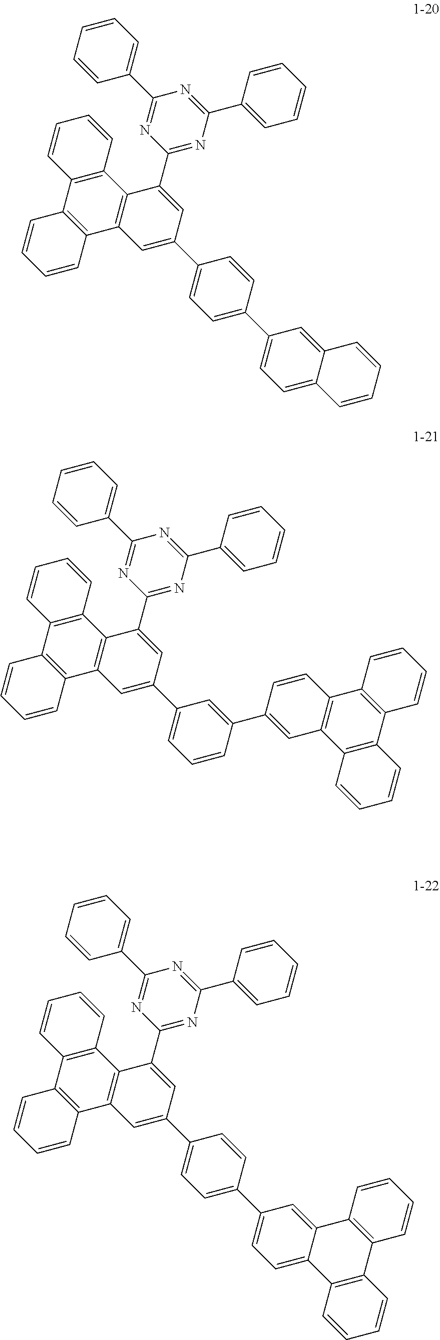 [SMILES: c1ccc(-c2nc(-c3ccccc3)nc(-c3cc(-c4ccc(-c5ccc6ccccc6c5)cc4)cc4c5ccccc5c5ccccc5c34)n2)cc1 ; c1ccc(-c2nc(-c3ccccc3)nc(-c3cc(-c4cccc(-c5ccc6c7ccccc7c7ccccc7c6c5)c4)cc4c5ccccc5c5ccccc5c34)n2)cc1 ; c1ccc(-c2nc(-c3ccccc3)nc(-c3cc(-c4ccc(-c5ccc6c7ccccc7c7ccccc7c6c5)cc4)cc4c5ccccc5c5ccccc5c34)n2)cc1]

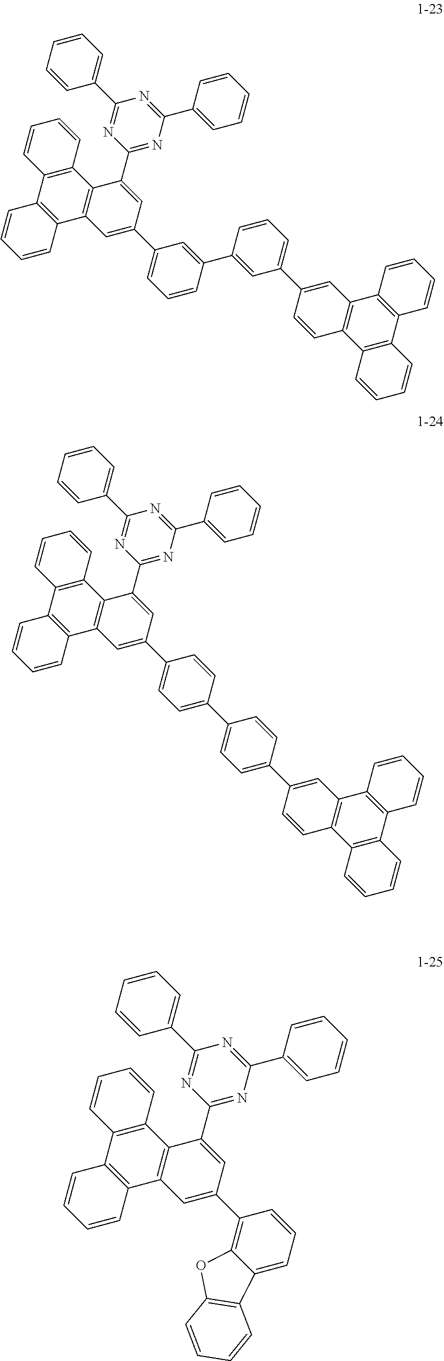 [SMILES: c1ccc(-c2nc(-c3ccccc3)nc(-c3cc(-c4cccc(-c5cccc(-c6ccc7c8ccccc8c8ccccc8c7c6)c5)c4)cc4c5ccccc5c5ccccc5c34)n2)cc1 ; c1ccc(-c2nc(-c3ccccc3)nc(-c3cc(-c4ccc(-c5ccc(-c6ccc7c8ccccc8c8ccccc8c7c6)cc5)cc4)cc4c5ccccc5c5ccccc5c34)n2)cc1 ; c1ccc(-c2nc(-c3ccccc3)nc(-c3cc(-c4cccc5c4oc4ccccc45)cc4c5ccccc5c5ccccc5c34)n2)cc1]

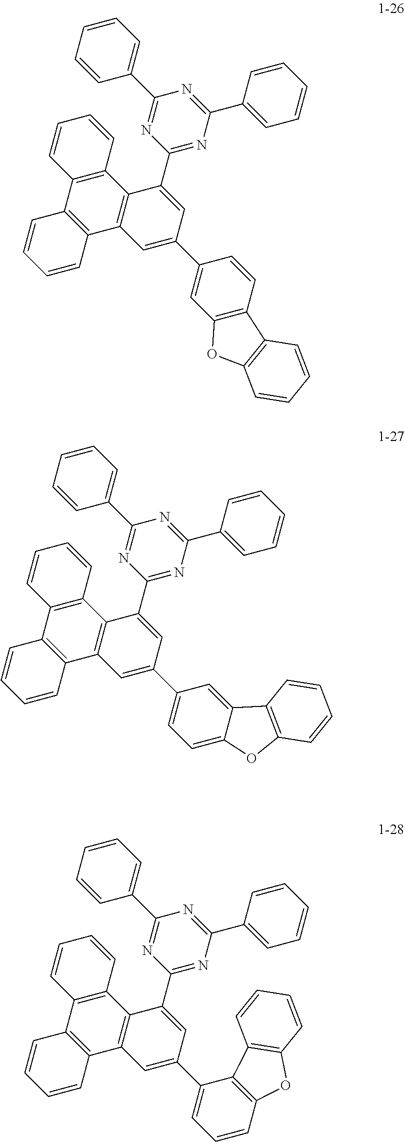 [SMILES: c1ccc(-c2nc(-c3ccccc3)nc(-c3cc(-c4ccc5c(c4)oc4ccccc45)cc4c5ccccc5c5ccccc5c34)n2)cc1 ; c1ccc(-c2nc(-c3ccccc3)nc(-c3cc(-c4ccc5oc6ccccc6c5c4)cc4c5ccccc5c5ccccc5c34)n2)cc1 ; c1ccc(-c2nc(-c3ccccc3)nc(-c3cc(-c4cccc5oc6ccccc6c45)cc4c5ccccc5c5ccccc5c34)n2)cc1]

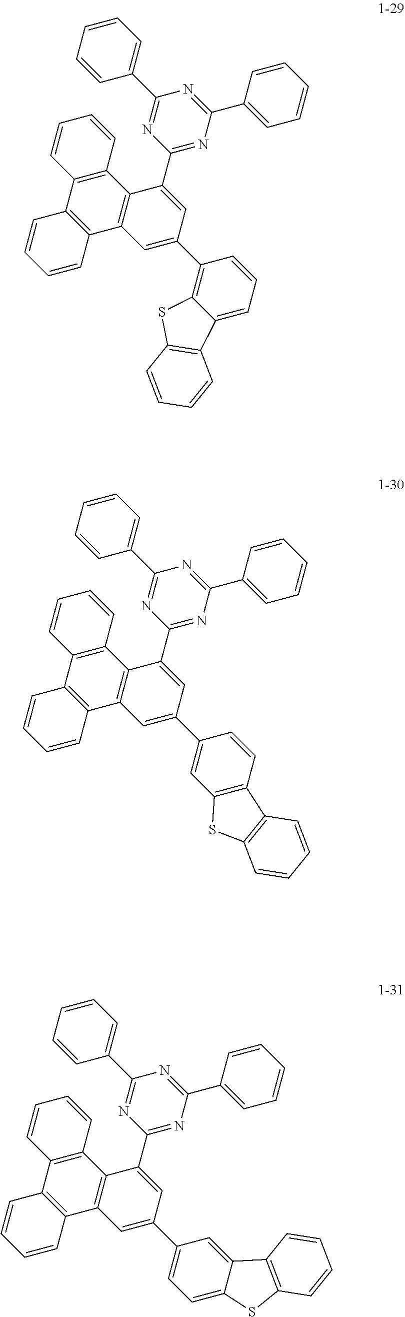 [SMILES: c1ccc(-c2nc(-c3ccccc3)nc(-c3cc(-c4cccc5c4sc4ccccc45)cc4c5ccccc5c5ccccc5c34)n2)cc1 ; c1ccc(-c2nc(-c3ccccc3)nc(-c3cc(-c4ccc5c(c4)sc4ccccc45)cc4c5ccccc5c5ccccc5c34)n2)cc1 ; c1ccc(-c2nc(-c3ccccc3)nc(-c3cc(-c4ccc5sc6ccccc6c5c4)cc4c5ccccc5c5ccccc5c34)n2)cc1]

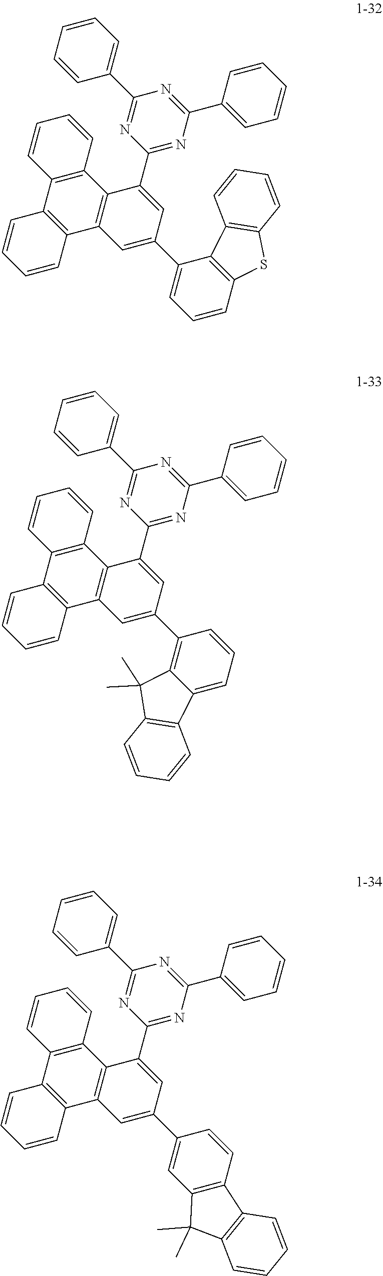 [SMILES: c1ccc(-c2nc(-c3ccccc3)nc(-c3cc(-c4cccc5sc6ccccc6c45)cc4c5ccccc5c5ccccc5c34)n2)cc1 ; CC1(C)c2ccccc2-c2cccc(-c3cc(-c4nc(-c5ccccc5)nc(-c5ccccc5)n4)c4c5ccccc5c5ccccc5c4c3)c21 ; CC1(C)c2ccccc2-c2ccc(-c3cc(-c4nc(-c5ccccc5)nc(-c5ccccc5)n4)c4c5ccccc5c5ccccc5c4c3)cc21]

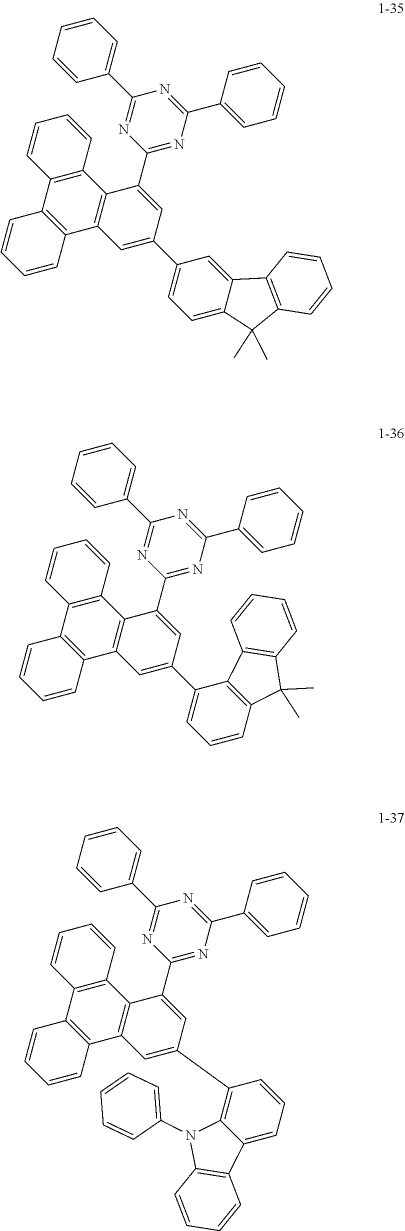 [SMILES: CC1(C)c2ccccc2-c2cc(-c3cc(-c4nc(-c5ccccc5)nc(-c5ccccc5)n4)c4c5ccccc5c5ccccc5c4c3)ccc21 ; CC1(C)c2ccccc2-c2c(-c3cc(-c4nc(-c5ccccc5)nc(-c5ccccc5)n4)c4c5ccccc5c5ccccc5c4c3)cccc21 ; c1ccc(-c2nc(-c3ccccc3)nc(-c3cc(-c4cccc5c6ccccc6n(-c6ccccc6)c45)cc4c5ccccc5c5ccccc5c34)n2)cc1]

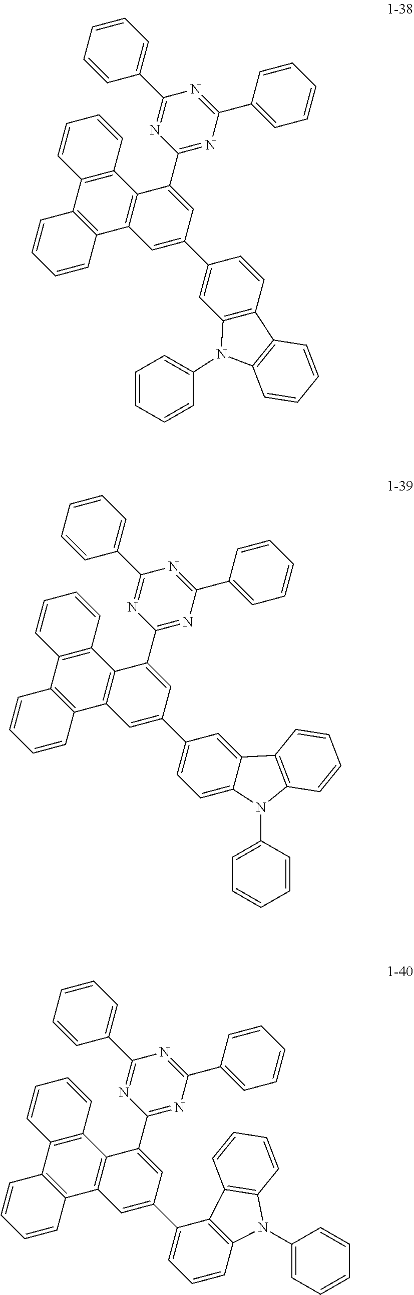 [SMILES: c1ccc(-c2nc(-c3ccccc3)nc(-c3cc(-c4ccc5c6ccccc6n(-c6ccccc6)c5c4)cc4c5ccccc5c5ccccc5c34)n2)cc1 ; c1ccc(-c2nc(-c3ccccc3)nc(-c3cc(-c4ccc5c(c4)c4ccccc4n5-c4ccccc4)cc4c5ccccc5c5ccccc5c34)n2)cc1 ; c1ccc(-c2nc(-c3ccccc3)nc(-c3cc(-c4cccc5c4c4ccccc4n5-c4ccccc4)cc4c5ccccc5c5ccccc5c34)n2)cc1]

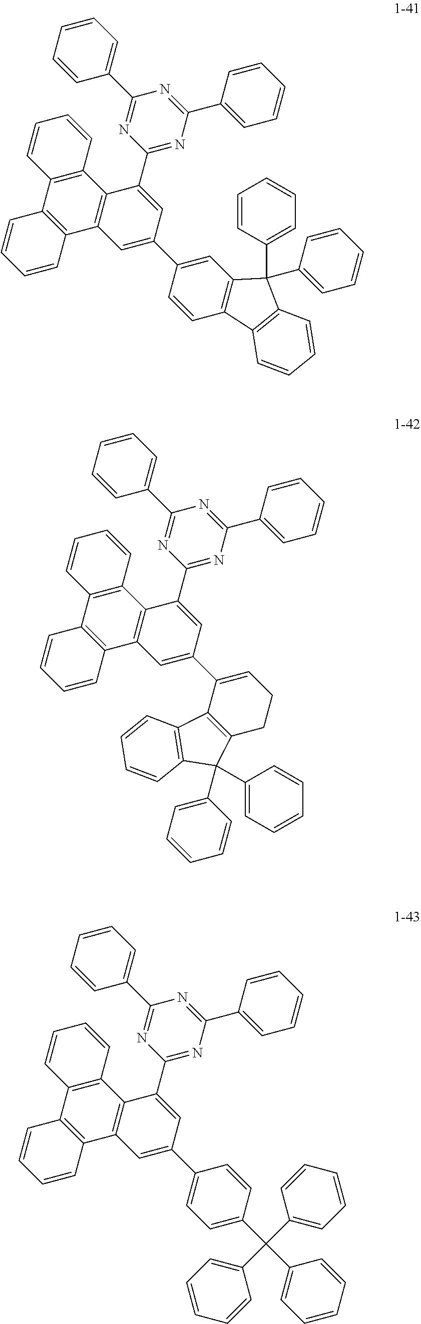 [SMILES: c1ccc(-c2nc(-c3ccccc3)nc(-c3cc(-c4ccc5c(c4)C(c4ccccc4)(c4ccccc4)c4ccccc4-5)cc4c5ccccc5c5ccccc5c34)n2)cc1 ; C1=C(c2cc(-c3nc(-c4ccccc4)nc(-c4ccccc4)n3)c3c4ccccc4c4ccccc4c3c2)C2=C(CC1)C(c1ccccc1)(c1ccccc1)c1ccccc12 ; c1ccc(-c2nc(-c3ccccc3)nc(-c3cc(-c4ccc(C(c5ccccc5)(c5ccccc5)c5ccccc5)cc4)cc4c5ccccc5c5ccccc5c34)n2)cc1]

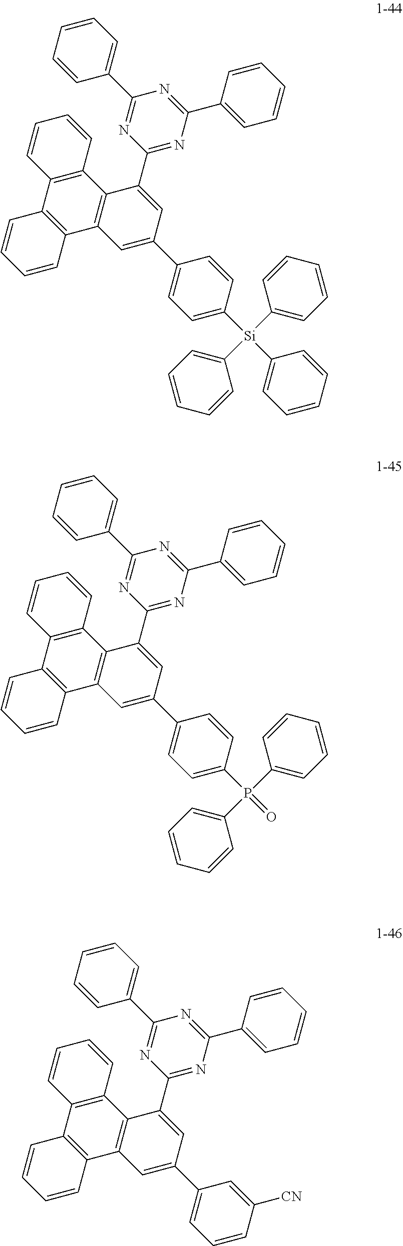 [SMILES: c1ccc(-c2nc(-c3ccccc3)nc(-c3cc(-c4ccc([Si](c5ccccc5)(c5ccccc5)c5ccccc5)cc4)cc4c5ccccc5c5ccccc5c34)n2)cc1 ; O=P(c1ccccc1)(c1ccccc1)c1ccc(-c2cc(-c3nc(-c4ccccc4)nc(-c4ccccc4)n3)c3c4ccccc4c4ccccc4c3c2)cc1 ; N#Cc1cccc(-c2cc(-c3nc(-c4ccccc4)nc(-c4ccccc4)n3)c3c4ccccc4c4ccccc4c3c2)c1]

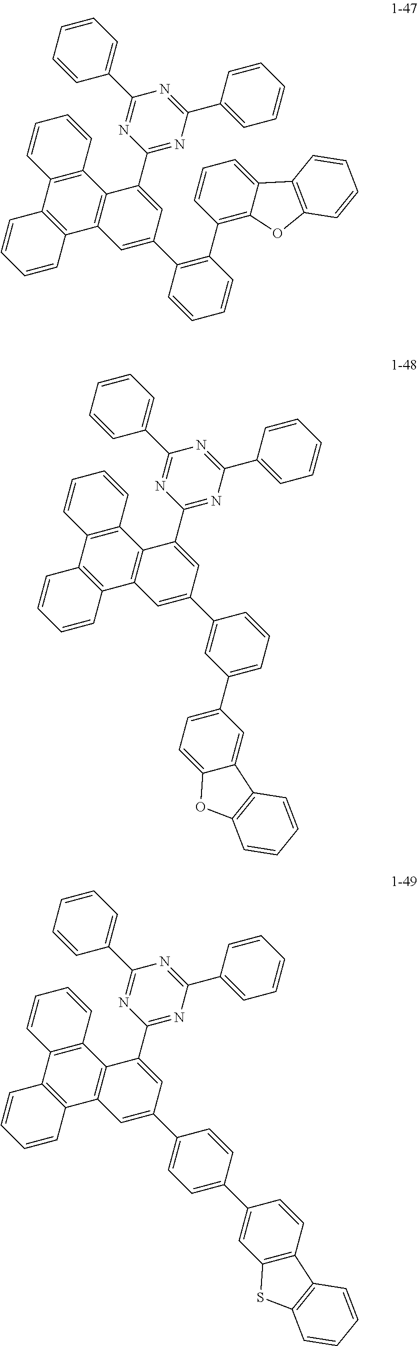 [SMILES: c1ccc(-c2nc(-c3ccccc3)nc(-c3cc(-c4ccccc4-c4cccc5c4oc4ccccc45)cc4c5ccccc5c5ccccc5c34)n2)cc1 ; c1ccc(-c2nc(-c3ccccc3)nc(-c3cc(-c4cccc(-c5ccc6oc7ccccc7c6c5)c4)cc4c5ccccc5c5ccccc5c34)n2)cc1 ; c1ccc(-c2nc(-c3ccccc3)nc(-c3cc(-c4ccc(-c5ccc6c(c5)sc5ccccc56)cc4)cc4c5ccccc5c5ccccc5c34)n2)cc1]

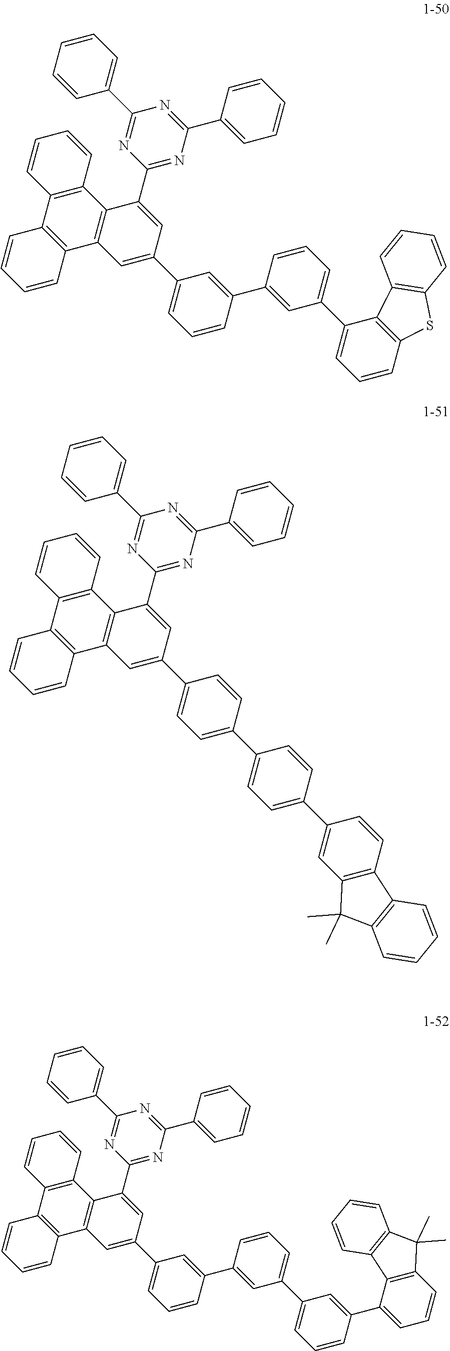 [SMILES: c1ccc(-c2nc(-c3ccccc3)nc(-c3cc(-c4cccc(-c5cccc(-c6cccc7sc8ccccc8c67)c5)c4)cc4c5ccccc5c5ccccc5c34)n2)cc1 ; CC1(C)c2ccccc2-c2ccc(-c3ccc(-c4ccc(-c5cc(-c6nc(-c7ccccc7)nc(-c7ccccc7)n6)c6c7ccccc7c7ccccc7c6c5)cc4)cc3)cc21 ; CC1(C)c2ccccc2-c2c(-c3cccc(-c4cccc(-c5cccc(-c6cc(-c7nc(-c8ccccc8)nc(-c8ccccc8)n7)c7c8ccccc8c8ccccc8c7c6)c5)c4)c3)cccc21]

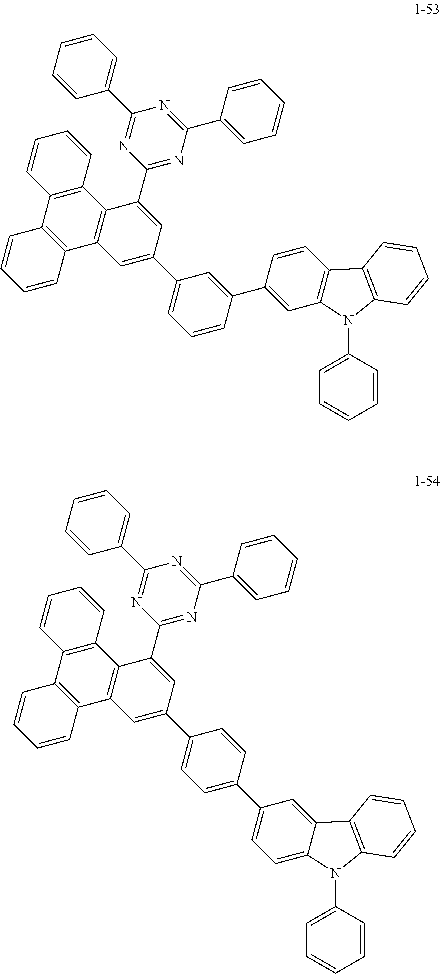 [SMILES: c1ccc(-c2nc(-c3ccccc3)nc(-c3cc(-c4cccc(-c5ccc6c7ccccc7n(-c7ccccc7)c6c5)c4)cc4c5ccccc5c5ccccc5c34)n2)cc1 ; c1ccc(-c2nc(-c3ccccc3)nc(-c3cc(-c4ccc(-c5ccc6c(c5)c5ccccc5n6-c5ccccc5)cc4)cc4c5ccccc5c5ccccc5c34)n2)cc1]

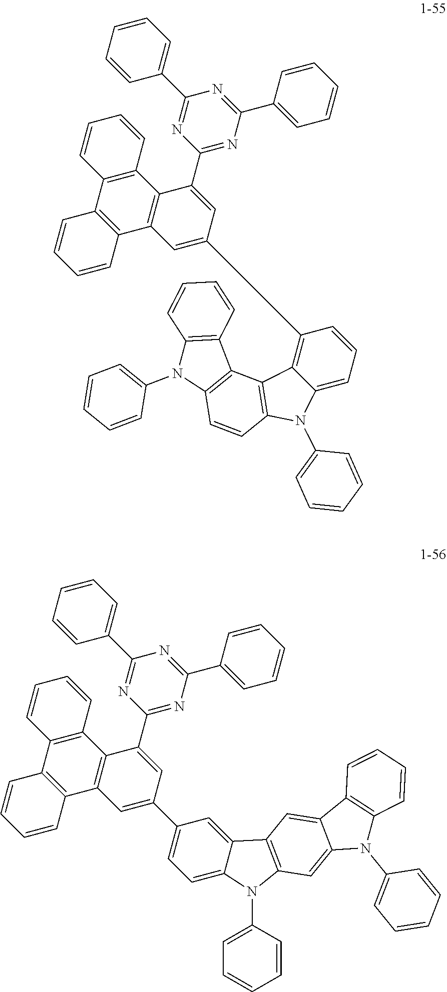 [SMILES: c1ccc(-c2nc(-c3ccccc3)nc(-c3cc(-c4cccc5c4c4c6c7ccccc7n(-c7ccccc7)c6ccc4n5-c4ccccc4)cc4c5ccccc5c5ccccc5c34)n2)cc1 ; c1ccc(-c2nc(-c3ccccc3)nc(-c3cc(-c4ccc5c(c4)c4cc6c7ccccc7n(-c7ccccc7)c6cc4n5-c4ccccc4)cc4c5ccccc5c5ccccc5c34)n2)cc1]

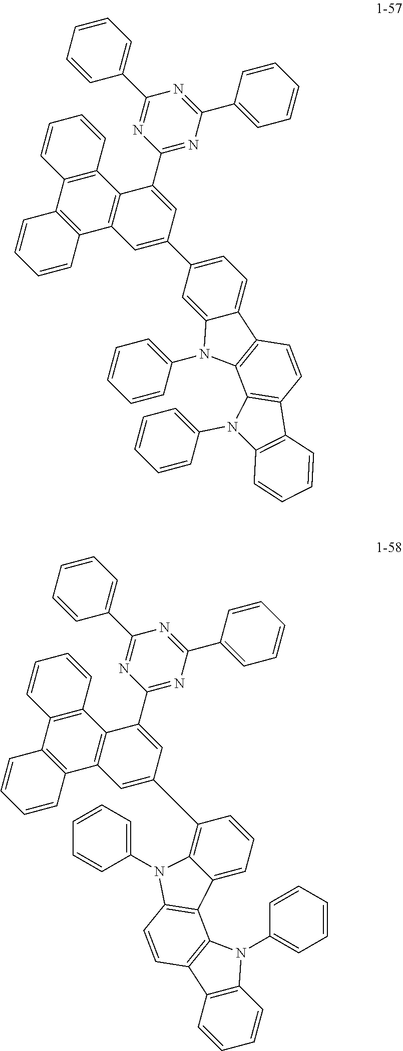 [SMILES: c1ccc(-c2nc(-c3ccccc3)nc(-c3cc(-c4ccc5c6ccc7c8ccccc8n(-c8ccccc8)c7c6n(-c6ccccc6)c5c4)cc4c5ccccc5c5ccccc5c34)n2)cc1 ; c1ccc(-c2nc(-c3ccccc3)nc(-c3cc(-c4cccc5c6c(ccc7c8ccccc8n(-c8ccccc8)c76)n(-c6ccccc6)c45)cc4c5ccccc5c5ccccc5c34)n2)cc1]

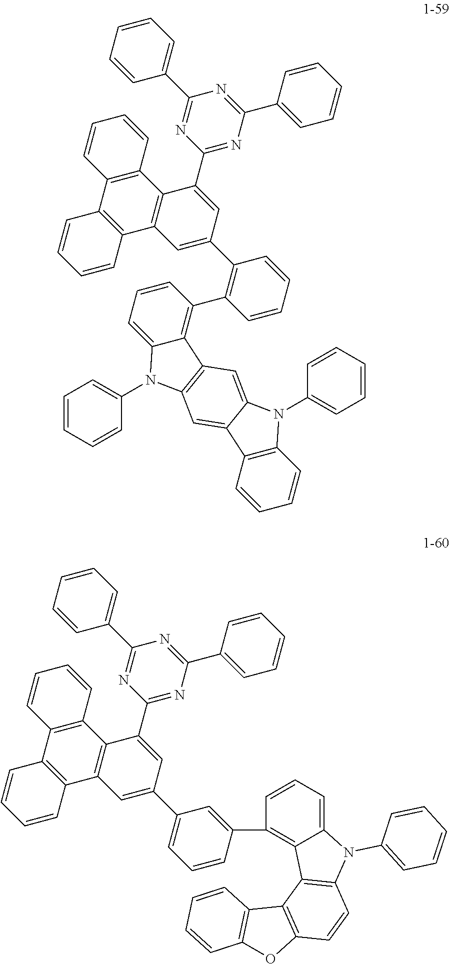 [SMILES: c1ccc(-c2nc(-c3ccccc3)nc(-c3cc(-c4ccccc4-c4cccc5c4c4cc6c(cc4n5-c4ccccc4)c4ccccc4n6-c4ccccc4)cc4c5ccccc5c5ccccc5c34)n2)cc1 ; c1ccc(-c2nc(-c3ccccc3)nc(-c3cc(-c4cccc(-c5cccc6c5c5c7c(ccc5n6-c5ccccc5)oc5ccccc57)c4)cc4c5ccccc5c5ccccc5c34)n2)cc1]

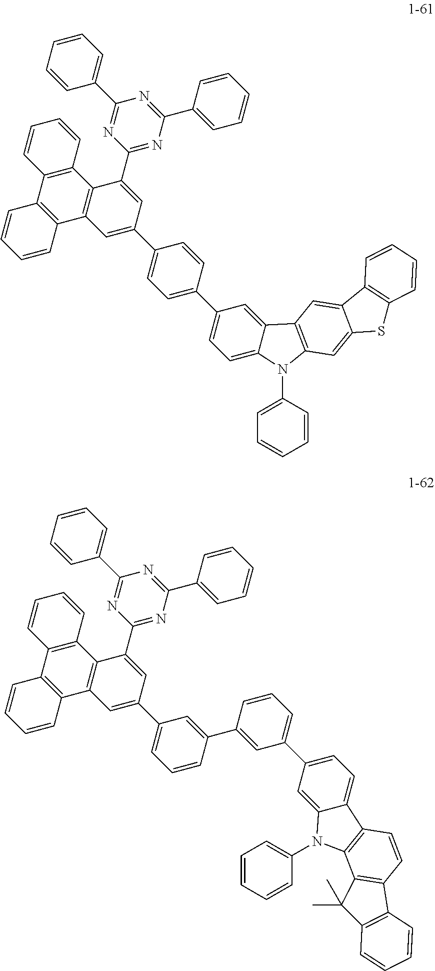 [SMILES: c1ccc(-c2nc(-c3ccccc3)nc(-c3cc(-c4ccc(-c5ccc6c(c5)c5cc7c(cc5n6-c5ccccc5)sc5ccccc57)cc4)cc4c5ccccc5c5ccccc5c34)n2)cc1 ; CC1(C)c2ccccc2-c2ccc3c4ccc(-c5cccc(-c6cccc(-c7cc(-c8nc(-c9ccccc9)nc(-c9ccccc9)n8)c8c9ccccc9c9ccccc9c8c7)c6)c5)cc4n(-c4ccccc4)c3c21]

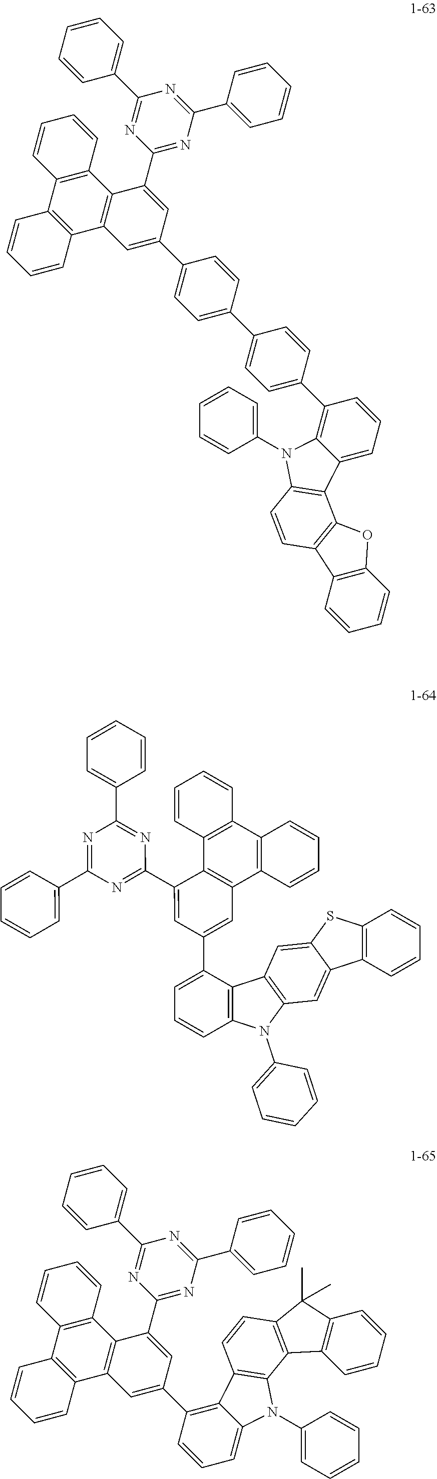 [SMILES: c1ccc(-c2nc(-c3ccccc3)nc(-c3cc(-c4ccc(-c5ccc(-c6cccc7c8c9oc%10ccccc%10c9ccc8n(-c8ccccc8)c67)cc5)cc4)cc4c5ccccc5c5ccccc5c34)n2)cc1 ; c1ccc(-c2nc(-c3ccccc3)nc(-c3cc(-c4cccc5c4c4cc6sc7ccccc7c6cc4n5-c4ccccc4)cc4c5ccccc5c5ccccc5c34)n2)cc1 ; CC1(C)c2ccccc2-c2c1ccc1c3c(-c4cc(-c5nc(-c6ccccc6)nc(-c6ccccc6)n5)c5c6ccccc6c6ccccc6c5c4)cccc3n(-c3ccccc3)c21]

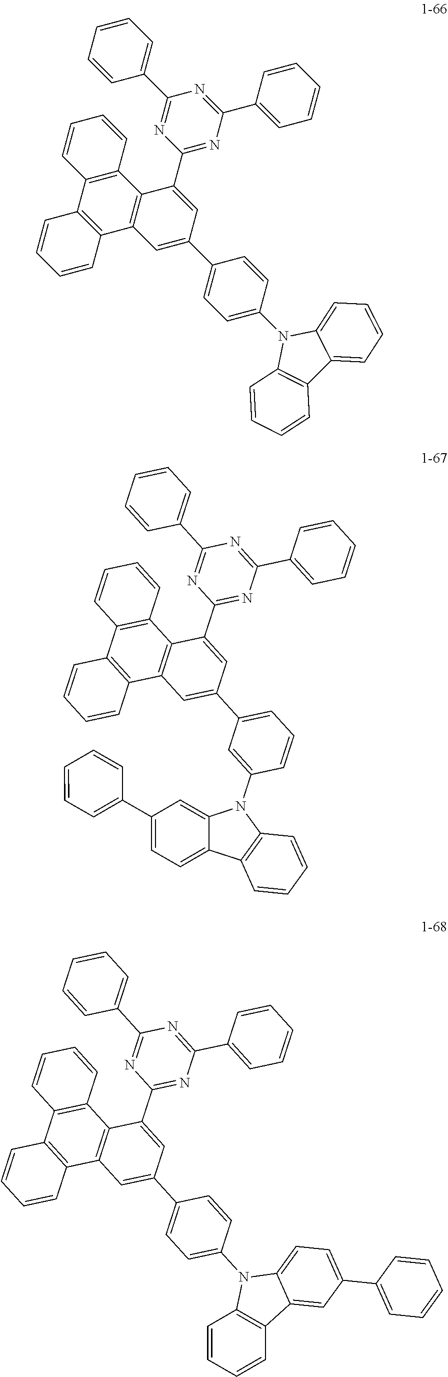 [SMILES: c1ccc(-c2nc(-c3ccccc3)nc(-c3cc(-c4ccc(-n5c6ccccc6c6ccccc65)cc4)cc4c5ccccc5c5ccccc5c34)n2)cc1 ; c1ccc(-c2ccc3c4ccccc4n(-c4cccc(-c5cc(-c6nc(-c7ccccc7)nc(-c7ccccc7)n6)c6c7ccccc7c7ccccc7c6c5)c4)c3c2)cc1 ; c1ccc(-c2ccc3c(c2)c2ccccc2n3-c2ccc(-c3cc(-c4nc(-c5ccccc5)nc(-c5ccccc5)n4)c4c5ccccc5c5ccccc5c4c3)cc2)cc1]

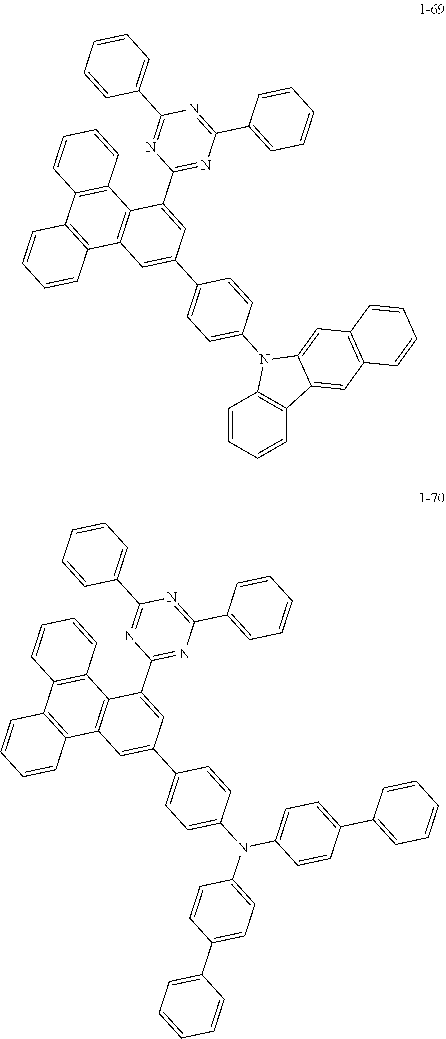 [SMILES: c1ccc(-c2nc(-c3ccccc3)nc(-c3cc(-c4ccc(-n5c6ccccc6c6cc7ccccc7cc65)cc4)cc4c5ccccc5c5ccccc5c34)n2)cc1 ; c1ccc(-c2ccc(N(c3ccc(-c4ccccc4)cc3)c3ccc(-c4cc(-c5nc(-c6ccccc6)nc(-c6ccccc6)n5)c5c6ccccc6c6ccccc6c5c4)cc3)cc2)cc1]

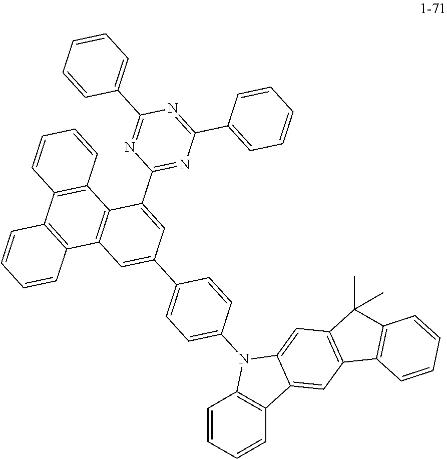 [SMILES: CC1(C)c2ccccc2-c2cc3c4ccccc4n(-c4ccc(-c5cc(-c6nc(-c7ccccc7)nc(-c7ccccc7)n6)c6c7ccccc7c7ccccc7c6c5)cc4)c3cc21]

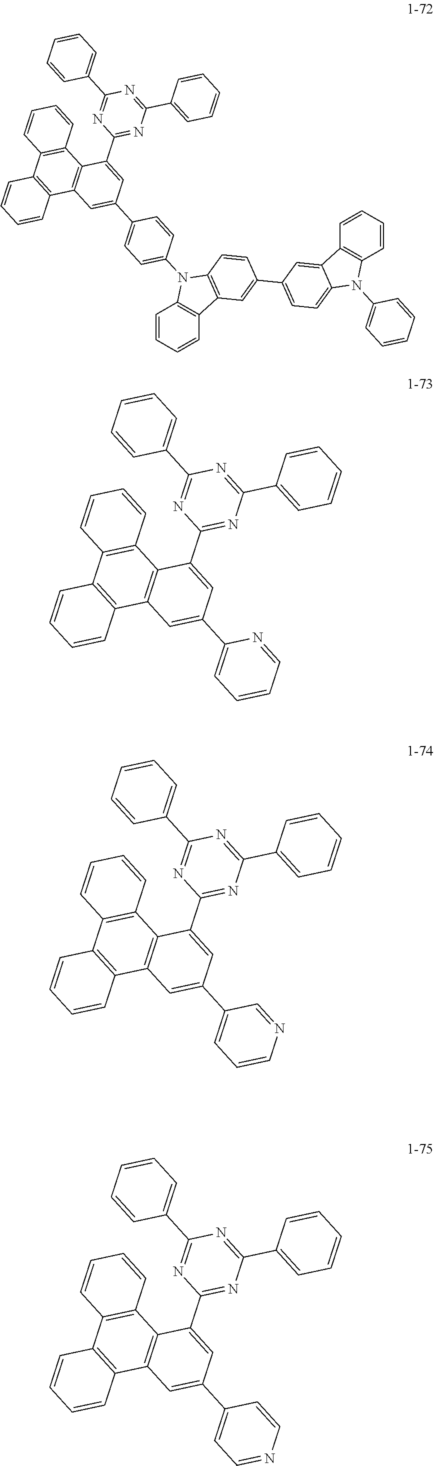 [SMILES: c1ccc(-c2nc(-c3ccccc3)nc(-c3cc(-c4ccc(-n5c6ccccc6c6cc(-c7ccc8c(c7)c7ccccc7n8-c7ccccc7)ccc65)cc4)cc4c5ccccc5c5ccccc5c34)n2)cc1 ; c1ccc(-c2nc(-c3ccccc3)nc(-c3cc(-c4ccccn4)cc4c5ccccc5c5ccccc5c34)n2)cc1 ; c1ccc(-c2nc(-c3ccccc3)nc(-c3cc(-c4cccnc4)cc4c5ccccc5c5ccccc5c34)n2)cc1 ; c1ccc(-c2nc(-c3ccccc3)nc(-c3cc(-c4ccncc4)cc4c5ccccc5c5ccccc5c34)n2)cc1]

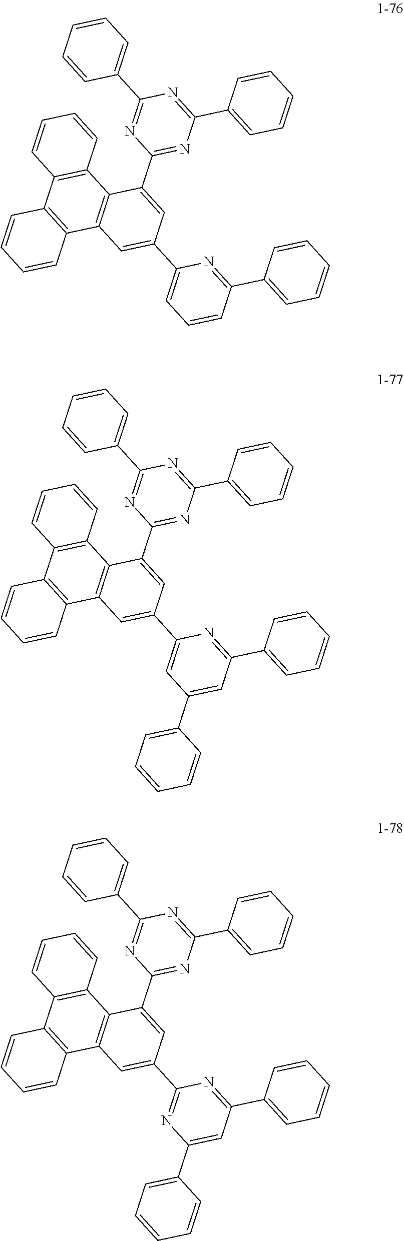 [SMILES: c1ccc(-c2cccc(-c3cc(-c4nc(-c5ccccc5)nc(-c5ccccc5)n4)c4c5ccccc5c5ccccc5c4c3)n2)cc1 ; c1ccc(-c2cc(-c3ccccc3)nc(-c3cc(-c4nc(-c5ccccc5)nc(-c5ccccc5)n4)c4c5ccccc5c5ccccc5c4c3)c2)cc1 ; c1ccc(-c2cc(-c3ccccc3)nc(-c3cc(-c4nc(-c5ccccc5)nc(-c5ccccc5)n4)c4c5ccccc5c5ccccc5c4c3)n2)cc1]

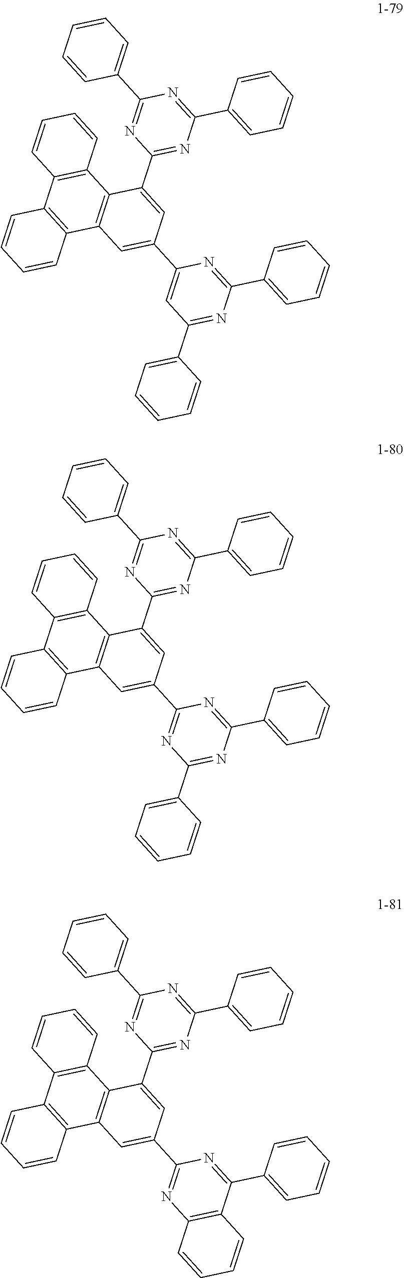 [SMILES: c1ccc(-c2cc(-c3cc(-c4nc(-c5ccccc5)nc(-c5ccccc5)n4)c4c5ccccc5c5ccccc5c4c3)nc(-c3ccccc3)n2)cc1 ; c1ccc(-c2nc(-c3ccccc3)nc(-c3cc(-c4nc(-c5ccccc5)nc(-c5ccccc5)n4)c4c5ccccc5c5ccccc5c4c3)n2)cc1 ; c1ccc(-c2nc(-c3ccccc3)nc(-c3cc(-c4nc(-c5ccccc5)c5ccccc5n4)cc4c5ccccc5c5ccccc5c34)n2)cc1]

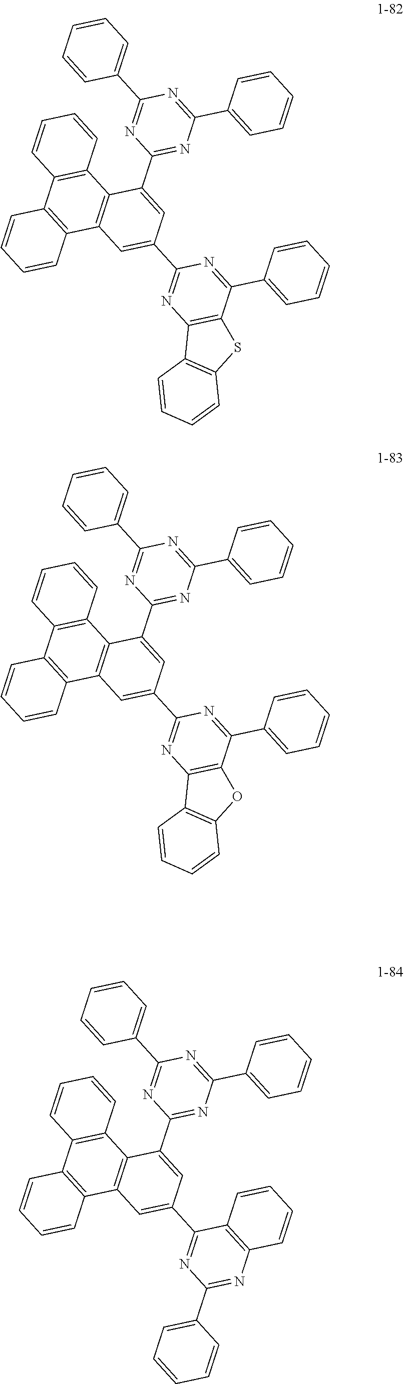 [SMILES: c1ccc(-c2nc(-c3ccccc3)nc(-c3cc(-c4nc(-c5ccccc5)c5sc6ccccc6c5n4)cc4c5ccccc5c5ccccc5c34)n2)cc1 ; c1ccc(-c2nc(-c3ccccc3)nc(-c3cc(-c4nc(-c5ccccc5)c5oc6ccccc6c5n4)cc4c5ccccc5c5ccccc5c34)n2)cc1 ; c1ccc(-c2nc(-c3ccccc3)nc(-c3cc(-c4nc(-c5ccccc5)nc5ccccc45)cc4c5ccccc5c5ccccc5c34)n2)cc1]

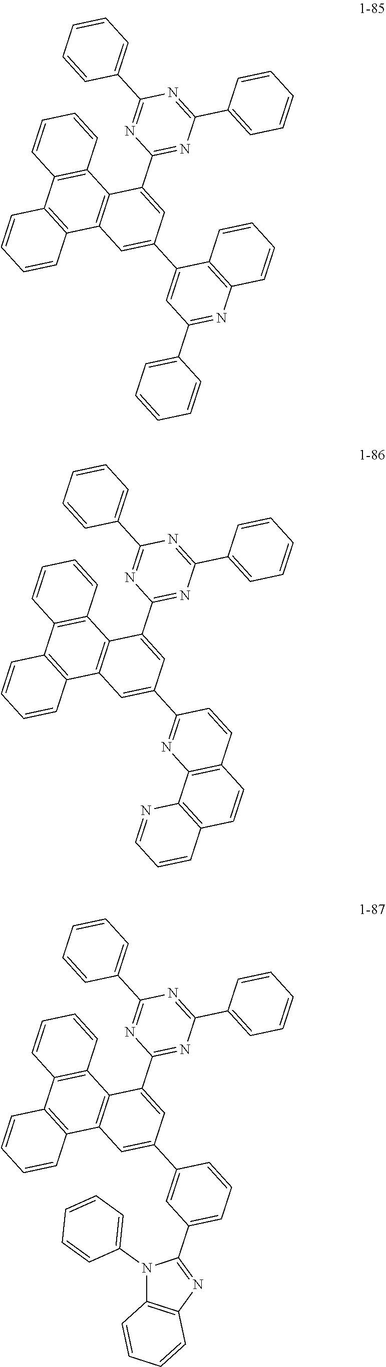 [SMILES: c1ccc(-c2cc(-c3cc(-c4nc(-c5ccccc5)nc(-c5ccccc5)n4)c4c5ccccc5c5ccccc5c4c3)c3ccccc3n2)cc1 ; c1ccc(-c2nc(-c3ccccc3)nc(-c3cc(-c4ccc5ccc6cccnc6c5n4)cc4c5ccccc5c5ccccc5c34)n2)cc1 ; c1ccc(-c2nc(-c3ccccc3)nc(-c3cc(-c4cccc(-c5nc6ccccc6n5-c5ccccc5)c4)cc4c5ccccc5c5ccccc5c34)n2)cc1]

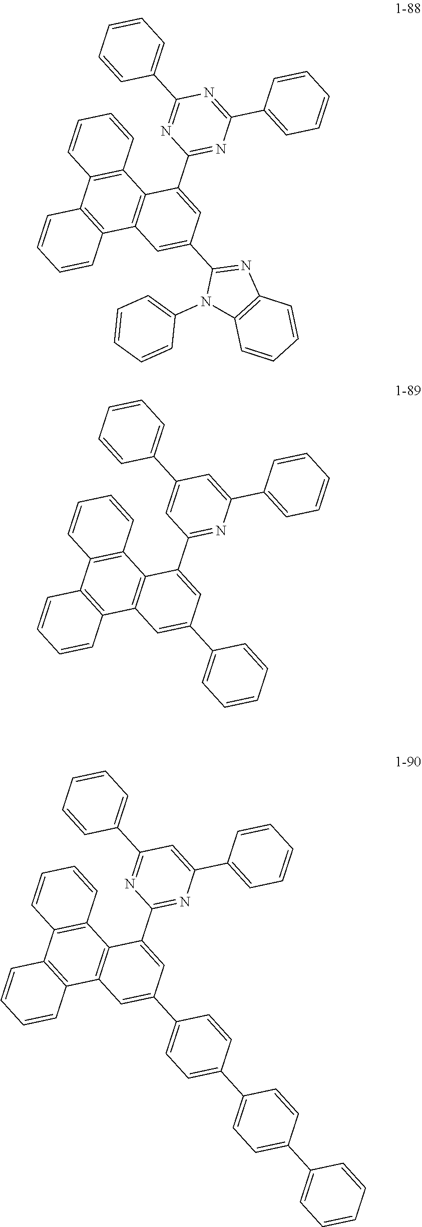

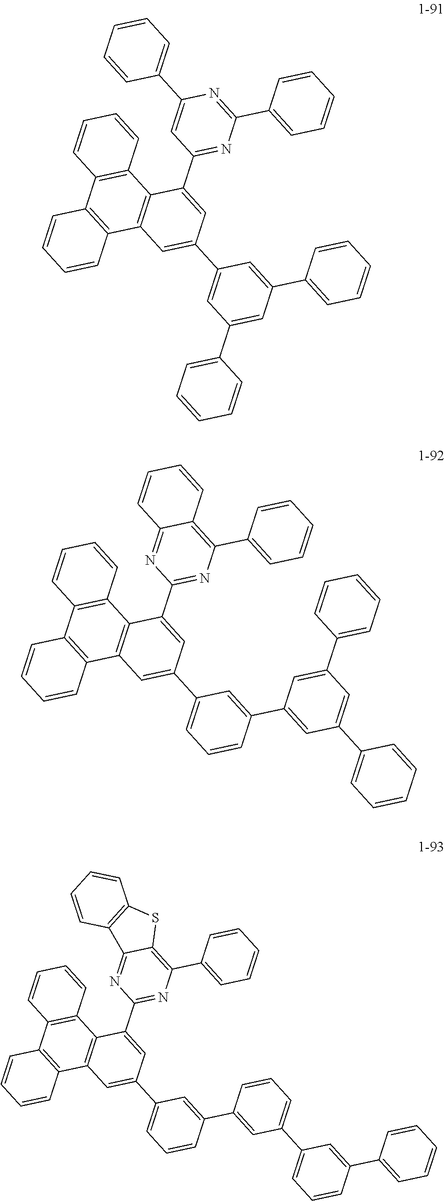 [SMILES: c1ccc(-c2cc(-c3ccccc3)cc(-c3cc(-c4cc(-c5ccccc5)nc(-c5ccccc5)n4)c4c5ccccc5c5ccccc5c4c3)c2)cc1 ; c1ccc(-c2cc(-c3ccccc3)cc(-c3cccc(-c4cc(-c5nc(-c6ccccc6)c6ccccc6n5)c5c6ccccc6c6ccccc6c5c4)c3)c2)cc1 ; c1ccc(-c2cccc(-c3cccc(-c4cccc(-c5cc(-c6nc(-c7ccccc7)c7sc8ccccc8c7n6)c6c7ccccc7c7ccccc7c6c5)c4)c3)c2)cc1]

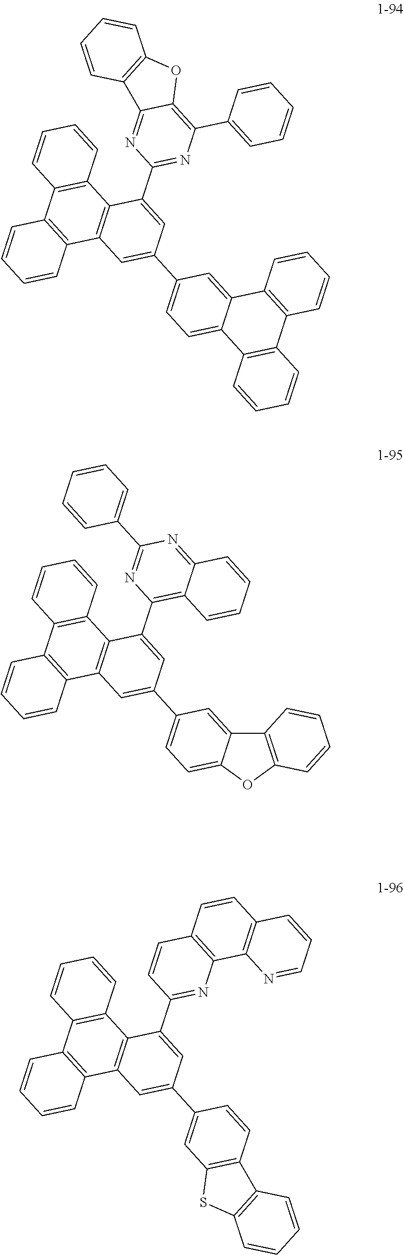 [SMILES: c1ccc(-c2nc(-c3cc(-c4ccc5c6ccccc6c6ccccc6c5c4)cc4c5ccccc5c5ccccc5c34)nc3c2oc2ccccc23)cc1 ; c1ccc(-c2nc(-c3cc(-c4ccc5oc6ccccc6c5c4)cc4c5ccccc5c5ccccc5c34)c3ccccc3n2)cc1 ; c1cnc2c(c1)ccc1ccc(-c3cc(-c4ccc5c(c4)sc4ccccc45)cc4c5ccccc5c5ccccc5c34)nc12]

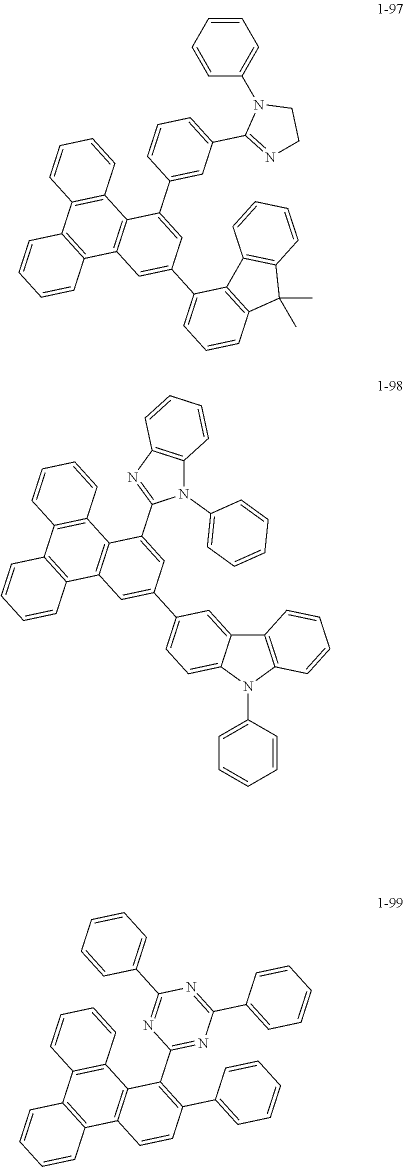 [SMILES: CC1(C)c2ccccc2-c2c(-c3cc(-c4cccc(C5=NCCN5c5ccccc5)c4)c4c5ccccc5c5ccccc5c4c3)cccc21 ; c1ccc(-n2c(-c3cc(-c4ccc5c(c4)c4ccccc4n5-c4ccccc4)cc4c5ccccc5c5ccccc5c34)nc3ccccc32)cc1 ; c1ccc(-c2nc(-c3ccccc3)nc(-c3c(-c4ccccc4)ccc4c5ccccc5c5ccccc5c34)n2)cc1]

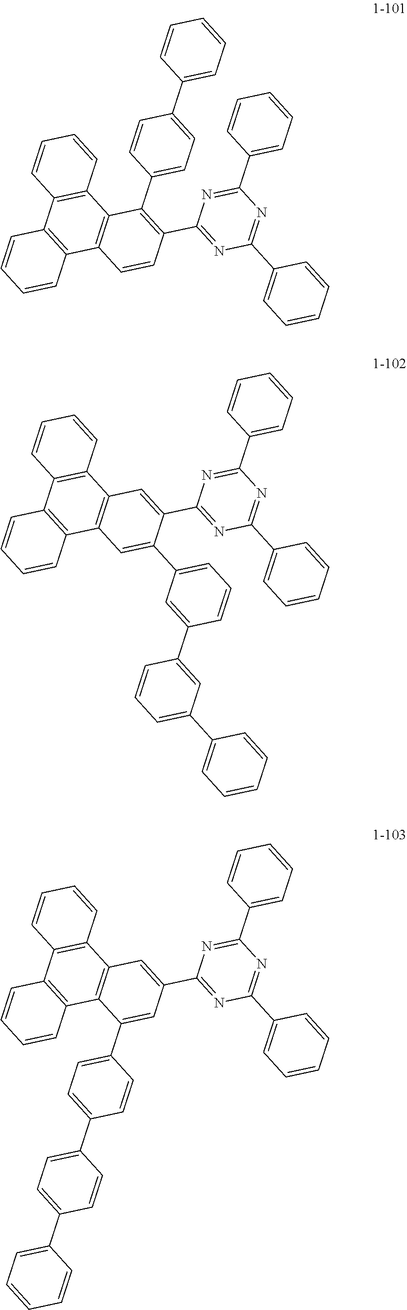 [SMILES: c1ccc(-c2ccc(-c3c(-c4nc(-c5ccccc5)nc(-c5ccccc5)n4)ccc4c5ccccc5c5ccccc5c34)cc2)cc1 ; c1ccc(-c2cccc(-c3cccc(-c4cc5c6ccccc6c6ccccc6c5cc4-c4nc(-c5ccccc5)nc(-c5ccccc5)n4)c3)c2)cc1 ; c1ccc(-c2ccc(-c3ccc(-c4cc(-c5nc(-c6ccccc6)nc(-c6ccccc6)n5)cc5c6ccccc6c6ccccc6c45)cc3)cc2)cc1]

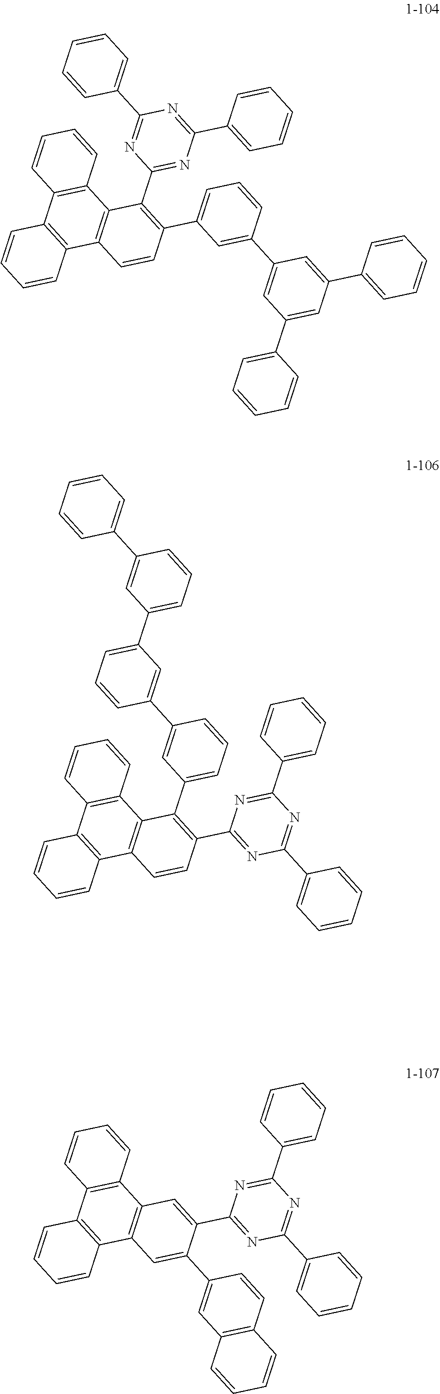 [SMILES: c1ccc(-c2cc(-c3ccccc3)cc(-c3cccc(-c4ccc5c6ccccc6c6ccccc6c5c4-c4nc(-c5ccccc5)nc(-c5ccccc5)n4)c3)c2)cc1 ; c1ccc(-c2cccc(-c3cccc(-c4cccc(-c5c(-c6nc(-c7ccccc7)nc(-c7ccccc7)n6)ccc6c7ccccc7c7ccccc7c56)c4)c3)c2)cc1 ; c1ccc(-c2nc(-c3ccccc3)nc(-c3cc4c5ccccc5c5ccccc5c4cc3-c3ccc4ccccc4c3)n2)cc1]

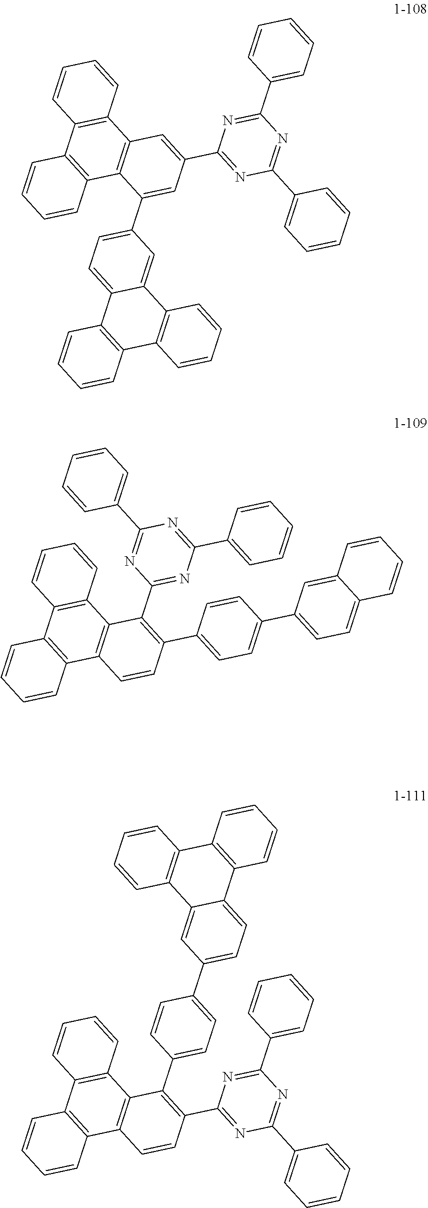 [SMILES: c1ccc(-c2nc(-c3ccccc3)nc(-c3cc(-c4ccc5c6ccccc6c6ccccc6c5c4)c4c5ccccc5c5ccccc5c4c3)n2)cc1 ; c1ccc(-c2nc(-c3ccccc3)nc(-c3c(-c4ccc(-c5ccc6ccccc6c5)cc4)ccc4c5ccccc5c5ccccc5c34)n2)cc1 ; c1ccc(-c2nc(-c3ccccc3)nc(-c3ccc4c5ccccc5c5ccccc5c4c3-c3ccc(-c4ccc5c6ccccc6c6ccccc6c5c4)cc3)n2)cc1]

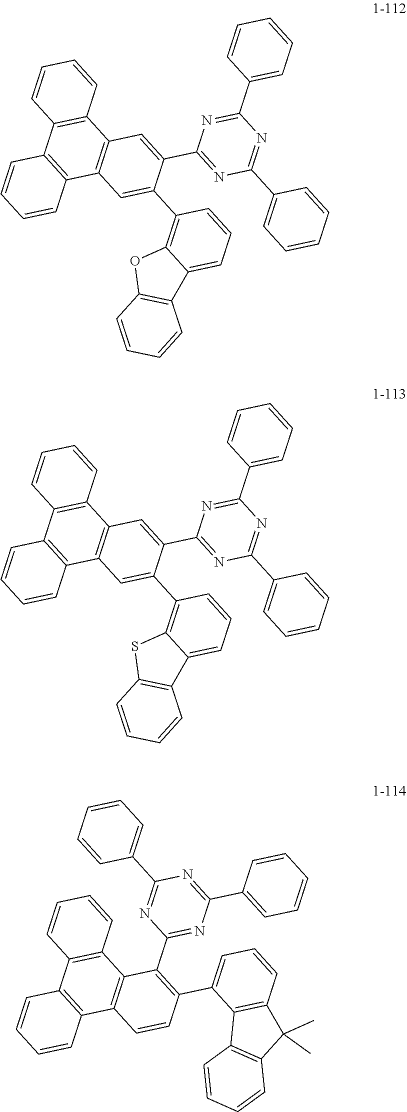 [SMILES: c1ccc(-c2nc(-c3ccccc3)nc(-c3cc4c5ccccc5c5ccccc5c4cc3-c3cccc4c3oc3ccccc34)n2)cc1 ; c1ccc(-c2nc(-c3ccccc3)nc(-c3cc4c5ccccc5c5ccccc5c4cc3-c3cccc4c3sc3ccccc34)n2)cc1 ; CC1(C)c2ccccc2-c2c(-c3ccc4c5ccccc5c5ccccc5c4c3-c3nc(-c4ccccc4)nc(-c4ccccc4)n3)cccc21]

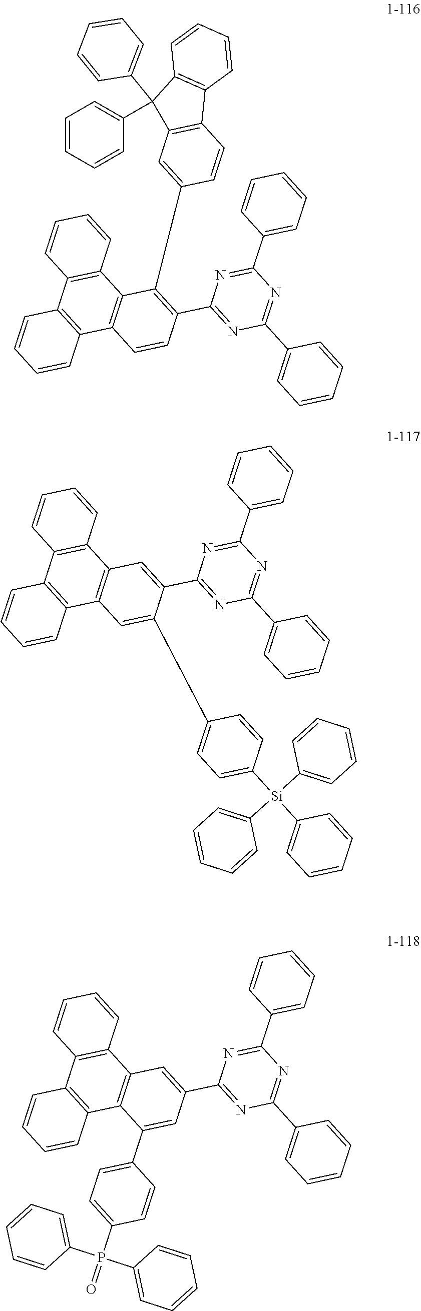 [SMILES: c1ccc(-c2nc(-c3ccccc3)nc(-c3ccc4c5ccccc5c5ccccc5c4c3-c3ccc4c(c3)C(c3ccccc3)(c3ccccc3)c3ccccc3-4)n2)cc1 ; c1ccc(-c2nc(-c3ccccc3)nc(-c3cc4c5ccccc5c5ccccc5c4cc3-c3ccc([Si](c4ccccc4)(c4ccccc4)c4ccccc4)cc3)n2)cc1 ; O=P(c1ccccc1)(c1ccccc1)c1ccc(-c2cc(-c3nc(-c4ccccc4)nc(-c4ccccc4)n3)cc3c4ccccc4c4ccccc4c23)cc1]

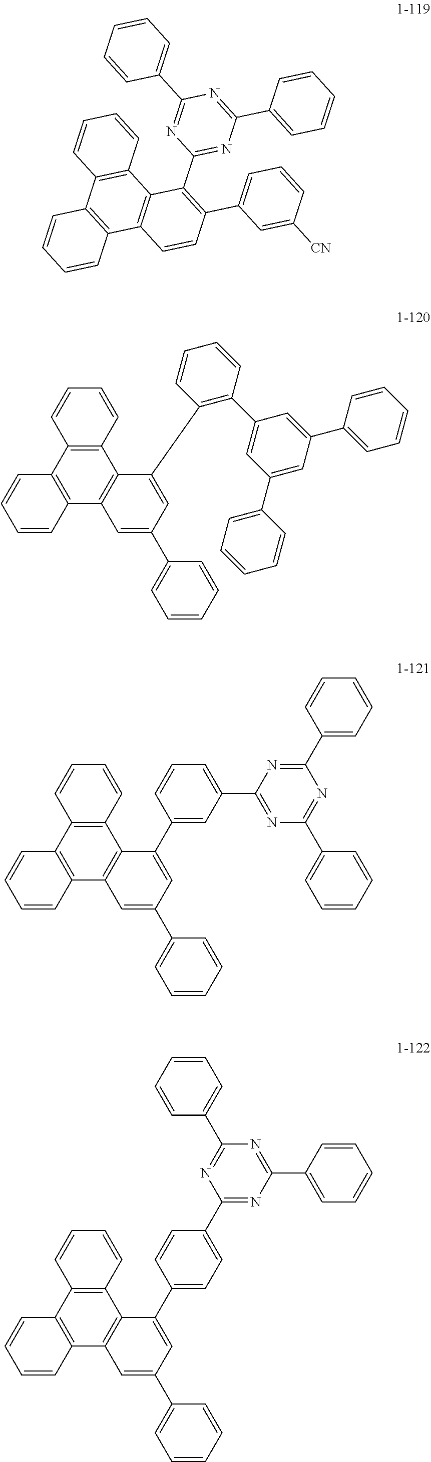

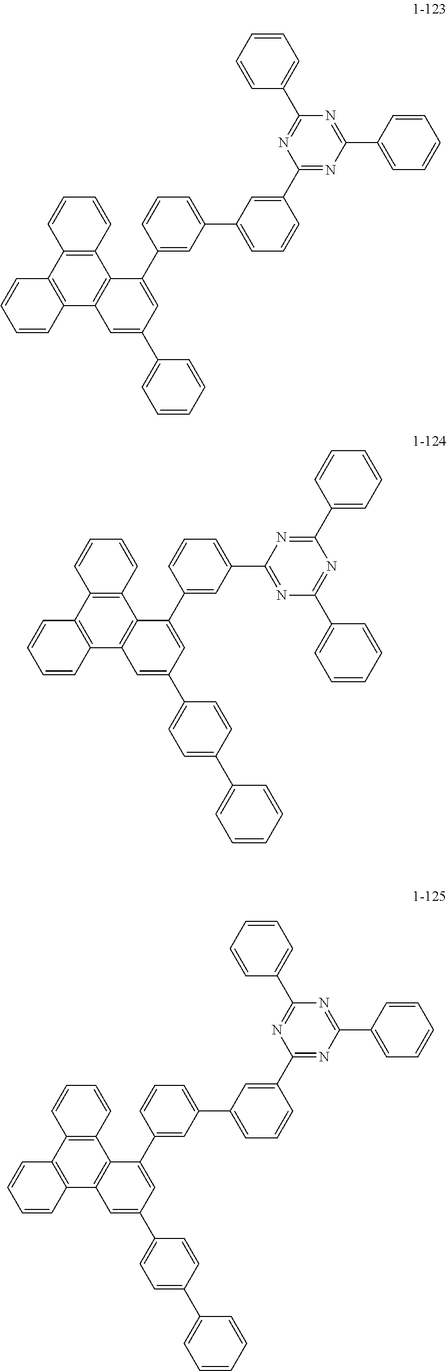 [SMILES: c1ccc(-c2cc(-c3cccc(-c4cccc(-c5nc(-c6ccccc6)nc(-c6ccccc6)n5)c4)c3)c3c4ccccc4c4ccccc4c3c2)cc1 ; c1ccc(-c2ccc(-c3cc(-c4cccc(-c5nc(-c6ccccc6)nc(-c6ccccc6)n5)c4)c4c5ccccc5c5ccccc5c4c3)cc2)cc1 ; c1ccc(-c2ccc(-c3cc(-c4cccc(-c5cccc(-c6nc(-c7ccccc7)nc(-c7ccccc7)n6)c5)c4)c4c5ccccc5c5ccccc5c4c3)cc2)cc1]

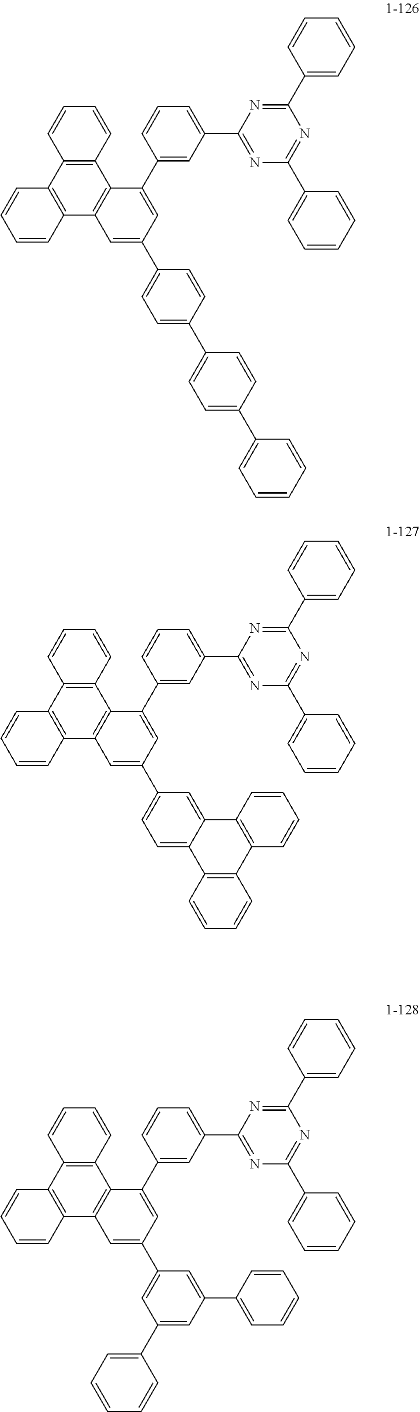 [SMILES: c1ccc(-c2ccc(-c3ccc(-c4cc(-c5cccc(-c6nc(-c7ccccc7)nc(-c7ccccc7)n6)c5)c5c6ccccc6c6ccccc6c5c4)cc3)cc2)cc1 ; c1ccc(-c2nc(-c3ccccc3)nc(-c3cccc(-c4cc(-c5ccc6c7ccccc7c7ccccc7c6c5)cc5c6ccccc6c6ccccc6c45)c3)n2)cc1 ; c1ccc(-c2cc(-c3ccccc3)cc(-c3cc(-c4cccc(-c5nc(-c6ccccc6)nc(-c6ccccc6)n5)c4)c4c5ccccc5c5ccccc5c4c3)c2)cc1]

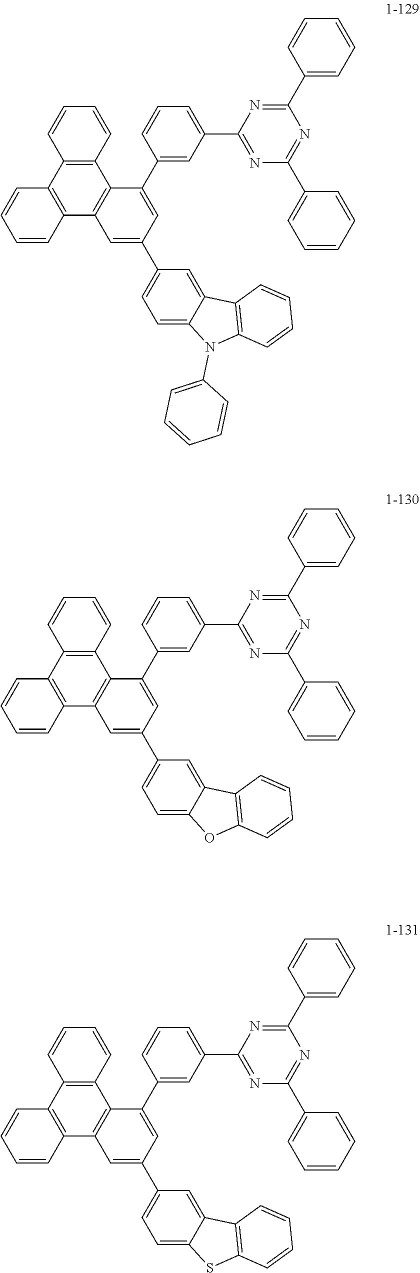 [SMILES: c1ccc(-c2nc(-c3ccccc3)nc(-c3cccc(-c4cc(-c5ccc6c(c5)c5ccccc5n6-c5ccccc5)cc5c6ccccc6c6ccccc6c45)c3)n2)cc1 ; c1ccc(-c2nc(-c3ccccc3)nc(-c3cccc(-c4cc(-c5ccc6oc7ccccc7c6c5)cc5c6ccccc6c6ccccc6c45)c3)n2)cc1 ; c1ccc(-c2nc(-c3ccccc3)nc(-c3cccc(-c4cc(-c5ccc6sc7ccccc7c6c5)cc5c6ccccc6c6ccccc6c45)c3)n2)cc1]

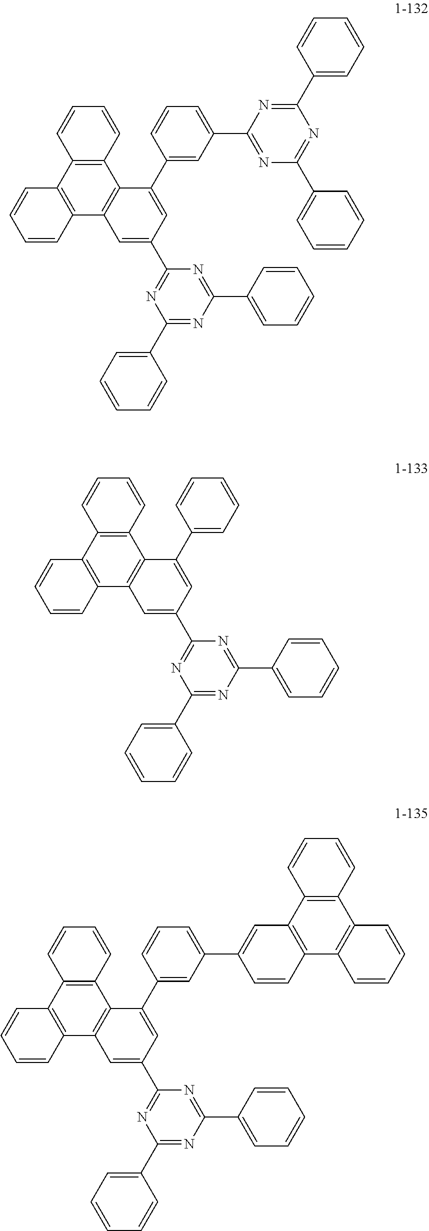 [SMILES: c1ccc(-c2nc(-c3ccccc3)nc(-c3cccc(-c4cc(-c5nc(-c6ccccc6)nc(-c6ccccc6)n5)cc5c6ccccc6c6ccccc6c45)c3)n2)cc1 ; c1ccc(-c2nc(-c3ccccc3)nc(-c3cc(-c4ccccc4)c4c5ccccc5c5ccccc5c4c3)n2)cc1 ; c1ccc(-c2nc(-c3ccccc3)nc(-c3cc(-c4cccc(-c5ccc6c7ccccc7c7ccccc7c6c5)c4)c4c5ccccc5c5ccccc5c4c3)n2)cc1]

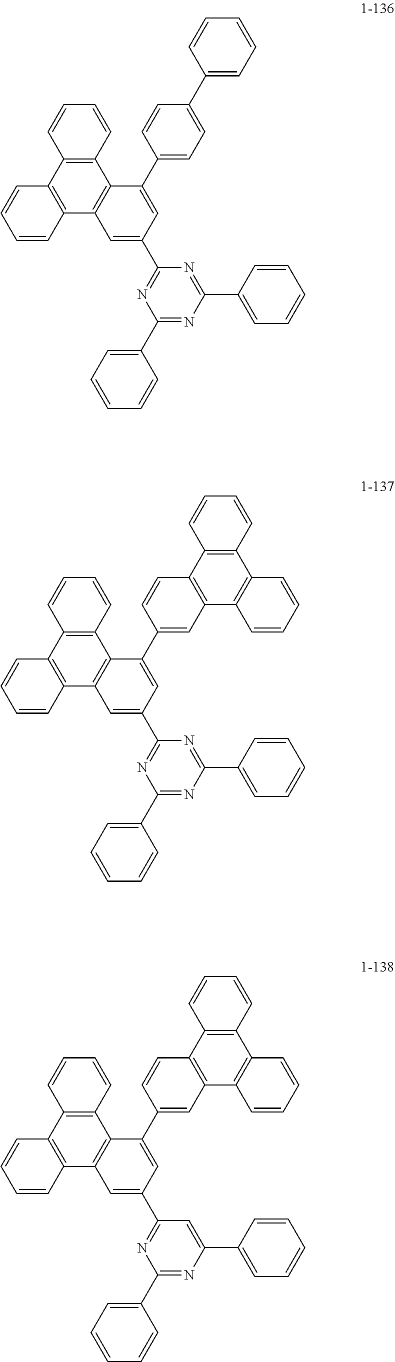 [SMILES: c1ccc(-c2ccc(-c3cc(-c4nc(-c5ccccc5)nc(-c5ccccc5)n4)cc4c5ccccc5c5ccccc5c34)cc2)cc1 ; c1ccc(-c2nc(-c3ccccc3)nc(-c3cc(-c4ccc5c6ccccc6c6ccccc6c5c4)c4c5ccccc5c5ccccc5c4c3)n2)cc1 ; c1ccc(-c2cc(-c3cc(-c4ccc5c6ccccc6c6ccccc6c5c4)c4c5ccccc5c5ccccc5c4c3)nc(-c3ccccc3)n2)cc1]

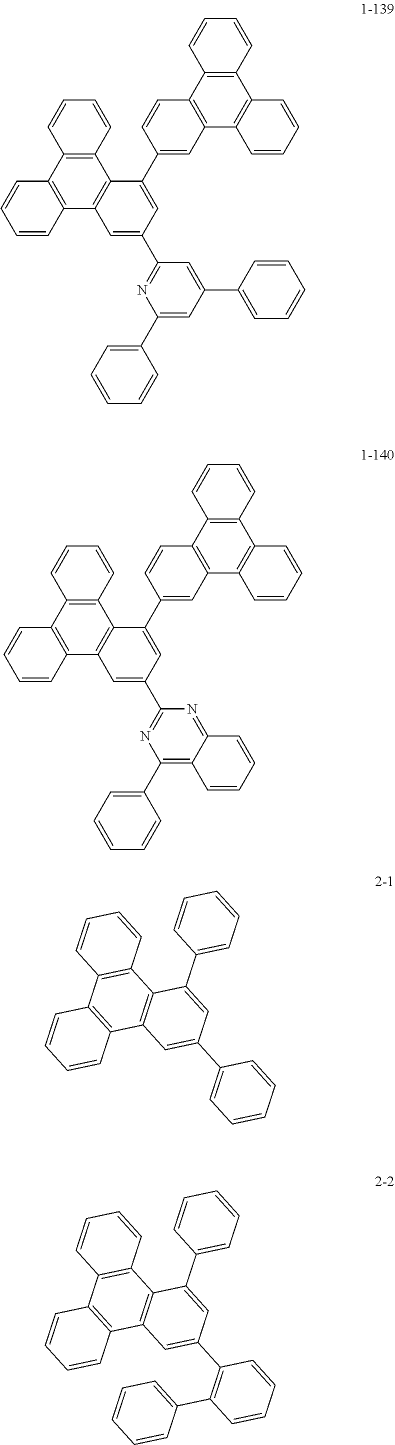

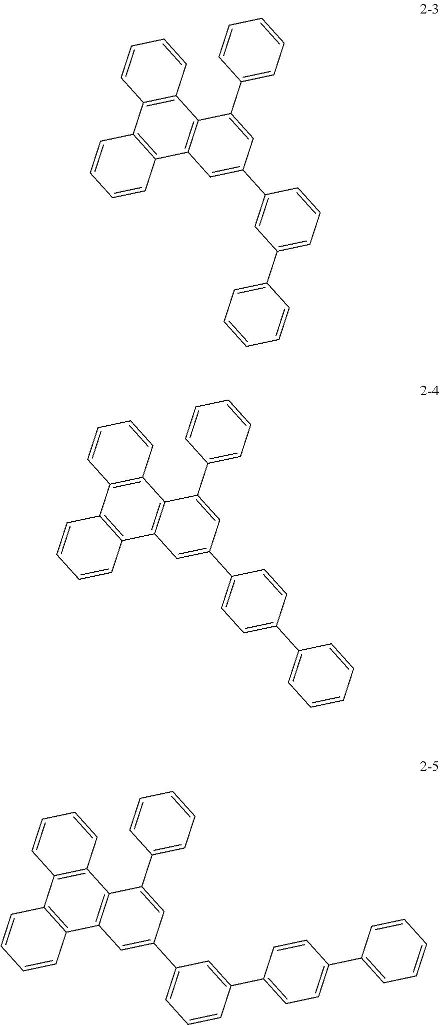

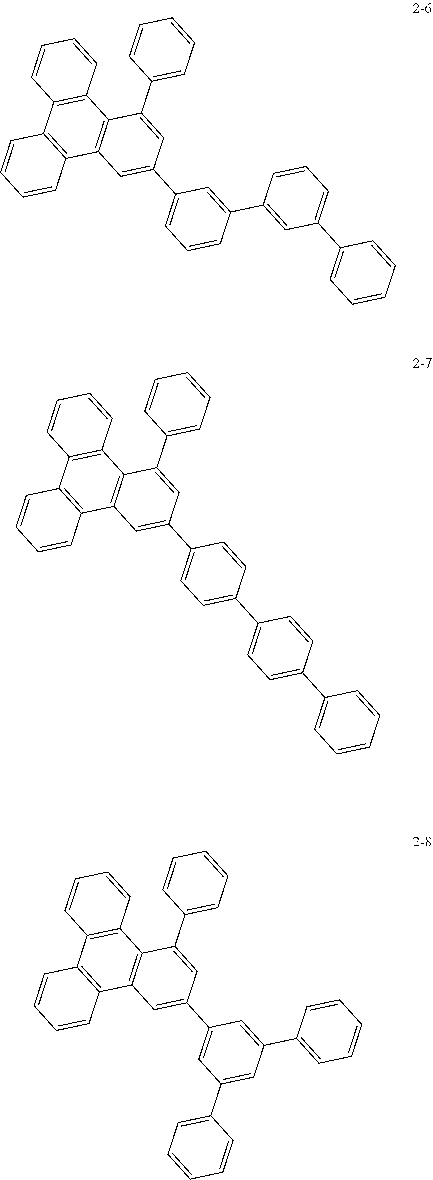

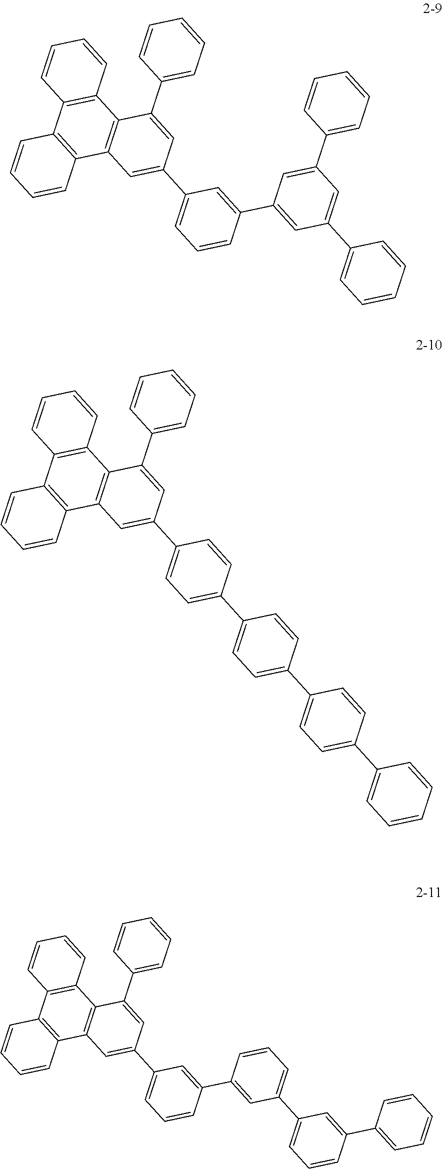

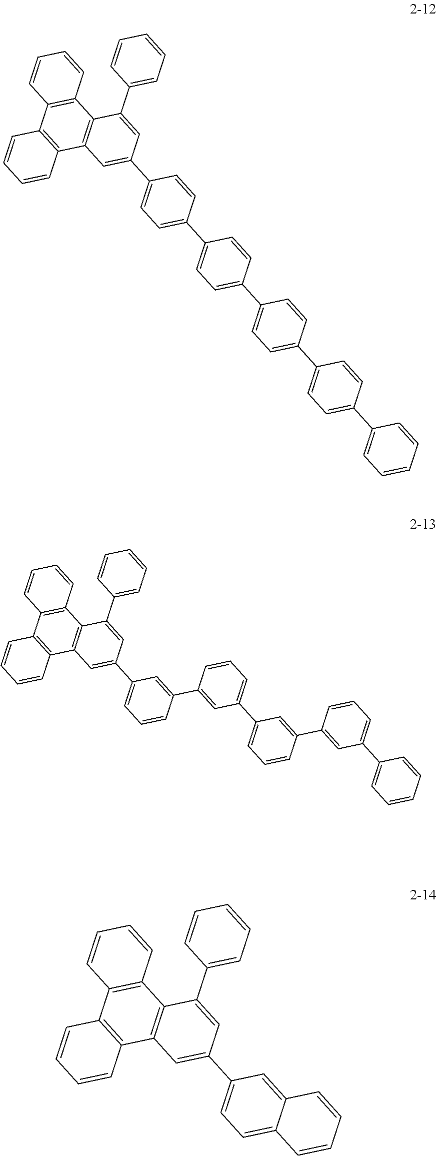

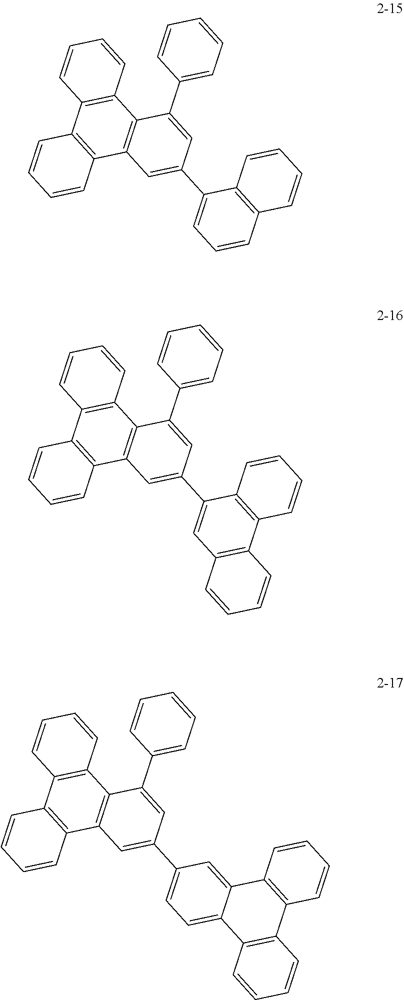

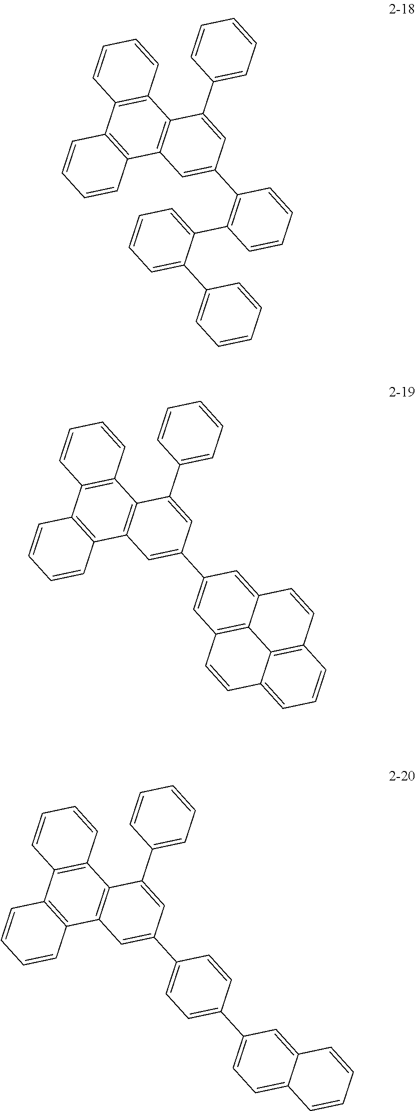

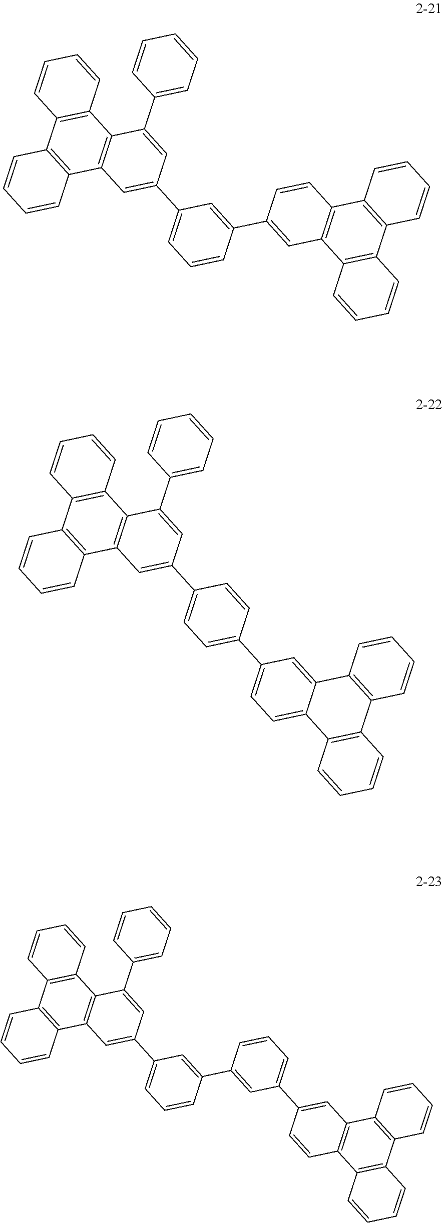

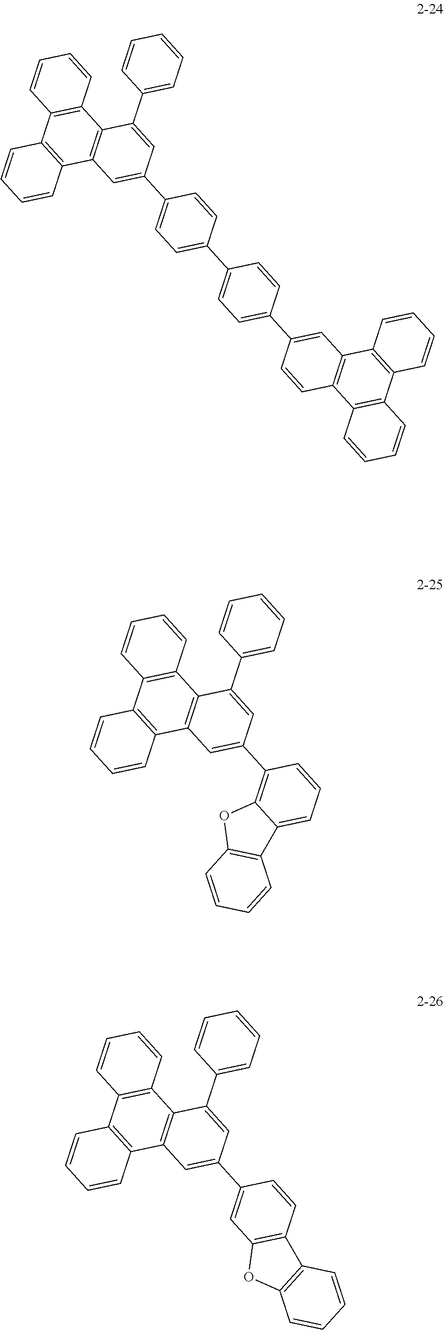

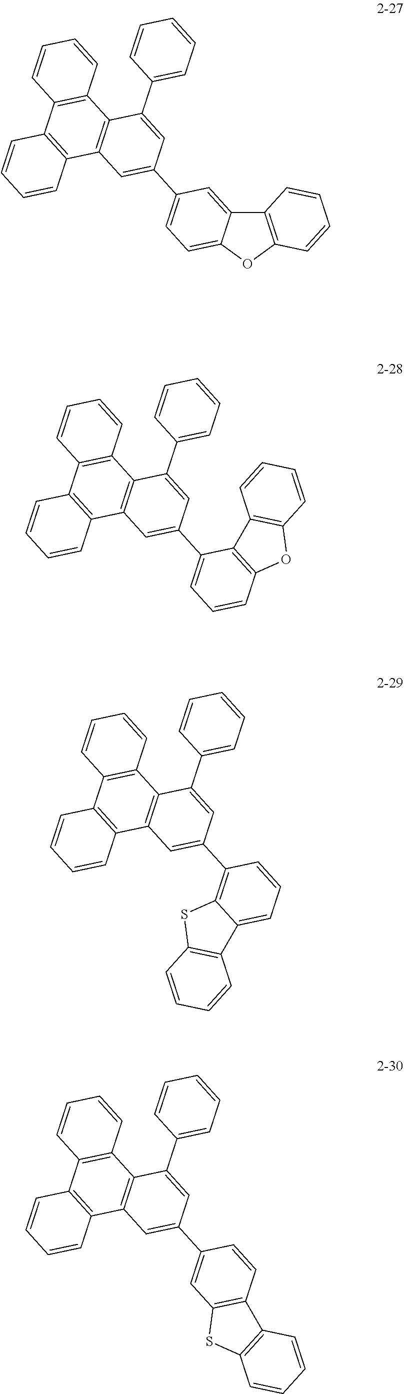 [SMILES: c1ccc(-c2cc(-c3ccc4oc5ccccc5c4c3)cc3c4ccccc4c4ccccc4c23)cc1 ; c1ccc(-c2cc(-c3cccc4oc5ccccc5c34)cc3c4ccccc4c4ccccc4c23)cc1 ; c1ccc(-c2cc(-c3cccc4c3sc3ccccc34)cc3c4ccccc4c4ccccc4c23)cc1 ; c1ccc(-c2cc(-c3ccc4c(c3)sc3ccccc34)cc3c4ccccc4c4ccccc4c23)cc1]

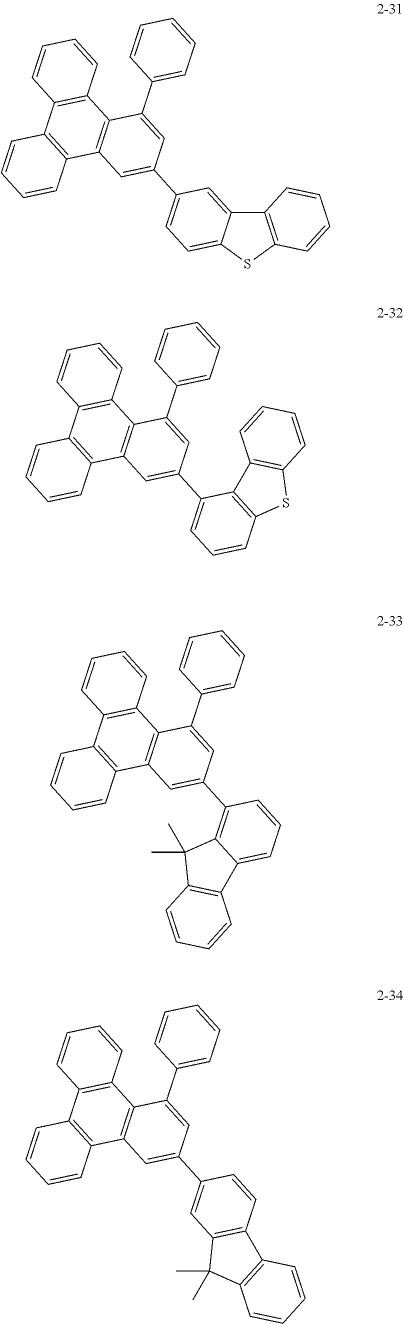

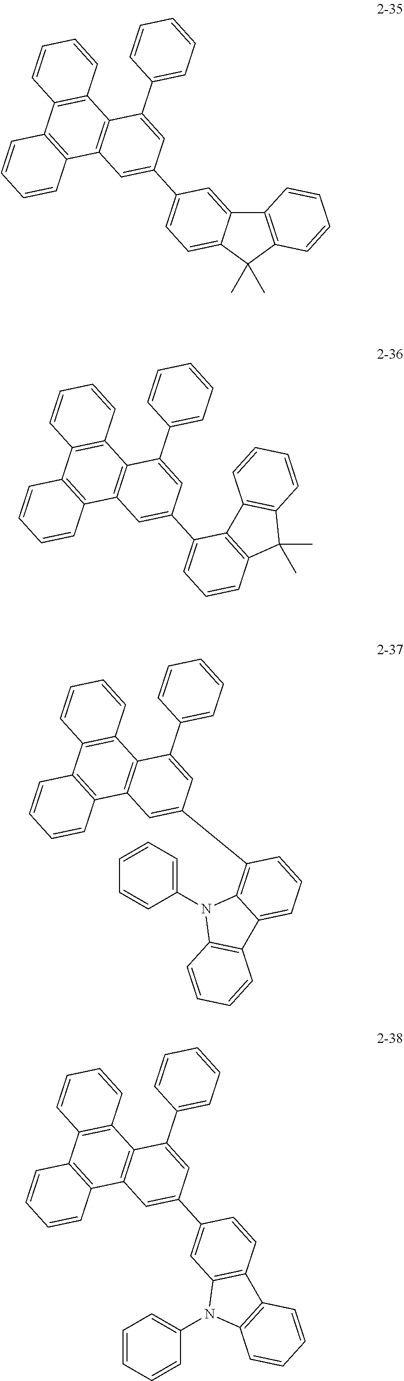

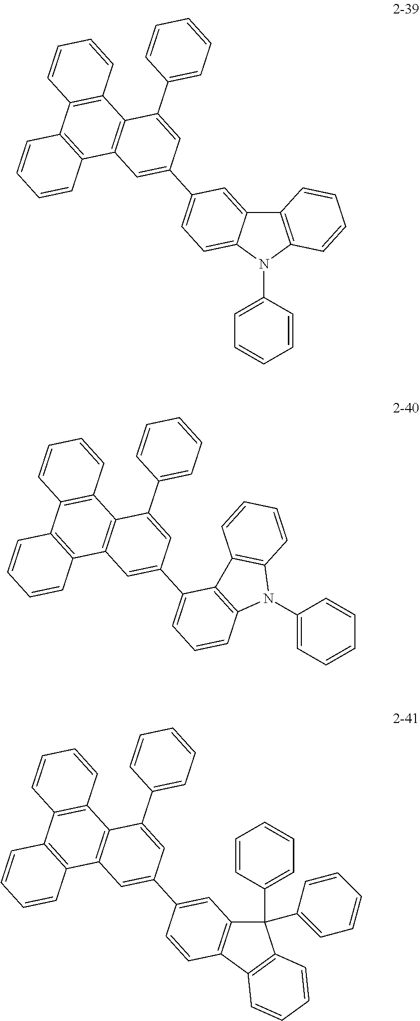

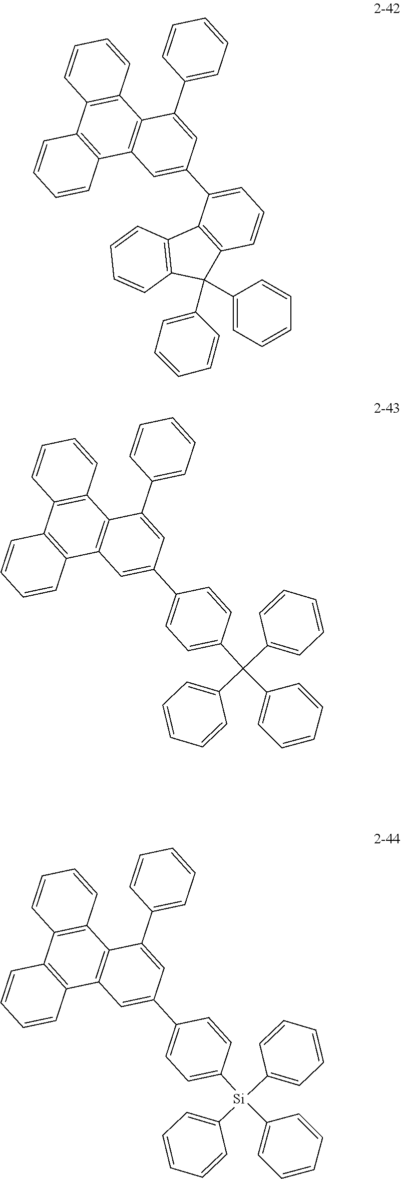

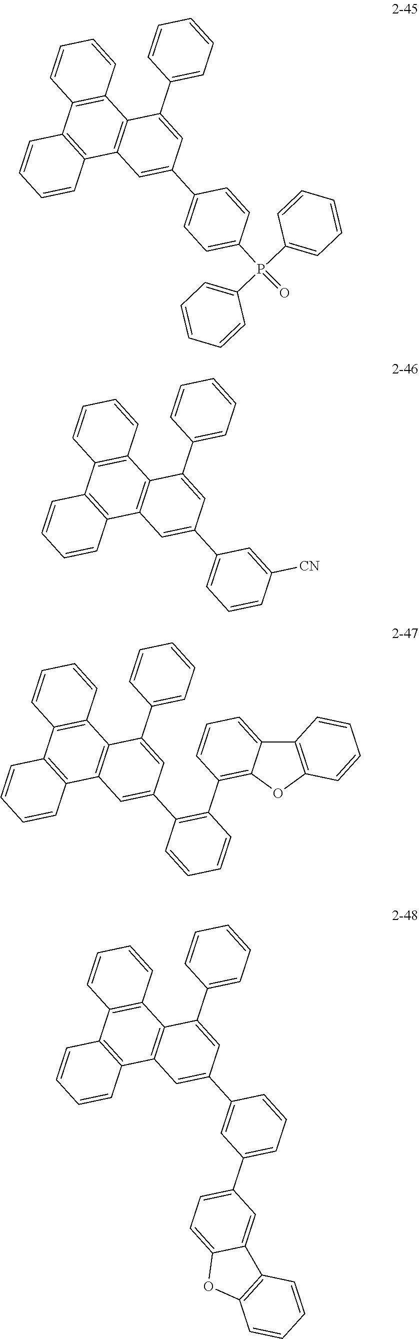

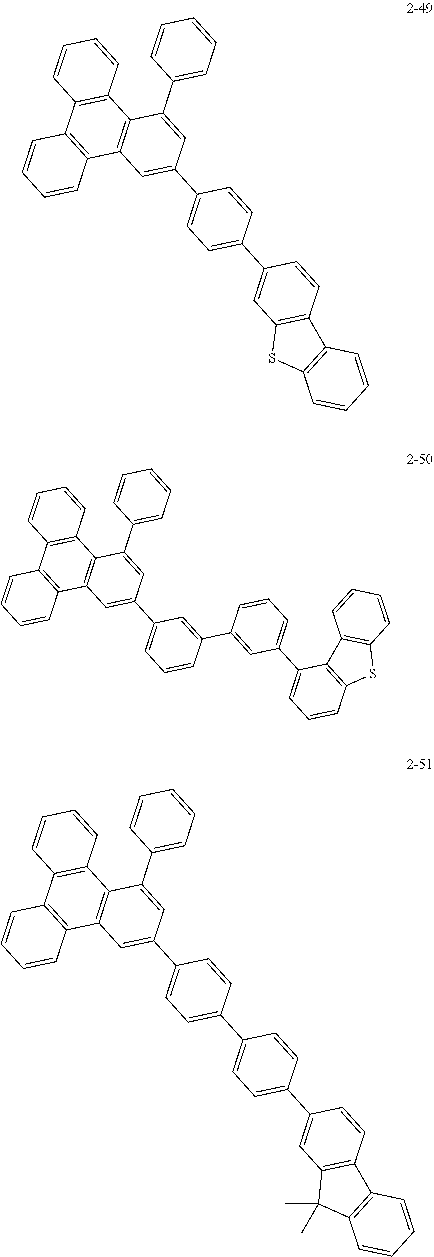

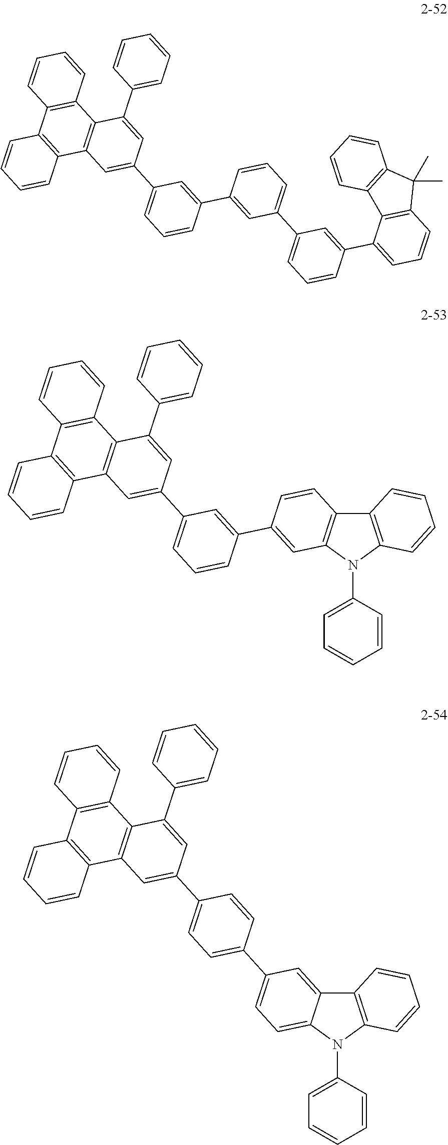

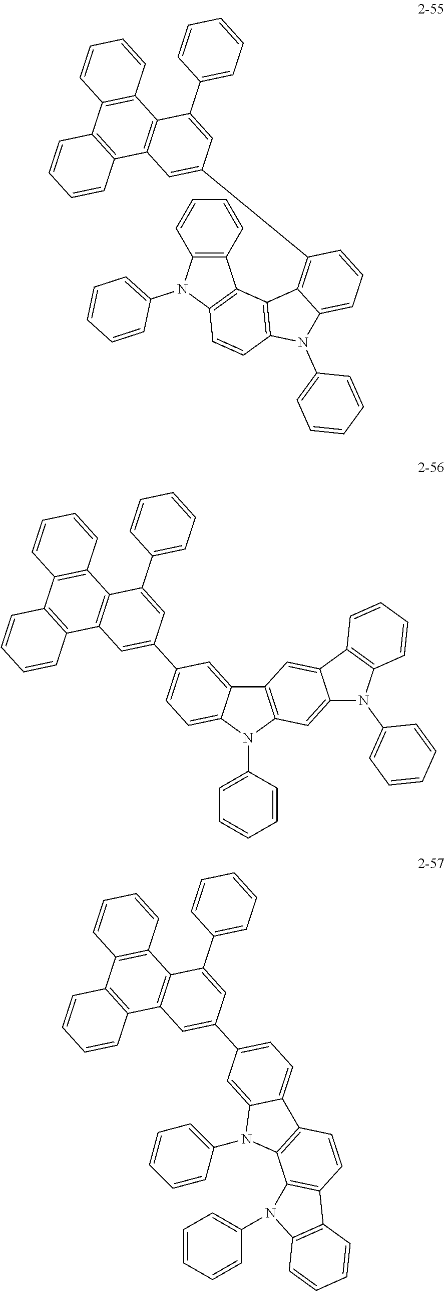

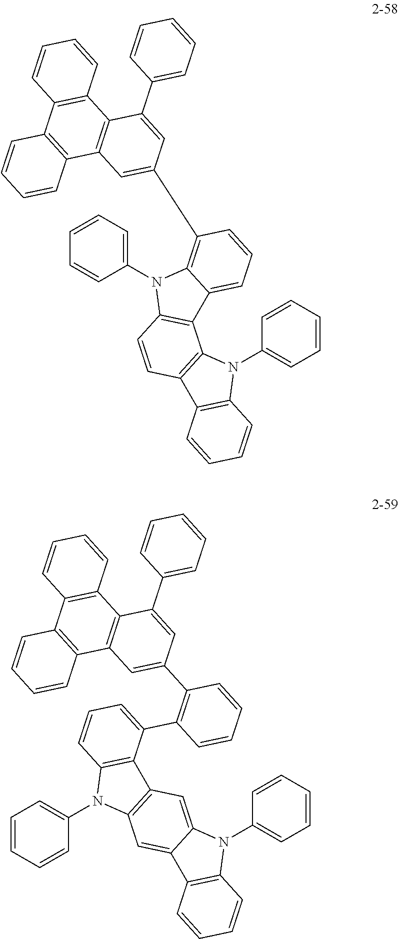 [SMILES: c1ccc(-c2cc(-c3cccc4c5c(ccc6c7ccccc7n(-c7ccccc7)c65)n(-c5ccccc5)c34)cc3c4ccccc4c4ccccc4c23)cc1 ; c1ccc(-c2cc(-c3ccccc3-c3cccc4c3c3cc5c(cc3n4-c3ccccc3)c3ccccc3n5-c3ccccc3)cc3c4ccccc4c4ccccc4c23)cc1]

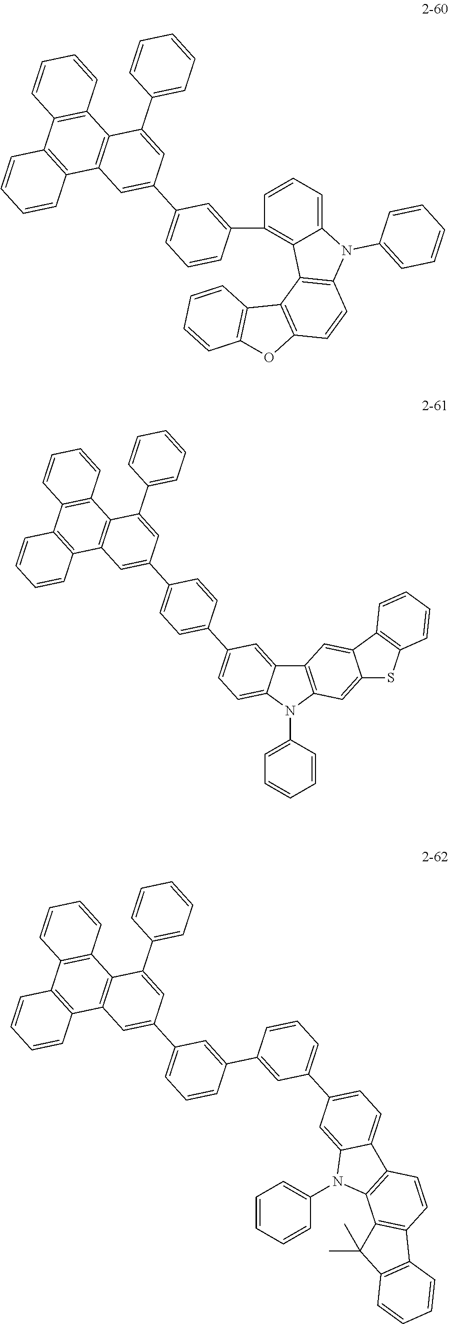 [SMILES: c1ccc(-c2cc(-c3cccc(-c4cccc5c4c4c6c(ccc4n5-c4ccccc4)oc4ccccc46)c3)cc3c4ccccc4c4ccccc4c23)cc1 ; c1ccc(-c2cc(-c3ccc(-c4ccc5c(c4)c4cc6c(cc4n5-c4ccccc4)sc4ccccc46)cc3)cc3c4ccccc4c4ccccc4c23)cc1 ; CC1(C)c2ccccc2-c2ccc3c4ccc(-c5cccc(-c6cccc(-c7cc(-c8ccccc8)c8c9ccccc9c9ccccc9c8c7)c6)c5)cc4n(-c4ccccc4)c3c21]

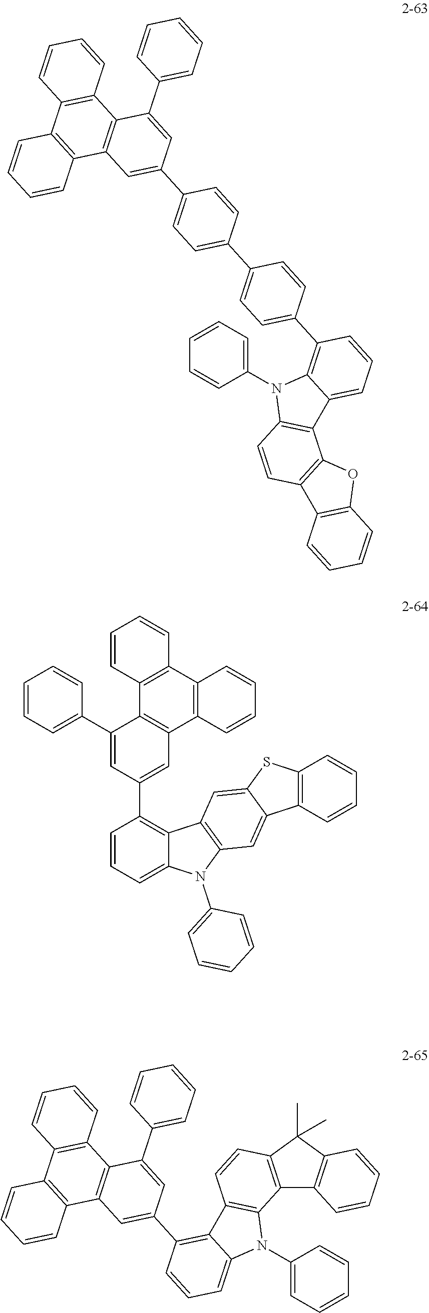 [SMILES: c1ccc(-c2cc(-c3ccc(-c4ccc(-c5cccc6c7c8oc9ccccc9c8ccc7n(-c7ccccc7)c56)cc4)cc3)cc3c4ccccc4c4ccccc4c23)cc1 ; c1ccc(-c2cc(-c3cccc4c3c3cc5sc6ccccc6c5cc3n4-c3ccccc3)cc3c4ccccc4c4ccccc4c23)cc1 ; CC1(C)c2ccccc2-c2c1ccc1c3c(-c4cc(-c5ccccc5)c5c6ccccc6c6ccccc6c5c4)cccc3n(-c3ccccc3)c21]

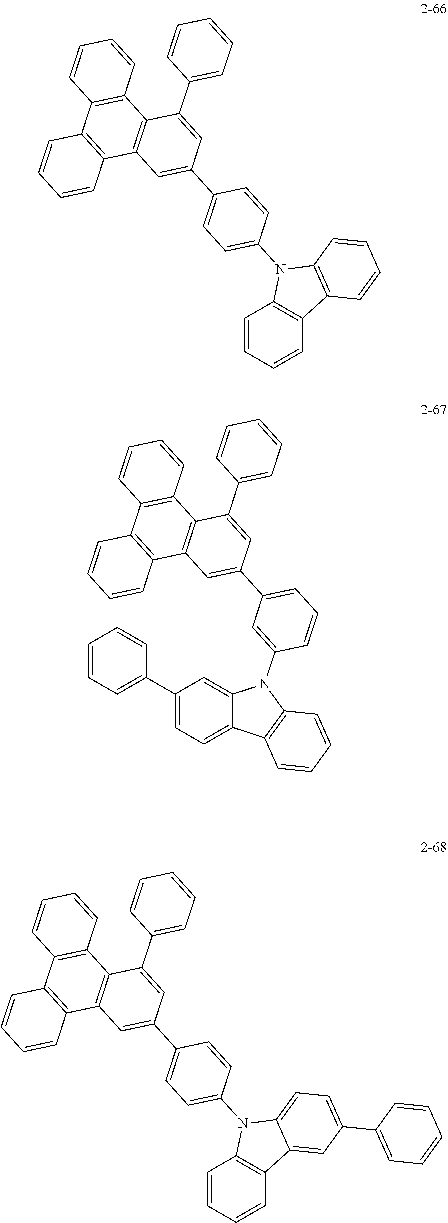 [SMILES: c1ccc(-c2cc(-c3ccc(-n4c5ccccc5c5ccccc54)cc3)cc3c4ccccc4c4ccccc4c23)cc1 ; c1ccc(-c2ccc3c4ccccc4n(-c4cccc(-c5cc(-c6ccccc6)c6c7ccccc7c7ccccc7c6c5)c4)c3c2)cc1 ; c1ccc(-c2ccc3c(c2)c2ccccc2n3-c2ccc(-c3cc(-c4ccccc4)c4c5ccccc5c5ccccc5c4c3)cc2)cc1]

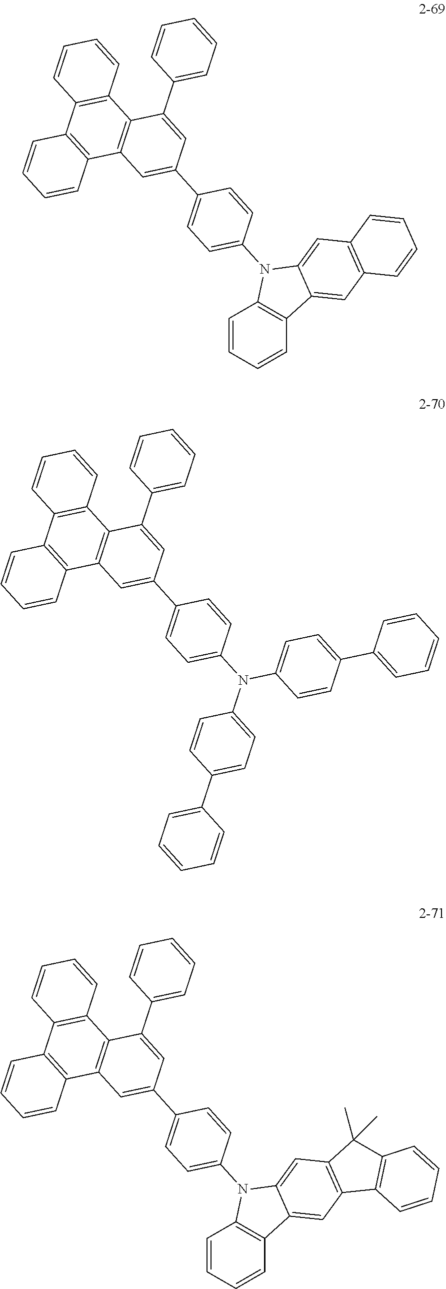 [SMILES: c1ccc(-c2cc(-c3ccc(-n4c5ccccc5c5cc6ccccc6cc54)cc3)cc3c4ccccc4c4ccccc4c23)cc1 ; c1ccc(-c2ccc(N(c3ccc(-c4ccccc4)cc3)c3ccc(-c4cc(-c5ccccc5)c5c6ccccc6c6ccccc6c5c4)cc3)cc2)cc1 ; CC1(C)c2ccccc2-c2cc3c4ccccc4n(-c4ccc(-c5cc(-c6ccccc6)c6c7ccccc7c7ccccc7c6c5)cc4)c3cc21]

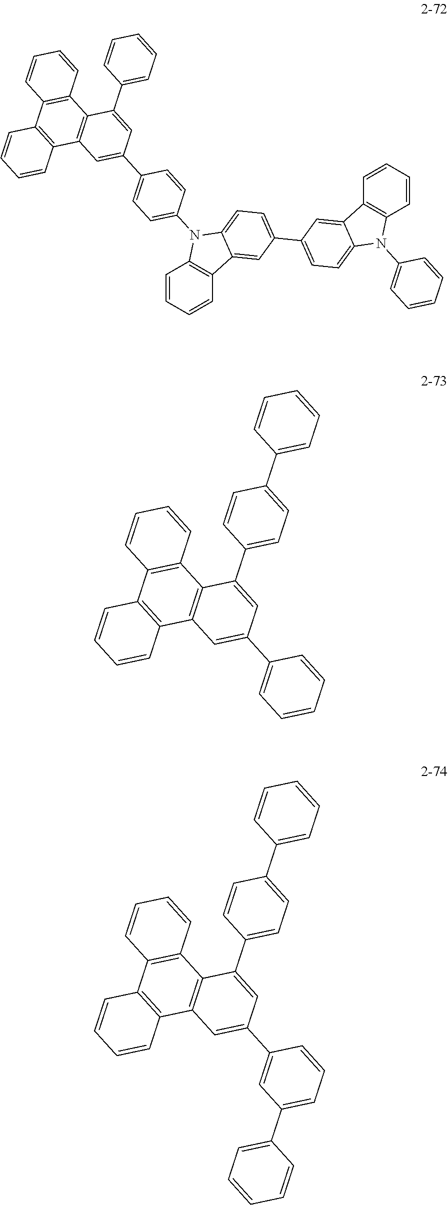

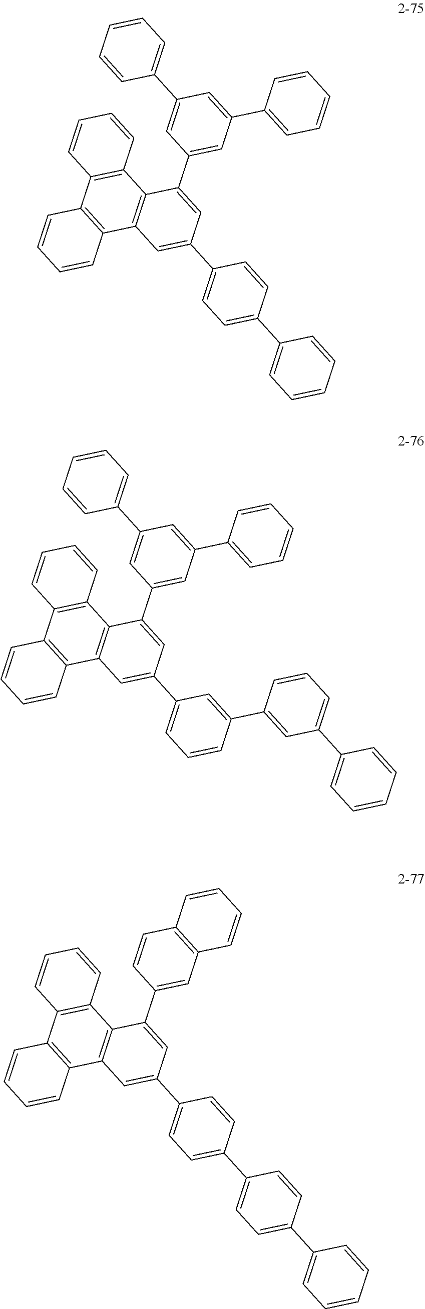

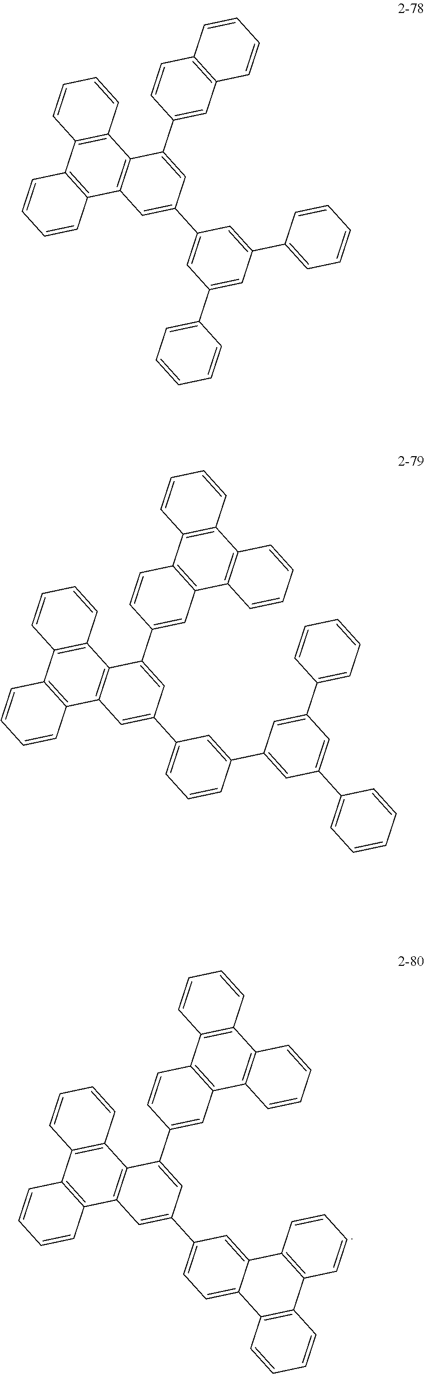

6. An organic light emitting device comprising:
a first electrode;
a second electrode; and
an organic material layer provided between the first electrode and the second electrode,
wherein the organic material layer includes one or more types of the compound of claim 1.

7. The organic light emitting device of claim 6, wherein the organic material layer includes two types of the compound.

8. The organic light emitting device of claim 6, wherein the organic material layer further includes a compound represented by the following Chemical Formula 2: 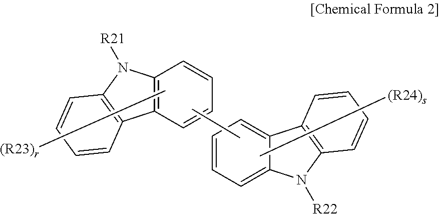
in Chemical Formula 2,
R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
R23 and R24 are each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
r and s are each an integer of 0 to 7; and
when r and s are each 2 or greater, substituents in the parentheses are the same as or different from each other.

9. The organic light emitting device of claim 8, wherein Chemical Formula 2 is represented by any one of the following compounds:

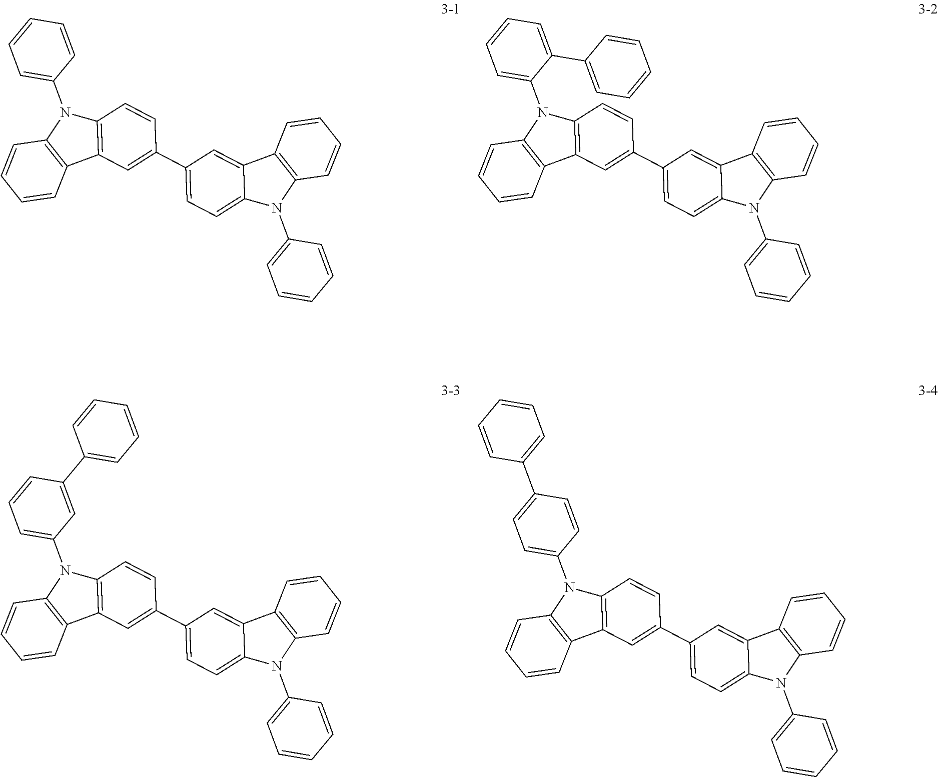 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

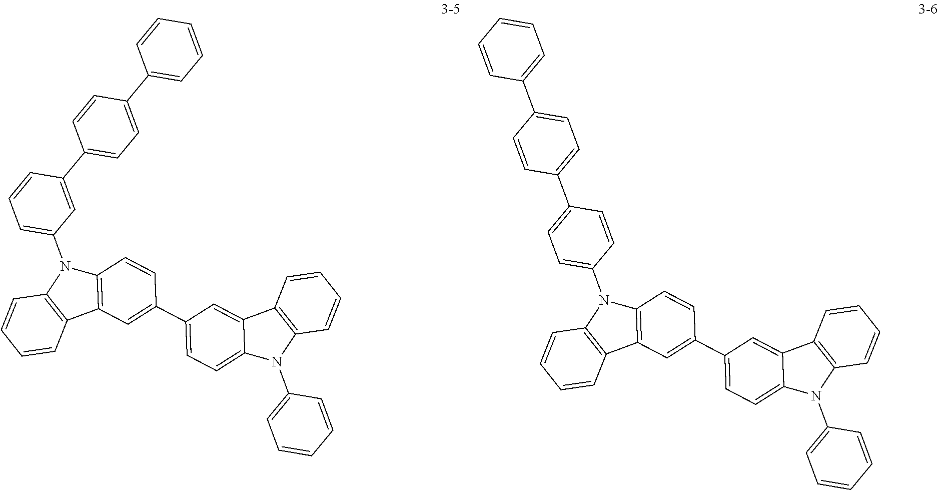 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

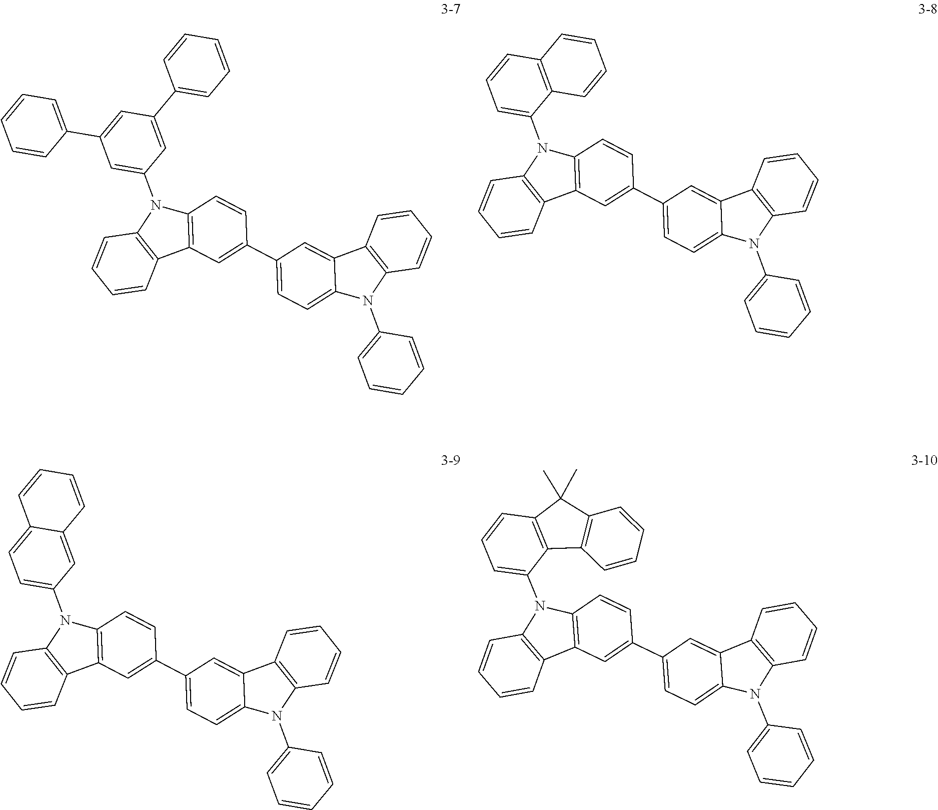 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21]

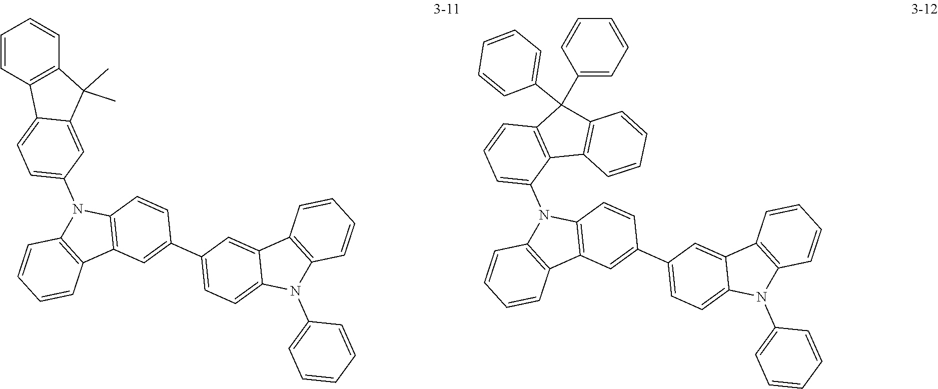 [SMILES: CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

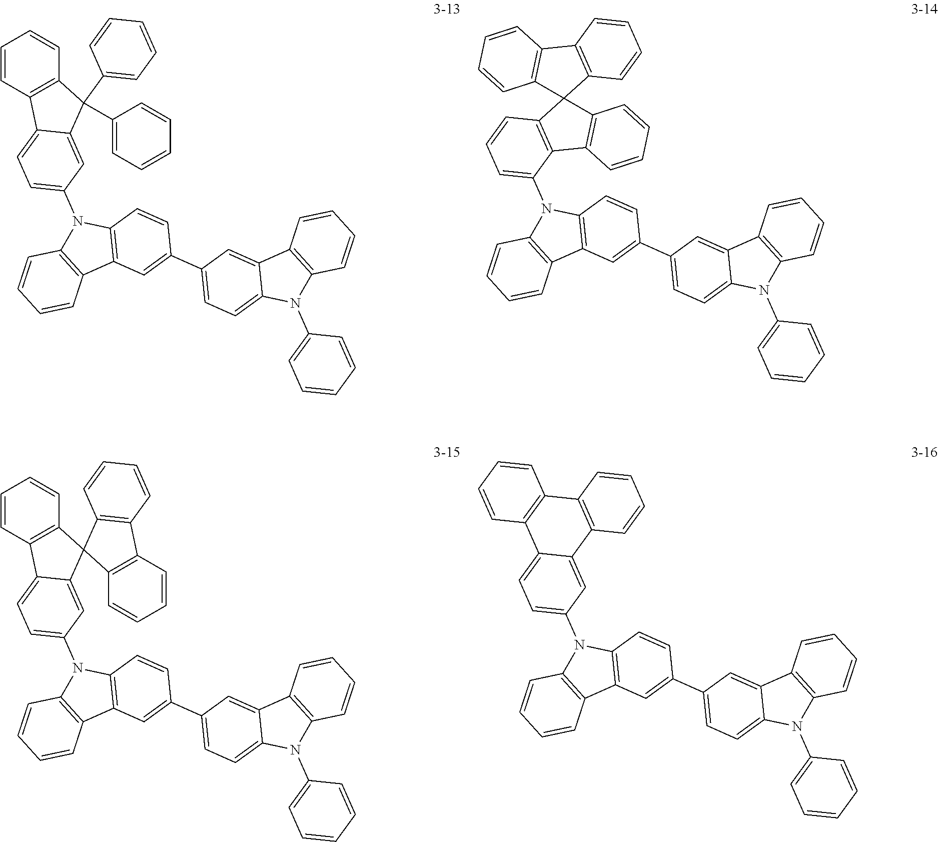 [SMILES: c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

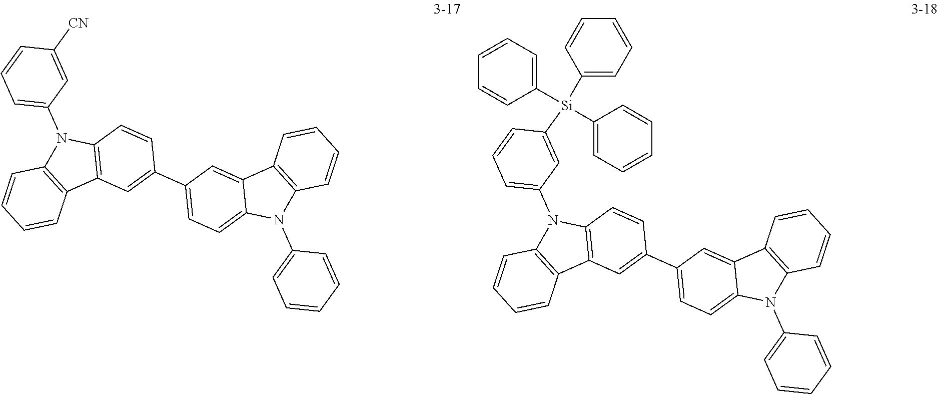 [SMILES: N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

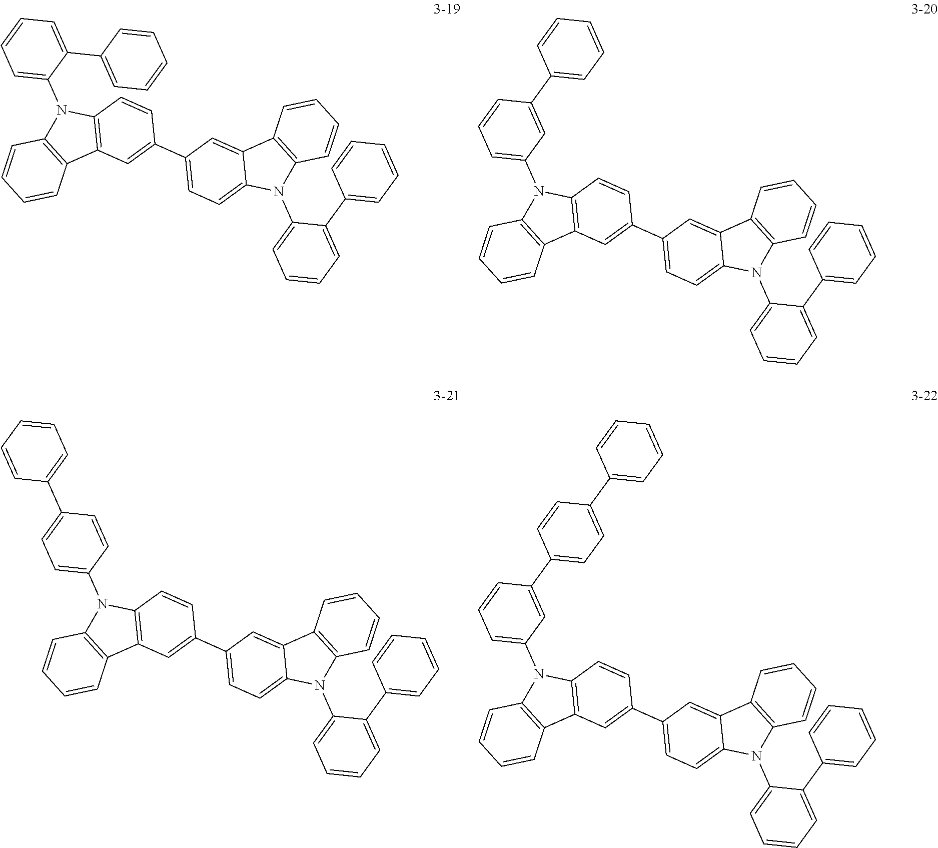 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1]

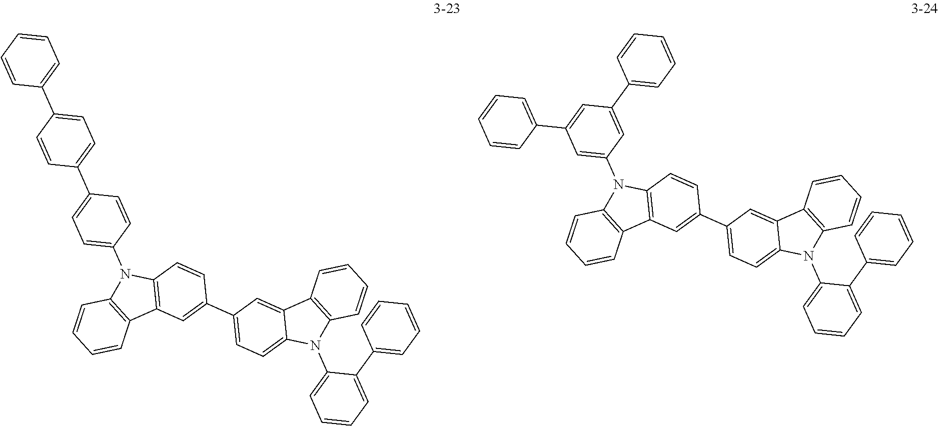 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

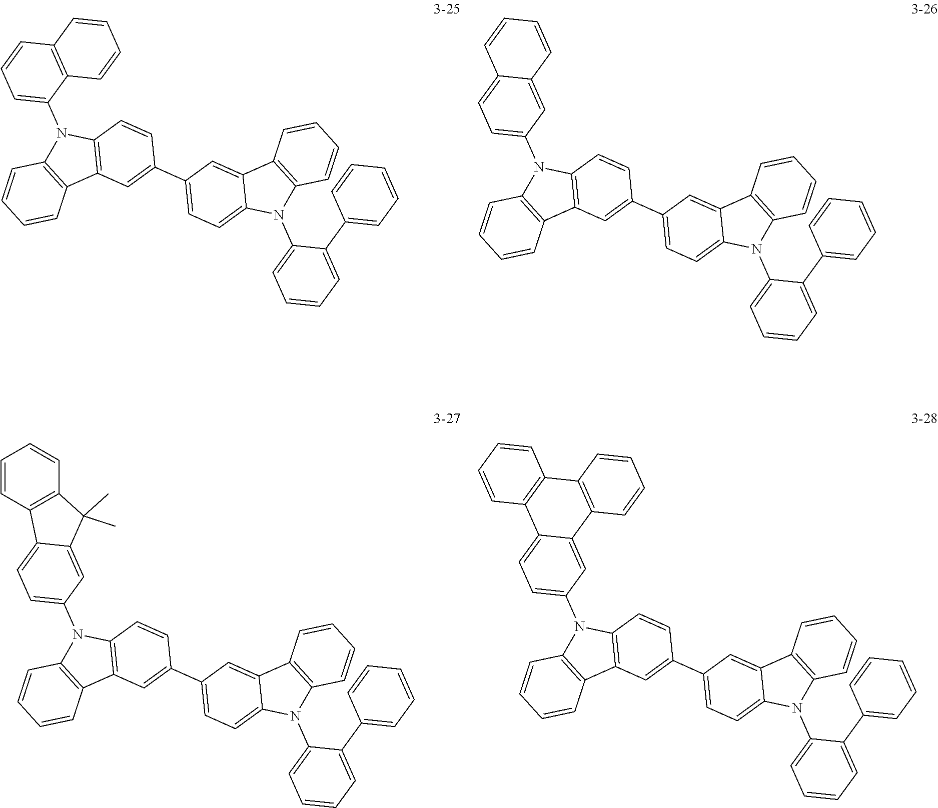 [SMILES: c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1 ; CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccccc2-n2c3ccccc3c3cc(-c4ccc5c(c4)c4ccccc4n5-c4ccc5c6ccccc6c6ccccc6c5c4)ccc32)cc1]

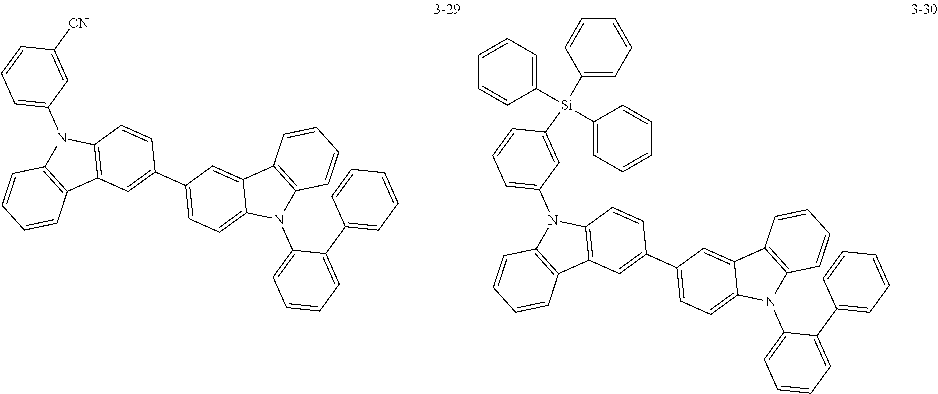 [SMILES: N#Cc1cccc(-n2c3ccccc3c3cc(-c4ccc5c(c4)c4ccccc4n5-c4ccccc4-c4ccccc4)ccc32)c1 ; c1ccc(-c2ccccc2-n2c3ccccc3c3cc(-c4ccc5c(c4)c4ccccc4n5-c4cccc([Si](c5ccccc5)(c5ccccc5)c5ccccc5)c4)ccc32)cc1]

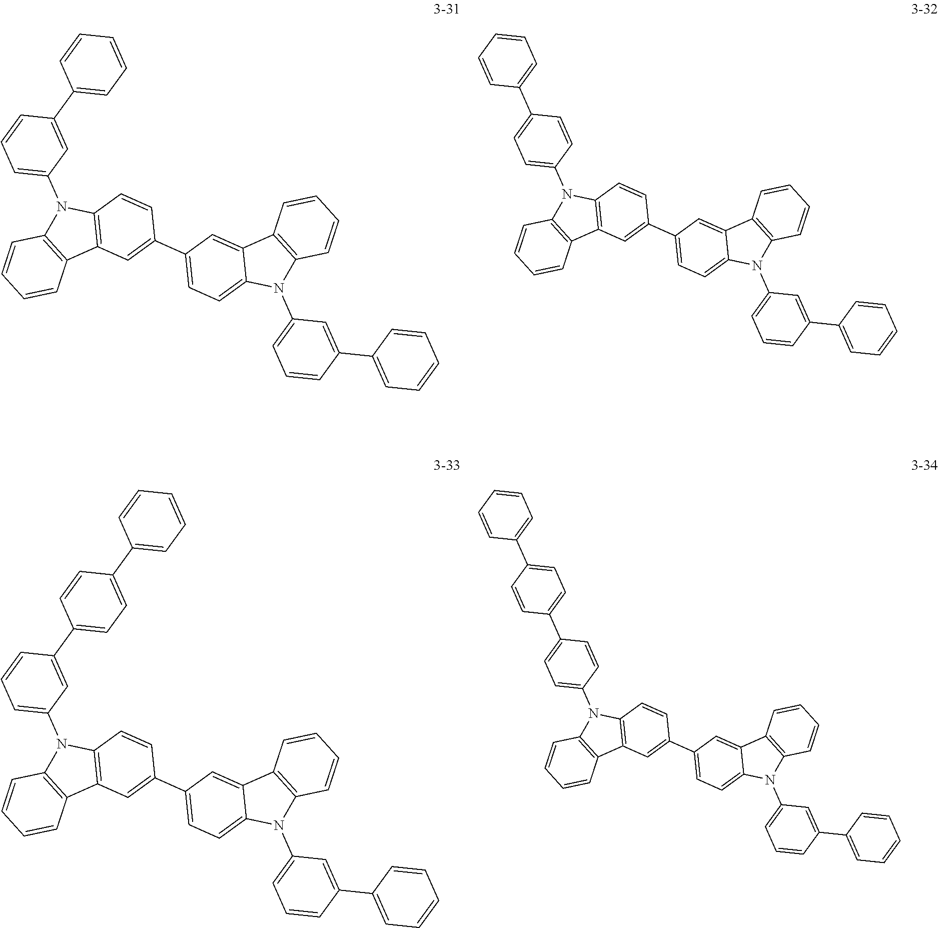 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

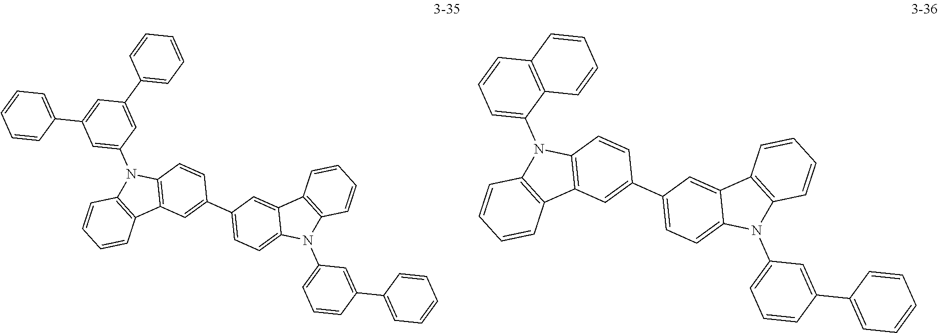 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cccc6ccccc56)ccc43)c2)cc1]

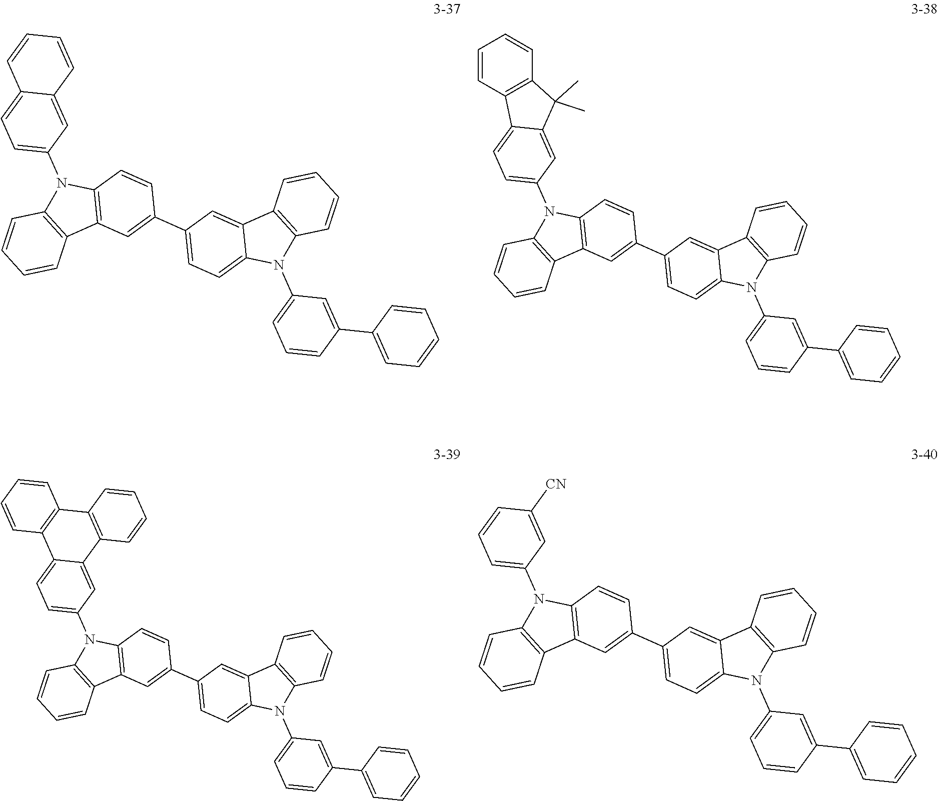 [SMILES: c1ccc(-c2cccc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cccc(-c6ccccc6)c5)ccc43)cc21 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(-c5ccccc5)c4)ccc32)c1]

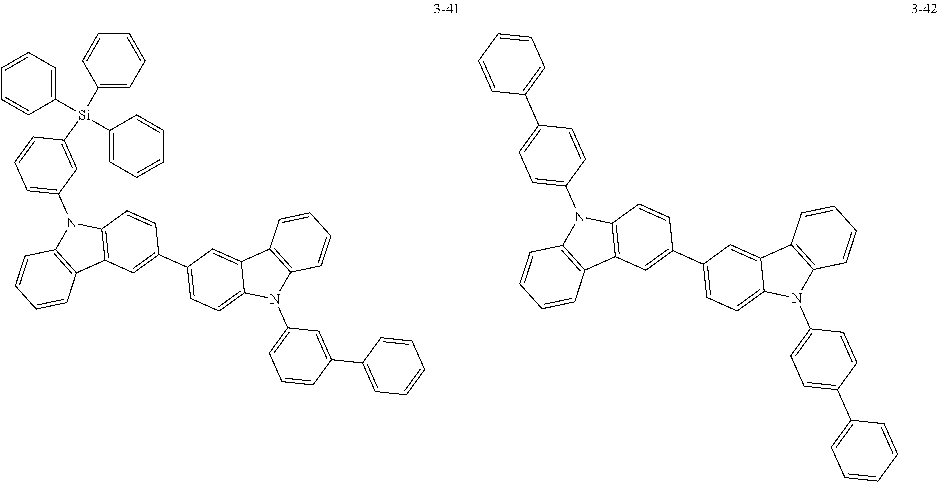 [SMILES: c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

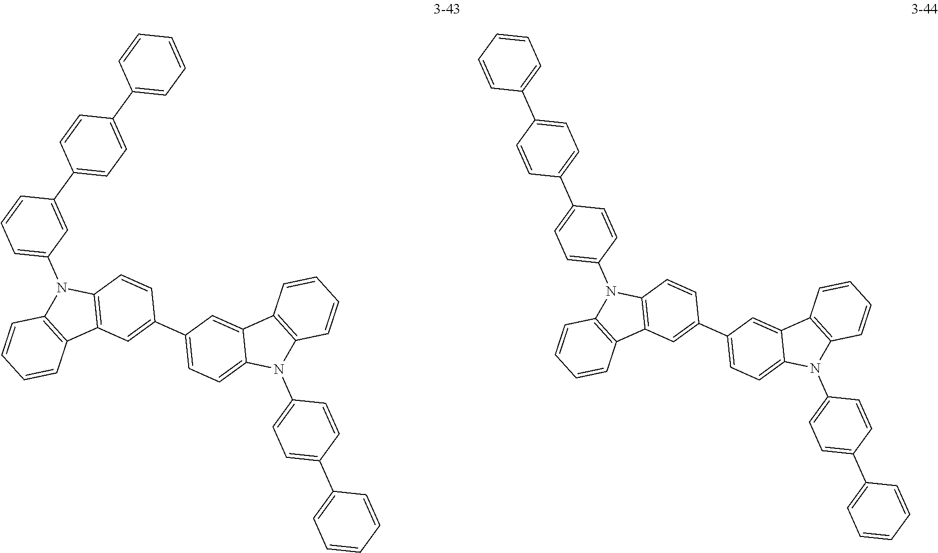 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

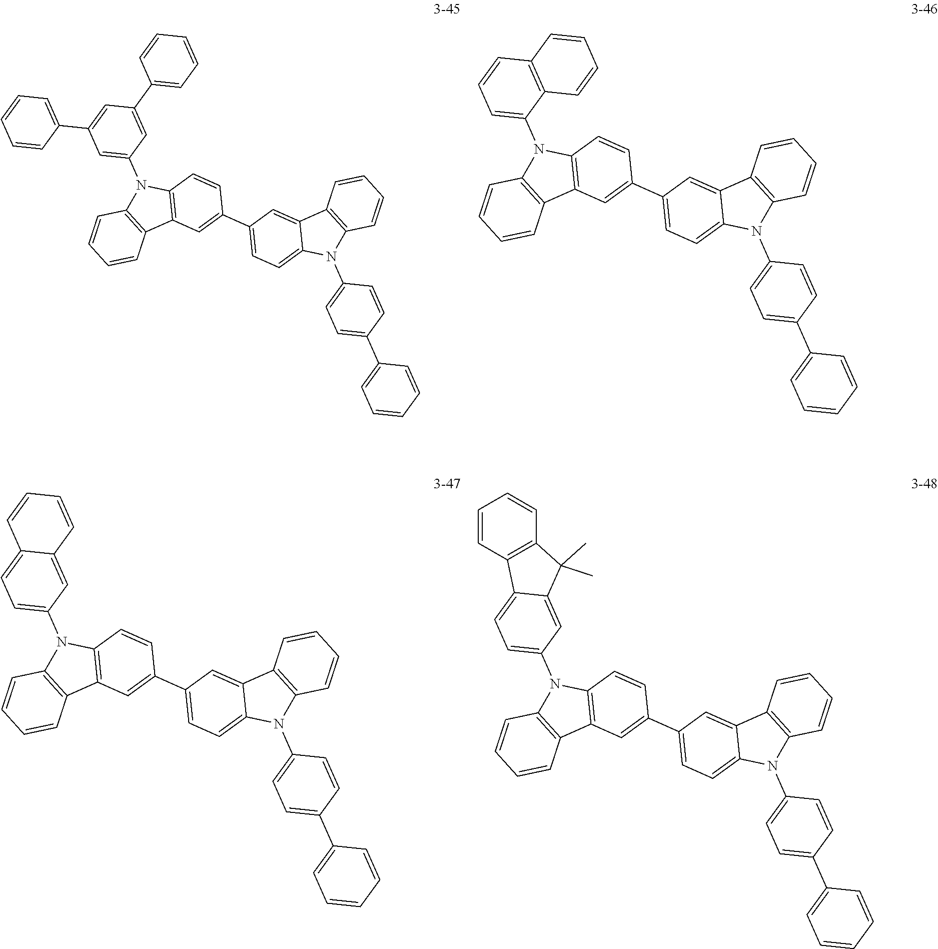 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

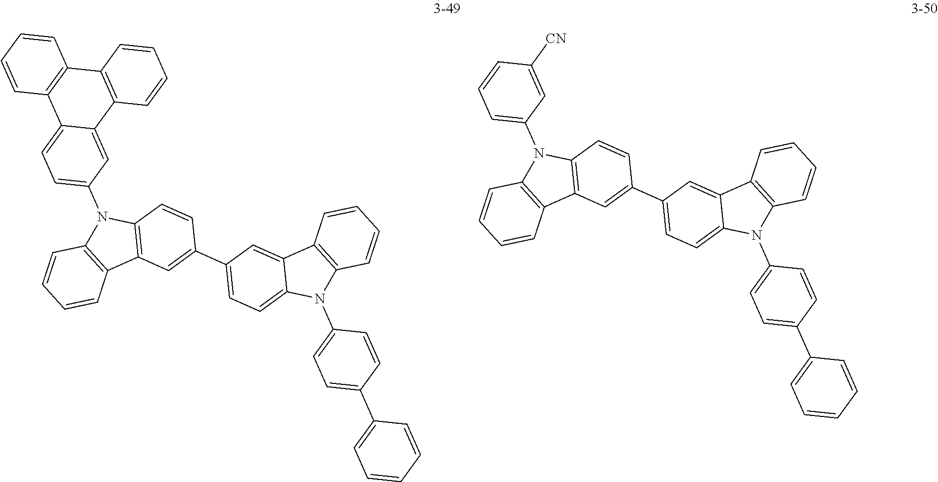 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1]

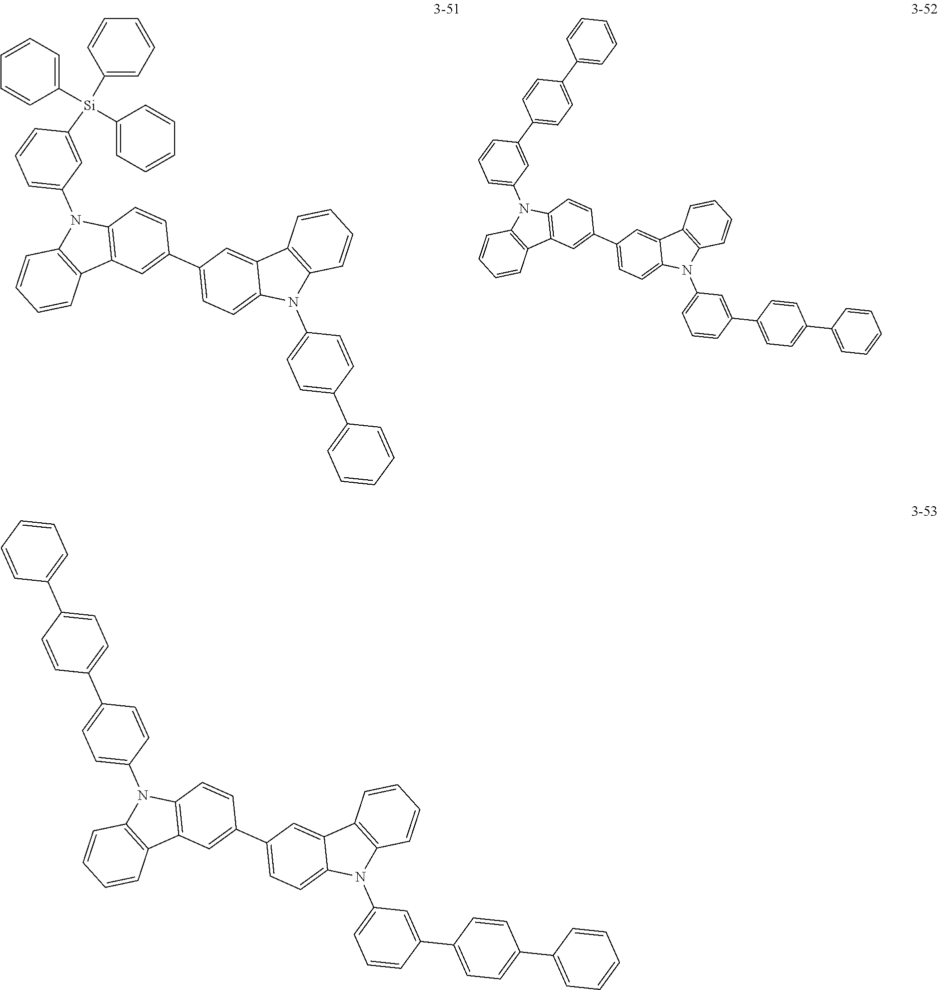 [SMILES: c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1]

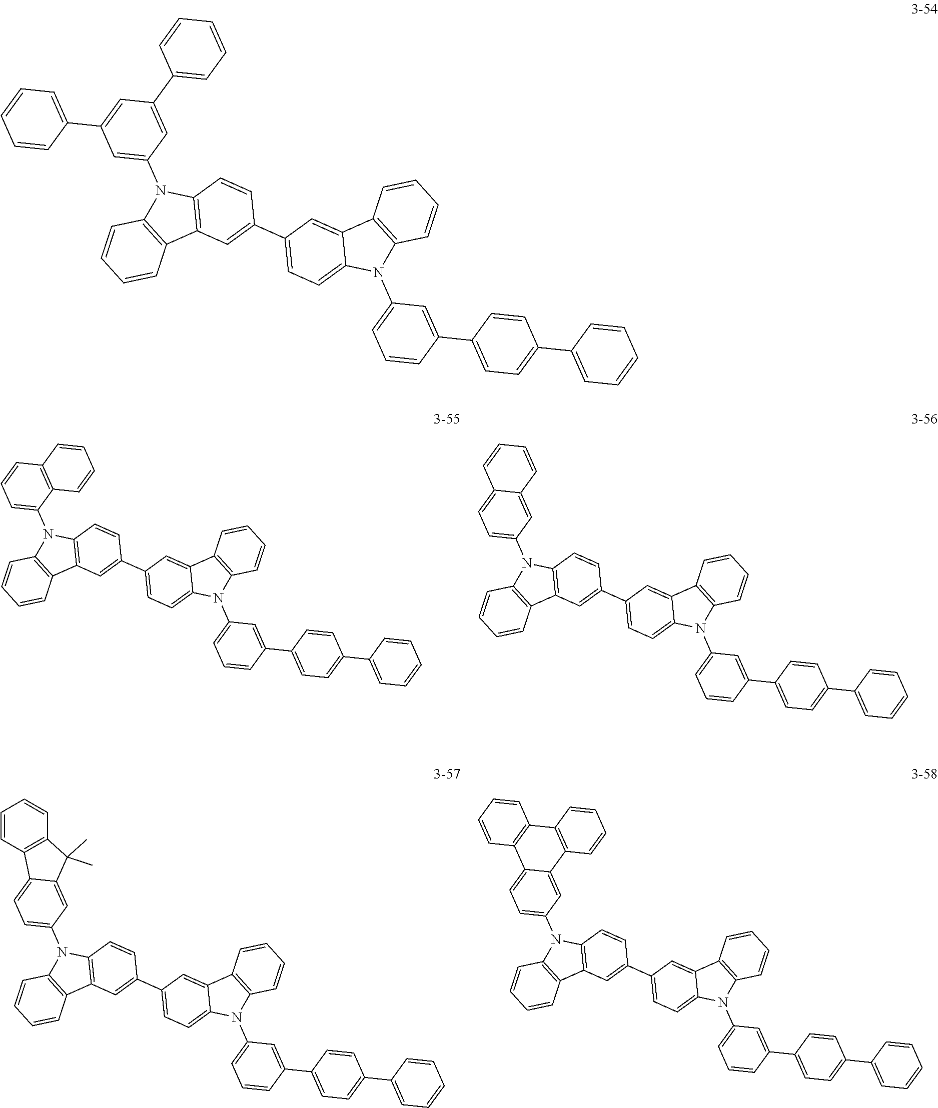

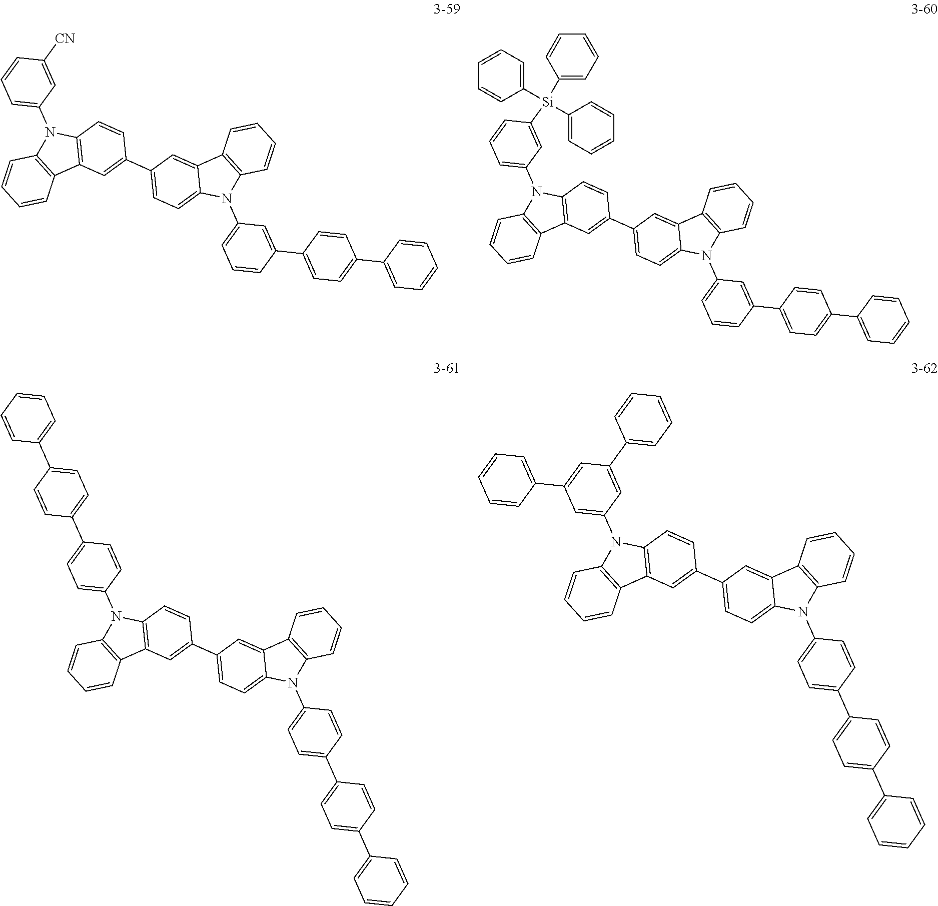 [SMILES: N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

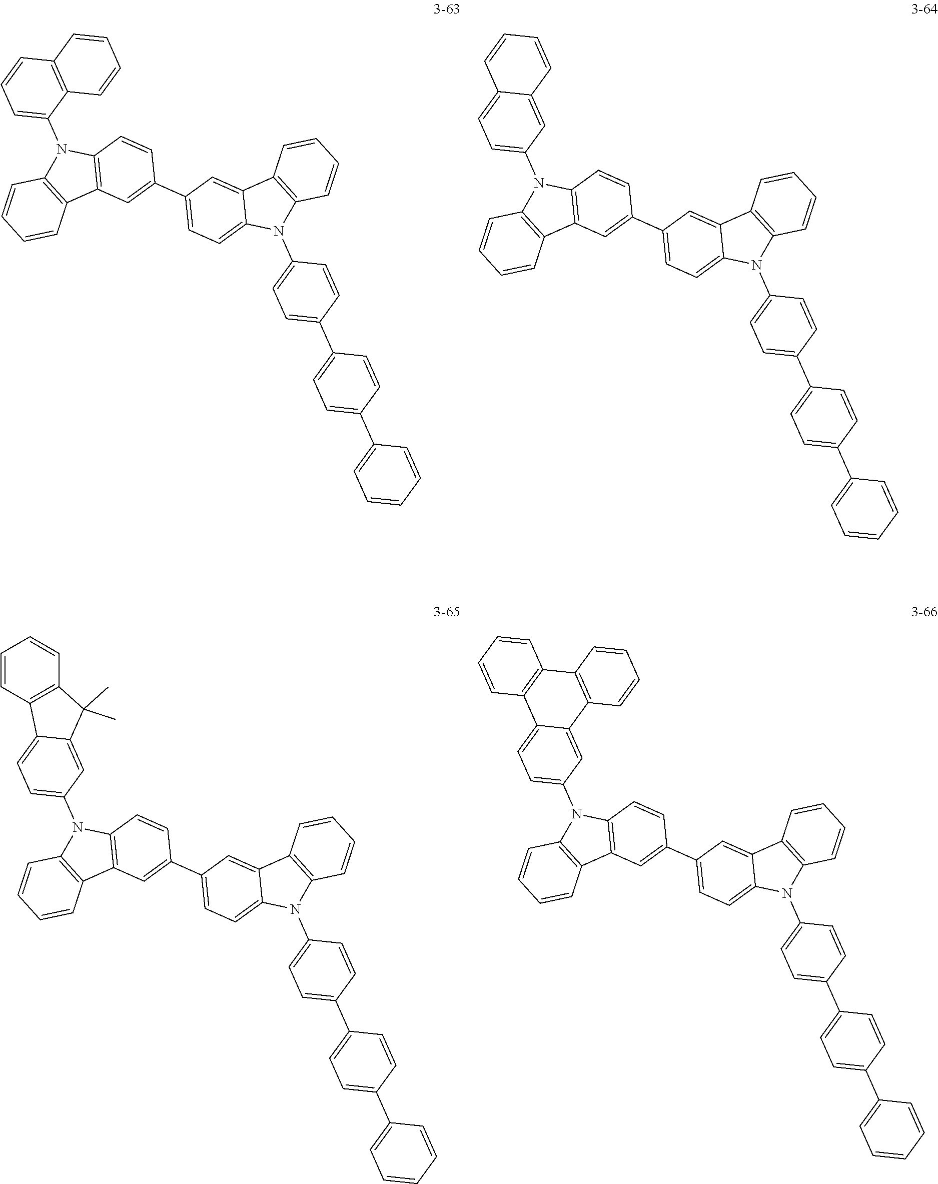 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

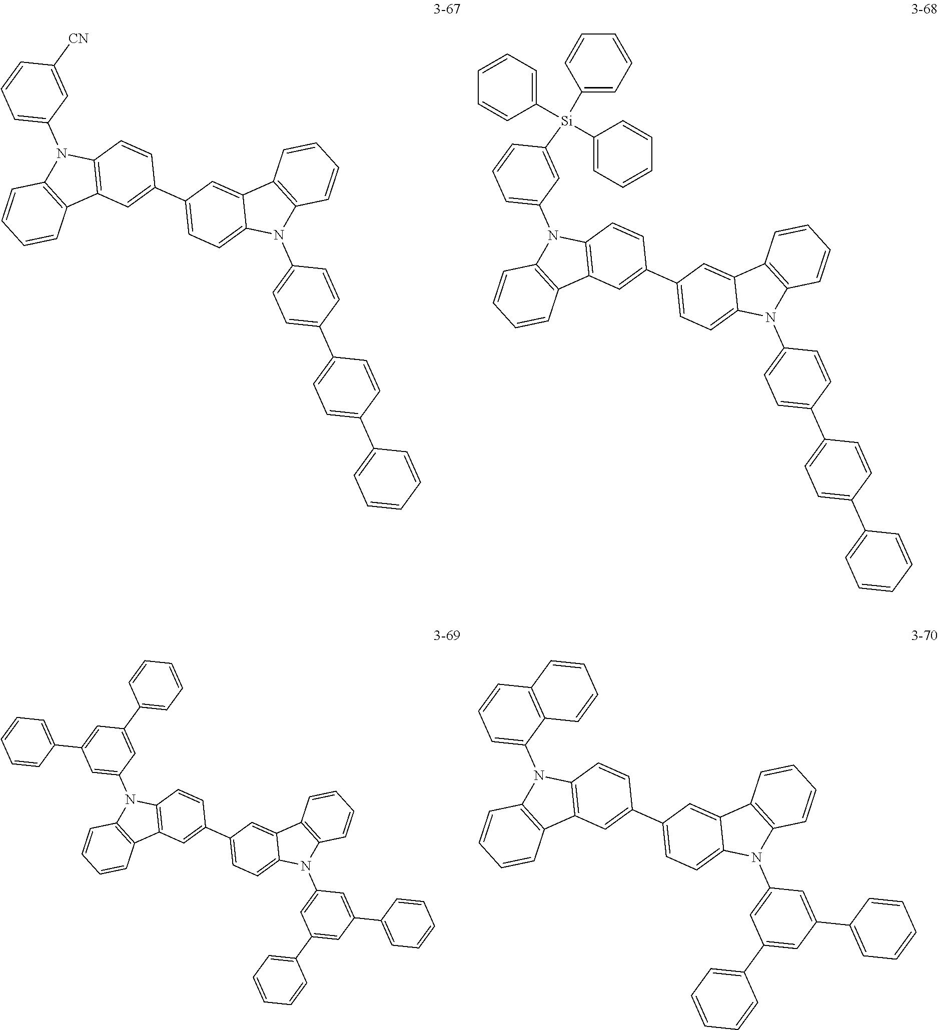 [SMILES: N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

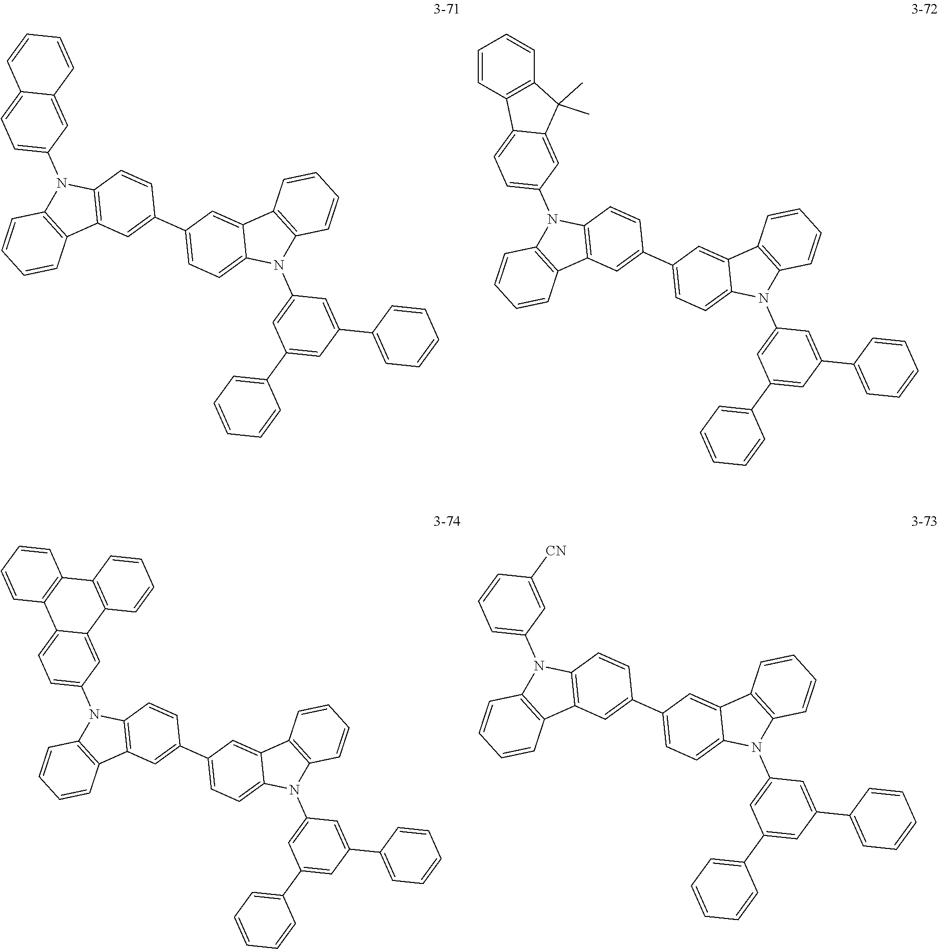 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cc(-c5ccccc5)cc(-c5ccccc5)c4)ccc32)c1]

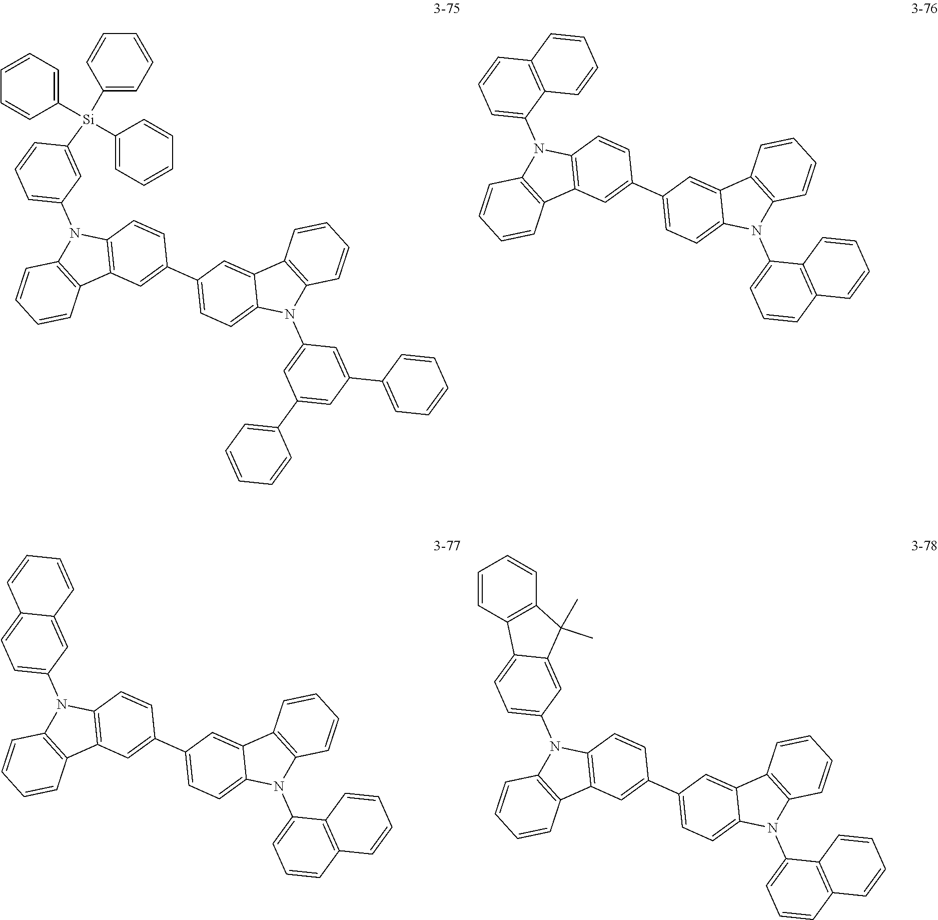 [SMILES: c1ccc(-c2cc(-c3ccccc3)cc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc2c(-n3c4ccccc4c4cc(-c5ccc6c(c5)c5ccccc5n6-c5cccc6ccccc56)ccc43)cccc2c1 ; c1ccc2cc(-n3c4ccccc4c4cc(-c5ccc6c(c5)c5ccccc5n6-c5cccc6ccccc56)ccc43)ccc2c1 ; CC1(C)c2ccccc2-c2ccc(-n3c4ccccc4c4cc(-c5ccc6c(c5)c5ccccc5n6-c5cccc6ccccc56)ccc43)cc21]

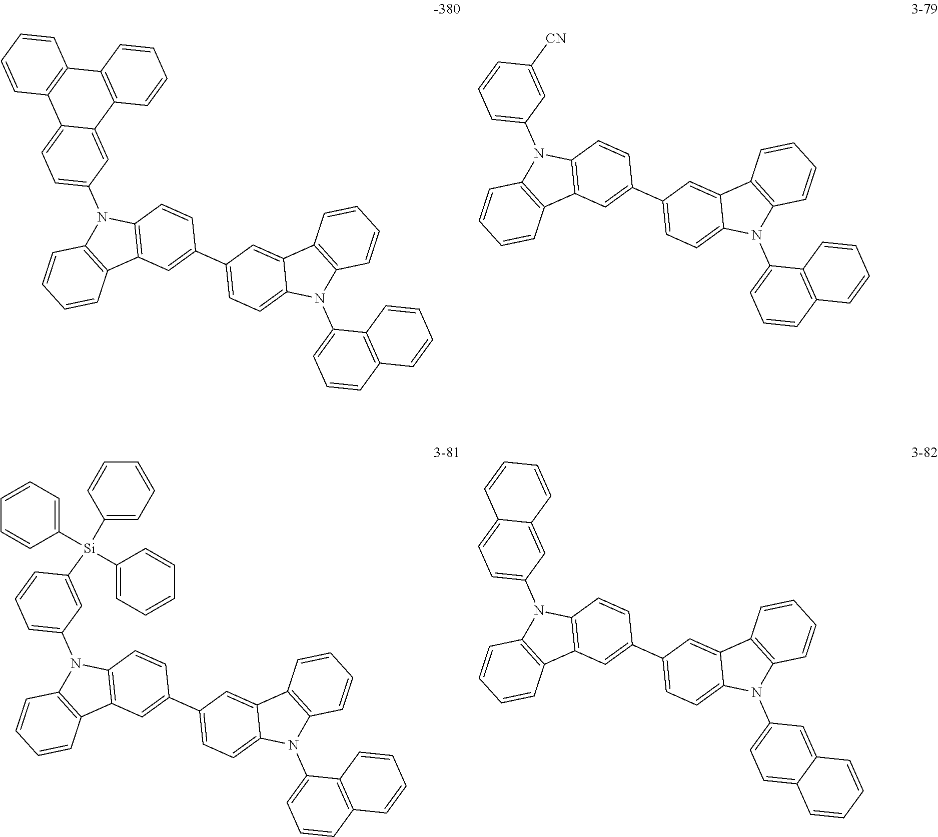 [SMILES: c1ccc2c(-n3c4ccccc4c4cc(-c5ccc6c(c5)c5ccccc5n6-c5ccc6c7ccccc7c7ccccc7c6c5)ccc43)cccc2c1 ; N#Cc1cccc(-n2c3ccccc3c3cc(-c4ccc5c(c4)c4ccccc4n5-c4cccc5ccccc45)ccc32)c1 ; c1ccc([Si](c2ccccc2)(c2ccccc2)c2cccc(-n3c4ccccc4c4cc(-c5ccc6c(c5)c5ccccc5n6-c5cccc6ccccc56)ccc43)c2)cc1 ; c1ccc2cc(-n3c4ccccc4c4cc(-c5ccc6c(c5)c5ccccc5n6-c5ccc6ccccc6c5)ccc43)ccc2c1]

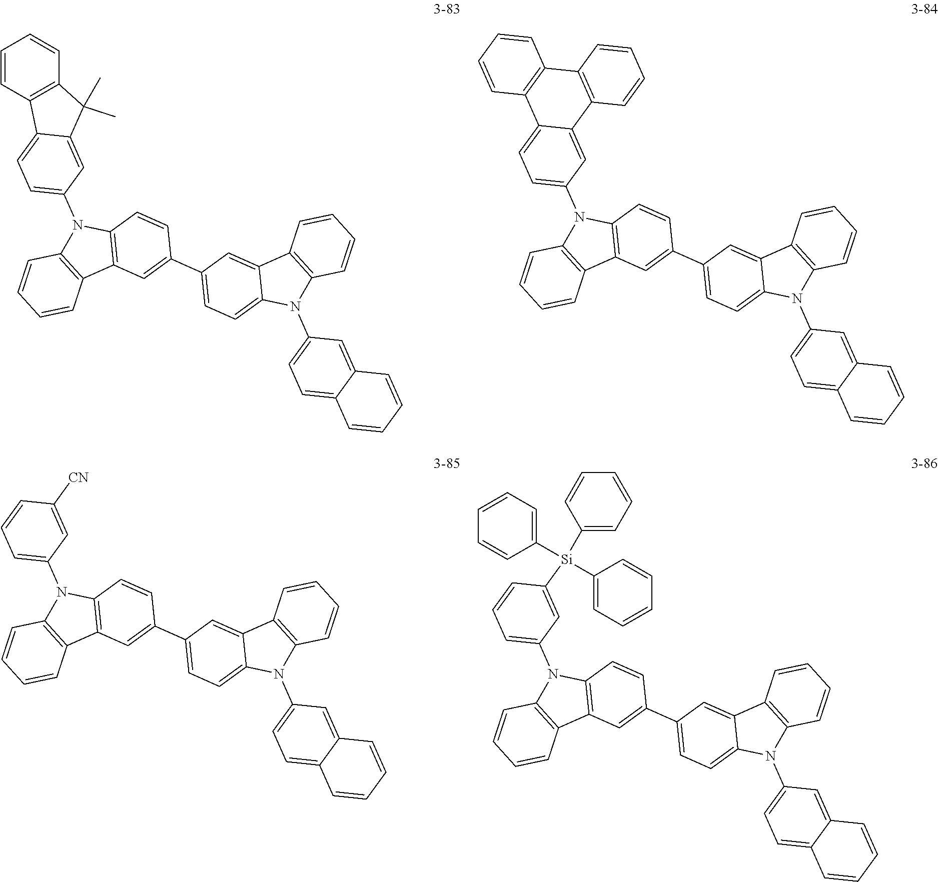 [SMILES: CC1(C)c2ccccc2-c2ccc(-n3c4ccccc4c4cc(-c5ccc6c(c5)c5ccccc5n6-c5ccc6ccccc6c5)ccc43)cc21 ; c1ccc2cc(-n3c4ccccc4c4cc(-c5ccc6c(c5)c5ccccc5n6-c5ccc6c7ccccc7c7ccccc7c6c5)ccc43)ccc2c1 ; N#Cc1cccc(-n2c3ccccc3c3cc(-c4ccc5c(c4)c4ccccc4n5-c4ccc5ccccc5c4)ccc32)c1 ; c1ccc([Si](c2ccccc2)(c2ccccc2)c2cccc(-n3c4ccccc4c4cc(-c5ccc6c(c5)c5ccccc5n6-c5ccc6ccccc6c5)ccc43)c2)cc1]

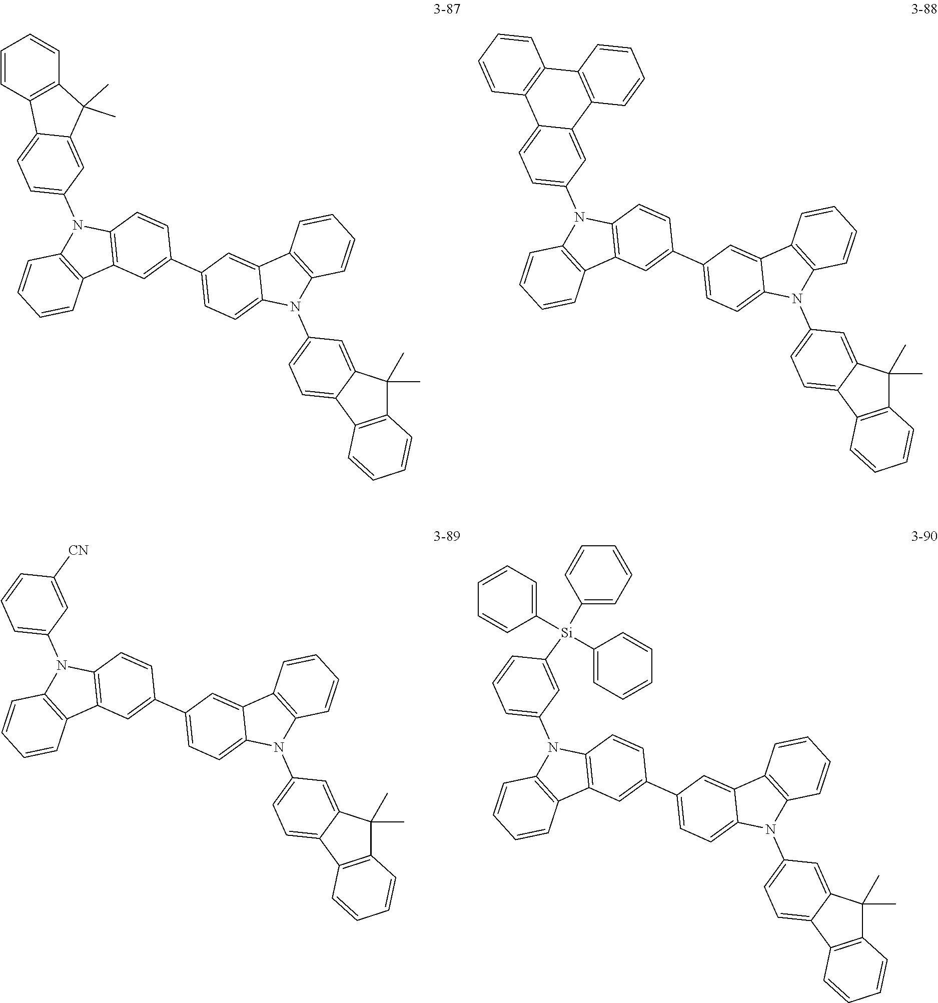 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

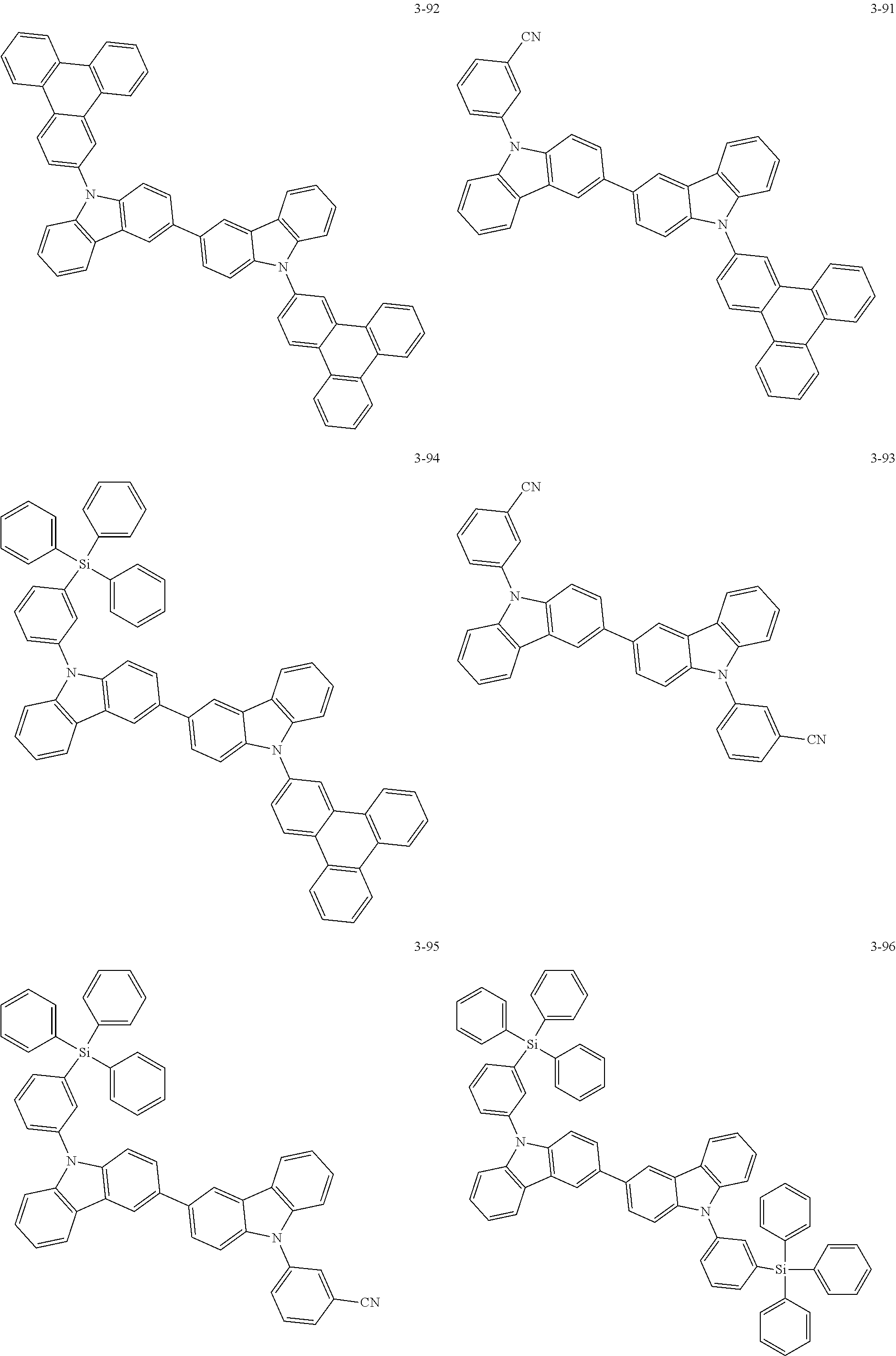

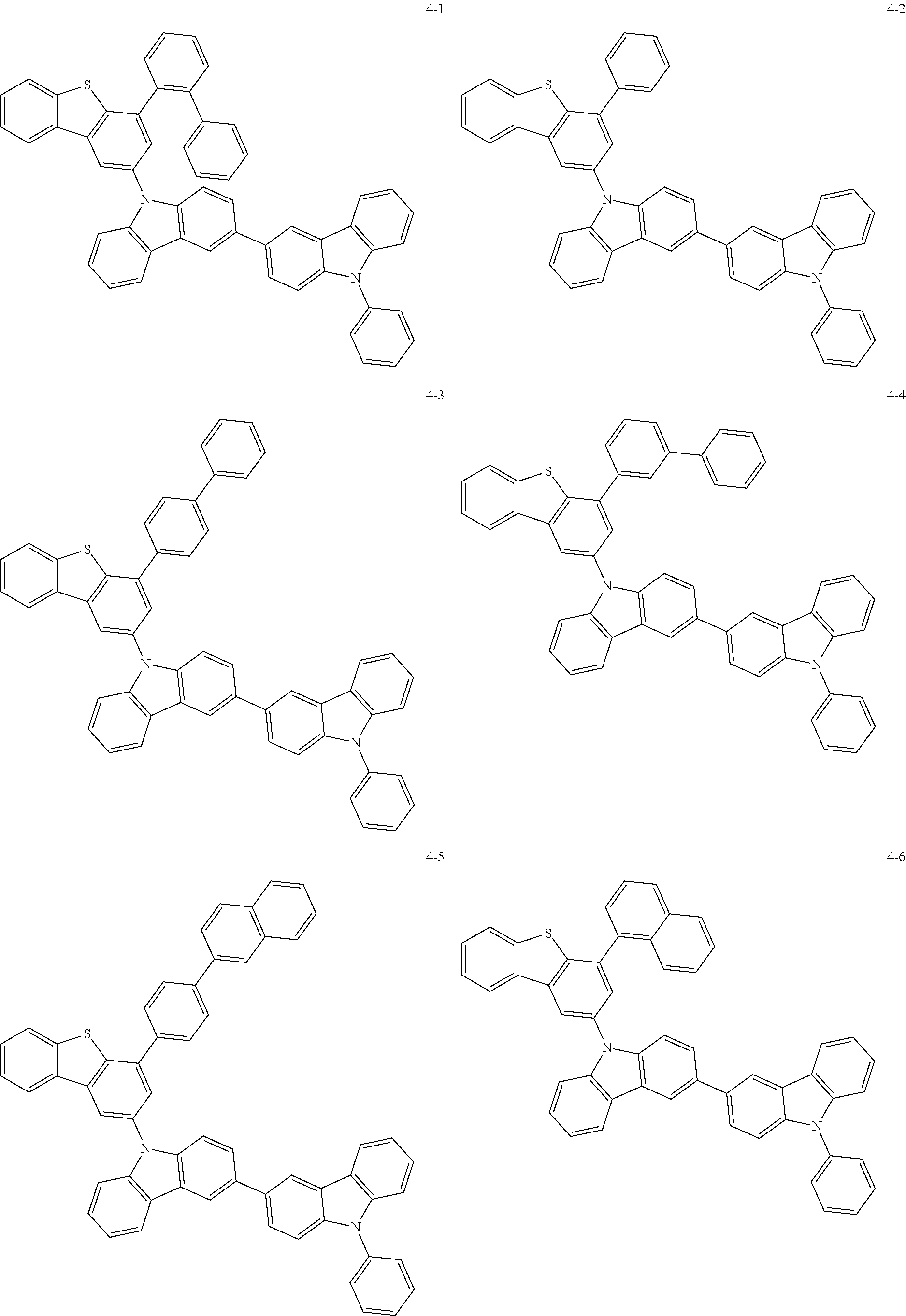

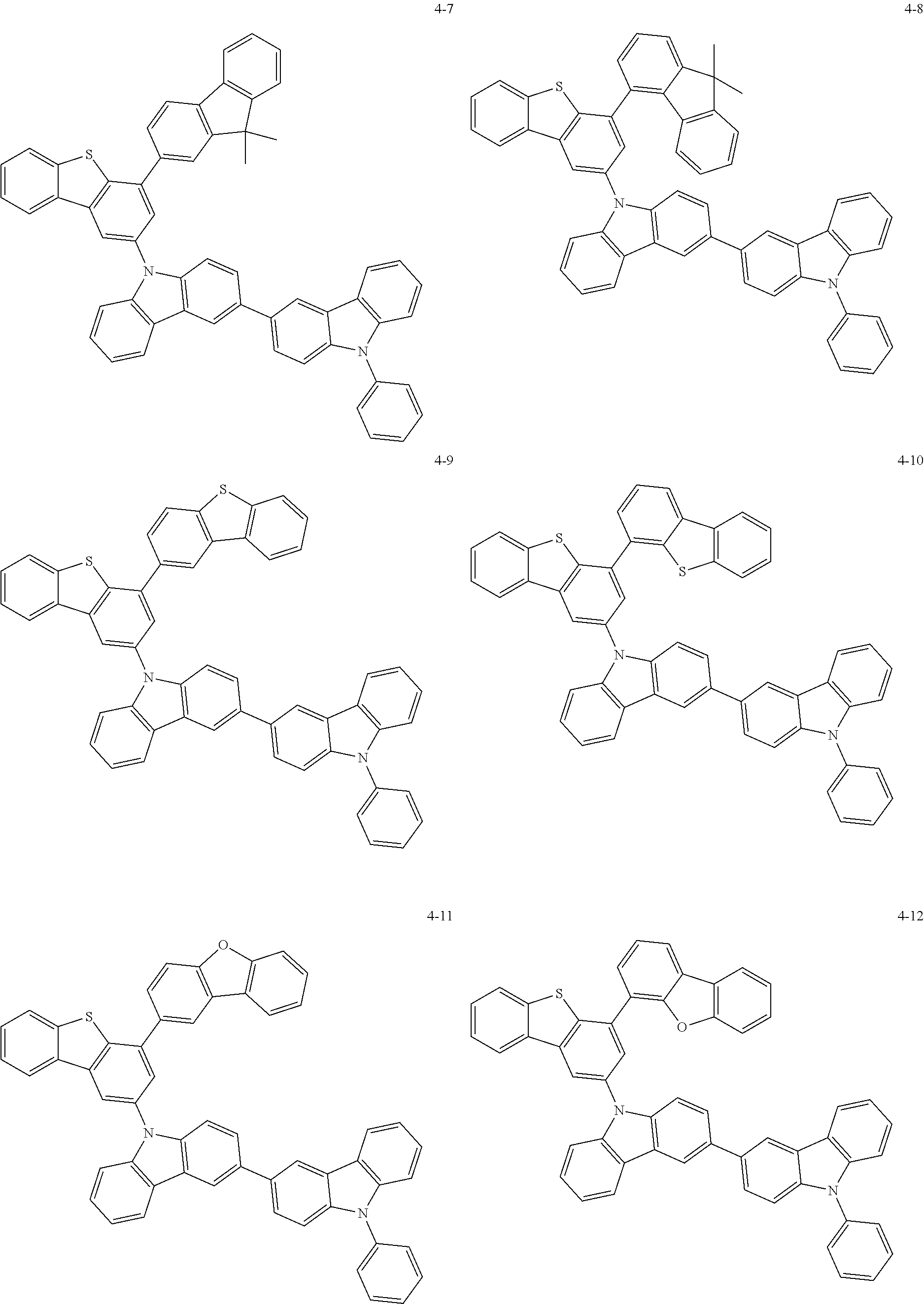

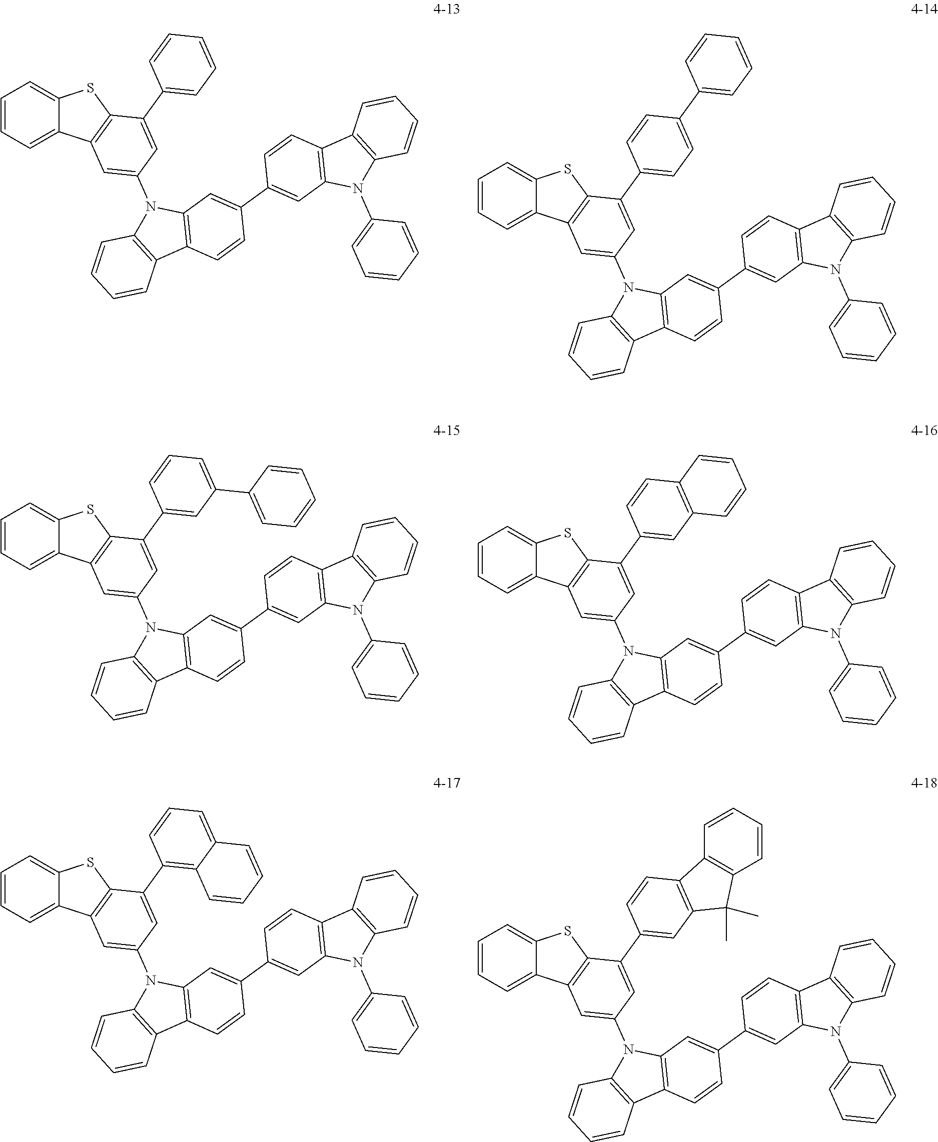

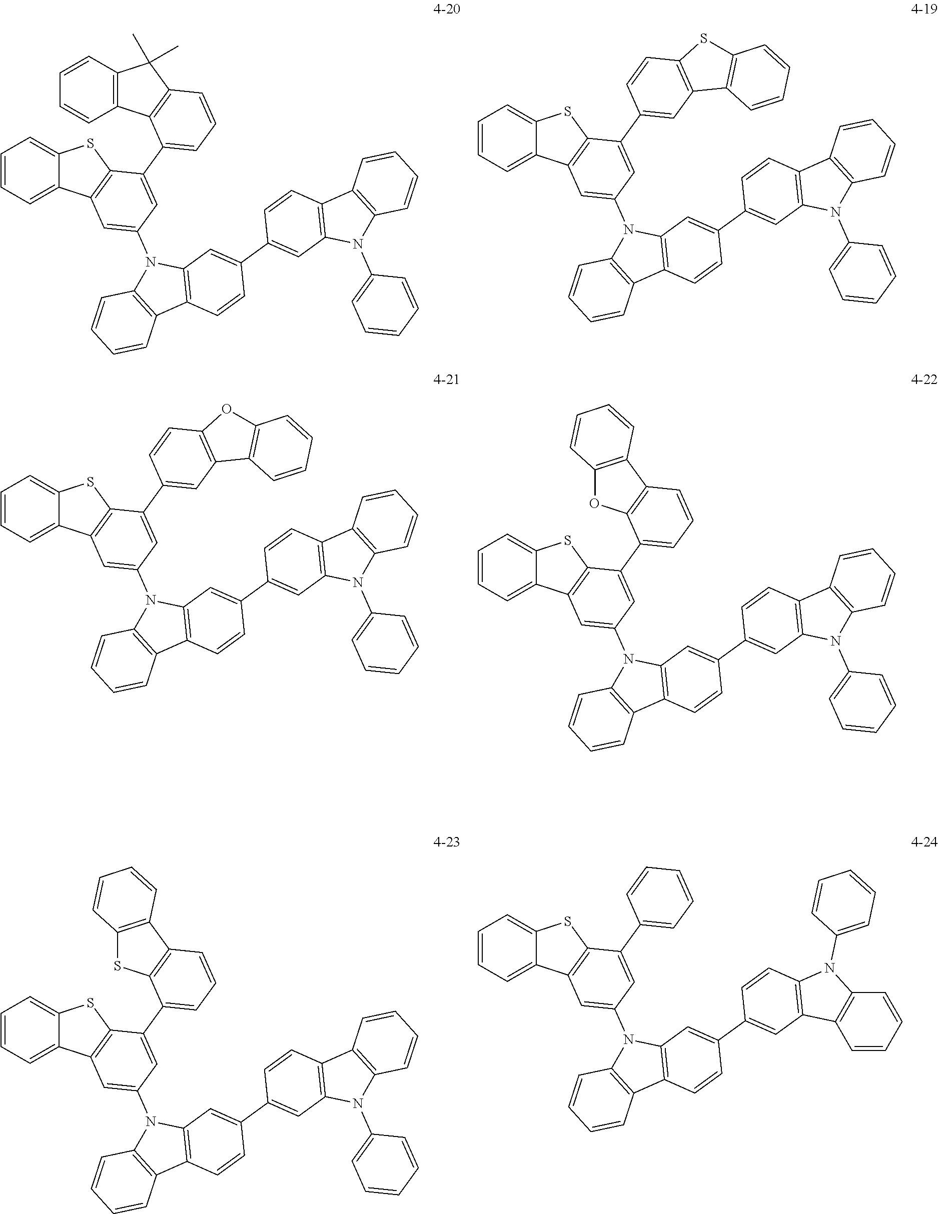

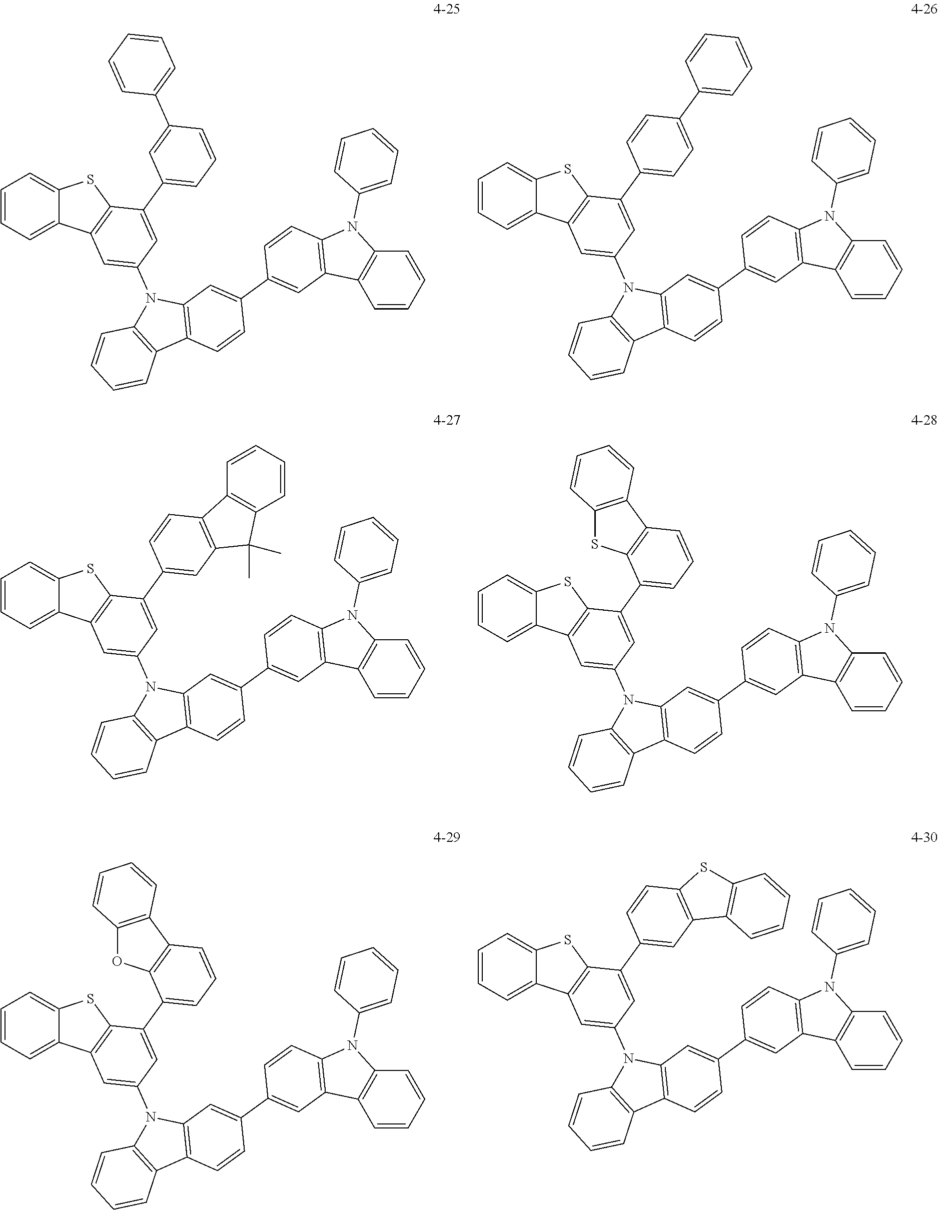

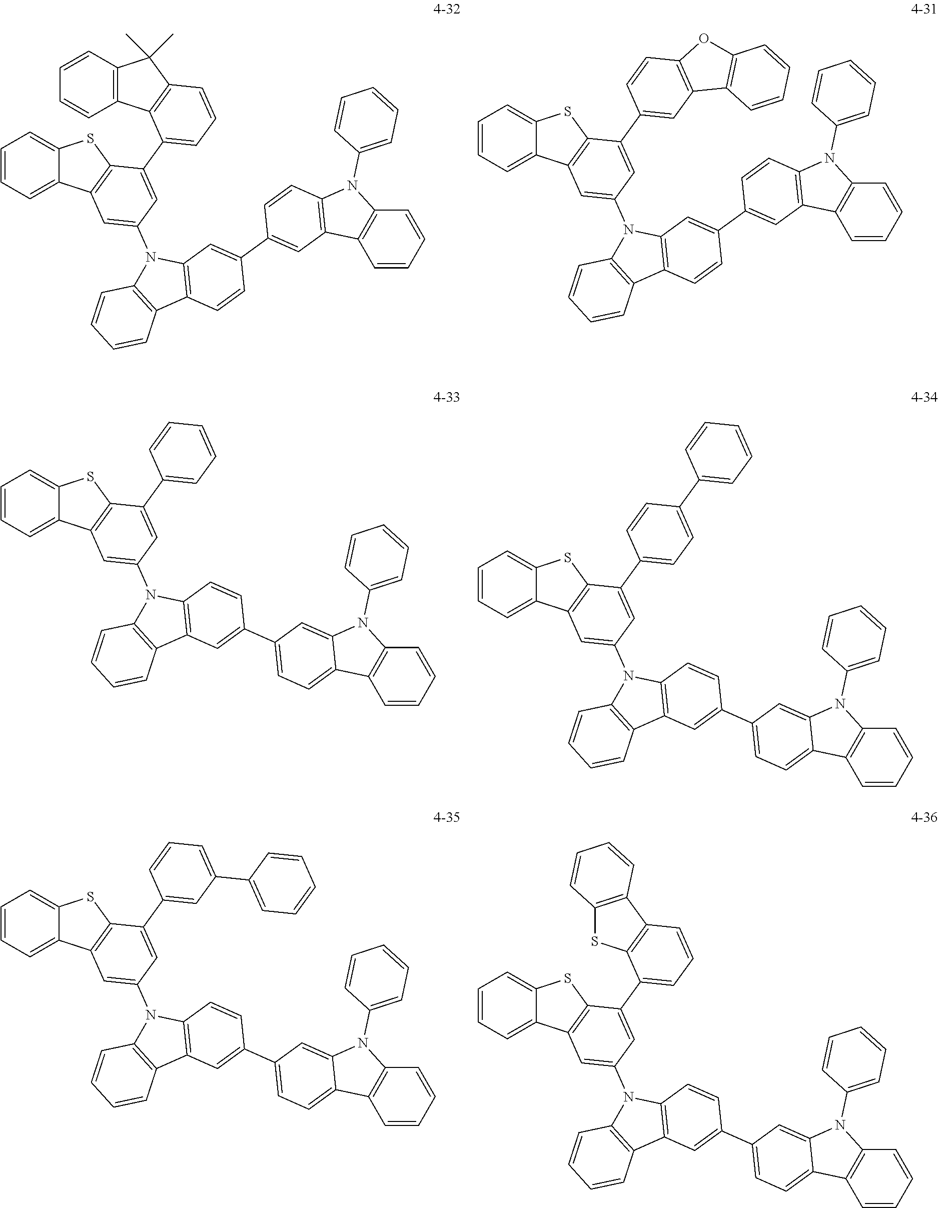

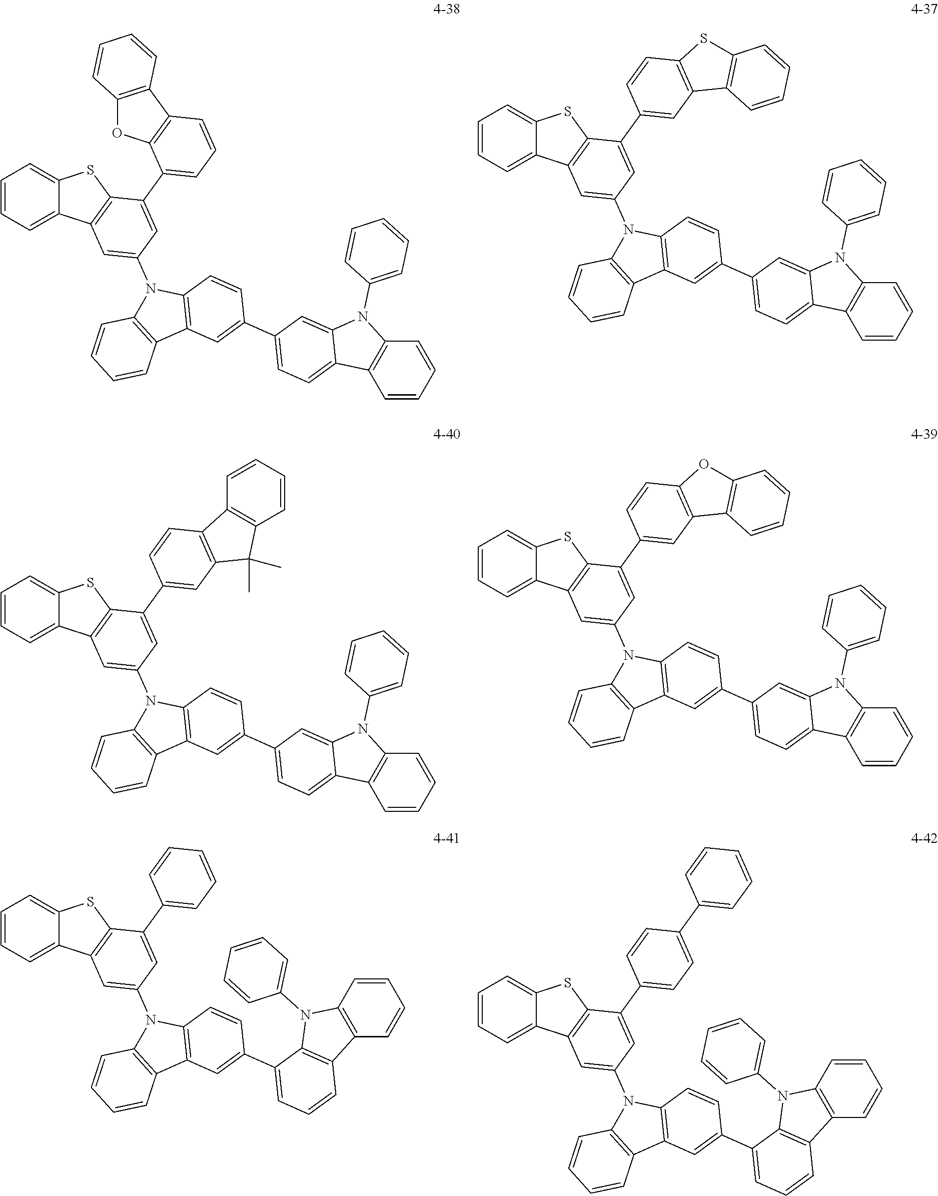

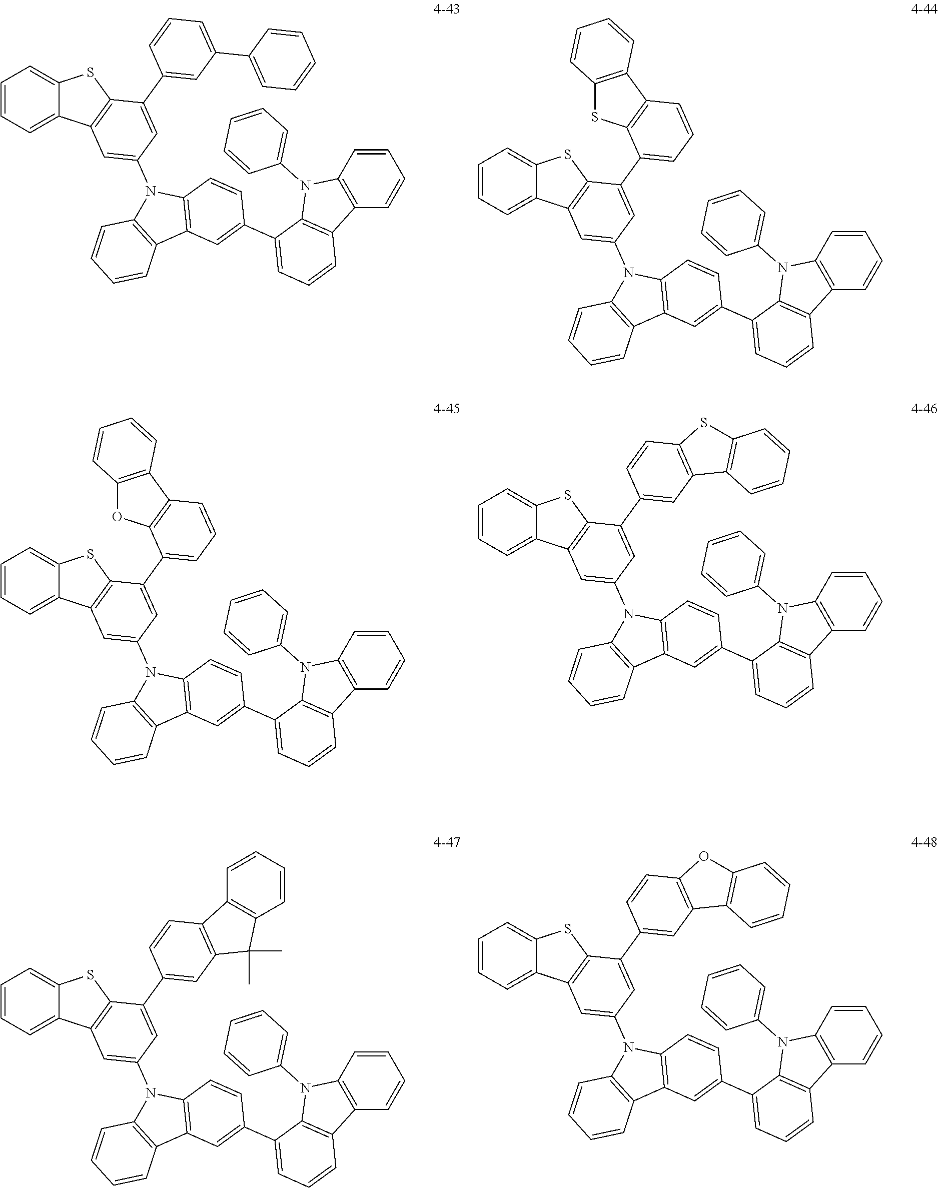

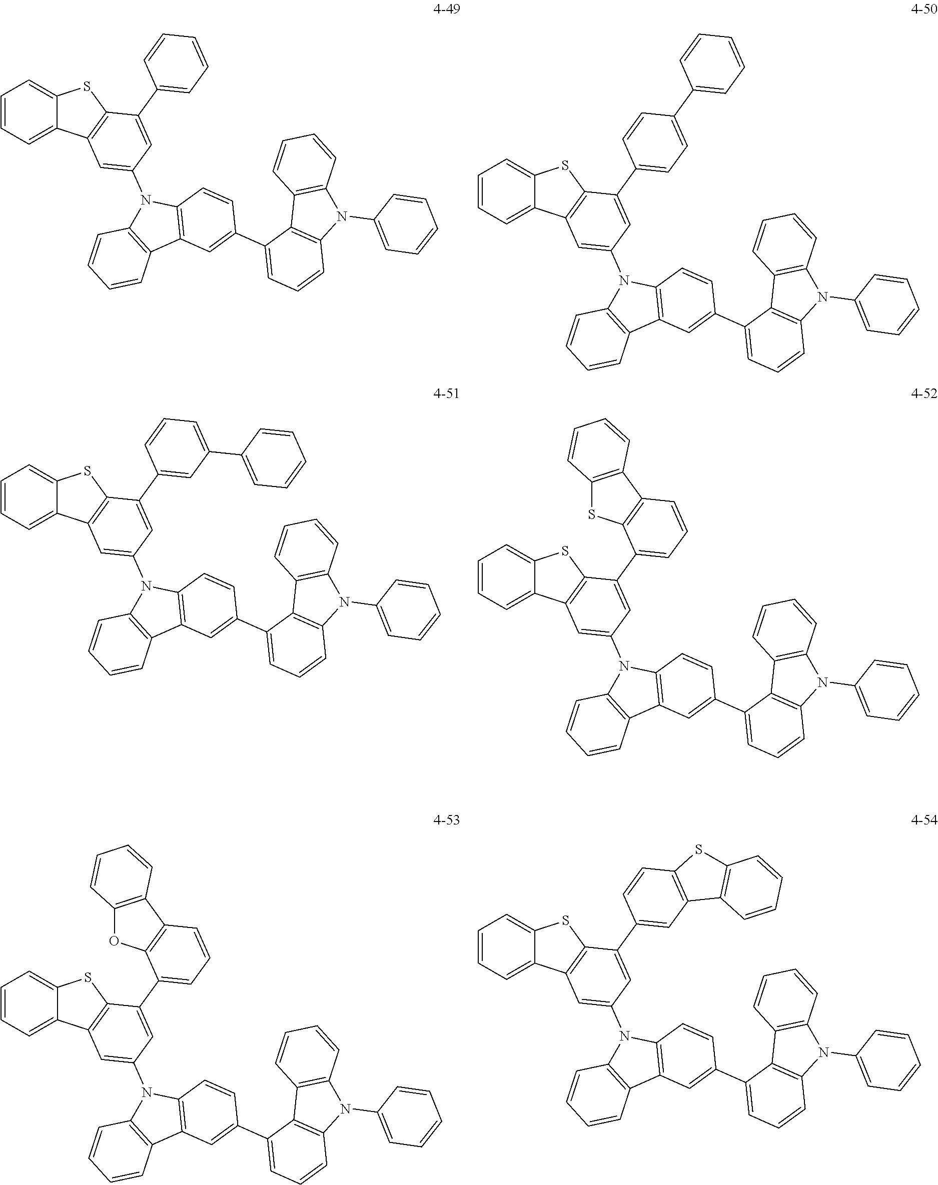

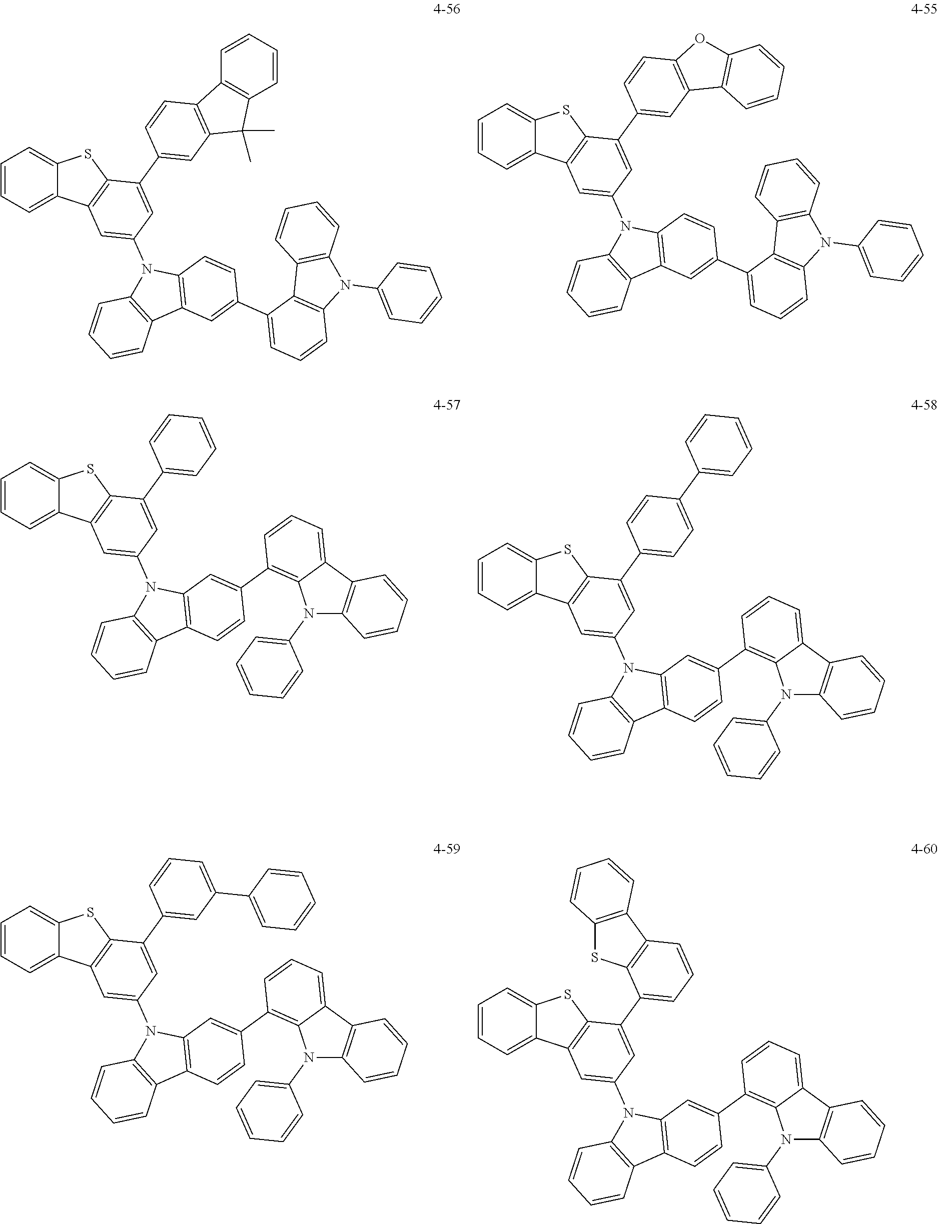

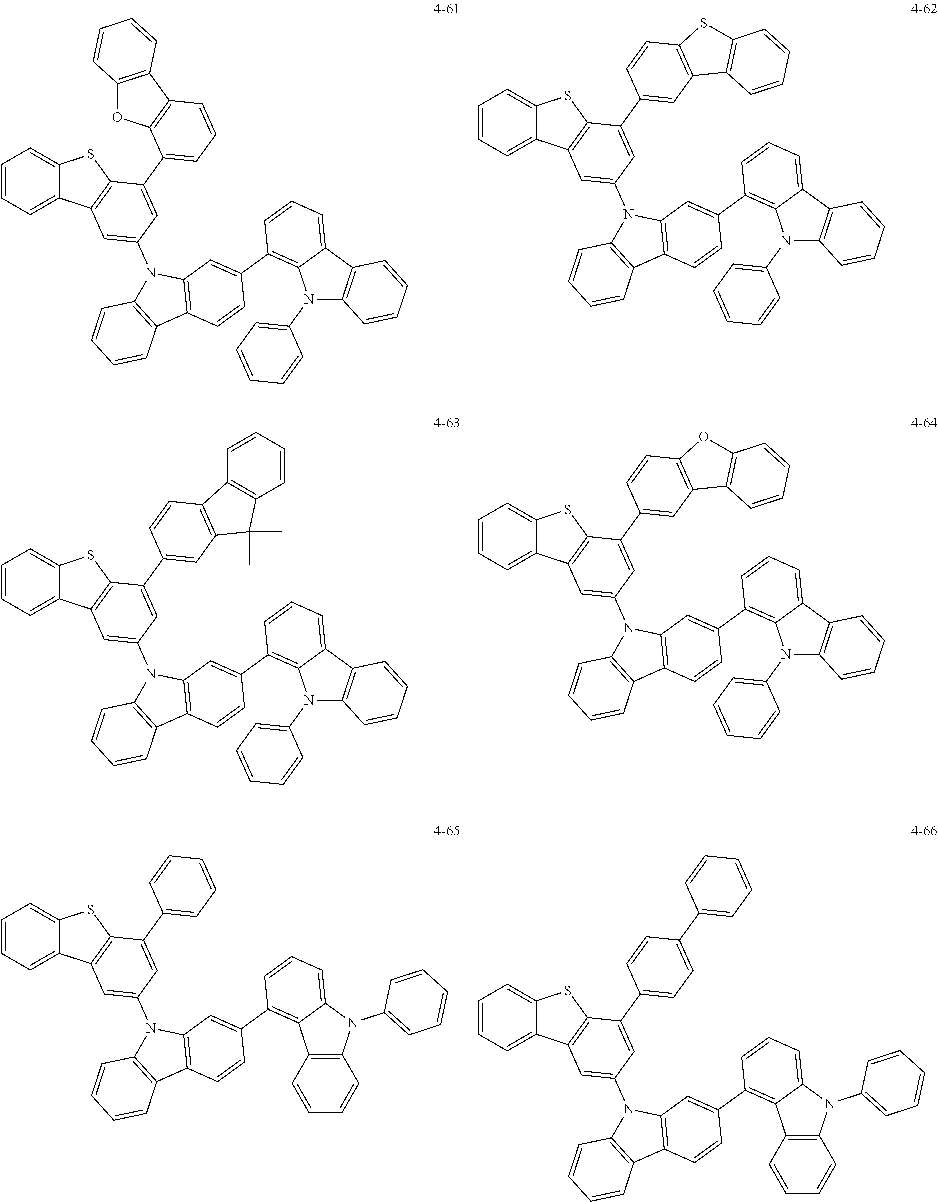

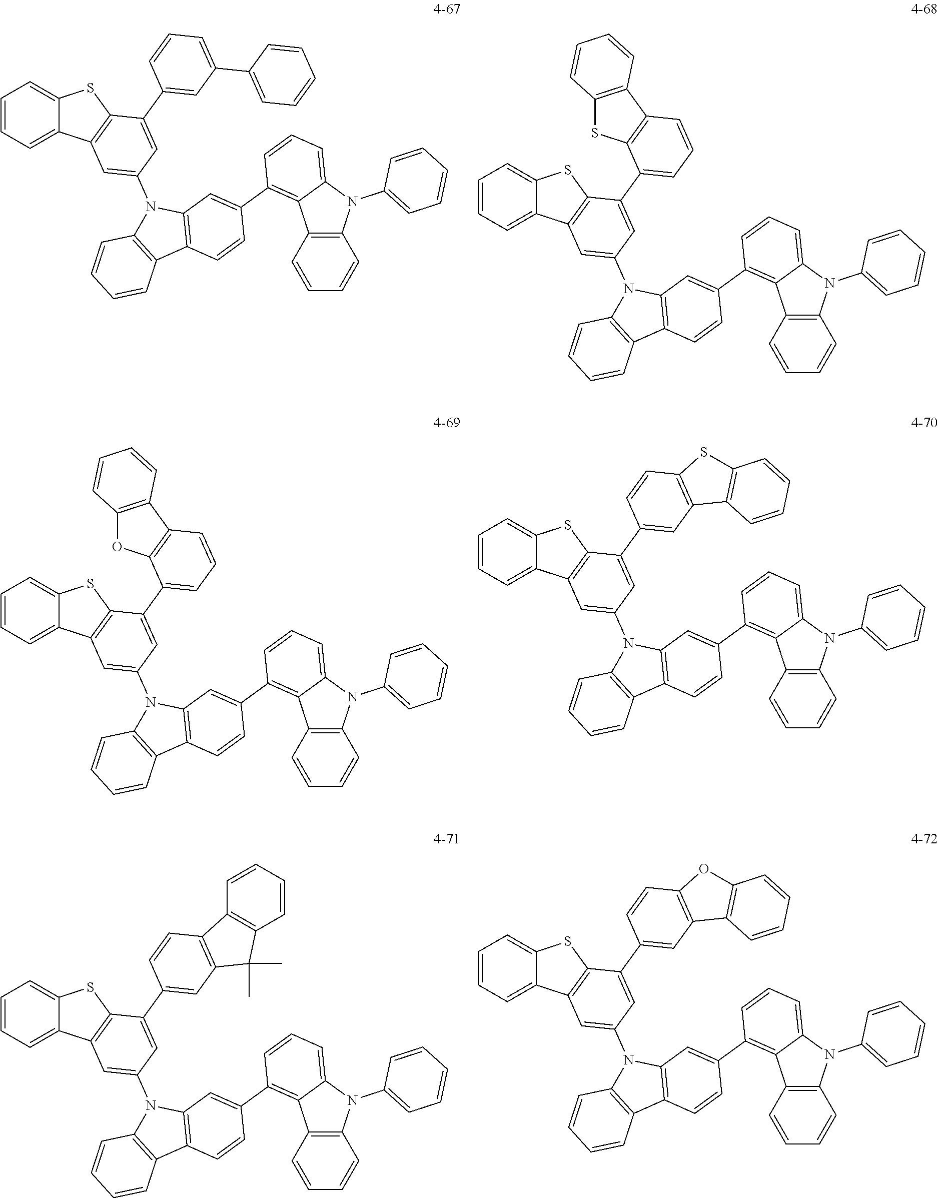

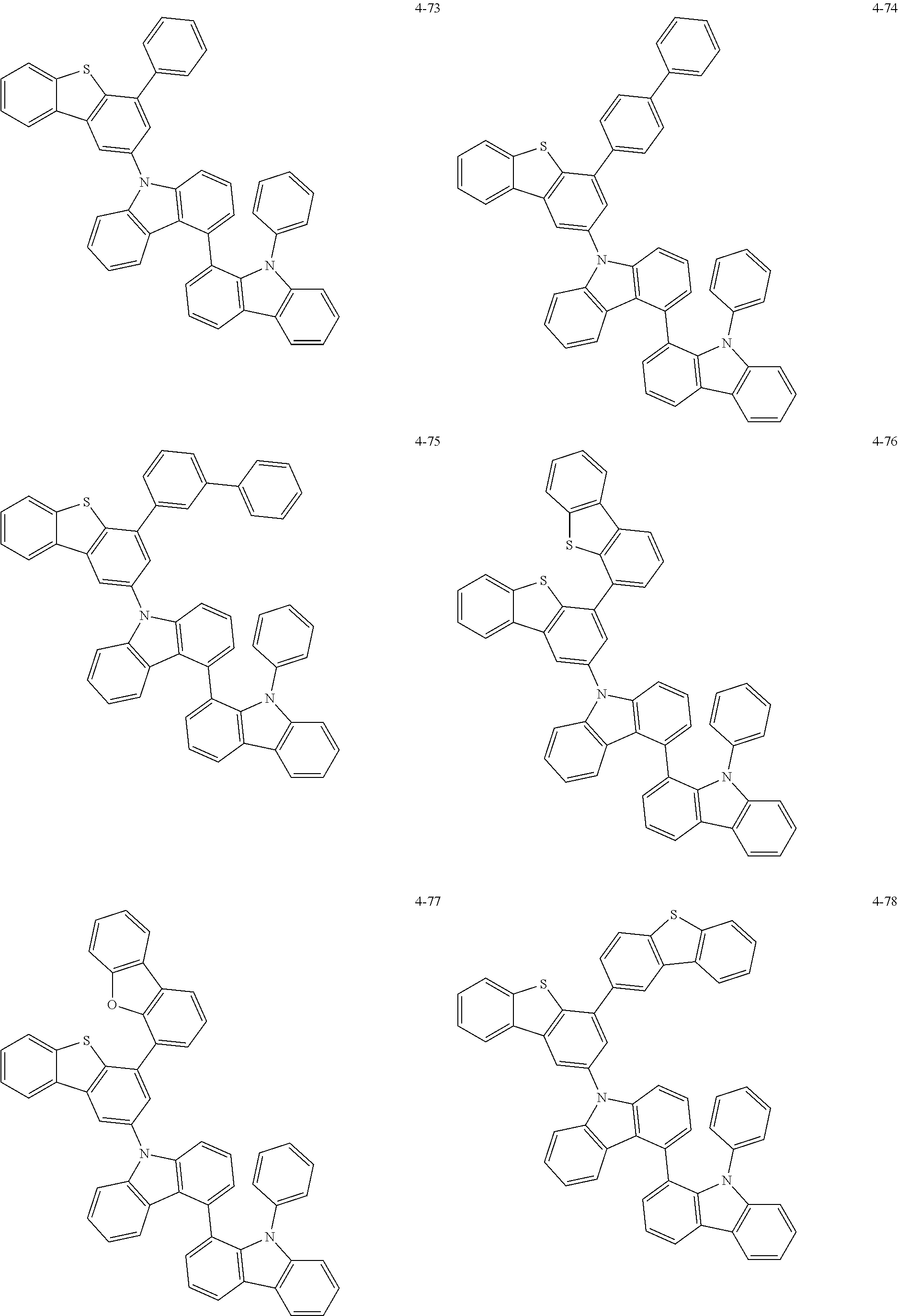

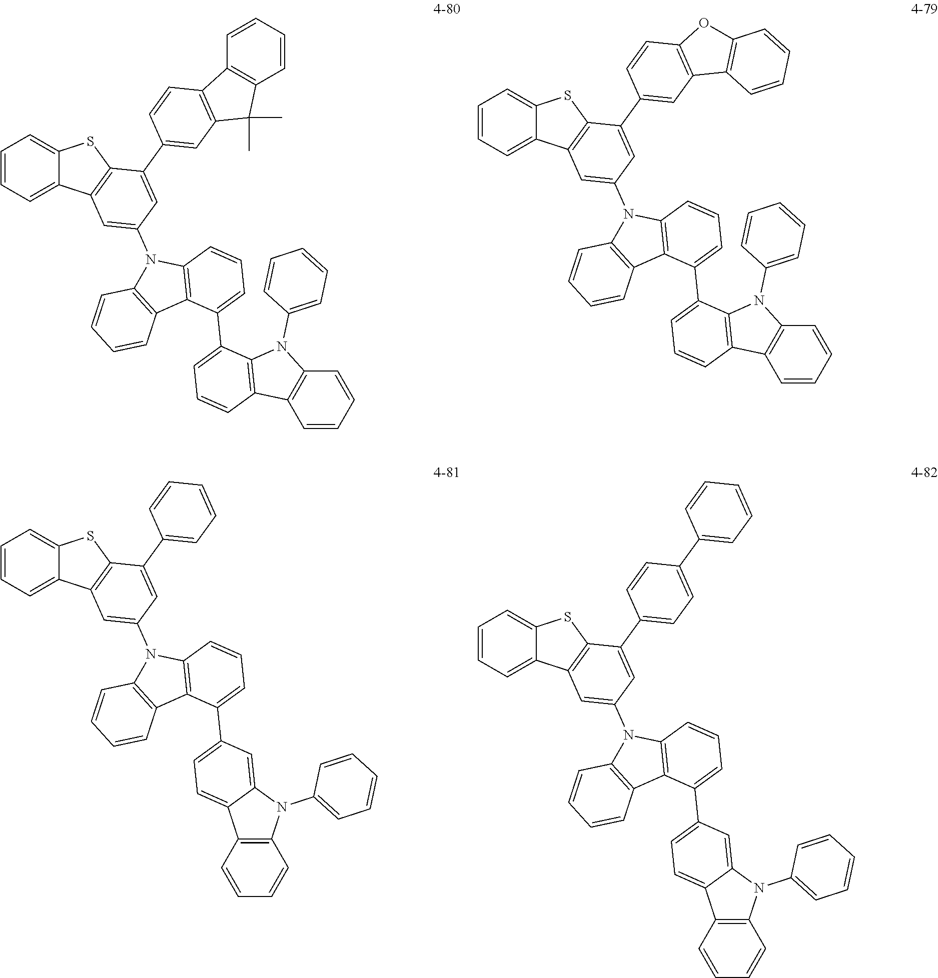 [SMILES: CC1(C)c2ccccc2-c2ccc(-c3cc(-n4c5ccccc5c5c(-c6cccc7c8ccccc8n(-c8ccccc8)c67)cccc54)cc4c3sc3ccccc34)cc21 ; c1ccc(-n2c3ccccc3c3cccc(-c4cccc5c4c4ccccc4n5-c4cc(-c5ccc6oc7ccccc7c6c5)c5sc6ccccc6c5c4)c32)cc1 ; c1ccc(-c2cc(-n3c4ccccc4c4c(-c5ccc6c7ccccc7n(-c7ccccc7)c6c5)cccc43)cc3c2sc2ccccc23)cc1 ; c1ccc(-c2ccc(-c3cc(-n4c5ccccc5c5c(-c6ccc7c8ccccc8n(-c8ccccc8)c7c6)cccc54)cc4c3sc3ccccc34)cc2)cc1]

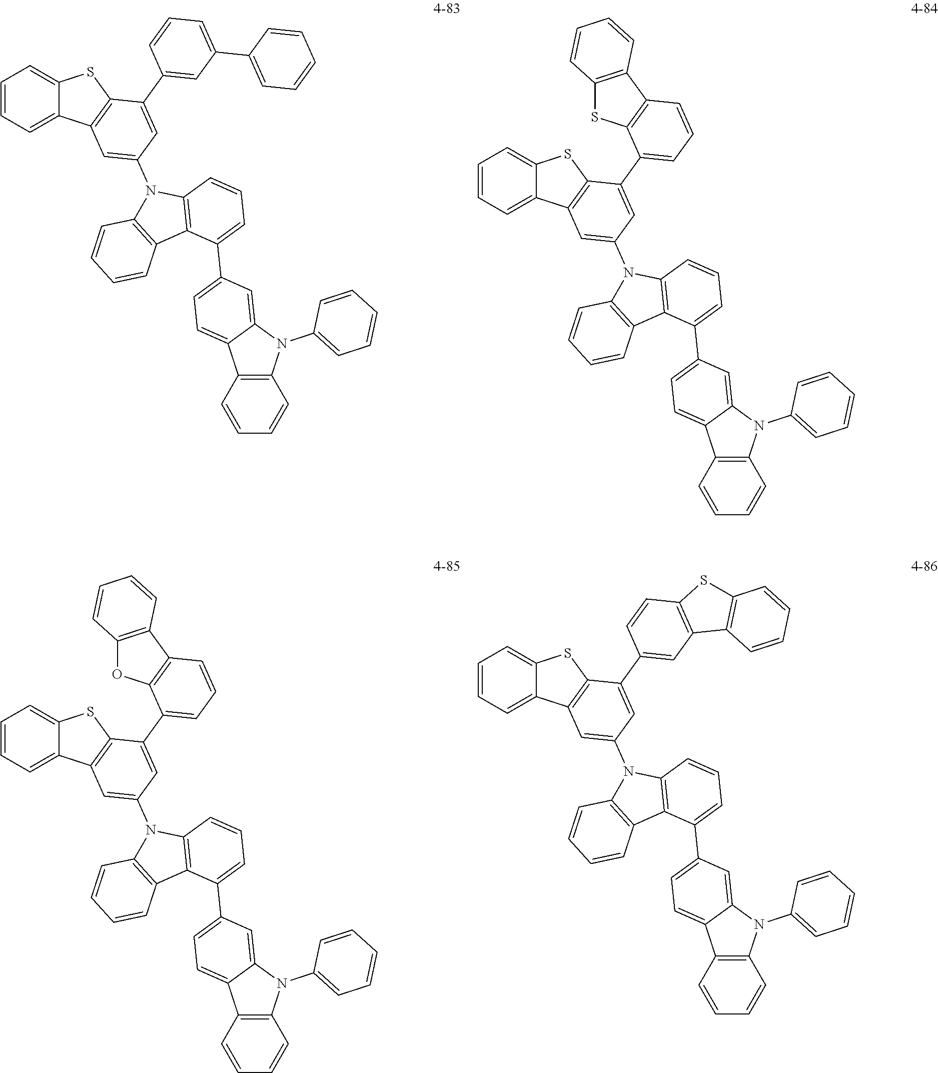 [SMILES: c1ccc(-c2cccc(-c3cc(-n4c5ccccc5c5c(-c6ccc7c8ccccc8n(-c8ccccc8)c7c6)cccc54)cc4c3sc3ccccc34)c2)cc1 ; c1ccc(-n2c3ccccc3c3ccc(-c4cccc5c4c4ccccc4n5-c4cc(-c5cccc6c5sc5ccccc56)c5sc6ccccc6c5c4)cc32)cc1 ; c1ccc(-n2c3ccccc3c3ccc(-c4cccc5c4c4ccccc4n5-c4cc(-c5cccc6c5oc5ccccc56)c5sc6ccccc6c5c4)cc32)cc1 ; c1ccc(-n2c3ccccc3c3ccc(-c4cccc5c4c4ccccc4n5-c4cc(-c5ccc6sc7ccccc7c6c5)c5sc6ccccc6c5c4)cc32)cc1]

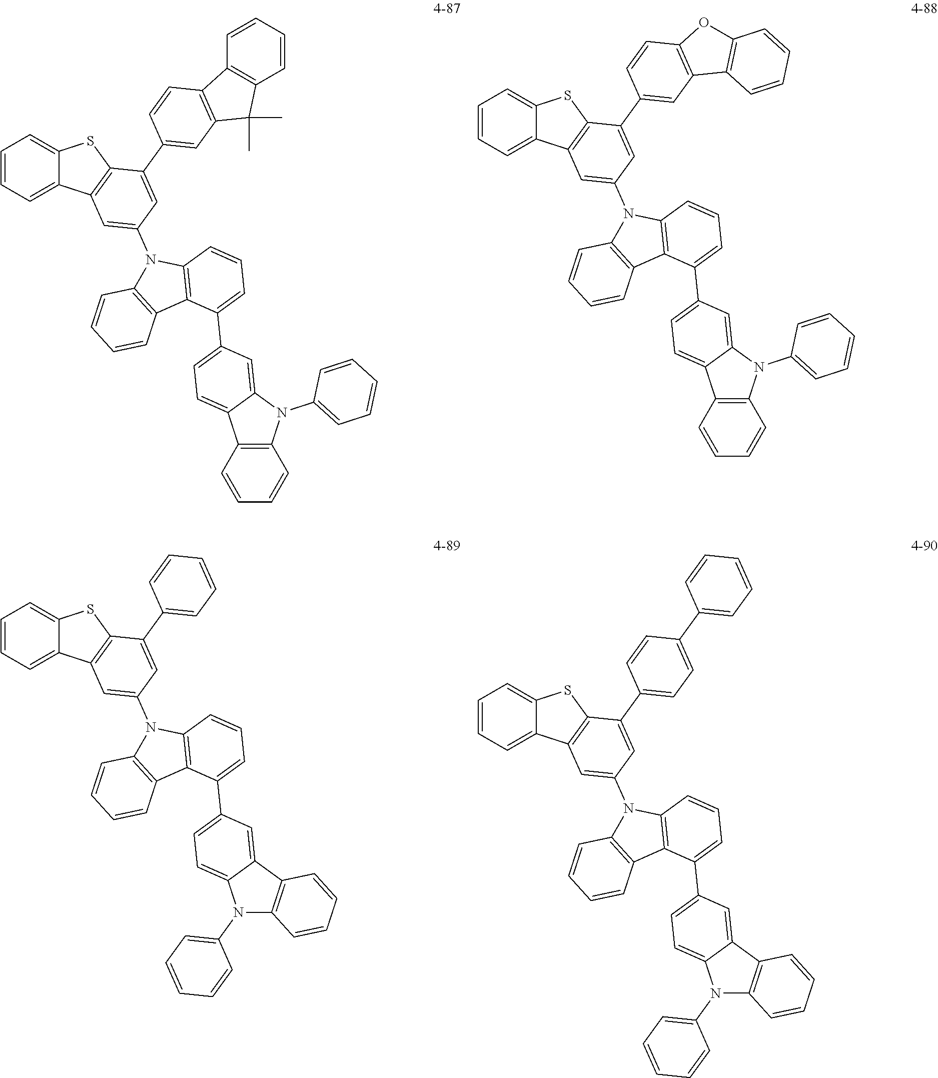 [SMILES: CC1(C)c2ccccc2-c2ccc(-c3cc(-n4c5ccccc5c5c(-c6ccc7c8ccccc8n(-c8ccccc8)c7c6)cccc54)cc4c3sc3ccccc34)cc21 ; c1ccc(-n2c3ccccc3c3ccc(-c4cccc5c4c4ccccc4n5-c4cc(-c5ccc6oc7ccccc7c6c5)c5sc6ccccc6c5c4)cc32)cc1 ; c1ccc(-c2cc(-n3c4ccccc4c4c(-c5ccc6c(c5)c5ccccc5n6-c5ccccc5)cccc43)cc3c2sc2ccccc23)cc1 ; c1ccc(-c2ccc(-c3cc(-n4c5ccccc5c5c(-c6ccc7c(c6)c6ccccc6n7-c6ccccc6)cccc54)cc4c3sc3ccccc34)cc2)cc1]

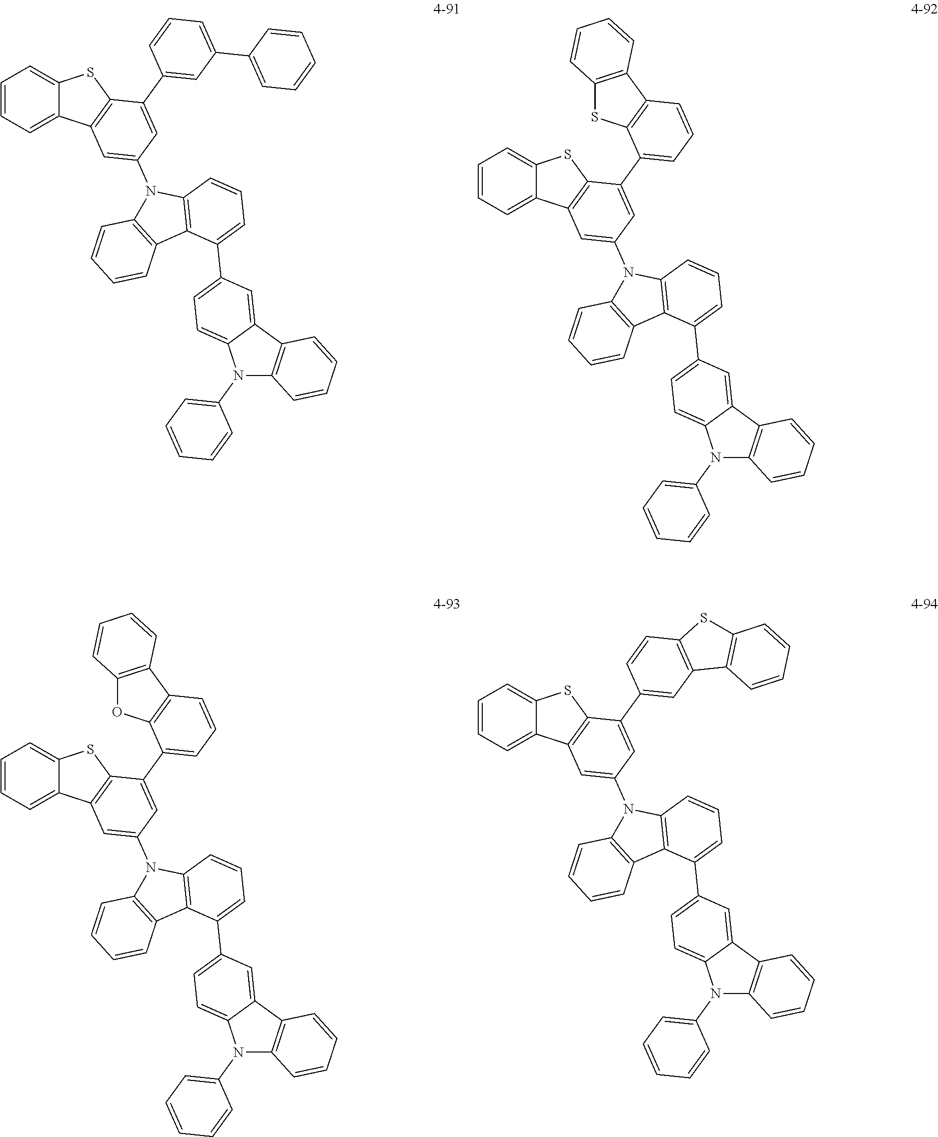 [SMILES: c1ccc(-c2cccc(-c3cc(-n4c5ccccc5c5c(-c6ccc7c(c6)c6ccccc6n7-c6ccccc6)cccc54)cc4c3sc3ccccc34)c2)cc1 ; c1ccc(-n2c3ccccc3c3cc(-c4cccc5c4c4ccccc4n5-c4cc(-c5cccc6c5sc5ccccc56)c5sc6ccccc6c5c4)ccc32)cc1 ; c1ccc(-n2c3ccccc3c3cc(-c4cccc5c4c4ccccc4n5-c4cc(-c5cccc6c5oc5ccccc56)c5sc6ccccc6c5c4)ccc32)cc1 ; c1ccc(-n2c3ccccc3c3cc(-c4cccc5c4c4ccccc4n5-c4cc(-c5ccc6sc7ccccc7c6c5)c5sc6ccccc6c5c4)ccc32)cc1]

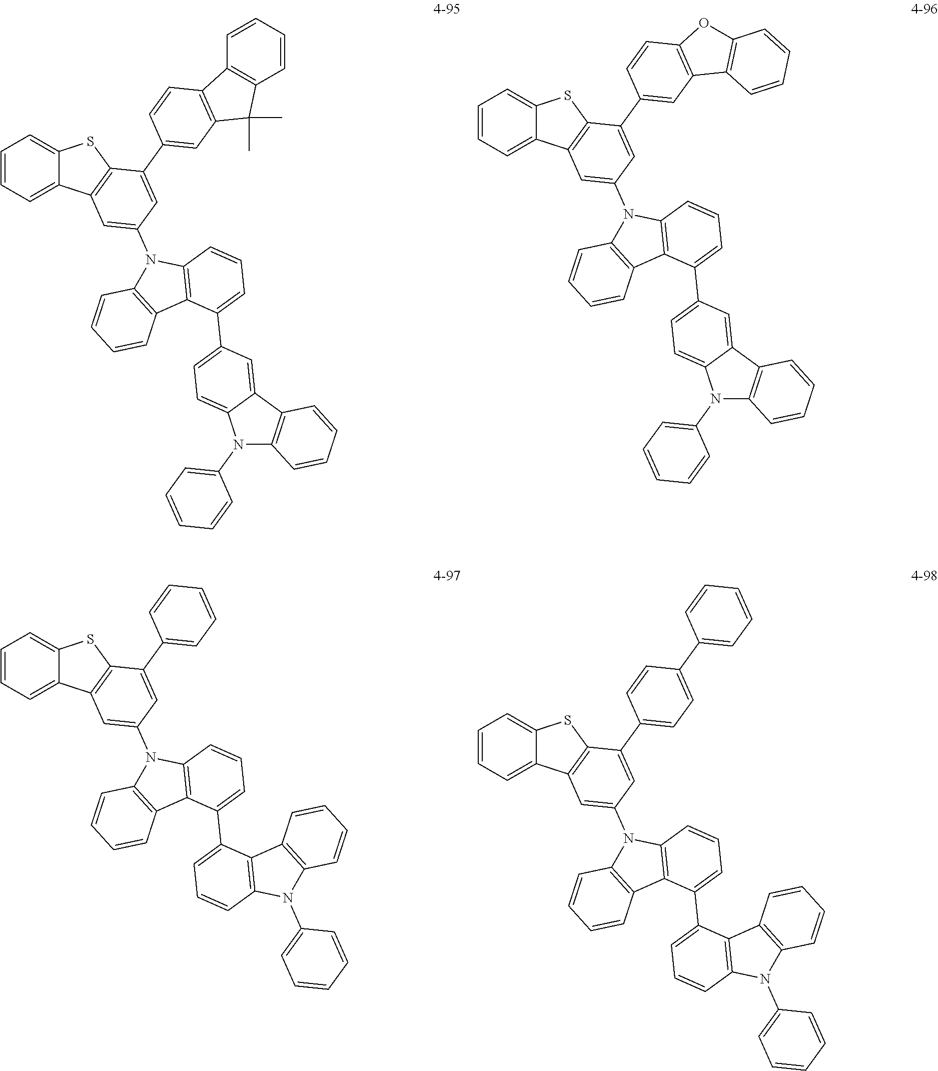 [SMILES: CC1(C)c2ccccc2-c2ccc(-c3cc(-n4c5ccccc5c5c(-c6ccc7c(c6)c6ccccc6n7-c6ccccc6)cccc54)cc4c3sc3ccccc34)cc21 ; c1ccc(-n2c3ccccc3c3cc(-c4cccc5c4c4ccccc4n5-c4cc(-c5ccc6oc7ccccc7c6c5)c5sc6ccccc6c5c4)ccc32)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-c3cc(-n4c5ccccc5c5c(-c6cccc7c6c6ccccc6n7-c6ccccc6)cccc54)cc4c3sc3ccccc34)cc2)cc1]

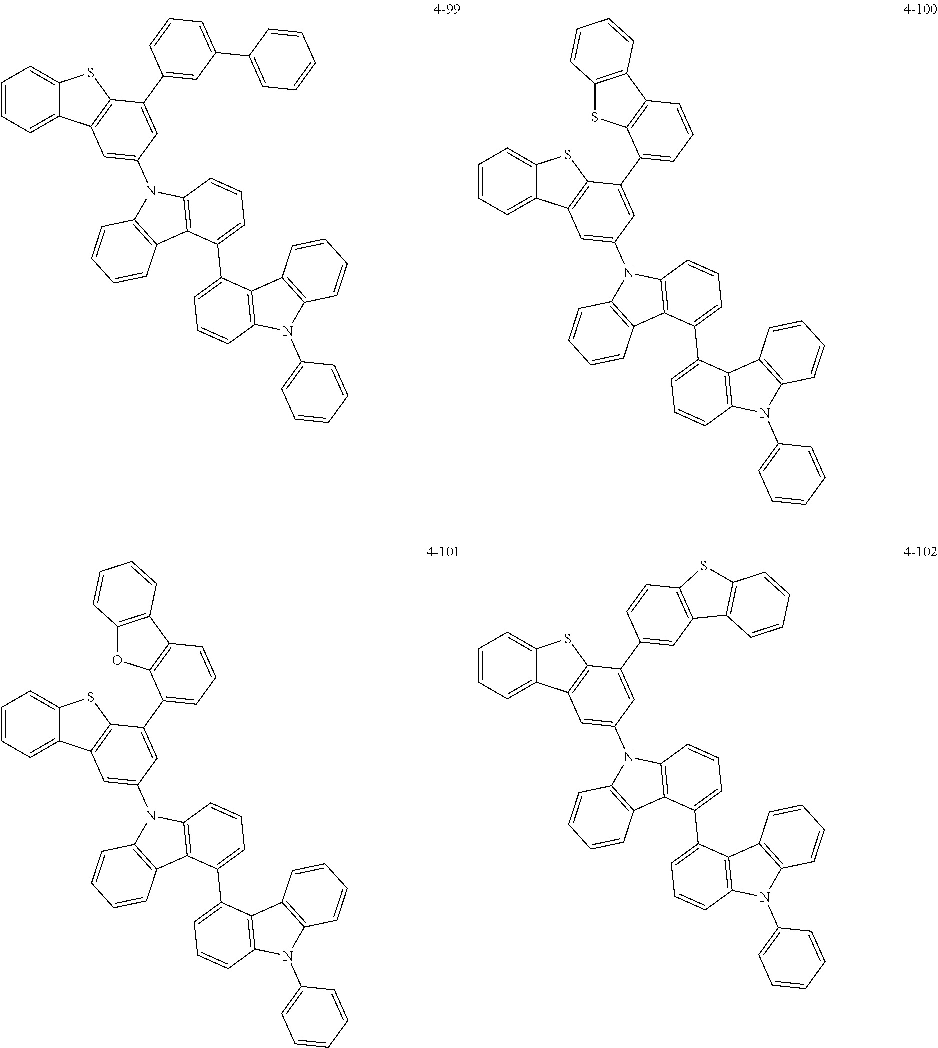 [SMILES: c1ccc(-c2cccc(-c3cc(-n4c5ccccc5c5c(-c6cccc7c6c6ccccc6n7-c6ccccc6)cccc54)cc4c3sc3ccccc34)c2)cc1 ; c1ccc(-n2c3ccccc3c3c(-c4cccc5c4c4ccccc4n5-c4cc(-c5cccc6c5sc5ccccc56)c5sc6ccccc6c5c4)cccc32)cc1 ; c1ccc(-n2c3ccccc3c3c(-c4cccc5c4c4ccccc4n5-c4cc(-c5cccc6c5oc5ccccc56)c5sc6ccccc6c5c4)cccc32)cc1 ; c1ccc(-n2c3ccccc3c3c(-c4cccc5c4c4ccccc4n5-c4cc(-c5ccc6sc7ccccc7c6c5)c5sc6ccccc6c5c4)cccc32)cc1]

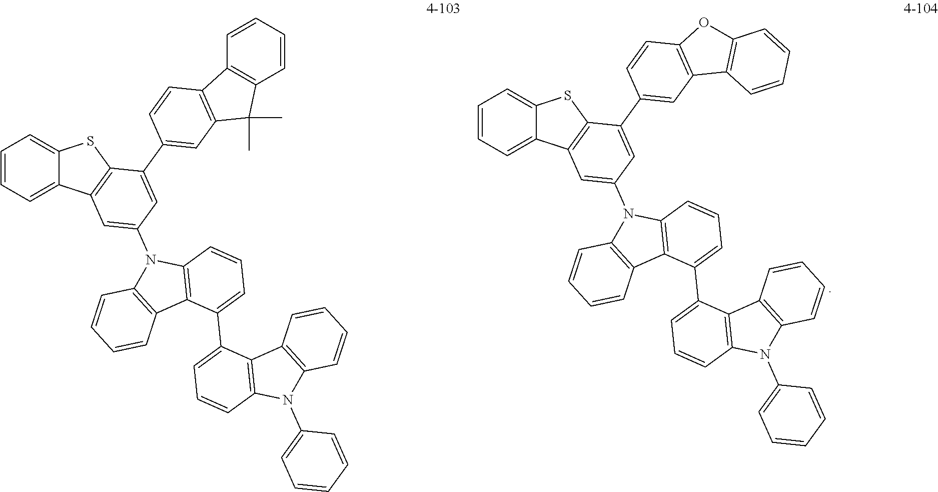 [SMILES: CC1(C)c2ccccc2-c2ccc(-c3cc(-n4c5ccccc5c5c(-c6cccc7c6c6ccccc6n7-c6ccccc6)cccc54)cc4c3sc3ccccc34)cc21 ; c1ccc(-n2c3ccccc3c3c(-c4cccc5c4c4ccccc4n5-c4cc(-c5ccc6oc7ccccc7c6c5)c5sc6ccccc6c5c4)cccc32)cc1]

10. The organic light emitting device of claim 6, wherein the organic material layer includes a light emitting layer, and the light emitting layer includes one or more types of the compound.

11. The organic light emitting device of claim 6, wherein the organic material layer includes a light emitting layer, the light emitting layer includes a host material, and the host material includes one or more types of the compound.

12. The organic light emitting device of claim 6, wherein the organic material layer includes a light emitting layer, the light emitting layer includes a host material, and the host material includes the compound and a compound of the following Chemical Formula 2: 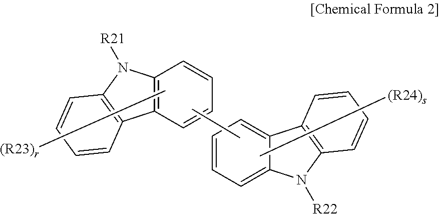
in Chemical Formula 2,
R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
R23 and R24 are each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
r and s are each an integer of 0 to 7; and
when r and s are each 2 or greater, substituents in the parentheses are the same as or different from each other.

13. The organic light emitting device of claim 6, further comprising one layer selected from the group consisting of a light emitting layer, a hole injection layer, a hole transfer layer, an electron injection layer, an electron transfer layer, an electron blocking layer and a hole blocking layer.

14. A composition for forming an organic material layer, the composition comprising:
a) a compound of the following Chemical Formula N; and
b) a compound of the following Chemical Formula P or Chemical Formula 2: 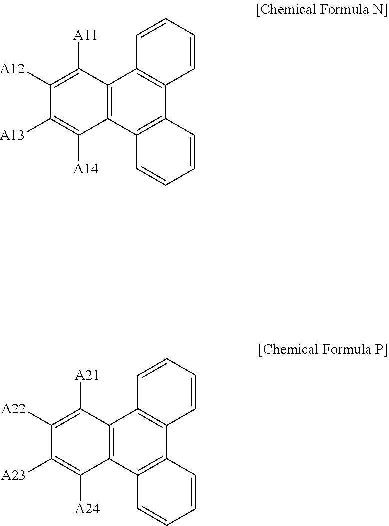
wherein, in Chemical Formula N and Chemical Formula P,
at least one of A11 to A14 includes at least one heteroaryl group including a pyridine ring, a pyrimidine ring, a triazine ring or an imidazole ring;
A21 to A24 do not include a heteroaryl group including a pyridine ring, a pyrimidine ring, a triazine ring or an imidazole ring;
one of A11 and A12 is (L1)ₐ-Q1, the other one of A11 and A12, and A13 and A14 are each independently hydrogen or (L2)_{b}-Q2, and at least one thereof is (L2)_{b}-Q2;
one of A21 and A22 is (1)ₐ-Q1, the other one of A21 and A22, and A23 and A24 are each independently hydrogen or (L2)_{b}-Q2, and at least one thereof is (L2)_{b}-Q2;
a and b are each independently an integer of 1 to 5;
when a and b are each 2 or greater, substituents in the parentheses are the same as or different from each other;
L1 and L2 are each independently a direct bond; a substituted or unsubstituted C6 to C60 arylene group; or a substituted or unsubstituted C2 to C60 heteroarylene group;
Q1 is a substituted or unsubstituted C6 to C20 aryl group; or a substituted or unsubstituted C2 to C20 heteroaryl group including N;
Q2 is a cyano group; a substituted or unsubstituted silyl group; a substituted or unsubstituted amine group; a substituted or unsubstituted C1 to C20 alkyl group; a substituted or unsubstituted C6 to C30 aryl group; a substituted or unsubstituted C2 to C30 heteroaryl group; or a substituted or unsubstituted phosphine oxide group;
when A12 and A13 are hydrogen, Q1 is a phenyl group and Q2 includes pyridine or triazine, L1 is a substituted or unsubstituted C6 to C60 arylene group; or a substituted or unsubstituted C2 to C60 heteroarylene group; and
when Q1 and Q2 are all an aryl group, one of i) Q1 and Q2 are all a phenyl group, L1 and L2 are a direct bond, and A2 and A4 are hydrogen, ii) Q1 and Q2 are all a phenyl group, at least one of L1 and L2 is a substituted or unsubstituted dicyclic or lower arylene group; or a substituted or unsubstituted C2 to C60 heteroarylene group, and iii) at least one of Q1 and Q2 is a dicyclic or higher aryl group unsubstituted or substituted with an alkyl group or an aryl group is satisfied, 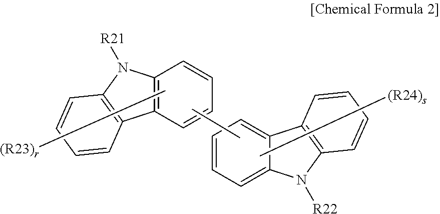
in Chemical Formula 2,
R21 and R22 are each independently a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
R23 and R24 are each independently hydrogen; deuterium; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group;
r and s are each an integer of 0 to 7; and
when r and s are each 2 or greater, substituents in the parentheses are the same as or different from each other.

15. The composition for forming an organic material layer of claim 14, wherein a) the compound of Chemical Formula N and b) the compound of Chemical Formula P or Chemical Formula 2 have a weight ratio of 1:10 to 10:1.
